(19) United States
(12) Patent Application Publication
(10) Pub. No.: US 2016/0082015 A1
(43) Pub. Date: Mar. 24, 2016

(54) METHODS, COMPOSITIONS AND KITS FOR PROMOTING MOTOR NEURON SURVIVAL AND TREATING AND DIAGNOSING NEURODEGENERATIVE DISORDERS

(71) Applicant: PRESIDENT AND FELLOWS OF HARVARD COLLEGE, Cambridge, MA (US)

(72) Inventor: Lee L. Rubin, Wellesley, MA (US)

(21) Appl. No.: 14/785,153

(22) PCT Filed: Apr. 18, 2014
(86) PCT No.: PCT/US14/34618

§ 371 Date: Oct. 16, 2015

Related U.S. Application Data
(60) Provisional application No. 61/813,539, filed on Apr. 18, 2013.

(51) Int. Cl. A61K 31/55; A61K 31/52; G01N 33/573; C12N 15/113; G01N 33/50; A61K 45/06; A61K 31/7105

(52) U.S. Cl. CPC A61K 31/55; A61K 45/06; A61K 31/52; A61K 31/7105; C12N 15/1137; G01N 33/5058; G01N 33/573; C12N 2310/531; C12N 2310/14; C12N 2320/30; C12N 2320/31; G01N 2500/10; G01N 2500/04; G01N 2440/14; G01N 2333/912

(57) ABSTRACT

Methods, compositions, and kits for promoting motor neuron survival and for treatment and diagnosis of neurodegenerative disorders such as Amyotrophic lateral sclerosis (ALS) and Spinal muscular atrophy (SMA) are described herein.

### RELATED APPLICATIONS

This application claims the benefit of U.S. Provisional Application No. 61/813,539, filed Apr. 18, 2013, the entire teachings of which are incorporated herein by reference.

### GOVERNMENT SUPPORT

This invention was made with government support under grant no. 5P01 NS066888 awarded by the National Institutes of Health. The government has certain rights in the invention.

### FIELD OF INVENTION

The invention relates to methods, compositions, and kits for promoting motor neuron survival, and more particularly to methods, compositions and kits that are useful for the diagnosis and treatment of neurodegenerative disorders such as Amyotrophic lateral sclerosis (ALS) and spinal muscular atrophy (SMA).

### BACKGROUND

Amyotrophic lateral sclerosis (ALS) is a late-onset, progressive, neurodegenerative disorder that affects motor neuron survivaloth upper and lower motor neurons (MNs) and ultimately leads to death. Although the rate at which ALS progresses can be quite variable, the mean survival time is between three and five years. In the United States, 90 to 95% of all ALS cases are idiopathic (Brown, 1997; Boillëe et al., 2006). However, among the familial forms of ALS, approximately 20% are caused by mutations in the SOD1 gene. Although only accounting for about 2% of all ALS cases, SOD1-associated ALS has been the most studied form of ALS, due to the early discovery of the disease-causing mutations and the availability of mouse models. Mutations in SOD gene are gain-of-function mutations that cause autosomal dominant inheritance of ALS. It is the toxicity of the mutant SOD1 protein, rather than a defect in the function of the normal SOD1 protein, that is thought to lead to the disease. Exactly how mutations in the SOD1 gene cause MN death is still unclear, but it is now well accepted that cell autonomous and non-cell autonomous mechanisms can contribute to degeneration (Di Giorgio et al., 2007; Nagai et al., 2007; reviewed in Ilieva et al., 2009). A more recent breakthrough in ALS research came when the DNA RNA-binding protein transactivating response element DNA binding protein-43 (TDP-43) was identified as a major component of protein aggregates found in sporadic ALS and non-SOD1 familial ALS cases (Arai et al., 2006; Neumann et al., 2006). Later, mutations in TARDBP, the gene encoding TDP-43, were identified in ˜4% of familial ALS cases (Van Deerlin et al., 2008). The very recent identification of a hexanucleotide repeat expansion within the C9orf72 gene points to it as potentially the most frequent pathogenic cause of ALS identified thus far, accounting overall for about 6% of sporadic ALS cases, and about 36-40% of familial ALS cases, in Europe and the USA (Renton et al., 2011; Majounie at al., 2012). Thus, it may be that there are numerous pathogenic initiators of ALS, potentially including mitochondrial dysfunction, oxidative stress, protein misfolding and aggregation, excitotoxicity, neuroinflammation, axonal transport defects, and neurotrophin depletion (Joyce et al., 2011). Riluzole is currently the only approved treatment for ALS. It may act by reducing an excitotoxic component of the disease, but it prolongs life by only 2 to 3 months and provides little functional improvement (Miller et al., 2007). While better treatments for ALS are urgently needed, it has been challenging to conduct research geared towards therapeutic discovery, partly because of the diverse causes of ALS.

Therefore, there is need in the art for methods of identifying compounds and compositions for promoting motor neuron survival and compositions and methods for treatment of motor neuron diseases such as ALS and SMA.

### SUMMARY

Disclosed herein are one or more solutions to the needs outlined above. Accordingly, in one aspect, a method of promoting motor neuron survival is disclosed, the method comprising contacting a motor neuron with an effective amount of an agent that inhibits HPK/GC kinase-like kinase (HGK).

In another aspect, a method of treating or preventing a neurodegenerative disorder in a subject in need thereof is disclosed, the method comprising administering an effective amount of an agent that inhibits HGK to the subject.

In yet another aspect, a method of treating or preventing a disorder characterized by neuronal cell death in a subject in need thereof is disclosed, the method comprising administering an effective amount of an agent that inhibits HGK to the subject.

In still another aspect, a method of treating or preventing amyotrophic lateral sclerosis (ALS) in a subject in need thereof is disclosed, the method comprising administering an effective amount of an agent that inhibits HGK to the subject.

In one aspect, a method of treating or preventing spinal muscular atrophy (SMA) in a subject in need thereof comprises administering an effective amount of an agent that inhibits HGK to the subject.

In another aspect, a method of promoting motor neuron survival comprises contacting a motor neuron with an effective amount of an agent that decreases activation of the HGK-Tak1-MKK4-JNK-c-Jun cell death signaling cascade.

In yet another aspect, a method of treating or preventing a neurodegenerative disorder in a subject in need thereof comprises administering an effective amount of an agent that decreases activation of the HGK-Tak1-MKK4-JNK-c-Jun cell death signaling cascade.

In still yet another aspect, a method of treating or preventing a disorder characterized by neuronal cell death in a subject in need thereof comprises administering an effective amount of an agent that decreases activation of the HGK-Tak1-MKK4-JNK-c-Jun cell death signaling cascade to the subject.

In an aspect, a method of treating or preventing amyotrophic lateral sclerosis (ALS) in a subject in need thereof, comprising administering an effective amount of an agent that decreases activation of the HGK-Tak1-MKK4-JNK-c-Jun cell death signaling cascade.

In another aspect, a method of treating or preventing spinal muscular atrophy (SMA) in a subject in need thereof, comprising administering an effective amount of an agent that decreases activation of the HGK-Tak1-MKK4-JNK-c-Jun cell death signaling cascade.

In some embodiments, the agent decreases activation of the HGK-Tak1-MKK4-JNK-c-Jun cell death signaling cascade.

In some embodiments, the agent decreases phosphorylation of a protein kinase in the HGK-Tak1-MKK4-JNK-c-Jun cell death signaling cascade. In some embodiments, the protein kinase is selected from the group consisting of HGK, Tak1, MKK4, and JNK. In some embodiments, the agent inhibits HGK. In some embodiments, the agent is an inhibitor of glycogen synthase kinase 3 (GSK-3). In some embodiments, the agent is a dual-inhibitor of HGK and GSK-3. In some embodiments, the agent is a dual-inhibitor of HGK and GSK-3β. In some embodiments, the agent inhibits GSK3. In some embodiments, the agent is kenpaullone or an analog or derivative thereof.

In some embodiments, the agent is selected from the group consisting of small organic or inorganic molecules; saccharines; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

In some embodiments, the methods further comprise contacting the motor neuron with an additional agent that inhibits GSK3.

In some embodiments, the methods further comprise administering to the subject an effective amount of an additional agent that inhibits GSK3.

In some embodiments, the neuronal cell is selected from the group consisting of a motor neuron and a sensory neuron.

In some embodiments, the motor neuron comprises a mutation in a gene encoding survival of motor neuron 1 (SMN1). In some embodiments, the motor neuron comprises a mutation in a gene encoding superoxide dismutase 1 (SOD1). In some embodiments, the mutation is selected from the group consisting of a A4V mutation, a G85R mutation, and a G93A mutation. In some embodiments, the motor neuron comprises mutations in a gene encoding SMN1 and a gene encoding SOD1.

In some embodiments, the contact is in vitro.

In some embodiments, the contact is in vivo. In some embodiments, the in vivo contact is in a subject selected for treatment of a neurodegenerative disorder or disorder characterized by neuronal cell death.

In some embodiments, the subject is at risk of developing a neurodegenerative disorder or a disorder characterized by neuronal cell death. In some embodiments, the subject is suspected of having a neurodegenerative disorder or a disorder characterized by neuronal cell death. In some embodiments, the subject is a mammal. In some embodiments, the subject is a human.

In some embodiments, the neurodegenerative disorder is characterized by mutation of a SMN gene. In some embodiments, the neurodegenerative disorder is characterized by decreased levels of SMN protein. In some embodiments, the neurodegenerative disorder is characterized by neuronal cell death. In some embodiments, the neurodegenerative disorder is ALS. In some embodiments, the neurodegenerative disorder is SMA.

In another aspect, a method of promoting motor neuron survival comprises contacting a motor neuron with an effective amount of a compound selected from the group consisting of ethaverine hydrochloride, crinamine, gedunin, pomiferin, dactinomycin, 3-alpha-hydroxydeoxygedinin, totarol, totaral acetate, ginkgetin potassium salt, hygromycin B, blasticidin S, lynestrenol, hippeastrine hydrobromide, and combinations thereof.

In some aspects, a composition comprises an effective amount of an HGK inhibitor and an effective amount of a GSK3 inhibitor. In some embodiments, the composition is useful for promoting survival of motor neurons. In some embodiments, the composition is useful for treating or preventing a neurodegenerative disorder. In some embodiments, the neurodegenerative disorder is selected from the group consisting of ALS and SMA. In some embodiments, the composition further comprises a pharmaceutically acceptable excipient, diluent or carrier.

In some aspects, disclosed herein is a method of identifying a candidate agent that promotes motor neuron survival, the method comprising (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent that promotes motor neuron survival, wherein the test agent is a candidate agent for promoting motor neuron survival if the test agent (i) decreases the level or activity of HGK or (ii) decreases activation of the HGK-Tak1-MKK4-JNK-c-Jun cell death signaling cascade, in the presence of the test agent.

In other aspects, disclosed herein is a method of identifying a candidate agent for treating or preventing a neurodegenerative disorder, the method comprising (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent for treating a neurodegenerative disorder, wherein the test agent is a candidate agent for treating a neurodegenerative disorder if the test agent (i) decreases the level or activity of HGK or (ii) decreases activation of the HGK-Tak1-MKK4-JNK-c-Jun cell death signaling cascade, in the presence of the test agent.

In certain aspects, disclosed herein is a method of identifying a candidate agent for treating or preventing ALS, comprising (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent for treating ALS, wherein the test agent is a candidate agent for treating ALS if the test agent (i) decreases the level or activity of HGK or (ii) decreases activation of the HGK-Tak1-MKK4-JNK-c-Jun cell death signaling cascade, in the presence of the test agent.

In some aspects, disclosed herein is a method of identifying a candidate agent for treating or preventing SMA, the method comprising (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent for treating SMA, wherein the test agent is a candidate agent for treating SMA if the test agent (i) decreases the level or activity of HGK or (ii) decreases activation of the HGK-Tak1-MKK4-JNK-c-Jun cell death signaling cascade, in the presence of the test agent.

In some embodiments, the contacting is performed in the absence of trophic factors. In some embodiments, the motor neuron comprises an in vitro-differentiated motor neuron. In some embodiments, the motor neurons are derived from pluripotent cells selected from the group consisting of embryonic stem cells (ESCs) and induced pluripotent stem cells (iPSCs). In some embodiments, the test agent is selected from the group consisting of small organic or inorganic molecules; saccharines; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

In some embodiments, the identification methods further comprise quantifying the number of motor neurons surviving in the presence of the test agent. In some embodiments, the surviving motor neurons express a detectable reporter. In some embodiments, the detectable reporter is a fluorescent protein selected from the group consisting of green fluorescent protein (GFP) and red fluorescent protein (RFP).

In certain aspects, disclosed herein is a method of diagnosing a neurodegenerative disorder in a subject, the method comprising: (a) obtaining a biological sample from the subject comprising neuronal cells; (b) conducting at least one binding assay on the neuronal cells to detect the level or activity of HGK in the neuronal cells; and (c) diagnosing the subject as having the neurodegenerative disorder if the level or activity of HGK in the neuronal cells is increased relative to a control level or activity of HGK.

In another aspect, disclosed herein is a method of diagnosing a neurodegenerative disorder in a subject, the method comprising: (a) obtaining a biological sample from the subject comprising neuronal cells; (b) conducting at least one kinase assay on the neuronal cells to detect activation of the HGK-Tak1-MKK4-JNK-c-Jun cell death signaling cascade in the neuronal cells; and (c) diagnosing the subject as having the neurodegenerative disorder if the HGK-Tak1-MKK4-JNK-c-Jun cell death is activated in the neuronal cells.

In some embodiments, the HGK-Tak1-MKK4-JNK-c-Jun cell death signaling cascade in the neuronal cells is activated if the phosphorylation level or activity of a protein kinase in the HGK-Tak1-MKK4-JNK-c-Jun cell death signaling cascade is increased relative to the phosphorylation level or activity of a protein kinase in the HGK-Tak1-MKK4-JNK-c-Jun cell death signaling cascade in a control.

In some embodiments, the at least one binding assay comprises a protein kinase assay to detect the phosphorylation activity of HGK. In some embodiments, the at least one binding assay comprises a protein kinase assay to detect the level of phosphorylation of a protein kinase downstream to HGK in the HGK-Tak1-MKK4-JNK-c-Jun cell death signaling cascade. In some embodiments, the diagnostic methods further comprise selecting a subject suspected of having a neurodegenerative disorder.

In some embodiments, the neuronal cells comprise motor neurons. In some embodiments, the neuronal cells comprise sensory neurons. In some embodiments, the neurodegenerative disorder is ALS.

In one aspect, disclosed herein is a method of diagnosing amyotrophic lateral sclerosis (ALS) in a subject, the comprising: (a) obtaining a biological sample from the subject comprising neuronal cells; (b) conducting at least one kinase assay on the neuronal cells to detect activation of the HGK-Tak1-MKK4-JNK-c-Jun cell death signaling cascade in the neuronal cells; and (c) diagnosing the subject as having ALS if the HGK-Tak1-MKK4-JNK-c-Jun cell death is activated in the neuronal cells.

In some embodiments, the HGK-Tak1-MKK4-JNK-c-Jun cell death signaling cascade in the neuronal cells is activated if the phosphorylation level or activity of a protein kinase in the HGK-Tak1-MKK4-JNK-c-Jun cell death signaling cascade is increased relative to the phosphorylation level or activity of a protein kinase in the HGK-Tak1-MKK4-JNK-c-Jun cell death signaling cascade in a control. In some embodiments, the method of diagnosing ALS further comprises selecting a subject suspected of having ALS. In some embodiments, the neuronal cells comprise motor neurons.

In one aspect, disclosed herein is a method of promoting motor neuron survival, the method comprising contacting a motor neuron with a compound of formula (I):

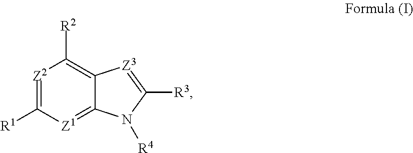

and an additional agent that modulates a biological pathway or a target described herein. In some embodiments, the additional agent can modulate a biological pathway or a target that is different from the one that is modulated (e.g., inhibited) by the compound of formula (I).

In some embodiment, the compound of formula (I) has a structure shown in formula (Ia):

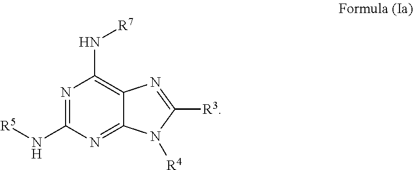

In some embodiments, the compound of formula (I) is an inhibitor of a Cyclin-dependent kinase (CDK) or a p21 activated kinase (PAK). In some embodiments, the compound of formula (I) is purvalanol A.

Accordingly, present herein is also a method of promoting motor neuron survival, the method comprising contacting a motor neuron with an inhibitor of a PAK or a CDK and an additional agent that modulates a biological pathway or a target described herein. In some embodiments, the CDK or the PAK inhibitor is of formula (I).

In some embodiments, the additional agent is a GSK-3 inhibitor.

In some embodiments, the additional agent is allsterpaullone.

The term “synergistic” as used herein is defined to mean a combination of components wherein the activity of the combination is greater than the additive of the individual activities of each component of the combination. In some embodiments, the activity of the combination is at least 5%, at least 10%, at least 15%, at least 20%, at least 25%, at least 30%, at least 40%, at least 50%, at least 60%, at least 70%, at least 80%, at least 90%, at least 1-fold, at least 2-fold, at least 3-fold, at least 4-fold, at least 5-fold, at least 10-fold, at least 50-fold, at least 100-fold or greater than the additive of the individual activities of each component of the combination.

The CDK inhibitors, PAK inhibitors and the compounds that modulate the biological pathway or target described herein can be a small molecules, peptides, antibodies, antibody fragments, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. In some embodiments, the compound binds to at least one component in the pathway.

In another aspect, provided herein is a synergistic composition comprising a compound of formula (I) and an additional agent. The additional agent can modulate a biological pathway or target different from the biological pathway or target modulated (e.g. inhibited) by a compound of formula (I). In some embodiments, the additional agent modulates a non-CDK biological pathway, a non-CDK target, a non-PAK biological pathway or a non-PAK target, e.g., a biological pathway or a target described herein. The compound of formula (I) and the additional agent can be present. In any ratio in the synergistic composition.

In yet another aspect, provided herein is a method of treating a neurodegenerative disorder. The method encompases administering a compound of formula (I) and an additional agent to the subject in need thereof. The additional agent can modulate a biological pathway or target different from the biological pathway or target modulated (e.g. inhibited) by a compound of formula (I). In some embodiments, the second compound can modulates modulate a CDK biological pathway, a non-CDK target, a non-PAK biological pathway or a non-PAK target, e.g., a biological pathway or a target described herein.

In yet still another aspect, disclosed herein is a method for identifying a compound that can increase motro neuron survival, the method comprising determining the effect of a test compound on a motor neuron, wherein test agent is incubated with the motor neuron in the absence of one or more trophic factors.

### BRIEF DESCRIPTION OF THE DRAWINGS

FIGS. 1A-1E show identification of small molecules that promote the survival of ESC-derived MNs. FIG. 1A shows an overview of directed differentiation and MN screening flow. Ag. Shh agonist; RA, retinoic acid. Scale bar represents 200 μm. FIG. 1B shows plate layout in the form of a heat map of a 384-well screening plate. Positive controls are in left top (−TF/+cycloheximide) and right (+TF) columns and negative controls (−TF) are in left bottom. Representative images of HB9::GFP MNs are shown for controls and for a hit well in which cells were treated with kenpaullone. Each image shown represents a field taken within an individual well (approximately one twelfth of the well). Scale bar represents 200 μm. FIG. 1C shows scatter plots showing screening results (points in red correspond to individual library compounds). FIG. 1D shows dose curves of a compound that scored only in HB9::GFP MNs (Tyrphostin A9), a compound that scored only in SOD1^{G93A}/HB9:GFP MNs (MDL 28170), and two compounds that scored in both HB9::GFP and SOD1^{G93A}/HB9:GFP MNs (kenpaullone and CP55940). Data are presented as mean±SD.

FIG. 1E shows image-based analyses allowed for excluding putative hit compounds that did not preserve MN integrity. Images shown here are (left) of a compound that preserved normal cell morphology including an intact neurite network (arrows: examples of visible neuronal processes) and (right) of a compound that resulted in an increased number of MNs, but mostly with small soma and few neurites. Scale bar represents 100 μm.

FIGS. 2A-2C show that Kenpaullone promotes MN survival through a cell autonomous mechanism. FIG. 2A is line graph showing dose curves of kenpaullone and other GSK-3 inhibitors that improve survival of both types of MNs to varying degrees. Kenpaullone clearly has particularly strong activity. FIG. 2B shows that reducing the number of progenitor cells using Ara-C does not diminish the effectiveness of kenpaullone. Olig2 staining was used to identify MN progenitors. Ara-C treatment (between days 2 and 4) reduced the number of Olig2⁺ cells, but did not reduce the number of MNs in kenpaullone treated wells. Scale bar represents 200 μm. FIG. 2C shows that the effect of kenpaullone remains essentially unchanged in highly enriched Hb9::GFP MN cultures. FACS purified MNs were treated with Ara-C to further ensure that any residual progenitor cell proliferation would be minimized. Images show that these cultures had very few non-MNs. In spite of that, kenpaullone was still able to improve MN survival to a comparable degree to that observed in mixed cultures. Scale bar represents 100 μm. Data in this figure are presented as mean±SEM.

FIGS. 3A-3C show that Kenpaullone treatment improves survival when MN death is initiated in other ways. FIGS. 3A and 3B show line graphs showing effects of kenpaullone and GSK-3 inhibitor VIII on MN death induced by treatment with a PI3K inhibitor. LY294002 was added to cultures on day 4, with or without concurrent treatment with 10 μM kenpaullone or 10 μM GSK-3 inhibitor VIII. Cells were fixed on day 7 for survival analysis. Survival is measured with respect to cells treated with DMSO alone. FIG. 3C is a bar graph showing that Kenpaullone also blocks death of MNs accelerated by co-culture with SOD1^{G93A} astrocytes. Hb9:GFP MNs were FACS sorted and plated on a confluent monolayer of wild-type or SOD1^{G93A} astrocytes. Kenpaullone was added to cultures 24 hrs later and was refreshed every three days by replacing half of the old medium with fresh medium containing IX concentration of the compound. Cells were fixed after nine days of compound treatment and MN survival was determined. Trophic factors were present throughout the experiment. Data are presented as percent survival normalized to that of DMSO treated MNs plated on wild-type astrocytes. Again, kenpaullone was effective in decreasing the extent of death. * p<0.05, ** p<0.01, *** p<0.001 (Student's t test, two-tailed). Data are presented as mean±SEM.

FIGS. 4A-4B show that Kenpaullone preserves morphological and functional integrity of MNs even after long treatment periods FIG. 4A shows that Kenpaullone (5 μM) increases the number of synapses per MN, when analyzed on day 21 (after 17 days of kenpaullone treatment). Images show synapsin (yellow) and PSD95 (red) staining on a kenpaullone-treated MN. Arrows indicate examples of synapses on MNs. Data are presented as mean±SEM. Scale bar represents 50 μm. FIG. 4B shows that Kenpaullone-treated MNs are electrophysiologically similar to those kept in +TF condition. HB9::GFP MNs treated for 3 days in the absence of trophic factors (−TF/+kenpaullone) were compared to MNs that were grown in complete medium (+TF) in terms of functional sodium and potassium currents [1], action potentials [2], response to excitatory neurotransmitter (kainate, 100 μM) [3], and response to inhibitory neurotransmitter (GABA, 100 μM) [4].

FIGS. 5A-5C show that Kenpaullone treatment decreases mutant SOD1 protein levels. In FIG. 5A cultures grown for 21 days in the indicated conditions were analyzed for levels of human SOD1 protein. The antibody used was specific for human SOD1, as is seen by the lack of staining on wild-type mouse MNs (HB9::GFP). When analyzed on day 21, levels of mutant SOD1^{G93A} protein, but not wt SOD1 protein, were decreased when cells were treated with kenpaullone in the absence of trophic factors. When analyzed on day 21, SOD1^{G93A} protein, but not wt SOD1 protein, was decreased when cultures were treated with kenpaullone in the presence of trophic factors as well (FIG. 5B). To confirm that the level of SOD1^{G93A} protein was also decreased in MNs, cultures were stained with an anti-human SOD1 antibody and analyzed by single cell imaging of GFP⁺ cells. By this analysis, again on day 21, the average amount of SOD1 per MN was decreased by kenpaullone treatment in both +TF and −TF conditions. Scale bar represents 50 μm (FIG. 5C). Data are presented as mean±SEM. * p<0.05, *** p<0.001 (Student's t test, two-tailed).

FIGS. 6A-6C show that Kenpaullone promotes the survival of human MNs. FIG. 6A is a line graph showing Kenpaullone (Ken), but not alsterpaullone (Alst), improves the survival of FACS purified human MNs derived from HuES-3/Hb9::GFP cells. Trophic factors were removed, compounds were added on day 4, and cells were fixed after an additional 14 days. MN survival was determined by counting GFP⁺ cells. FIGS. 6B and 6C are line grpahs showing Kenpaullone, but not alsterpaullone, increases survival of human MNs derived from control iPSCs (B) and fALS (SOD1^{1.144F}) iPSCs (C). Trophic factors were removed and compounds added as in (A). MN survival was determined by counting Isl⁺/MAP2⁺ cells. Data are presented as mean±SEM.

FIGS. 7A-7D show that Kenpaullone's effects cannot be explained by GSK-3 inhibition. FIG. 7A shows Islet/MAP2 staining on GSK-3α^{+/-}/GSK-3β^{-/-} (3/4 KO) MN culture. Scale bar represents 100 μm. FIG. 7B is bar graph showing comparison of MNs derived from wild-type, GSK-3α-KO, GSK-3β-KO and 3/4-KO mouse ESC lines shows that GSK-3 reduction has a small survival promoting effect when trophic factors are withdrawn. MN differentiation rates across lines were comparable on the day of plating, based on Islet/MAP2 staining (data not shown). However, kenpaullone treatment can still increase survival in all of the ESC lines, regardless of their genotypes. ** p<0.01, *** p<0.0001, compared to −TF of the same line (Student's t test, two-tailed). FIG. 7C is a line graph showing Purvalanol A, although toxic by itself, dramatically increased survival when added at 3 μM or 10 μM to alsterpaullone which, on its own, had weak effects. However, synergistic effect of Purvalanol with kenpaullone (FIG. 7D) was not as great as with alsterpaullone. Data are presented as mean±SEM.

FIGS. 8A-8C show trophic factor withdrawal-induced MN death. In FIGS. 8A and 8B trophic factor (TF) withdrawal induced about 80% MN death, i.e. a five-fold screening window, in Hb9::GFP (FIG. 8A) an SOD1^{G93A}/HB9::GFP (FIG. 8B) MN cultures. Freshly dissociated EBs were plated at 8,000 GFP⁺ cells per well in poly-D-lysine coated 384-well plates. TFs were removed four days later, and plates were fixed after another three days. Surviving MN numbers were obtained by counting GFP⁺ cells. Data are presented as mean: SEM. ***p<0.001 (student's t-test, two-tailed). In FIG. 8C cycloheximide (CHX), a protein synthesis inhibitor, was used as the positive control for the screen. CHX at concentration of 100 ng/ml showed no toxicity and was able to prevent MNs from dying in the −TF condition. Scale bar represents 200 μm.

FIGS. 9A-9C show the survival effect of kenpaullone and GSK-3 inhibitors. FIG. 9A is bar graph showing dose curves of GSK-3 inhibitors, including selective ones, that do not promote MN survival. Data are represented as average±SEM. FIGS. 9B and 9C show MNs surviving after kenpaullone (ken) treatment were not generated from BrdU-labelod progenitor cells. FIG. 9B show an overview of BrdU labeling method. Trophic factors were removed and kenpaullone was added to HB9::GFP MNs on day 4. BrdU was added to cultures 2 hrs, 24 hrs, 48 hrs and 72 hrs before fixing on day 7. FIG. 9C, upper panel, shows images of +TF conditions from different lengths of BrdU incubation. FIG. 9C, lower panel, shows images of +TF, −TF and −TF+ken cells after 72 hours of BrdU incubation. There were few, if any, BrdU⁺/GFP⁺ double-positive cells in any of the cultures examined, suggesting that none of the surviving MNs were newly generated during the course of the experiment Kenpaullone-treated cultures had many BrdU-positive clusters of cells that were not seen under other conditions, suggesting that kenpaullone may also stimulate progenitor cell proliferation or support survival or proliferating progenitors. Scale bar represents 200 μm.

FIGS. 10A-10G show the Morphological and electrophysiological analyses. FIGS. 10A-10F show that Kenpaullone (ken) enhances MN neuritearborization. Trophic factors (TFs) were removed and kenpaullone (5 μM) was added on day 4, and neuronal arborization was analyzed by imaging on day 21. FIG. 10A shows an overview of traces of neuronal processes as defined by imaging software. Kenpaullone increases all aspects of neuronal arborization analyzed, including total neurite length per MN (FIG. 10B), maximum neurite length per MN (FIG. 10C), numbers of extremities per MN (FIG. 10D), numbers of roots per MN (FIG. 10E), and numbers of nodes per MN (FIG. 10F). Data are presented as mean±SEM. Scale bar represents 100 μm. FIG. 10G shows a summary of the quantification of electrophysiological properties of HB9::GFPMNs in +TF and in −TF/+kenpaullone conditions analyzed after three days of kenpaullone treatment. The two types of cells were similar by these measurements. p values all >0.1 by two-tailed t-test. pF—picofarads, mV—millivos, and pA—picoamperes. Data are presented as mean±SD.

FIGS. 11A-11C show that Kenpaullone promotes long-term survival of both types of MNs in medium with and without trophic factors. In FIG. 11A, number of MNs was determined at various times after TF withdrawal. Cells were fed and kenpaullone was refreshed every two days. TF=trophic factors; ken=kenpaullone. FIG. 11B shows that three days of kenpaullone treatment has a small effect on MN survival even in the presence of trophic factors. FIG. 11C shows long-term survival effect of kenpaullone in complete medium (+TF). Kenpaullone was added to cells on day 4, and survival was quantified at the indicated times. Cells were refed with fresh medium and compound every two days. Data are presented as mean±SEM.

FIGS. 12A and 12B show that Kenpaullone reduces ubiquitin protein levels at later times. FIG. 12A shows that no significant decreases in SOD1^{G93A} protein levels in cultures were observed by Western blotting on day 15. FIG. 12B shows image analysis demonstrates that kenpaullone treatment reduces ubiquitin levels in SOD1^{G93-A}/HB9::GFP MNs on day 21. Insets show higher magnification of cells contained within white circles. Data are presented as mean±SEM. * p<0.05 (Student's t test, two-tailed). Scale bar represents 50 μm.

FIGS. 13A and 138 show the differentiation of MNs from human ESCs and iPSCs. FIG. 13A is an overview of differentiation protocol for human MNs. Also see materials and methods. FIG. 13B shows images of EBs (top panel) and MNs (middle panel) from Hues-3/Hb9::GFP cells and MNs derived from human healthy control iPSCs (bottom panel). Islet and MAP2 staining were used to mark MNs derived from iPSCs. Scale bar represents 200 μm.

FIGS. 14A-14C show survival effects of GSK-3α and GSK-3β knockdown and of purvalanol A treatment. FIG. 14A is a Western blot showing the levels of knockdown of GSK-3α and GSK-3β in HB9::GFP MN cultures after infection with either non-silencing shRNA or shRNAs against both GSK-3α and GSK-3β. Two days after virus addition, puromycin was added to eliminate non-infected cells, and four days later the cultures were harvested for Western blotting. At the time of harvest approximately 20-30% of all cells were MNs (data not shown). About 43% knockdown of GSK-3α and 60% knockdown of GSK-30 were generated by these shRNAs. FIG. 14B shows survival after GSK-3 knockdown by shRNAs. HB9::GFP MN cultures were infected with lentivirus on day 2. Trophic factors (TFs) were removed on day 4, and survival was analyzed on day 7. MN survival was quantified with respect to that in cultures kept in +TF conditions. FIG. 14C shows that Purvalanol A was highly toxic to MNs. About 50% of HB9::GFP MNs died when treated with 0.5 and 1 μM, and most MNs died when incubated in 3 and 10 μM. Data are presented as mean±SEM.

FIGS. 15A-15E show the results of mechanistic studies of kenpaullone activity. FIG. 15A is a comparison of MNs derived from wild-type, GSK-3α-KO, GSK-3β-KO and 3/4-KO mouse ESC lines which shows that GSK-3 reduction has a small survival promoting effect when trophic factors are withdrawn. Statistics performed comparing MN survival in −TF condition of each genotype to wildtype. However, kenpaullone treatment further increased survival in all of the ESC lines, regardless of their genotype. Statistics performed comparing MN survival in +Ken treatment to −TF of the same genotype. ** p<0.01, *** p<0.0001 (Student's t test, two-tailed). Note that MN differentiation rates across lines were comparable on the day of plating, based on Islet/MAP2 staining (data not shown). FIGS. 15B-15E demonstrate that Kenpaullone blocks the JNK/c-Jun mediated cell death signaling cascade in MNs by inhibiting activity of an upstream activator, HGK. (B) Kenpaullone, but not CHIR 99021, blocks phosphorylation and therefore activation of c-Jun, JNK, MKK4, and Tak1 in SOD1G93A/Hb9::GFP MNs as early as 1 h after treatment. * p<0.05, ** p<0.01 *** p<0.001, **** p<0.0001 (Two-way ANOVA) (C) Examples of phospho-cJun staining (red) 24 h after trophic factor withdrawal in SOD1G9JA/Hb9::GFP MNs (green) treated with DMSO, 1 μM CHIR 99021, or 10 μM kenpaullone. The images demonstrate that kenpaullone inhibits c-Jun phosphorylation. (D) Results from a kinase activity assay demonstrate that kenpaullone strongly inhibits HGK activity while CHIR99020 does not. (E) A schematic of the HGK-mediated cell death pathway shows where kenpaullone acts in the signaling cascade. Data are presented as mean±SEM.

FIG. 16A-16D shows the effect of Kenpaullone and other ALS drugs on the survival of human MNs. Fold increase in the survival of human MNs derived from (A) HuES-3/Hb9::GFP ESCs, (B) wildtype IPSCs, (C) SOD1L144F iPSCs, or (D) TDP-43M3337V iPSC treated with DMSO, Kenpaullone, Olesoxime (Oles), or Dexpramipexole (Dex). Either HB9::GPF positive or Isl/Tuj1 double positive cells were counted as MNs. Data are presented as mean±SEM. * p<0.05, ** p<0.01, n.s., not significant, compared to −TF control (Student's t test, two-tailed).

### DETAILED DESCRIPTION

The present invention relates to methods and compositions for promoting motor neuron survival, treating or preventing neurodegenerative disorders (e.g., amyotropic lateral sclerosis (ALS) and spinal muscular atrophy (SMA)), methods for identifying agents that promote survival of motor neurons, and methods of identifying agents that are useful for treating neurodgenerative disorders.

The work described herein demonstrates, inter alia, that HPK/GC kinase-like kinase (HGK) inhibition can promote motor neuron survival.

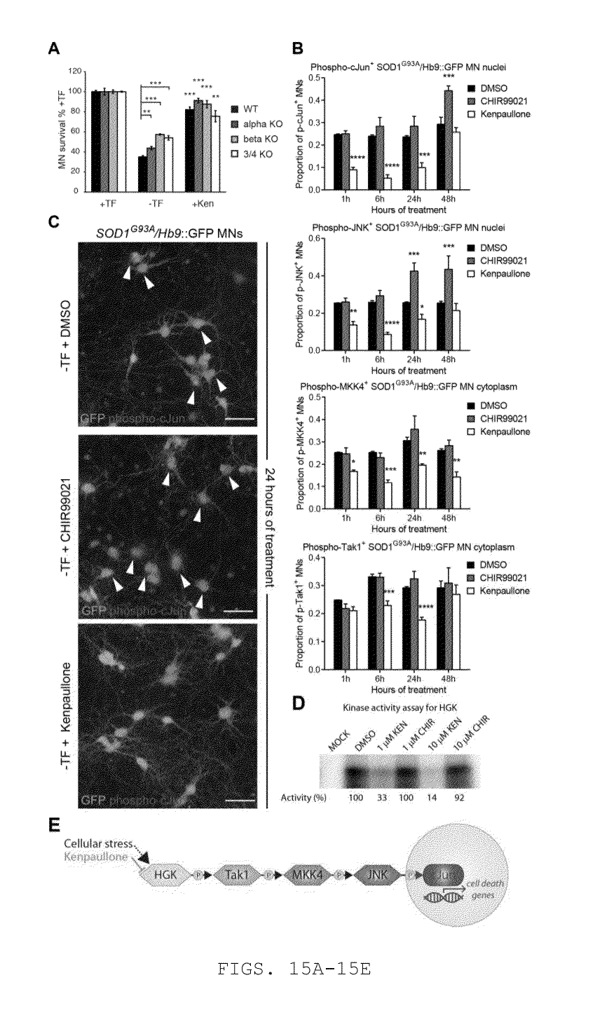

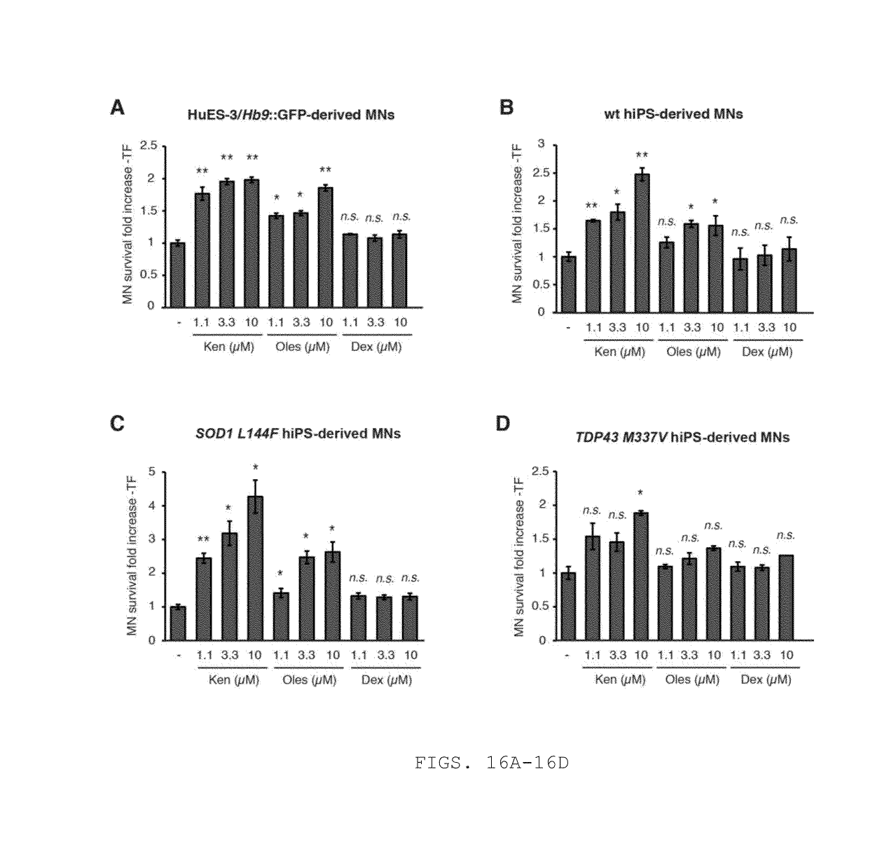

Accordingly, in one aspect, disclosed herein is a method of promoting motor neuron survival, comprising contacting a motor neuron with an effective amount of an agent that inhibits HPK/GC kinase-like kinase (also known as mitogen-activated protein kinase kinase kinase kinase 4 (MAP4K4); NCBI GeneID: 9448; hereinafter HGK). HGK encodes a serine/threonine protein kinase that acts upstream in the JNK/c-Jun mediated cell death signaling cascade (FIG. 15E). In some embodiments, the agent decreases activation of the HGK-Tak1-MKK4-JNK-c-Jun cell death signaling cascade.

As used herein, “agent that inhibits HPK/GC kinase-like kinase” refers to an agent that decreases the level of HGK kinase mRNA or protein, an activity of HGK kinase, the half-life of HGK kinase mRNA or protein, or the binding of HGK kinase to another molecule (e.g., a substrate for a HGK kinase, e.g., transforming growth factor-O activated kinase (Tak1)), as measured using standard methods (see, for example, Ausubel et al., Current Protocols in Molecular Biology, Chapter 9, John Wiley & Sons, New York, 2000). For example, the agent may directly or indirectly inhibit the ability of HGK kinase to phosphorylate Tak1. Expression levels of mRNA can be determined using standard RNase protection assays or in situ hybridization assays, and the level of protein can be determined using standard Western or immunohistochemistry analysis. The phosphorylation level of signal transduction proteins downstream of Tak1 activity can also be measured using standard assays. In some embodiments, a HGK inhibitor decreases HGK kinase activity by at least 20, 40, 60, 80, or 90%. In some embodiments, the level of HGK kinase activity is at least 2, 3, 5, 10, 20, or 50-fold lower in the presence of the HGK inhibitor.

In some embodiments, the agent inhibits HGK (e.g., an HGK inhibitor).

An HGK inhibitor or agent that inhibits HGK can be small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

In some embodiments, the agent is an inhibitor of glycogen synthase kinase 3 (GSK-3). In some embodiments, the agent inhibits GSK-3. In some embodiments, the agent is an agent that inhibits GSK-3 as described herein. In some embodiments, that agent exhibits dual inhibition of GSK-3 and HGK. In some embodiments, a method of promoting motor neuron survival further includes contacting the motor neuron with an additional agent that inhibits GSK-3.

Without limitations, the agent that inhibits HGK and the additional agent that inhibits GSK-3 can be used in any ratio. Accordingly, in some embodiments, the agent that inhibits HGK and the additional agent that inhibits GSK-3 are in a ratio from 20:1 to 1:20. In some embodiments, the agent that inhibits HGK and the additional agent that inhibits GSK-3 are in ratio from 10:1 to 1:10. In some embodiments, the agent that inhibits HGK and the additional agent that inhibits GSK-3 are in ratio from 5:1 to 1:5. In some embodiments, the agent that inhibits HGK and the additional agent that inhibits GSK-3 are in a ratio from 15:1 to 1:5. In some embodiments, the agent that inhibits HGK and the additional agent that inhibits GSK-3 are in a ratio from 10:1 to 1:1. In one embodiment, the agent that inhibits HGK and the additional agent that inhibits GSK-3 are used in a 1:1 ratio.

An exemplary agent that inhibits HGK is kenpaullone and pharmaceutically acceptable salts thereof. The present invention also contemplates analogs and derivatives of kenpaullone as described herein that are capable of inhibiting HGK. Those skilled in the art will appreciate that a variety of routine methods are available for determining suitable kenpaullone analogs and derivatives (e.g., any of the kinase assays described in Anastassiadis et al. 2011).

In some embodiments, a HGK inhibitor comprises a dominant negative mutant form of HGK (e.g., a catalytically inactive HGK mutant (Yao et al. 1999)). In an embodiment, a catalytically inactive HGK mutant is a HGK polypeptide in which lysine 54 is substituted with arginine (K54R mutant). In an embodiment, a catalytically inactive HGK mutant is a HGK polypeptide in which lysine 54 is substituted with glutumic acid (K54E mutant). Without wishing to be bound by theory, it is believed that contacting a motor neuron with catalytically inactive HGK mutants can result in inhibition of HGK induced activation of the HGK-Tak1-MKK4-JNK-cJun cell death signaling cascade, thereby promoting survival of the motor neuron.

Additional exemplary agents that inhibit HGK are shown in Table 1 below. The value column of Table 1 indicates the average percentage of remaining kinase activity of HGK in the presence of the inhibitor listed in Table 1 relative to a solvent control (see Anastassiadis et al. 2011).

TABLE 1
HGK inhibitors

| InhibitorName | InhibitorCAS | Value |
|---|---|---|
| Alsterpaullone | 237430-03-4 | 4.47 |
| Alsterpaullone 2-Cyanoethyl | 852527-97-0 | 6.82 |
| AMPK Inhibitor- Compound C | 866405-64-3 | 28.98 |
| Cdk1/2 Inhibitor III | 443798-55-8 | 4.46 |
| Cdk4 Inhibitor | 546102-60-7 | 29.26 |
| Flt-3 Inhibitor II | 896138-40-2 | 6.63 |
| Flt-3 Inhibitor III | 852045-46-6 | 35.5 |
| GSK-3 Inhibitor IX | 667463-62-9 | 28.64 |
| GSK-3 Inhibitor X | 740841-15-0 | 30.16 |
| GSK-3b Inhibitor XI | 626604-39-5 | 36.44 |
| GSK3b Inhibitor XII- TWS119 | 601514-19-6 | 12.46 |
| GSK-3 Inhibitor XIII | 404828-08-6 | 25.68 |
| G″ 6976 | 136194-77-9 | 0.43 |
| G″ 6983 | 133053-19-7 | 37.75 |
| GTP-14564 | 34823-86-4 | 36.09 |
| Isogranulatimide | 244148-46-7 | 21.23 |
| H-89- Dihydrochloride | 127243-85-0 | 43.85 |
| HA 1077- Dihydrochloride | 103745-39-7 | 44.11 |
| Fasudil | | |
| Indirubin Derivative E804 | 854171-35-0 | 11.58 |
| Indirubin-3?-monoxime | 160807-49-8 | 41.97 |
| JAK Inhibitor I | 457081-03-7 | 38.41 |
| JAK3 Inhibitor VI | 856436-16-3 | 0.63 |
| K-252a- Nocardiopsis sp. | 97161-97-2 | 0.12 |
| Kenpaullone | 142273-20-9 | 6.61 |
| Met Kinase Inhibitor | 658084-23-2 | 40.9 |
| PD 169316 | 152121-53-4 | 44.94 |
| PKR Inhibitor | 608512-97-6 | 5.36 |
| PKR Inhibitor- Negative | 852547-30-9 | 4.48 |
| Control | | |
| PP1 Analog II- 1NM-PP1 | 221244-14-0 | 43.46 |
| Staurosporine- N-benzoyl- | 120685-11-2 | 7.25 |
| SB 218078 | 135897-06-2 | 0.99 |
| Staurosporine- Streptomyces | 62996-74-1 | 1.91 |
| sp. | | |
| STO-609 | 52029-86-4 | 43.41 |
| SU6656 | 330161-87-0 | 46.15 |
| SU9516 | 666837-93-0 | 19.36 |
| SU11652 | 326914-10-7 | 4.33 |
| Syk Inhibitor | 622387-85-3 | 22.51 |
| Syk Inhibitor II | 227449-73-2 | 48.41 |
| Tpl2 Kinase Inhibitor | 871307-18-5 | 44.43 |
| TGF-b RI Kinase Inhibitor | 396129-53-6 | 48.31 |
| VEGF Receptor 2 Kinase | 288144-20-7 | 6.84 |
| Inhibitor II | | |
| VEGF Receptor 2 Kinase | 204005-46-9 | 48.78 |
| Inhibitor III | | |
| VEGF Receptor 2 Kinase | 216661-57-3 | 28.4 |
| Inhibitor IV | | |
| Bosutinib | 380843-75-4 | 1.03 |
| Dasatinib | 302962-49-8 | 49.2 |
| Dovitinib | 405169-16-6 | 2.96 |
| Sunitinib | 557795-19-4 | 12.44 |

In some embodiments, an agent that inhibits HGK (e.g., a HGK inhibitor) inhibits/lowers the activity of HGK by at least 5%, 10%, 20%, 30%, 40%, 50%, 60%, 70%, 80%, 90%, or 95% relative to a control. While not required, an agent that inhibits HGK (e.g., a HGK inhibitor) can completely inhibit the HGK activity relative to a control.

In some embodiments, a TNF-α inhibitor can be used as an agent that inhibits HGK. TNF-α (NCBI Gene ID: 7124) has been reported to stimulate HGK kinase activity (Yao et al. 1999). Accordingly, without wishing to be bound by theory, it is believed that inhibition of TNF-α can result in diminished or reduced HGK kinase activity.

As used herein, “TNF-α inhibitor” and “agent that inhibits TNF-α” are used interchangeably to refer to an agent that decreases the level of TNF-α mRNA or protein, an activity of TNF-α, the half-life of TNF-α mRNA or protein, or the binding of TNF-α to another molecule (e.g., a substrate for TNF-α), as measured using standard methods (see, for example, Ausubel et al., Current Protocols in Molecular Biology, Chapter 9, John Wiley & Sons, New York, 2000). For example, the agent may directly or indirectly inhibit the ability of TNF-α to stimulate activation of HGK kinase activity. Expression levels of mRNA can be determined using standard RNase protection assays or in situ hybridization assays, and the level of protein can be determined using standard Western or immunohistochemistry analysis.

In some embodiments, a TNF-α inhibitor decreases HGK kinase activity by at least 20, 40, 60, 80, or 90%. In some embodiments, the level of HGK kinase activity is at least 2, 3, 5, 10, 20, or 50-fold lower in the presence of the TNF-α inhibitor.

A TNF-α inhibitor can be small organic or inorganic molecules; saccharines; oligosaccharides polysaccharides; biological macromolecules, e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

Exemplary TNF-α inhibitors include, but are not limited to metalloproteinase (MMP) inhibitors, tetracyclines, chemically modified tetracyclines, quinolones, corticosteroids, thalidomide, lazaroids, pentoxifylline, hydroxamic acid derivatives, napthopyrans, soluble cytokine receptors, monoclonal antibodies towards TNF-α (e.g., Etanercept (aka ENBREL®, described in WO 91/03553 and WO 09/406476), infliximab (described in U.S. Pat. No. 5,656,272), CDP571 (a humanized monoclonal anti-TNF-alpha IgG4 antibody), CDP 870 (a humanized monoclonal anti-TNF-alpha antibody fragment), certolizumab pegol (CIMZIA®). D2E7/HUMIRA® (a human anti-TNF mAb), golimumab (SIMPONIL)), amrinone, pimobendan, vesnarinone, phosphodlesterase inhibitors, lactoferrin and lactoferrin derived analogous, and melatonin in the form of bases or addition salts optionally together with a pharmaceutically acceptable carrier.

The work described herein further demonstrates, inter alia, that decreasing activation of the HGK-Tak1-MKK4-JNK-c-Jun cell death signaling cascade (also referred to herein as the JNK/c-Jun mediated cell death signaling cascade or pathway) in a motor neuron can promote survival of the motor neuron.

Accordingly, in one aspect, disclosed herein is a method of promoting motor neuron survival, comprising contacting a motor neuron with an effective amount of an agent that decreases activation of the HGK-Tak1-MKK4-JNK-c-Jun cell death signaling cascade.

As used herein, “HGK-Tak1-MKK4-JNK-c-Jun cell death signaling cascade inhibitor” and “agent that inhibits the HGK-Tak1-MKK4-JNK-c-Jun cell death signaling cascade” are used interchangeably to refer to an agent that decreases the level of mRNA or protein involved in activation of the HGK-Tak1-MKK4-JNK-c-Jun cell death signaling cascade, an activity of a protein involved in activation of the HGK-Tak1-MKK4-JNK-c-Jun cell death signaling cascade, the half-life of mRNA or protein involved in activation of the HGK-Tak1-MKK4-JNK-c-Jun cell death signaling cascade, or the binding of a protein involved in activation of the HGK-Tak1-MKK4-JNK-c-Jun cell death signaling cascade to another molecule in the HGK-Tak1-MKK4-JNK-c-Jun cell death signaling cascade, as measured using standard methods (see, for example, Ausubel et al., Current Protocols in Molecular Biology, Chapter 9, John Wiley & Sons, New York, 2000). For example, the agent may directly or indirectly inhibit the ability of a protein in the HGK-Tak1-MKK4-JNK-c-Jun cell death signaling cascade to phosphorylate another protein in the cascade or another protein that interacts with a kinase in the cascade. Expression levels of mRNA can be determined using standard RNase protection assays or in situ hybridization assays, and the level of protein can be determined using standard Western or immunohistochemistry analysis. The phosphorylation levels of signal transduction proteins in the HGK-Tak1-MKK4-JNK-c-Jun cell death signaling cascade can also be measured using standard assays. In some embodiments, a HGK-Tak1-MKK4-JNK-c-Jun cell death signaling cascade inhibitor decreases activation of the HGK-Tak1-MKK4-JNK-c-Jun cell death signaling cascade by at least 20, 40, 60, 80, or 90%. In some embodiments, the level of HGK-Tak1-MKK4-JNK-c-Jun cell death signaling cascade activation is at least 2, 3, 5, 10, 20, or 50-fold lower in the presence of the HGK-Tak1-MKK4-JNK-c-Jun cell death signaling cascade inhibitor.

A HGK-Tak1-MKK4-JNK-c-Jun cell death signaling cascade inhibitor can be small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

An exemplary HGK-Tak1-MKK4-JNK-c-Jun cell death signaling cascade inhibitor is kenpaullone and pharmaceutically acceptable salts thereof. The present invention also contemplates analogs and derivatives of kenpaullone as described herein that are capable of decreasing activation of the HGK-Tak1-MKK4-JNK-c-Jun cell death signaling cascade inhibitors. Those skilled in the art will appreciate that a variety of routine methods are available for determining suitable kenpaullone analogs and derivatives (e.g., any of the kinase assays described in Anastassiadis et al. 2011).

In some embodiments, a HGK-Tak1-MKK4-JNK-c-Jun cell death signaling cascade inhibitor lowers the activation of the HGK-Tak1-MKK4-JNK-c-Jun cell death signaling cascade by at least 5%. 10%. 20%, 30%, 40%, 50%, 60%, 70%, 80%, 90%, or 95% relative to a control. While not required, a HGK-Tak1-MKK4-JNK-c-Jun cell death signaling cascade inhibitor can completely inhibit activation of the HGK-Tak1-MKK4-JNK-c-Jun cell death signaling cascade relative to a control.

In some embodiments of this and other aspects of the Invention, the agent decreases phosphorylation of a protein kinase in the HGK-Tak1-MKK4-JNK-c-Jun cell death signaling cascade.

In some embodiments, the protein kinase is HGK. In some embodiments, an HGK inhibitor as described herein can be used as a HGK-Tak1-MKK4-JNK-c-Jun cell death signaling cascade inhibitor.

In some embodiments, the protein kinase is Tak1. In some embodiments, a Tak1 inhibitor can be used as a HGK-Tak1-MKK4-JNK-c-Jun cell death signaling cascade inhibitor.

A Tak1 inhibitor can be small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and apatamers); an extract made from biological materials such as bacteria plants, fungi, or animal cells; animal tissues; naturally occurring or synthetic compositions; and any combinations thereof.

Exemplary Tak1 inhibitors include, but are not limited to zearalenones disclosed in WO 2002048135, TAK1 short interfering RNA (siRNA) described in Takaesu et al J Mol Biol. 2003; 326(1): 105-15, and an inactive mutant of TAK1 is described in Thiefes et. al., J Biol Chem. 2005; 280(30):27728-41, each of which is incorporated herein by reference in its entirety. In some embodiments, a Tak1 inhibitor comprises a dominant negative mutant of Tak1. In some embodiments, a dominant negative mutant form of Tak1 comprises the dominant negative mutant TAK-K63W (Yao et al. 1999). In some embodiments, a Tak1 inhibitor comprises (5Z)-7-Oxozeaenol (see Wu et al 2013). In some embodiments, a Tak1 inhibitor comprises an analog of (Z)-7-Oxozeanol as described in PCT International Application No. PCT/US2012/047314, incorporated herein by reference in its entirety. In some embodiments, a Tak1 inhibitor comprises a derivative of (5Z)-7-Oxozeanol as described by Strippoli et a. (Strippoli et al. 2012). In some embodiments, a Tak1 inhibitor comprises LYTAK1 (see Melisi el al. 2011). In some embodiments, a Tak1 inhibitor comprises an HGK inhibitor or agent that inhibits HGK as described herein (e.g., kenpaullone and analogs or derivatives described herein, and their pharmaceutically acceptable salts).

In some embodiments, a Tak1 inhibitor inhibits/decreases the activation of the HGK-Tak1-MKK4-JNK-c-Jun cell death signaling cascade by at least 5%, 10%, 20%, 30%, 40%, 50%, 60%, 70%, 80%, 90%, or 95% relative to a control. While not required, a Tak1 inhibitor can completely inhibit activation of the HGK-Tak1-MKK4-JNK-c-Jun cell death signaling cascade relative to a control.

In some embodiments, an agent that decreases activation of the HGK-Tak1-MKK4-JNK-c-Jun cell death signaling cascade is not a Tak1 inhibitor (e.g., an inhibitor that is selective and/or specific for, and/or directly inhibits Tak1). In some embodiments, an agent that decreases activation of the HGK-Tak1-MKK4-JNK-c-Jun cell death signaling cascade is not a Tak1 inhibitor as described herein.

In some embodiments, the protein kinase is MKK4. In some embodiments, a MKK4 inhibitor can be used as a HGK-Tak1-MKK4-JNK-c-Jun cell death signaling cascade inhibitor.

A MKK4 inhibitor can be small organic or inorganic molecules; saccharines; oligosaccharides; polysaccharides; biological macromolecules. e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

Exemplary MKK4 inhibitors include the protein kinase inhibiting compounds of formula I as described in U.S. Pat. No. 7,745,437 (incorporated by reference herein), e.g., N-[4-methyl-3-(1-{6-[4-(2-morpholin-4-yl-ethyl)-phenylamino]-pyrimidin-4-yl}-1H-imidazol-2-ylamino)-phenyl]-3-trifluoromethyl-benzamide, N-{3-[1-(6-cyclopropylamino-pyrimidin-4-yl)-1H-imidazol-2-ylamino]-4-methyl-phenyl}-3-(4-ethyl-piperazin-1-yl)-5-trifluoromethyl-benzamide, N-(4-methyl-3-({1-[6-(2-morpholin-4-yl-ethylamino)-pyrimidin-4-yl]-1H-imidazol-2-ylamino}phenyl)-3-trifluoromethyl-benzamide, N-{3-[1-(6-cyclopropylamino-pyrimidin-4-yl)-1H-imidazol-2-ylamino]-4-methyl-phenyl}-3-trifluoromethyl-benzamide, N-{4-methyl-3-[(6-methylamino-pyrimidin-4-yl)-1H-imidazol-2-ylamino]-phenyl}-3-trifluoromethyl-benzamide, N-[4-methyl-3-(1-{6-[3-(2-oxo-pyrrolidin-1-yl)-propylamino]-pyrimidin-4-yl}-1H-imidazol-2-ylamino)-phenyl]-3-trifluoromethyl-benzamide, N-(3-{1-[6-(benzo[1,3]dioxol-5-ylamino)-pyrimidin-4-yl]-1H-imidazol-2-ylamino}-4-methyl-phenyl)-3-trifluoromethyl-benzamide. N-[4-methyl-3-(1-{6-[3-(4-methyl-piperazin-1-yl)-propylamino]-pyrimidin-4-yl}-1H-imidazol-2-ylamino)-phenyl]-3-trifluoromethyl-benzamide, {6-[2-(2-chloro-phenylamino)-imidazol-1-yl]-pyrimidin-4-yl}-cyclopropyl-amine, (3-{6-[2-(2-chloro-phenylamino)-imidazol-1-yl]-pyrimidin-4-ylamino}-phenyl)-methanol, 1-(3-{6-[2-(2-chloro-phenylamino)-imidazol-1-yl]-pyrimidin-4-ylamino}-phenyl)-ethanol, {6-[2-(2-chlorophenylamino)-imidazol-1-yl]-pyrimidin-4-yl}-[4-(2-morpholin-4-yl-ethyl)-phenyl]-amine, {6-[2-(2-chloro-phenylamino)-imidazol-1-yl]-pyrimidin-4-yl}-(4-morpholin-4-yl-phenyl)-amine, N-(3-{6-[2-(2-chloro-phenylamino)-imidazol-1-yl]-pyrimidin-4-ylamino}-phenyl)-acetamide, 1-(3-{6-[2-(2-chloro-phenylamino)-imidazol-1-yl]-pyrimidin-4-ylamino}-propyl)-pyrrolidin-2-one, benzo[1,3]dioxol-5-yl-{6-[2-(2-chloro-phenylamino)-imidazol-1-yl]-pyrimidin-4-yl}-amine. {6-[2-(2-chloro-phenylamino)-imidazol-1-yl]-pyrimidin-4-yl}-pyridin-3-yl-amine, N-(4-methyl-3-{1-[6-(pyridin-3-ylamino)-pyrimidin-4-yl]-1H-imidazol-2-ylamino}-phenyl)-3-trifluoromethyl-benzamide, {6-[2-(2-chloro-phenylamino)-imidazol-1-yl]-pyrimidin-4-yl}-(6-methyl-pyridin-3-yl)-amine, N-(4-methyl-3-{1-[6-(6-methyl-pyridin-3-ylamino)-pyrimidin-4-yl]-1H-imidazol-2-ylamino}-phenyl)-3-trifluoromethyl-benzamide, N-{3-[1-(6-cyclopropylamino-pyrimidin-4-yl)-1H-imidazol-2-ylamino]-4-methyl-phenyl}-3-morpholin-4-yl-5-trifluromethyl-benzamide, N-{3-[1-(6-cyclopropylamino-pyrimidin-4-yl)-1H-imidazol-2-ylamino]-4-methyl-phenyl}-3-(4-methyl-piperazin-1-yl)-5-trifluoromethyl-benzamide, N-{3-[1-(6-cyclopropylamino-pyrimidin-4-yl)-1H-imidazol-2-ylamino]-4-methyl-phenyl}-3-(4-ethyl-piperazin-1-yl)-5-trifluoromethyl-benzamide, N-{3-[1-(6-cyclopropylamino-pyrimidin-4-yl)-1H-imidazol-2-ylamino]-4-methyl-phenyl}-4-morpholin-4-yl-3-trifluoromethyl-benzamide. N-{3-[1-(6-cyclopropylamino-pyrimidin-4-yl)-1H-imidazol-2-ylamino]-4-methyl-phenyl}-4-(4-ethyl-piperazin-1-ylmethyl)-3-trifluoromethyl-benzamide, N-{3-[1-(6-cyclopropylamino-pyrimidin-4-yl)-1H-imidazol-2-ylamino]-4-methyl-phenyl}-4-(4-methyl-piperazin-1-ylmethyl)-3-trifluoromethyl-benzamide, N-{3-[1-(6-cyclopropylamino-pyrimidin-4-yl)-1H-imidazol-2-ylamino]-4-methyl-phenyl}-3-(4-methyl-imidazol-1-yl)-5-trifluoromethyl-benzamide, 3-(4-methyl-imidazol-1-yl)-N-{4-methyl-3-[1-(6-methylamino-pyrimidin-4-yl)-1H-imidazol-2-ylamino]-phenyl}-5-trifluoromethyl-benzamide, N-{4-methyl-3-[1-(6-methylamino-pyrimidin-4-yl)-1H-imidazol-2-ylamino]-phenyl}-4-morpholin-4-yl-3-trifluoromethyl-benzamide, 3-(4-ethyl-piperazin-1-yl)-N-{4-methyl-3-[1-(6-methylamino-pyrimidin-4-yl)-1H-imidazol-2-ylamino]-phenyl}-5-trifluoromethyl-benzamide, N-{4-methyl-3-[1-(6-methylamino-pyrimidin-4-yl)-1H-imidazol-2-ylamino]-phenyl}-3-(4-methyl-piperazin-1-yl)-5-trifluoromethyl-benzamide, N-{3-[1-(6-methylamino-pyrimidin-4-yl)-1H-imidazol-2-ylamino]-phenyl}-4-(4-methyl-piperazin-1-ylmethyl)-3-trifluoromethyl-benzamide, 3-(4-ethyl-piperazin-1-ylmethyl)-N-{4-methyl-3-[1-(6-methylamino-pyrimidin-4-yl)-1H-imidazol-2-ylamino]-phenyl}-5-trifluoromethyl-benzamide, N-{3-[1-(6-cyclopropylamino-pyrimidin-4-yl)-1H-imidazol-2-ylamino]-4-methyl-phenyl}-4-morpholin-4-ylmethyl-3-trifluoromethyl-benzamide, N-{4-methyl-3-[1-(6-methylamino-pyrimidin-4-yl)-1H-imidazol-2-ylamino]-phenyl}-4-morpholin-4-ylmethyl-3-trifluoromethyl-benzamide, N-{3-[1-(6-amino-pyrimidin-4-yl)-1H-imidazol-2-ylamino]-4-methyl-phenyl}-4-(4-ethyl-piperazin-1-ylmethyl)-3-trifluoromethyl-benzamide, N-{3-[1-(6-amino-pyrimidin-4-yl)-1H-imidazol-2-ylamino]-4-methyl-phenyl}-4-(4-methyl-piperazin-1-ylmethyl)-3-trifluoromethyl-benzamide, N-{3-[1-(6-amino-pyrimidin-4-yl)-1H-imidazol-2-ylamino]-phenyl}-3-(4-methyl-imidazol-1-yl)-5-trifluoromethyl-benzamide, N-{3-[1-(6-amino-pyrimidin-4-yl)-1H-imidazol-2-ylamino]-4-methyl-phenyl}-4-morpholin-4-Yl-3-trifluoromethyl-benzamide, N-{3-[1-(6-amino-pyrimidin-4-yl)-1H-imidazol-2-ylamino]-4-methyl-phenyl}-3-(4-ethyl-piperazin-1-yl)-5-trifluoromethyl-benzamide, N-{3-[1-(6-amino-pyrimidin-4-yl)-1H-imidazol-2-ylamino]-4-methyl-phenyl}-3-(4-methyl-piperazin-1-yl)-5-trifluoromethyl-benzamide, N-{3-[1-(6-amino-pyrimidin-4-yl)-1H-imidazol-2-ylamino]-4-methyl-phenyl}-3-morpholin-4-ylmethyl-5-trifluoromethyl-benzamide, 3-[1-(6-cyclopropylamino-pyrimidin-4-yl)-1H-imidazol-2-ylamino]5-methoxy-N-[4-(2-methyl-imidazol-1-yl)-3-trifluoromethyl-phenyl]-benzamide, N-{3-[1-(6-acetylamino-pyrimidin-4-yl)-1H-imidazol-2-ylamino]-4-methyl-phenyl}-3-trifluoromethyl-benzamide, N-{3-[1-(6-methanesulfonylamino-pyrimidin-4-yl)-1H-imidazo-2-ylamino]-4-methyl-phenyl}-3-trifluoromethyl-benzamide, N-{3-[1-(2-cyclopropylamino-pyrimidin-4-yl)-1H-imidazol-2-ylamino]-4-methyl-phenyl}-3-trifluoromethyl-benzamide, 3-[1-(2-cyclopropylamino-pyrimidin-4-yl)-1H-imidazol-2-ylamino]-4-methyl-N-[4-(2-methyl-imidazol-1-yl)-3-trifluoromethyl-phenyl]-benzamide, N-{3-[1-(6-cyclopropylamino-pyrimidin-4-yl)-1H-imidazol-2-ylamino]-4-methyl-phenyl}-3-piperazin-1-ylmethyl-5-trifluoromethyl-benzamide, N-{3-[1-(6-amino-pyrimidin-4-yl)-1H-imidazol-2-ylamino]-4-methyl-phenyl}-3-piperazin-1-ylmethyl-5-trifluoromethyl-benzamide, N-{3-[1-(6-cyclopropylamino-pyrimidin-4-yl)-1H-imidazol-2-ylamino]-5-methoxy-phenyl}-4-(4-methyl-piperazin-1-ylmethyl)-3-trifluoromethyl-benzamide, N-{3-[1-(6-cyclopropylamino-pyrimidin-4-yl)-1H-imidazol-2-ylamino]-5-methoxy-phenyl}-3-(4-ethyl-piperazin-1-ylmethyl)-5-trifluoromethyl-benzamide, N-{3-[1-(6-cyclopropylamino-pyrimidin-4-yl)-1H-imidazol-2-ylamino]-4-methyl-phenyl}-3-(piperidin-4-yloxy)-5-trifluoromethyl-benzamide, N-({3-[1-(6-amino-pyrimidin-4-yl)-1H-imidazol-2-ylamino]-4-methyl-phenyl}-3-(piperidin-4-yloxy)-5-trifluoromethyl-benzamide, N-{3-[1-(6-cyclopropylamino-pyrimidin-4-yl)-1H-imidazol-2-ylamino]-5-methoxy-phenyl}-3-(4-pyrrolidin-1-yl-piperidin-1-yl)-5-trifluoromethyl-benzamide, 1-{3-[1-(6-cyclopropylamino-pyrimidin-4-yl)-1H-imidazol-2-ylamino]-4-methyl-phenyl}-3-[4-(2-methyl-imidazol-1-yl)-3-trifluoromethyl-phenyl]-urea, 1-{3-[1-(6-cyclopropylamino-pyrimidin-4-yl)-1H-imidazol-2-ylamino]-4-methyl-phenyl}-3-[4-(4-ethyl-piperazin-1-ylmethyl)-3-trifluoromethyl-phenyl]-urea, pyridazine-4-carboxylic acid {3-[1-(6-cyclopropylamino-pyrimidin-4-yl)-1H-imidazol-2-ylamino]-4-methyl-phenyl}-amide, 2-chloro-N-{3-[1-(6-cyclopropylamino-pyrimidin-4-yl)-1H-imidazol-2-ylamino]-4-methyl-phenyl}-isonicotinamide, furan-2-carboxylic acid {3-[1-(6-cyclopropylamino-pyrimidin-4-yl)-1H-imidazol-2-ylamino]-4-methyl-phenyl}-amide, 1-methyl-1H-pyrrole-2-carboxylic acid {3-[1-(6-cyclopropylamino-pyrimidin-4-yl)-1H-imidazol-2-ylamino]-4-methyl-phenyl}-amide, 1H-imidazole-2-carboxylic acid {3-[1-(6-cyclopropylamino-pyrimidin-4-yl)-1H-imidazol-2-ylamino]-4-methyl-phenyl}-amide, N-{3-[1-(6-cyclopropylamino-pyrimidin-4-yl)-1H-imidazol-2-ylamino]-4-methyl-phenyl}-4-trifluoromethyl-benzamide, N-{3-[1-(6-cyclopropylamino-pyrimidin-4-yl)-1H-imidazol-2-ylamino]-4-methyl-phenyl}-6-methyl-nicotinamide, 1-tert-butyl-5-methyl-1H-pyrazole-3-carboxylic acid {3-[1-(6-cyclopropylamino-pyrimidin-4-yl)-1H-imidazol-2-ylamino]-4-methyl-phenyl}-amide, N-[4-(4-ethyl-piperazin-1-ylmethyl)-3-trifluoromethyl-phenyl]-4-methyl-3-{1-[6-(2-morpholin-4-yl-ethylamino)-pyrimidin-4-yl]-1H-imidazol-2-ylamino}-benzamide, 3-(4-methyl-imidazol-1-yl)-N-(4-methyl-3-{1-[6-(2-morpholin-4-yl-ethylamino)-pyrimidin-4-yl]-1H-imidazol-2-ylamino}-phenyl)-5-trifluoromethyl-benzamide, 4-methyl-N-[4-(2-methyl-imidazol-1-yl)-3-trifluoromethyl-phenyl]-3-{1-[6-(2-morpholin-4-yl-ethylamino)-pyrimidin-4-yl]-1H-imidazol-2-ylamino}-benzamide, N-(4-methyl-3-{1-[6-(2-morpholin-4-yl-ethylamino)-pyrimidin-4-yl]-1H-imidazol-2-ylamino}-phenyl)-3-(1-methyl-piperidin-4-yloxy)-5-trifluoromethyl-benzamide, pyrazine-2-carboxylic acid {3-[1-(6-cyclopropylamino-pyrimidin-4-yl)-1H-imidazol-2-ylamino]-4-methyl-phenyl}-amide, 5-methyl-pyrazine-2-carboxylic acid {3-[1-(6-cyclopropylamino-pyrimidin-4-yl)-1H-imidazol-2-ylamino]-4-methyl-phenyl}-amide, quinoxaline-6-carboxylic acid {3-[1-(6-cyclopropylamino-pyrimidin-4-yl)-1H-imidazol-2-ylamino]-4-methyl-phenyl}-amide, 5-tert-butyl-2-methyl-4-morpholin-4-ylmethyl-furan-3-carboxylic acid {3-[1-(6-cyclopropylamino-pyrimidin-4-yl)-1H-imidazol-2-ylamino]-4-methyl-phenyl}-amide, 5-tert-butyl-2-methyl-2H-pyrazole-3-carboxylic acid {3-[1-(6-cyclopropylamino-pyrimidin-4-yl)-1H-imidazol-2-ylamino]-4-methyl-phenyl}-amide, 2-benzyl-5-tert-butyl-2H-pyrazole-3-carboxylic acid {3-[1-(6-cyclopropylamino-pyrimidin-4-yl)-1H-imidazol-2-ylamino]-4-methyl-phenyl}-amide, N-{3-[1-(6-cyclopropylamino-pyrimidin-4-yl)-1H-imidazol-2-ylamino]-4-methyl-phenyl}-3-(piperidin-4-ylamino)-5-trifluoromethyl-benzamide, N-{3-[1-(6-cyclopropylamino-pyrimidin-4-yl)-1H-imidazol-2-ylamino]-4-methyl-phenyl}-3-(pyrrolidin-2-ylmethoxy)-5-trifluoromethyl-benzamide, 5-tert-butyl-2-methyl-furan-3-carboxylic acid {3-[1-(6-cyclopropylamino-pyrimidin-4-yl)-1H-imidazol-2-ylamino]-4-methyl-phenyl}-amide, 5-tert-butyl-3-methyl-furan-2-carboxylic acid {3-[1-(6-cyclopropylamino-pyrimidin-4-yl)-1H-imidazol-2-ylamino]-4-methyl-phenyl}-amide, 5-tert-butyl-2-diethylamino-furan-3-carboxylic acid {3-[1-(6-cyclopropylamino-pyrimidin-4-yl)-1H-imidazol-2-ylamino]-4-methyl-phenyl}-amide, 5-tert-butyl-4-diethylamino-2-methyl-furan-3-carboxylic acid {3-[1-(6-cyclopropylamino-pyrimidin-4-yl)-1H-imidazol-2-ylamino]-4-methyl-phenyl}-amide, 5-tert-butyl-thiophene-2-carboxylic acid {3-[1-(6-cyclopropylamino-pyrimidin-4-yl)-1H-imidazol-2-ylamino]-4-methyl-phenyl}-amide, 5-tert-butyl-3-methyl-furan-2-carboxylic acid (4-methyl-3-{1-[6-(2-morpholin-4-yl-ethylamino)-pyrimidin-4-yl]-1H-imidazol-2-ylamino}-phenyl)-amide, 5-tert-butyl-thiophene-2-carboxylic acid (4-methyl-3-{1-[6-(2-morpholin-4-yl-ethylamino)-pyrimidin-4-yl]-1H-imidazol-2-ylamino}-phenyl)-amide, 5-tert-butyl-2-methyl-furan-3-carboxylic acid (4-methyl-3-{1-[6-(2-morpholin-4-yl-ethylamino)-pyrimidin-4-yl]-1H-imidazol-2-ylamino}-phenyl)-amide, N-(3-{1-[6-(4-amino-cyclohexylamino)-pyrimidin-4-yl]-1H-imidazol-2-ylamino}-4-methyl-phenyl)-3-trifluoromethyl-benzamide, N-(4-methyl-3-{1-[6-(piperidin-4-ylamino)-pyrimidin-4-yl]-1H-imidazol-2-ylamino}-phenyl)-3-trifluoromethyl-benzamide, N-[4-methyl-3-(1-{6-[2-(4-methyl-piperazin-1-yl)-ethylamino]-pyrimidin-4-yl}-1H-imidazol-2-ylamino)-phenyl]-3-trifluoromethyl-benzamide, N-(3-{(1-[6-(2,5-dimethyl-2H-pyrazol-3-ylamino)-pyrimidin-4-yl]-1H-imidazol-2-ylamino}-4-methyl-phenyl)-3-trifluoromethyl-benzamide, N-(4-methyl-3-{1-[6-(4-methyl-piperazin-1-ylamino)-pyrimidin-4-yl]-1H-imidazol-2-ylamino}-phenyl)-3-trifluoromethyl-benzamide, N-(4-methyl-3-{1-[6-(2-methyl-2H-pyrazol-3-ylamino)-pyrimidin-4-yl]-1H-imidazol-2-ylamino}-phenyl)-3-trifluoromethyl-benzamide, N-(3-{1-[6-(2,6-dimethyl-pyridin-3-ylamino)-pyrimidin-4-yl]-1-imidazol-2-ylamino}-4-methyl-phenyl)-3-trifluromethyl-benzamide, N-(4-methyl-3-{1-[6-(2-methyl-pyridin-3-ylamino)-pyrimidin-4-yl]-1H-imidazol-2-ylamino}-phenyl)-3-trifluoromethyl-benzamide, N-{3-[1-(6-cyclopropylamino-pyrimidin-4-yl)-1H-imidazol-2-ylamino]-4-methyl-phenyl}-3-(4-methyl-piperazin-1-yl)-5-trifluoromethyl-benzamide, N-{3-[1-(6-cyclopropylamino-pyrimidin-4-yl)-1H-imidazol-2-ylamino]-4-methyl-phenyl}-3-[4-(2-hydroxy-ethyl)-piperazin-1-yl]-5-trifluoromethyl-benzamide, N-{3-[1-(6-cyclopropylamino-pyrimidin-4-yl)-1H-imidazol-2-ylamino]-4-methyl-phenyl}-3-piperazin-1-yl-5-trifluoromethyl-benzamide, N-{4-chloro-3-[1-(6-cyclopropylamino-pyrimidin-4-yl)-1H-imidazol-2-ylamino]-phenyl}-benzamide, N-{3-[1-(6-cyclopropylamino-pyrimidin-4-yl)-1H-imidazol-2-ylamino]-4-fluoro-phenyl}-benzamide, N-{3-[1-(6-cyclopropylamino-pyrimidin-4-yl)-1H-imidazol-2-ylamino]-2-methyl-phenyl}-benzamide, N-{5-[1-(6-cyclopropylamino-pyrimidin-4-yl)-1H-imidazol-2-ylamino]-2-fluoro-phenyl}-benzamide, N-{5-[1-(6-cyclopropylamino-pyrimidin-4-yl)-1H-imidazol-2-ylamino]-2-methyl-phenyl}-benzamide, N-{4-chloro-3-[1-(6-cyclopropylamino-pyrimidin-4-yl)-1H-imidazol-2-ylamino]-phenyl}-4-morpholin-4-ylmethyl-3-trifluoromethyl-benzamide, N-{3-[1-(6-cyclopropylamino-pyrimidin-4-yl)-1H-imidazol-2-ylamino]-4-fluoro-phenyl}-4-morpholin-4-ylmethyl-3-trifluoromethyl-benzamide, N-{3-[1-(6-cyclopropylamino-pyrimidin-4-yl)-1H-imidazol-2-ylamino]-2-methyl-phenyl}-4-morpholin-4-ylmethyl-3-trifluoromethyl-benzamide, N-{4-chloro-3-[1-(6-cyclopropylamino-pyrimidin-4-yl)-1H-imidazol-2-ylamino]-phenyl}-3-(4-methyl-imidazol-1-yl)-5-trifluoromethyl-benzamide, N-{3-[1-(6-cyclopropylamino-pyrimidin-4-yl)-1H-imidazol-2-ylamino]-4-fluoro-phenyl}-3-(4-methyl-imidazol-1-yl)-5-trifluoromethyl-benzamide, N-{3-[1-(6-cyclopropylamino-pyrimidin-4-yl)-1H-imidazol-2-ylamino]-2-methyl-phenyl}-3-(4-methyl-imidazol-1-yl)-5-trifluoromethyl-benzamide, N-{5-[1-(6-cyclopropylamino-pyrimidin-4-yl)-1H-imidazol-2-ylamino]-2-fluoro-phenyl}-3-(4-methyl-imidazol-1-yl)-5-trifluoromethyl-benzamide, N-{5-[1-(6-cyclopropylamino-pyrimidin-4-yl)-1H-imidazol-2-ylamino]-2-methyl-phenyl}-3-(4-methyl-imidazol-1-yl)-5-trifluoromethyl-benzamide, N-{3-[1-(6-cyclopropylamino-pyrimidin-4-yl)-1H-imidazol-2-ylamino]-4-methoxy-phenyl}-benzamide, N-{3-[1-(6-cyclopropylamino-pyrimidin-4-yl)-1H-imidazol-2-ylamino]-4-methyl-phenyl}-3-trifluoromethyl-benzenesulfonamide, 3-[1-(6-cyclopropylamino-pyrimidin-4-yl)-1H-imidazol-2-ylamino]-4-methyl-N-(3-trifluoromethyl-phenyl)-benzenesulfonamide, N-{3-[1-(6-cyclopropylamino-pyrimidin-4-yl)-1H-imidazol-2-ylamino]-4-fluoro-phenyl}-3-dimethylamino-5-trifluoromethyl-benzamide, N-{5-[1-(6-cyclopropylamino-pyrimidin-4-yl)-1H-imidazol-2-ylamino]-2-methyl-phenyl}-3-dimethylamino-5-trifluoromethyl-benzamide, N-{4-chloro-3-[1-(6-cyclopropylamino-pyrimidin-4-yl)-1H-imidazol-2-ylamino]-phenyl}-3-dimethylamino-5-trifluoromethyl-benzamide, 3-(4-methyl-imidazol-1-yl)-N-(4-methyl-3-{1-[6-(pyridin-2-ylamino)-pyrimidin-4-yl]-1H-imidazol-2-ylamino}-phenyl)-5-trifluoromethyl-benzamide, 4-(4-ethyl-piperazin-1-ylmethyl)-N-(4-methyl-3-{1-[6-(pyridin-2-ylamino)-pyrimidin-4-yl]-1H-imidazol-2-ylamino}-phenyl)-3-trifluoromethyl-benzamide, 3-(4-ethyl-piperazin-1-yl)-N-(4-methyl-3-{1-[6-(pyridin-2-ylamino)-pyrimidin-4-yl]-1H-imidazol-2-ylamino}-phenyl)-5-trifluoromethyl-benzamide, 4-chloro-N-(4-methyl-3-{1-[6-(pyridin-2-ylamino)-pyrimidin-4-yl]-1H-imidazol-2-ylamino}-phenyl)-3-trifluoromethyl-benzamide, N-[3-(1-{6-[5-(4-ethyl-piperazin-1-yl)-pyridin-2-ylamino]-pyrimidin-4-yl}-1H-imidazol-2-ylamino)-4-methyl-phenyl]-3-trifluoromethyl-benzamide, N-(4-methyl-3-{1-[6-(4-methyl-pyridin-2-ylamino)-pyrimidin-4-yl]-1H-imidazol-2-ylamino}-phenyl)-3-trifluoromethyl-benzamide, N-(3-{1-[6-(4,6-dimethyl-pyridin-2-ylamino)-pyrimidin-4-yl]-1H-imidazol-2-ylamino}-4-methyl-phenyl)-3-trifluoromethyl-benzamide, 6-(6-{2-[2-methyl-5-(3-trifluoromethyl-benzoylamino)-phenylamino]-imidazol-1-yl}-pyrimidin-4-ylamino)-nicotinamide, N-(4-methyl-3-{1-[6-(5-methyl-pyridin-2-ylamino)-pyrimidin-4-yl]-1H-imidazol-2-ylamino}-phenyl)-3-trifluoromethyl-benzamide, N-(3-{1-[6-(5-cyano-pyridin-2-ylamino)-pyrimidin-4-yl]-1H-imidazol-2-ylamino}-4-methyl-phenyl)-3-trifluoromethyl-benzamide, 4-chloro-N-{3-[1-(6-cyclopropylamino-pyrimidin-4-yl)-1H-imidazol-2-ylamino]-4-methyl-phenyl}-3-trifluoromethyl-benzamide, 4-chloro-N-(4-methyl-3-{1-[6-(2-morpholin-4-yl-ethylamino)-pyrimidin-4-yl]-1H-imidazol-2-ylamino}-phenyl)-3-trifluoromethyl-benzamide, 5-tert-butyl-2-methyl-2H-pyrazole-3-carboxylic acid (4-methyl-3-{1-[6-(2-morpholin-4-yl-ethylamino)-pyrimidin-4-yl]-1H-imidazol-2-ylamino}-phenyl)-amide, N-(3-{1-[6-(2-dimethylamino-ethylamino)-pyrimidin-4-yl]-1H-imidazol-2-ylamino}-4-methyl-phenyl)-3-trifluoromethyl-benzamide, N-(4-methyl-3-{1-[6-(3-morpholin-4-yl-propylamino)-pyrimidin-4-yl]-1H-imidazol-2-ylamino}-phenyl)-3-trifluoromethyl-benzamide, N-(3-{1-[6-(2-methoxy-ethylamino)-pyrimidin-4-yl]-1H-imidazol-2-ylamino}-4-methyl-phenyl)-3-trifluoromethyl-benzamide, N-[4-methyl-3-(1-{6-[2-(1-methyl-pyrrolidin-2-yl)-ethylamino]-pyrimidin-4-yl}-1H-imidazol-2-ylamino)-phenyl]-3-trifluoromethyl-benzamide, N-(4-methyl-3-{1-[6-(pyridin-2-ylamino)-pyrimidin-4-yl]-1H-imidazol-2-ylamino}-phenyl)-3-trifluoromethyl-benzamide, N-[4-methyl-3-(1-pyrimidin-4-yl-1H-imidazol-2-ylamino)-phenyl]-3-trifluoromethyl-benzamide, N-[4-methyl-3-(1-pyrimidin-2-yl-1H-imidazol-2-ylamino)-phenyl]-3-trifluoromethyl-benzamide, N-{4-methyl-3-[1-(4-methylamino-pyrimidin-2-yl)-1H-imidazol-2-ylamino]-phenyl}-3-trifluoromethyl-benzamide, N-{4-methyl-3-[1-(2-methylamino-pyrimidin-4-yl)-1H-imidazol-2-ylamino]-phenyl}-3-trifluoromethyl-benzamide, N-(4-methyl-3-{1-[4-(2-morpholin-4-yl-ethylamino)-pyrimidin-2-yl]-1H-imidazol-2-ylamino}-phenyl)-3-trifluoromethyl-benzamide, N-(4-methyl-3-{1-[2-(2-morpholin-4-yl-ethylamino)-pyrimidin-4-yl]-1H-imidazol-2-ylamino}-phenyl)-3-trifluoromethyl-benzamide, N-(3-{1-[6-(2-ethyl-2H-pyrazol-3-ylamino)-pyrimidin-4-yl]-1H-imidazol-2-ylamino}-4-methyl-phenyl)-3-trifluoromethyl-benzamide, N-(3-{1-[6-(3-dimethylamino-propylamino)-pyrimidin-4-yl]-1H-imidazol-2-ylamino}-4-methyl-phenyl)-3-trifluoromethyl-benzamide, N-{3-[1-(6-isopropylamino-pyrimidin-4-yl)-1H-imidazol-2-ylamino]-4-methyl-phenyl}-3-trifluoromethyl-benzamide, N-[3-(1-{6-[(furan-3-ylmethyl)-amino]-pyrimidin-4-yl}-1H-imidazol-2-ylamino)-4-methyl-phenyl]-3-trifluoromethyl-benzamide, N-[4-methyl-3-(1-{6-[3-(4-methyl-piperazin-1-yl)-propylamino]-pyrimidin-4-yl}-1H-imidazol-2-ylamino)-phenyl]-3-trifluoromethyl-benzamide, N-[3-(1-{6-[(benzo[1,3]dioxol-5-ylmethyl)-amino]-pyrimidin-4-yl}-1H-imidazol-2-ylamino)-4-methyl-phenyl]-3-trifluoromethyl-benzamide, N-(4-methyl-3-{1-[6-(2-methyl-6-morpholin-4-yl-pyridin-3-ylamino)-pyrimidin-4-yl]-1H-imidazol-2-ylamino}-phenyl)-3-trifluoromethyl-benzamide, N-(4-methyl-3-{1-[6-(4-methyl-pyrimidin-2-ylamino)-pyrimidin-4-yl]-1H-imidazol-2-ylamino}-phenyl)-3-trifluoromethyl-benzamide, N-(3-{1-[6-(6-methoxy-pyridin-3-ylamino)-pyrimidin-4-yl]-1H-imidazol-2-ylamino}-4-methyl-phenyl)-3-trifluoromethyl-benzamide, N-{4-methyl-3-[1-(6-morpholin-4-yl-pyrimidin-4-yl)-1H-imidazol-2-ylamino]-phenyl}-3-trifluoromethyl-benzamide, N-(3-{1-[6-(4-ethyl-piperazin-1-yl)-pyrimidin-4-yl]-1H-imidazol-2-ylamino}-4-methyl-phenyl)-3-trifluoromethyl-benzamide, N-(3-{1-[6-(3-fluoro-phenylamino)-pyrimidin-4-yl]-1H-imidazol-2-ylamino}-4-methyl-phenyl)-3-trifluoromethyl-benzamide, N-(3-{1-[6-(3-dimethylamino-ethyl-formamide-phenylamino)-pyrimidin-4-yl]-1H-imidazol-2-ylamino}-4-methyl-phenyl)-3-trifluoromethyl-benzamide, N-[4-methyl-3-(1-{6-[(pyridin-3-ylmethyl)-amino]-pyrimidin-4-yl}-1H-imidazol-2-ylamino)-phenyl]-3-trifluoromethyl-benzamide, N-[4-methyl-3-(1-{6-[(pyridin-4-ylmethyl)-amino]-pyrimidin-4-yl}-1H-imidazol-2-ylamino)-phenyl]-3-trifluoromethyl-benzamide, N-(4-methyl-3-{1-[6-(4-morpholin-4-yl-piperidin-1-yl)-pyrimidin-4-yl]-1H-imidazol-2-ylamino}-phenyl)-3-trifluoromethyl-benzamide, N-[4-methyl-3-(1-{6-[(thiazol-2-ylmethyl)-amino]-pyrimidin-4-yl}-1H-imidazol-2-ylamino)-phenyl]-3-trifluoromethyl-benzamide, N-[4-methyl-3-(1-{6-[(pyridin-2-ylmethyl)-amino]-pyrimidin-4-yl}-1H-imidazol-2-ylamino)-phenyl]-3-trifluoromethyl-benzamide, N-[4-methyl-3-(1-{6-[(6-methyl-pyrazin-2-ylmethyl)-amino]-pyrimidin-4-yl}-1H-imidazol-2-ylamino)-phenyl]-3-trifluoromethyl-benzamide, N-(4-methyl-3-{1-[6-(4-pyridin-2-yl-piperazin-1-yl)-pyrimidin-4-yl]-1H-imidazol-2-ylamino}-phenyl)-3-trifluoromethyl-benzamide, N-(3-{1-[6-(3-imidazol-1-yl-propylamino)-pyrimidin-4-yl]-1H-imidazol-2-ylamino}-4-methyl-phenyl)-3-trifluoromethyl-benzamide, N-(4-methyl-3-{1-[6-(pyrazin-2-ylamino)-pyrimidin-4-yl]-1H-imidazol-2-ylamino}-phenyl)-3-trifluoromethyl-benzamide, N-(3-{1-[6-(3-formamide-phenylamino)-pyrimidin-4-yl]-1H-imidazol-2-ylamino}-4-methyl-phenyl)-3-trifluoromethyl-benzamide, 4-methyl-3-{1-[6-(2-morpholin-4-yl-ethylamino)-pyrimidin-4-yl]-1H-imidazol-2-ylamino}-N-(3-trifluoromethyl-phenyl)-benzamide, 4-methyl-3-{1-[6-(4-methyl-piperazin-1-ylamino)-pyrimidin-4-yl]-1H-imidazol-2-ylamino}-N-(3-trifluoromethyl-phenyl)-benzamide, N-(4-chloro-3-trifluoromethyl-phenyl)-4-methyl-3-{1-[6-(2-morpholin-4-yl-ethylamino)-pyrimidin-4-yl]-1H-imidazol-2-ylamino}-benzamide, N-(4-chloro-3-trifluoromethyl-phenyl)-4-methyl-3-{1-[6-(4-methyl-piperazin-1-ylamino)-pyrimidin-4-yl]-1H-imidazol-2-ylamino}-benzamide, 1-tert-butyl-5-(4-methyl-piperazin-1-ylmethyl)-1H-pyrazole-3-carboxylic acid {3-[1-(6-cyclopropylamino-pyrimidin-4-yl)-1H-imidazol-2-ylamino]-4-methyl-phenyl}-amide, 1-tert butyl-5-morpholin-4-ylmethyl-1H-pyrazole-3-carboxylic acid {3-[1-(6-cyclopropylamino-pyrimidin-4-yl)-1H-imidazol-2-ylamino]-4-methyl-phenyl}-amide, N-(4-tert-butyl-thiazol-2-yl)-4-methyl-3-{1-[6-(2-morpholin-4-yl-ethylamino)-pyrimidin-4-yl]-1H-imidazol-2-ylamino}-benzamide, N-(4-tert-butyl-thiazol-2-yl)-4-methyl-3-{1-[6-(4-methyl-piperazin-1-ylamino)-pyrimidin-4-yl]-1H-imidazol-2-ylamino}-benzamide, N-(5-tert-butyl-2-methyl-2H-pyrazol-3-yl)-4-methyl-3-{1-[6-(4-methyl-piperazin-1-ylamino)-pyrimidin-4-yl]-1H-imidazol-2-ylamino}-benzamide, N-(4-methyl-3-{1-[6-(4-methyl-piperazin-1-ylamino)-pyrimidin-4-yl]-1H-imidazol-2-ylamino}-phenyl)-benzamide, N-(5-tert-butyl-2-methyl-2H-pyrazol-3-yl)-4-methyl-3-{1-[6-(2-morpholin-4-yl-ethylamino)-pyrimidin-4-yl]-1H-imidazol-2-ylamino}-benzamide, N-(4-methyl-3-{1-[2-(2-morpholin-4-yl-ethylamino)-pyridin-4-yl]-1H-imidazol-2-ylamino}-phenyl)-3-trifluoromethyl-benzamide, N-{3-[1-(4-acetylamino-pyridin-2-yl)-1H-imidazol-2-ylamino]-4-methyl-phenyl}-3-trifluoromethyl-benzamide and 2-{2-[2-Methyl-5-(3-trifluoromethyl-benzoylamino)-phenylamino]-imidazol-1-yl}-isonicotinamide.

In some embodiments, a MKK4 inhibitor comprises a TAK1 inhibitor describes herein. In some embodiments, a MKK4 inhibitor comprises myricetin (Brand et al. 2010) or an analog or derivative thereof.

In some embodiments, a MKK4 inhibitor inhibits/decreases the activation of the HGK-Tak1-MKK4-JNK-c-Jun cell death signaling cascade by at least 5%, 10%, 20%, 30%, 40%, 50%, 60%, 70%, 80%, 90%, or 95% relative to a control. While not required, a MKK4 inhibitor can completely inhibit activation of the HGK-Tak1-MKK4-JNK-c-Jun cell death signaling cascade relative to a control.

In some embodiments, an agent that decreases activation of the HGK-Tak1-MKK4-JNK-c-Jun cell death signaling cascade is not an MKK4 inhibitor (e.g., an inhibitor that is selective and/or specific for, and/or directly inhibits MKK4). In some embodiments, an agent that decreases activation of the HGK-Tak1-MKK4-JNK-c-Jun cell death signaling cascade is not an MKK4 inhibitor described herein.

In some embodiments, the protein kinase is INK. In some embodiments, a INK inhibitor can be used as a HGK-Tak1-MKK4-JNK-c-Jun cell death signaling cascade inhibitor.

JNK inhibitors can be small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

Exemplary INK inhibitors include, but are not limited to, the JNK inhibitors of formula I as described in U.S. Pat. No. 8,183,254; the benzothiazole derivatives according to formula I and tautomers, geometrical isomers, enantiomers, diastereomers, racemates and pharmaceutically acceptable salts thereof described in U.S. Patent Application Publication No. 2009/0176762; a JNK inhibitor sequence, chimeric peptide, or nucleic acid described in U.S. Pat. No. 6,610,820 and U.S. Patent Application Publication No. 2009/0305968, for example cell-permeable peptides that bind to JNK and inhibit JNK activity or c-Jun activation, including peptides having amino acid sequences of DTYRPKRPTT LNLFPQVPRS QDT (SEQ ID NO: 1); EEPHKHRPTT LRLTTLGAQD S (SEQ ID NO:2); TDQSRPVQPF LNLTTPRKPR YTD (SEQ ID NO:3); or SDQAGLTLR LTTPRHKHPE E (SEQ ID NO:4); c-Jun N-terminal kinase inhibiting compounds of formula I described in U.S. Pat. No. 7,612,086; a JNK inhibitor of formula I described in PCT International Application Publication No. WO/2011/018417; an agent that inhibits MKK4 as described herein (e.g., MKK4 inhibitors); an agent that inhibits JNK interacting protein (JIP) (see for example, Chen T, et al. Biochem J. 2009 May 13; 420(2):283-94, which is incorporated by reference, discloses small-molecules that disrupt the JIP-JNK interaction to provide an alternative approach for JNK inhibition); SP600125 (Anthra[1,9-cd]pyrazol-6(2H)-on; 1,9-Pyrazoloanthrone) (Calbiochem., La Jolla, Calif.); a compound based on the 6,7-dihydro-5H-pyrrolo[1,2-a]imidazole scaffold (e.g., ER-181304); SB203580; a selective inhibitor of JNK3 described in PCT International Application Publication WO 2010/039647; 7-(5-7V-Phenylaminopentyl)-2H-anthra[1,9-cd]pyrazol-6-one; 7-(7-7V-Benzoylaminoheptyl)amino-2H-anthra[1,9-cd]pyrazol-6-one; and 7-(5-(p-Tolyloxy)pentyl)amino-2H-anthra[1,9-cd]pyrazol-6-one; a dominant negative form of INK, e.g., a catalytically inactive JNK-1 molecule constructed by replacing the sites of activating Thr183 and Tyr185 phosphorylation with Ala and Phe respectively, which acts as a dominant inhibitor of the wild-type JNK-1 molecule as described in PCT International Application Publication No. WO 1996/036642; a JIP-1 polypeptide that binds JNK as described in U.S. Patent Publications 2007/0003517 and 2002/0119135, including a peptide having the amino acid sequence SGDTYRPKRPTTLNLFPQVPRSQDTLN (SEQ ID NO: 12).

In some embodiments, a JNK inhibitor inhibits/decreases the activation of the HGK-Tak1-MKK4-JNK-c-Jun cell death signaling cascade by at least 5%, 10%, 20%, 30%, 40%, 50%, 60%, 70%, 80%, 90%, or 95% relative to a control. While not required, a JNK inhibitor can completely inhibit activation of the HGK-Tak1-MKK4-JNK-c-Jun cell death signaling cascade relative to a control.

In some embodiments, an agent that decreases activation of the HGK-Tak1-MKK4-JNK-c-Jun cell death signaling cascade is not a JNK inhibitor (e.g., an inhibitor that is selective and/or specific for, and/or directly inhibits JNK). In some embodiments, an agent that decreases activation of the HGK-Tak1-MKK4-JNK-c-Jun cell death signaling cascade is not a JNK inhibitor described herein.

In some embodiments, a TNF-α inhibitor as described herein can be used as a HGK-Tak1-MKK4-JNK-c-Jun cell death signaling cascade inhibitor.

In some embodiments, an agent that decreases activation of the HGK-Tak1-MKK4-JNK-c-Jun cell death signaling cascade is not a TNF-α inhibitor.

In some embodiments, an agent that decreases activation of the HGK-Tak1-MKK4-JNK-c-Jun cell death signaling cascade is an inhibitor of glycogen synthase kinase 3 (GSK-3). In some embodiments, an agent that decreases activation of the HGK-Tak1-MKK4-JNK-c-Jun cell death signaling cascade inhibits GSK-3. In some embodiments, an agent that decreases activation of the HGK-Tak1-MKK4-JNK-c-Jun cell death signaling cascade is an agent that inhibits GSK-3 as described herein. In some embodiments, an agent that decreases activation of the HGK-Tak1-MKK4-JNK-c-Jun cell death signaling cascade exhibits dual inhibition of GSK-3 and HGK. In some embodiments, a method of promoting motor neuron survival further includes contacting the motor neuron with an additional agent that inhibits GSK-3 as described herein.

Without limitations, the agent that decreases activation of the HGK-Tak1-MKK4-JNK-c-Jun cell death signaling cascade and the additional agent can be used in any ratio. Accordingly, in some embodiments, the agent that decreases activation of the HGK-Tak1-MKK4-JNK-c-Jun cell death signaling cascade and the additional agent are in a ratio from 20:1 to 1:20. In some embodiments, the agent that decreases activation of the HGK-Tak1-MKK4-JNK-c-Jun cell death signaling cascade and the additional agent are in ratio from 10:1 to 1:10. In some embodiments, the agent that decreases activation of the HGK-Tak1-MKK4-JNK-c-Jun cell death signaling cascade and the additional agent are in ratio from 5:1 to 1:5. In some embodiments, the agent that decreases activation of the HGK-Tak1-MKK4-JNK-c-Jun cell death signaling cascade and the additional agent are in a ratio from 15:1 to 1:5. In some embodiments, the agent that decreases activation of the HGK-Tak1-MKK4-JNK-c-Jun cell death signaling cascade and the additional agent are in a ratio from 10:1 to 1:1. In one embodiment, the agent that decreases activation of the HGK-Tak1-MKK4-JNK-c-Jun cell death signaling cascade and the additional agent are used in a 1:1 ratio.

In one aspect, disclosed herein is a method of promoting motor neuron survival, comprising contacting a motor neuron with an effective amount of a compound selected from the group consisting of ethaverine hydrochloride, crinamine, gedunin, pomiferin, dactinomycin, 3-alpha-hydroxydeoxygedinin, totarol, totaral acetate, ginkgetin potassium salt, hygromycin B, blasticidin S, lynestrenol, hippeastrine hydrobromide, and combinations thereof.

In some embodiments, a method of promoting motor neuron survival comprises contacting a motor neuron with an effective amount of a compound selected from the compounds listed in Table 2 below.

TABLE 2
Compounds promoting survival of HB9::GFP and SOD1^{G93A}/HB9::GFP motor neurons

| | Compound name | CAS number | Molecular Structure |
|---|---|---|---|
| 1 | CP55940 | 83002-04-4 | 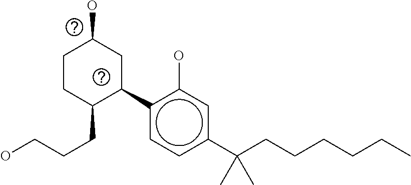 |
| 2 | FPL-64176 | 120934-96-5 | 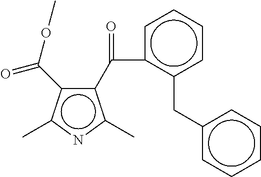 |
| 3 | Kenpaulione | 142273-20-9 | 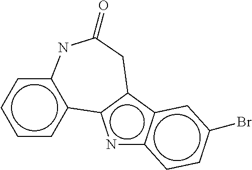 |
| 4 | Quinacrine dihydrochloride dihydrate | 6151-30-0 | 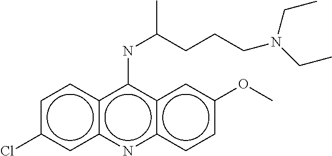 |
| 5 | 1,10-Phenanthroline monohydrate | 5144-89-8 | 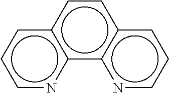 |
| 6 | 3-Tropanylindole-3- carboxylate methiodide | 89565-68-4 | 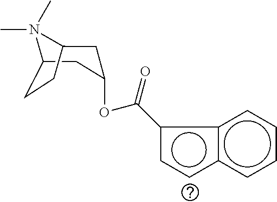 |
| 7 | Ethaverine hydrochloride | 985-13-7 | 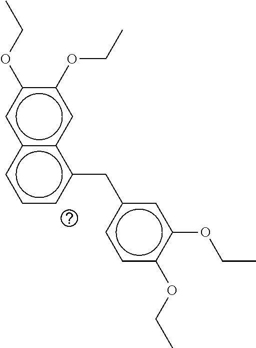 |
| 8 | Cycloheximide | 66-81-9 | 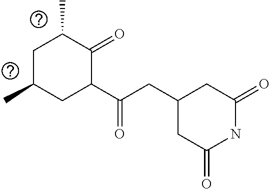 [SMILES: C[C@@H]1CC(C(=O)CC2CC(=O)NC(=O)C2)C(=O)[C@@H](C)C1] |
| 9 | Anisomycin | 22862-76-6 | 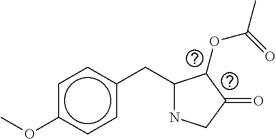 |
| 10 | Crinamine | 639-41-8 | 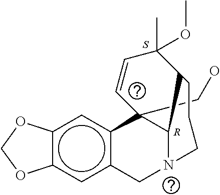 |
| 11 | Gedunin | 2753-30-2 | 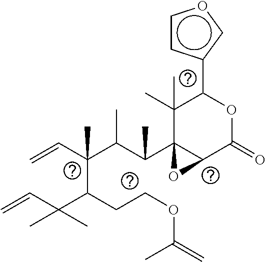 |
| 12 | Pomiferin | 572-03-2 | 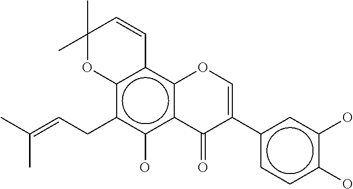 |
| 13 | Dactinomycin | 50-76-0 | 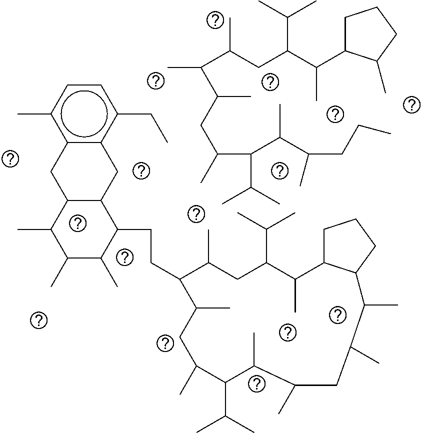 |
| 14 | 3-alpha-hydroxydeoxygedinin | 30437-71-9 | 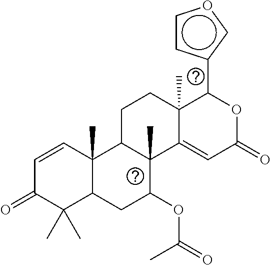 |
| 15 | Totarol acetate | 15340-82-6 | 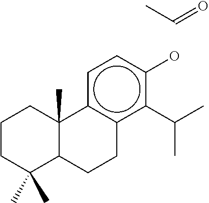 |
| 16 | Totarol | 511-15-9 | 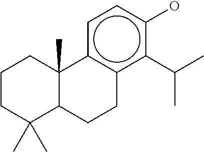 |
| 17 | Deacetoxy-7-oxisogedunin | 5506-92-3 | 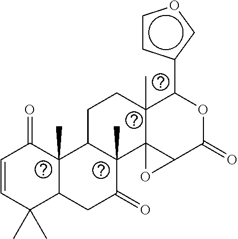 |
| 18 | Ginkgetin K salt | 481-46-9 | 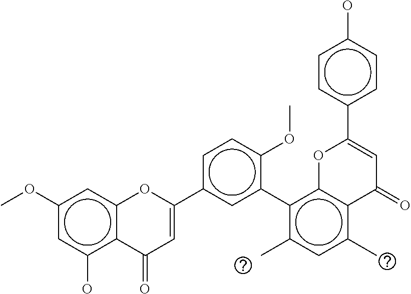 |
| 19 | Hygromycin B | 31282-04-9 | 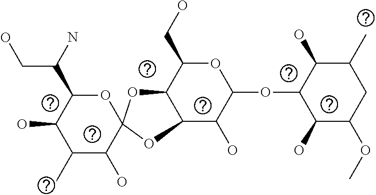 |
| 20 | Blasticidin S | 2079-00-7 | 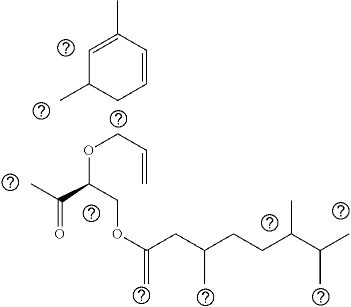 |
| 21 | Lynestrenol | 52-76-6 | 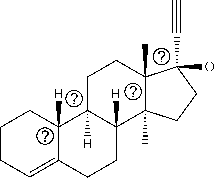 |
| 22 | Hippeastrine hydrobromide | 22352-41-6 | 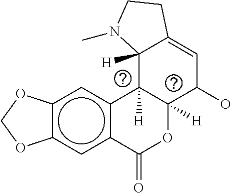 |

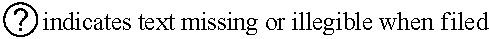

The work described herein also demonstrates, inter alia, that while compounds of formula (I), e.g., purvalanol A, generally inhibits motor neuron survival when used alone, it can promote motor neuron survival in a synergistic manner when used together with a compound that modulates a biological pathway or target that is different from the one that is modulated by a compound of formula (I).

Accordingly, in one aspect, disclosed herein is a method of promoting motor neuron survival, the method comprising contacting a motor neuron with a compound of formula (I):

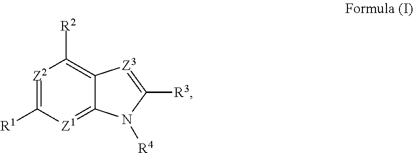

and an additional agent that modulates a biological pathway or a target described herein.

Compounds of formula (I) include analogues, derivatives, prodrugs, and pharmaceutically acceptable salts of the compounds.

In compounds of formula (I), each of Z¹, Z² and Z³ can be independently N or CH₂. In some embodiments, only one of Z¹, Z² and Z² is N, i.e., only Z¹, Z¹ or Z³ is N. In some other embodiments, two of Z¹, Z² and Z³ is N, i.e., Z¹ and Z², Z¹ and Z² or Z² and Z³ are N. In some embodiments, each of Z¹, Z², and Z³ are N.

In compounds of formula (I), R¹ can be hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂R⁹, C(O)R⁹, SO₂, CN, CF₃, or SO₂. In some embodiments, R¹ can be an optionally substituted linear or branched C1-C6 alkyl, e.g., methyl, ethyl, propyl, isopropyl, butyl, pentyl, hexyl, i-alkyl-butyl, 2-alkyl-butyl, 3-alkyl-butyl. In some embodiments, R¹ can be a substituted linear or branched C1-C6 alkyl. In some embodiments, R¹ can be an optionally substituted linear or branched C1-C6 alkynyl. In some embodiment, R¹ is 3-hydroxy, 3-methyl-pentynyl. In some embodiments, R¹ is NR⁵R⁶.

In compounds of formula (I), R² can be hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂R⁹, C(O)R⁹, SO₂, CN, CF₃, or SO₂. In some embodiments, R² can be an optionally substituted alkyl amino. In some embodiments. R² is NR⁷R⁸.

In compounds of formula (I), R³ can be hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂R⁹, C(O)R⁹, SO₂, CN, CF₃, or SO₂. In some embodiments, R³ is H.

In compounds of formula (I), R⁴ can be hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂R⁹, C(O)R⁹, SO₂, CN, CF₃, or SO₂. In some embodiments, R⁴ is an optionally substituted C1-C6. In some embodiments, R⁴ can be selected from H, methyl, ethyl, propyl, isopropyl, butyl, t-butyl, and pentyl.

In compounds of formula (I), R⁵ can be hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂R⁹, C(O)R⁹, SO₂, CN, CF₃, or SO₂. In some embodiments, R⁵ can be an optionally substituted linear or branched C1-C6 alkyl, e.g., methyl, ethyl, propyl, isopropyl, butyl, pentyl, hexyl, 1-alkyl-butyl, 2-alkyl-butyl, 3-alkyl-butyl. In some embodiments, R⁵ can be a substituted linear or branched C1-C6 alkyl. In some embodiments, R⁵ can be a substituted —CH(CH₂OH)CH(CH₃)₂.

In compounds of formula (I), R⁶ can be hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂R⁹, C(O)R⁹, SO₂, CN, CF₃, or SO₂. In some embodiments, R² is H.

In some embodiments, at least one of R⁵ and R⁶ is not H.

In compounds of formula (I), R⁷ can be hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂R⁹, C(O)R⁹, SO₂, CN, CF₃, or SO₂. In some embodiments, R² can be an optionally substituted yl or hetero aryl. When R⁷ is a substituted aryl or heteroaryl group, the aryl or the heteroaryl can be substituted by 1, 2, 3, 4, or 5 substituents. The substitutent can be selected from alkyl, alkenyl, alkynyl, aryl, heteroaryl, cyclyl, heterocyclyl, halogen, amino, alkyl amino, carbonyl, CF₃, CN, SO₂, CO₂H, CF₃, NO₂. In some embodiments, R⁷ is an optionally substituted phenyl group. In some embodiments, R⁷ is a phenyl or benzyl group substituted with a halogen. In some embodiments, R⁷ is an optionally substituted m-halo-phenyl, e.g., an optionally substituted m-chloro-phenyl. In some embodiments, R⁷ is aryl or heteroaryl group substituted with an alkoxy group, e.g., a C1-C6 alkoxy group. When R⁷ is an aryl or heteroaryl substituted with an alkoxy group, the alkoxy group can be methoxy, ethoxy, and the like. In some embodiments, R⁷ can be selected from the group consisting of phenyl; m-chloro-phenyl; p-chloro-phenyl; m-chloro-p-carboxylic-phenyl; p-methoxy-phenyl; p-methoxy-phenyl; m,p-dichloro-phenyl; m,p-dimethoxy-phenyl; p-dimethylemino-phenyl; m,p-OCH₂O-phenyl; benzyl; m-chloro-benzyl; p-chloro-benzyl; m-chloro-p-carboxylic-benzyl; p-methoxy-benzyl; p-methoxy-benzyl; m,p-dichloro-benzyl; m,p-dimethoxy-benzyl; p-dimethylamino-benzyl; and m,p-OCH₂O—.

In compounds of formula (I), R⁸ can be hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂R⁹, C(O)R⁹, SO₂, CN, CF₃, or SO₂. In some embodiments, R⁸ can be H.

In some embodiments, at least one of R⁷ and R⁸ is not H.

In compounds of formula (I) R⁹ can be hydrogen, halogen, optionally substituted linear or branched alkyl, optionally substituted linear or branched alkenyl optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂R⁹, C(O)R⁹, SO₂, CN, CF₃, or SO₂. In some embodiments, R⁹ is H.

In some embodiment, the compound of formula (I) has a structure shown in formula (Ia):

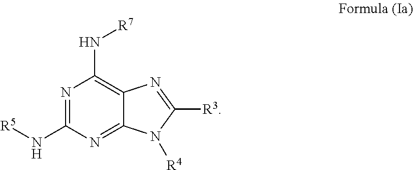

Exemplary compounds of formula (I) include, but are not limited to, (2R)-2-[[6-[(3-Chlorophenyl)amino]-9-(1-methylethyl)-9H-purin-2-yl]amino]-3-methyl-1-butanol (Purvalanol A); (2R)-2-[[6-[(3-Chloro-4-carboxyphenyl)amino]-9-(l-methylethyl)-9H-purin-2-yl]amino]-3-methyl-1-butanol (Purvalanol B), 6-Benzylamino-2[(R)-(1′-ethyl-2′-hydroxyethylamino)]-9-isopropylpurine (roscovitine); olomouscine; N9-isopropyl-olmoucine (NG75); and hymenialdisine. Other compounds of formula (I) are described in U.S. Pat. No. 6,303,618; U.S. Pat. No. 6,627,633; U.S. Pat. No. 6,667,311; U.S. Pat. No. 6,969,720; and U.S. Pat. No. 7,816,350 and U.S. Patent Publication No. 2003/0092909; 2003/0114672; 2003/018726; 2004/0110775; 2010/0143350; and 2011/0269178, content of all which is incorporated herein by reference. Additional compounds of formula (I) are described in Legraverend et al. (J. Med. Chem., 2000, 43(7): 1282-1292), content of which is incorporated herein by reference.

In some embodiments, the additional agent modulates a non-CDK biological pathway, a non-CDK target, a non-PAK biological pathway or a non-PAK target.

As used herein, the phrase “promoting motor neuron survival” refers to an increase in survival of motor neuron cells as compared to a control. In some embodiments, contacting of a motor neuron with a compound and/or agent described herein results in at least about 10%, 20%, 30%, 40%, 50% 60%, 70%, 80%, 90%, 95%, 100%. 2-fold, 3-fold, 4-fold, 5-fold or more increase in motor neuron survival relative to non treated control.

Motor neuron survival can be assessed by for example (i) increased survival time of motor neurons in culture; (ii) increased production of a neuron-associated molecule in culture or in vivo, e.g., choline acetyltransferase, acetylcholinesterase, SMN or GEMs; or (iii) decreased symptoms of motor neuron dysfunction in vivo. Such effects may be measured by any method known in the art. In one non-limiting example, increased survival of motor neurons may be measured by the method set forth in Arakawa et al. (1990, J. Neurosci. 10:3507-3515); increased production of neuron-associated molecules may be measured by bioassay, enzymatic assay, antibody binding Northern blot assay, etc., depending on the molecule to be measured; and motor neuron dysfunction may be measured by assessing the physical manifestation of motor neuron disorder. In one embodiment, the increase in motor neuron survival can be assessed by measuring the increase in SMN protein levels and/or GEM numbers. Cell survival can also be measured by uptake of calcein AM, an analog of the viable dye, fluorescein diacetate. Calcein is taken up by viable cells and cleaved intracellularly to fluorescent salts which are retained by intact membranes of viable cells. Microcopic counts of viable neurons correlate directly with relative fluorescence values obtained with the fluorimetric viability assay. This method thus provides a reliable and quantitive measurement of cell survival in the total cell population of a given culture (Bozyczko-Coyne et al., J. Neur. Meth. 50:205-216, 1993). Other methods of assessing cell survival are described in U.S. Pat. Nos. 5,972,639; 6,077,684 and 6,417,160, contents of which are incorporated herein by reference.

In vivo motor neuron survival can be assessed by an increase in motor neuron, neuromotor or neuromuscular function in a subject. In one non-limiting example, motor neuron survival in a subject can be assessed by reversion, allevation, amelioration, inhibition, slowing down or stopping of the progression, aggravation or severity of a condition associated with motor neuron dysfunction or death in a subject, e.g., SMA or ALS.

### p21 Activated Kinase

The p21-activated kinases (PAKs) are serinehthreonine kinases that function as downstream nodes for various oncogenic signalling pathways. PAKs are well-known regulators of cytoskeletal remodelling and cell motility, but have recently also been shown to promote cell proliferation, regulate apoptosis and accelerate mitotic abnormalities, which results in tumour formation and cell invasiveness. PAKs are regulated by many upstream signalling pathways, which include those that involve membrane-bound growth-factor receptors, integrin cell-adhesion complexes and G-protein-coupled receptors. Signalling activates guanine nucleotide-exchange factors (GEFs), which then stimulate GTP loading on CDC42 or RAC1. The kinase activity of PAKs is increased over the low level of basal activity after the binding of activated CDC42 or RAC1 to the Pak PBD domain. PAK1, which is a predominantly cytoplasmic protein, is recruited to the cell membrane, which results in its activation. This localization of PAKs occurs through interactions with adaptor proteins such as NCK, GRB2a and PIX, all of which are activated by growth-factor-receptor signalling. These adaptors bring PAK proteins into close proximity with various kinases and interacting proteins that activate PAK, which is important for relief from auto-inhibition as well as for maximal kinase activity. Activated PAKs in turn phosphorylate multiple substrates, which results in changes in cell survival, physiology and function. Members of PAK family include: PAK1, which plays a role in regulating cell motility and morphology; PAK2, which possibly plays a role in apoptosis; PAK3, which possibly plays a role in dendritic development and in the rapid cytoskeletal reorganization in dendritic spines associated with synaptic plasticity; and PAK4, which is a mediator of filopodia formation.

As used herein, by “PAK inhibitor” is meant an agent that decreases the level of a PAK kinase mRNA or protein, an activity of a PAK kinase, the half-life of a PAK kinase mRNA or protein, or the binding of a PAK kinase to another molecule (e.g., a substrate for a PAK kinase, a Rac protein, or a cdc42 protein), as measured using standard methods (see, for example, Ausubel et al., Current Protocols in Molecular Biology, Chapter 9, John Wiley & Sons, New York, 2000). For example, the agent may directly or indirectly inhibit the ability of a PAK kinase to phosphorylate merlin. Expression levels of mRNA can be determined using standard RNase protection assays or in situ hybridization assays, and the level of protein can be determined using standard Western or immunohistochemistry analysis. The phosphorylation levels of signal transduction proteins downstream of merlin activity can also be measured using standard assays. In some embodiments, a PAK inhibitor decreases PAK kinase activity by at least 20, 40, 60, 80, or 90%. In some embodiments, the level of PAK kinase activity is at least 2, 3, 5, 10, 20, or 50-fold lower in the presence of the PAK inhibitor.

In some embodiments, a PAK inhibitor is a Group I PAK inhibitor that inhibits, for example, one or more Group I PAK polypeptides, for example, PAK1, PAK2, and/or PAK3. In some embodiments, a PAK inhibitor is a PAK1 inhibitor. In some embodiments, a PAK inhibitor is a PAK2 inhibitor. In some embodiments, a PAK inhibitor is a PAK3 inhibitor. In some embodiments, a PAK inhibitor is a Group II PAK inhibitor that inhibits one or more Group II PAK polypeptides that inhibits, for example PAK-4, PAKS, and/or PAK6. In some embodiments, a PAK inhibitor is a PAK-4 inhibitor. In some embodiments, a PAK inhibitor is a PAK-4 inhibitor. In some embodiments, a PAK inhibitor is a PAKS inhibitor. In some embodiments, a PAK inhibitor is a PAK6 inhibitor.

A PAK inhibitor can be small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and aptamers); an extract made from biological materials such as bacteria, plants, fungi, or animal cells; animal tissues; naturally occurring or synthetic compositions; and any combinations thereof. Exemplary PAK inhbitors include, but are not limited to purvalnol A, staurosporin, aurosporine analogs, and pharmaceutically acceptable salts thereof (Zeng et al., J. Cell Sci. 113 (Pt 3): 471-82, 2000; Yu et al, J Biochem (Tokyo), 129(2): 243-51, 2001). Exemplary PAK kinase inhibitors which can inhibit PAK kinases indirectly include those reported by He et al. (Cancer J. 7(3):191-202, 2001, Cancer J. 6(4):243-8, 2000). Still other preferred PAK inhibitors include ATP analogs.

In some embodiments, a PAK inhibitor inhbitits/lowers the activity of a PAK by at least 5%, 10%, 20%, 30%, 40%, 50%, 60%, 70%, 80%, 90%, or 95% relative to a control. While not required, a PAK inhibitor can completely inhibit the PAK activity relative to a control.

Without limitations, a CDK can be selected from the group consisting of Cdk 1, Cdk 2, Cdk 3, Cdk 4, Cdk 5, Cdk 6, Cdk 7, Cdk 8, Cdk 9, Cdk 11, and any combinations thereof. Exemplary CDK inhibitors which can be used include, but are not limited to, purine and pyrimidine derivatives such as olomouscine, roscovitine, R-roacovitine (seliciclib), N9-isopropyl-olmoucine or NG75 and hymenialdisine. Other purine-based CDK inhibitors which may be used include CGP79807 and CGP74514 (which has cyclichydroxy or amino-alkyl amino groups present as C2 respectively, Imbach et al, Bioorg. Med. Chem. Lett., 9, 91-96 (1999); Dreyer et al, J. Med. Chem., 44, 524-530 (2001)), guanine derivatives such as NU2058 and NU6102 (Arris et al, J. Med. Chem, 43, 2797-2804 (2000), (Davies et al, Nat. Struct. Biol, 9, 745749 (2002), phenylaminopyridines such as CGP60474 (Furet et al, J. Comput. Aided Mol. Des, 14, 403-409 (2000), CINK4 (Soni et al, J. Nat. Cancer Inst, 93, 436-446 (2001). NU6027, thiazolopyrimidines (Fischer at al, Eur. J. Cancer, 38 (Suppl. 7), 124 (2002). Other CDK inhibitors which can be used include purvalanols, such as purvalanol A and purvalanol B, indirubin, oxindole based CDK inhibitors, (Kent at al, Biochem. Biophys. Res. Commun. 260, 768-774 (1999) such a phenylhydrazone oxindole and anilinomethylene oxindole, indenopyrazoles, (Nuglel et al, J. Med. Chem, 44, 1334-1336 (2001). Other CDK inhibitors which may be used include fiavopiridol and analogs thereof, such as 2-benzylidine-benzo-furan-3-ones (Kim et al J. Mod. Chem, 43,41264134 (2000); Schoepfer et al. J. Med. Chem, 45 1741-1747 (2002)), staurosporine, and analogues thereof, for example 7-hydroxystaurosporine, bryostatin-1, BMS-387032, SU9516, AZ703, E7070, amino imidazopyridine Id, NU 6140, flavopiridol, AG-024322, PD-0332991, PNU-252808, diarylureas, and paullones, such as kenpaullone, alsterpeullone, butyrolachtone-1, sangivamychin, SU9516 AZ703. Details of many of these inhibitors are described in J. Clin. Oncol. 23(36) 9408-9421 and J. Clin. Oncol. 24(11) 11701783. Further CDK inhibitors, which may be used in the present invention, are described in WO 01/44217, WO 99/24416, WO 01/44242, WO97/20842, WO98/05335, and WO99/07705.

Additionally, exemplary CDK inhibitors include, but are not limited to, 2-(3-Hydroxypropylamino)-6(O-hydroxybenzylamino)-9-isopropylpurine, 2-bromo-12,13-dihydro-5H-indolo[2,3-a]pyrrolo[3,4-c]carbazole-5,7(6H)-dione, 3-(2-Chloro-3-indolylmethylene)-1,3-dihydroindol-2-one, 2(bis-(Hydroxyethyl)amino)-6-(4methoxybenzylamino)-9-isopropyl-purine, 3-Amino-1Hpyrazolo[3,4-b]quinoxaline, 5-amino-3-((4-(aminosulfonyl)phenyl)amino)-N-(2,6-difluorophenyl)-1H1,2,4-triazole-1-carbothioamide, aloisine A, aloisine RPI06, alsterpaullone 2-cyanoethyl, alvocidib, aminopurvalanol A, bohemine, CGP74514A, ethyl-(6-hydroxy-4-phenylbenzo[4,5]furo[2,3-b])pyridine-3-carboxylate, fisetin, N4-(6-Aminopyrimidin-4-yl)-sulfanilamide, flavopiridol, kenpaullone, NSC 625987, NU6102, NU6140, olomoucine, olomoucine II, roscovitine, SU9516, WR 216174, p21Cip1 (CDKN1A), p27Kip1, (CDKN1B), and analogs thereof. Additional CDK inhibitors are described in U.S. Pat. Nos. 7,084,271, 7,078,591, 7,078,525, 7,074,924, 7,067,661, 6,992,080, 6,939,872, 6,919,341, 6,710,227, 6,683,095, 6,677,345, 6,610,684, 6,593,356, 6,569,878, 6,559,152, 6,1,477, 6,500,846, 6,448,264, and 6,107,305, the contents of which each are incorporated herein by reference in their entirety.

In some embodiments, a CDK inhibitor inhbitits/lowers the activity of a CDK by at least 5%, 10%, 20%, 30%, 40%, 50%, 60%, 70%, 80%, 90%, or 95% relative to a control. While not required, a CDK inhibitor can completely inhibit the CDK activity relative to a control.

Inventors have discovered that CDK or PAK inhibitor can inhibit motor neuron survival when used alone. However, using the CDK or PAK inhibitor with an additional agent that modulates a biological pathway or target promotes motor neuron survival.

Inventors have also discovered that agents which do not promote motor neuron survival when used alone can promote motor neuron survival when used in combination with a CDK or PAK inhibitor. Thus, the instant disclosure provides using a CDK or PAK inhibitor and an additional agent that modulates a biological pathway or a target for promoting motor neuron survival in a synergistic manner. In some embodiments, the CDK or PAK inhibitor is a compound of formula (I).

Without limitations, the compound of formula (I) and the additional agent can be used in any ratio. Accordingly, in some embodiments, the compound of formula (I) and the additional agent are in a ratio from 20:1 to 1:20. In some embodiments, the compound of formula (I) and the additional agent are in ratio from 10:1 to 1:10. In some embodiments, the compound of formula (I) and the additional agent are in ratio from 5:1 to 1:5. In some embodiments, the compound of formula (I) and the additional agent are in a ratio from 15:1 to 1:5. In some embodiments, compound of formula (I) and the additional agent are in a ratio from 10:1 to 1:1. In one embodiment, the compound of formula (I) and the additional agent are used in a 1:1 ratio.

In some embodiments, the biological pathway to be modulated is selected from the group consisting of JNK/c-Jun mediated cell death signaling, PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and combinations thereof. In some embodiments, the signaling pathway is the PI-3/AKT/GSK pathway. In some embodiments, the pathway comprises GSK-3b, CDK2, CDKS, PKR or IKK-2b. In some embodiments, the pathway comprises the HGK-Tak1-MKK4-JNK-c-Jun cell death signaling cascade.

In some embodiments, the target is selected from the group consisting of Na⁺/K⁺ channel, MAPK, cannobinoid receptor, GPCR, Ca²⁺ channel, K⁺ channel, PDE5, GSK/CDK, PKR, CDK2, IKK-2, proteasome, BMP/TGFbeta receptor and dopamine receptor, HGK, Tak1, MKK4, JNK, and c-Jun. In some embodiments, the target is glycogen synthase kinase 3 (GSK-3).

In some embodiments, the additional agent is selected from the group consisting of RTK activator, insulin, FGF (e.g. FGF2), EGF, NOF, TGF (e.g. TGFβ), MAPK activator, kinase inhibitor, GSK inhibitor, CDK inhibitor, PKR inhibitor, IKK inhibitor, an HGK inhibitor, a Tak1 inhibitor, a MKK4 inhibitor, a JNK inhibitor, c-Jun inhibitor, BMP/TGFβ ligand, cannabinoid or GPCR agonists, ion channel modulator (e.g. Na⁺/K⁺ channel modulator, Ca²⁺ channel modulator, K⁺ channel modulato), PDE5S inhibitor, HDAC inhibitor, proteasome inhibitor, dopamine receptor ligand, PDGF, and combinations thereof. In some embodiments, the additional agent is a GSK-3 inhibitor. In some embodiments, the additional agent is not a Tak1 inhibitor. In some embodiments, the additional agent is not a MKK4 inhibitor. In some embodiments, the additional agent is not a JNK inhibitor.

Without wishing to be bound by theory, the additional agent functions by increasing, inhibiting, preventing, blocking, stopping and/or reducing signaling activity in a biological pathway described herein. In some embodiments, an additional agent can alter the signaling activity by at least 5%, 10%, 20%, 30%, 40%, 50%, 60%, 70%, 80%, 90%, 95%, 100%, 2-fold, 3-fold, 4-fold, 5-fold, 10-fold or more relative to when pathway is not being modulated by the compound.

### Compounds for Modulating Biological Pathways and Targets

The biological pathway to be modulated can be selected from the group consisting of JNK/c-Jun mediated cell death signaling, PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and combinations thereof. In some embodiments, the signaling pathway is the PI-3/AKT/GSK pathway. In some embodiments, the pathway comprises GSK-3b, CDK2, CDKS, PKR or IKK-2b. In some embodiments, the pathway comprises the HGK-Tak1-MKK4-JNK-c-Jun cell death signaling cascade.

In some embodiments, the target is selected from the group consisting of Na⁺/K⁺ channel, MAPK, cannobinoid receptor, GPCR, Ca²⁺ channel, K⁺ channel, PDE5, GSK/CDK, PKR, CDK2, IKK-2, proleasome, BMP/TGFbeta receptor and dopamine receptor. In some embodiments, the target is glycogen synthase kinase 3 (GSK-3).

Additional agents for inhibiting a biological pathway or target can be selected from small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to microRNAs, siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

In some embodiments, the additional agent is selected from the group consisting of RTK activator, insulin, FGF (e.g. FGF2), EGF, NGF. TGF (e.g. TGFβ. MAPK activator, kinase inhibitor, GSK inhibitor, CDK inhibitor, PKR inhibitor, IKK inhibitor, an HGK inhibitor, a Tak1 inhibitor, a MKK4 inhibitor, a JNK inhibitor, c-Jun inhibitor, BMP/TGFβ ligand, cannabinoid or GPCR agonists, ion channel modulator (e.g. Na⁺/K⁺ channel modulator, Ca²⁺ channel modulator, K⁺ channel modulato), PDE5 inhibitor, HDAC inhibitor, proteasome inhibitor, dopamine receptor ligand, PDGF, and combinations thereof. In some embodiments, the additional agent is a GSK-3 inhibitor. In some embodiments, the additional agent is not a Tak1 inhibitor. In some embodiments, the additional agent is not a MKK4 inhibitor. In some embodiments, the additional agent is not a JNK inhibitor.

Without wishing to be bound by theory, the additional agent functions by increasing, inhibiting, preventing, blocking, stopping and/or reducing signaling activity in a biological pathway described herein. In some embodiments, an additional agent can alter the signaling activity by at least 5%, 10%, 20%, 30%, 40%, 50%, 60%, 70%, 80%, 90%, 95%, 100%, 2-fold, 3-fold, 4-fold, 5-fold, 10-fold or more relative to when pathway is not being modulated by the additional agent.

In some embodiments, the additional agent is a GSK inhibitor. In one embodiment, the additional agent is an inhibitor of GSK-3. GSK inhibitors are known widely in the art and can be grouped into different chemical classes such as pyrroloazepine, flavone, benrazepinone, bis-indole, pyrrolopyrazine, pyridyloxadiazole, pyrazolopyridine, pyrazolopyridazine, aminopyridine, pyrazoloquinoxaline, oxindole (indolinone), thiazole, bisindolylmaleimide, azainodolylmaleimide, arylindolemaleimide, aniliomaleimnde, phenylaminopyridine, triazole, pyrrolopyrimidine, pyrazolopyrimidine, and chloromethyl thienyl ketone.

In some embodiments, the additional agent is a GSK inhibitor selected from the group consisting of CHIR98014, CHIR99021, GSK1, GSK2, GSK6, GSK7, GSK8 (ARA014418), GSK 13, hymenialdisine, flavopiridol, aloisine A, aloisine B, compound 12, pyrazolopyridine 18, pyrazolopyridine 9, pyrazolopyridine 34, CT20026, compound 1, SU9516, staurosporine, compound 5a, compound 29, compound 46, GFI09203x, RO318220, SB216763, SB415286, 15, CGP60474, compound 8b, and combinations thereof.

GSK inhibitors amenable to the invention are also described in U.S. Pat. Nos. 7,056,939, 7,045,519, 7,037,918, 6,989,382, 6,949,547, 6,872,737, 6,800,632, 6,780,625, 6,608,063, 6,489,344, 6,479,490, 6,441,053, 6,417,185, 6,323,029, 6,316,259, and 6,057,117, the contents of which each are incorporated herein by reference in their entirety.

In some embodiments, the additional agent for modulating a biological pathway or target is a paullone or analog or derivative thereof. In some embodiment, the additional agent is a paullone or analog or derivative thereof selected from the group consisting of alsterpaullone; 2-cyanoethyl-alsterpullone; 1-aza-alsterpaullon; 1-aza-kenpaullone; 9-cyano-2,3-dimethoxypaullone; 2-iodopaullone; 2-bromo-9-nitropaullone; 2,3-dimethoxy-9-nitropaullone; 7-bromo-5-(4-nitrophenylhydrazono)-4,5-dihydro-1-H-[1]benzazepin2(3H)-one; 7,8-dimethoxy-5-(4-nitrophenylhydrazono)-4,5dihydro-1H-[1]benzazepin-2-(3H)-one; 9-cyanopaullone; 9-chloropaullone; 9-trifluoromethylpaullone; 2,3-dimethoxy-9-trifluoromethylpaullone; 9-bromo-2-methyloxycarbonylmethylpaullone; 9-fluoropaullone; 9-bromo-2,3-dimethoxypaullone; 9-bromo-2,3-dimethoxypaullone; 9-methylpaullone; 10-bromopaullone; 2-bromopaullone; 11-chloropaullone; 2-(3-hydroxy-1-propinyl)-9-trifluoromethylpaullone; 9-bromo-12-(2-hydroxyethyl)-paullone; kenpaullone; Alsterpaullone; 2-cyanoetyl-alsterpaullone; 1-aza-kenpaullone; 1-aza-alsterpaullone; 9-bromo-12-methylpaullone; 9-bromo-5-(methyloxycarbonylmethyl)paullone; 11-methylpaullone; paullone; 11-ethylpaullone; 9-bromo-7,12-dihydro-6-(hydroxyamino)-indolo[2-3-d][1]benzazepine; 2,9-dibromopaullone; 11-bromopaullone; 2,3-dimethoxypaullone; 9-bromo-7,12dihydro-6-methylthio-indolo[2-3-d][1]benzazepine; (E)-2(3-oxo-1-butenyl)-9-trifluoromethylpaullone; 9-bromo-12ethylpaullone; 9-bromo-7,12-dihydro-indolo[2-3-d][1]benzazepine-6(5H)-thione; 2-bromo-9-trifluoromethylpaullone; 2-[2-(1-hydroxycyclohexyl)ethinyl]-9-trifluoromethyl-paullone; 9-bromo-5methylpaullone; 9-methoxypaullone; 2-iodo-9-trifluoromethylpaullone; 9-bromo-12-(tert-butyloxycarbonyl)-paullone; 9-bromo-12-(2-propenyl)paullone; 9-bromo-4-hydroxypaullone; 8,10-dichloropaullone; 5-benzyl-9-bromopaullone; 9-bromo-4-methoxypaullone; 9-bromo-5-ethylpaullone; 9-bromo-5,7bis-(tert-butyloxycarbonyl)-paullone; 4-methoxypaullone; 9-bromo-5,6,7,12-tetrahydrobenzo[6-7]cyclohept[1,2,b]indole; 2-phenyl-4-(2-thienyl)-5H-pyrido[2-3-d][1]benzazepine-6(7H)-thione; 9-bromo-5,7,12-tri-(tert-butyloxycarbonyl)-paullone; 9-bromo-5,12-bis-(tert-butyloxycarbonyl)paullone; 4-(4-chlorophenyl)-2-(2-naphthyl)-5H-pyrido[23-d][1]benzazepine-6(7H)-thione; and 5,6,7,12-tetrahydrobenzo[6-7]cyclohept[1,2-b]indole.

Other known derivatives of paullone family such as those found in Leost at al., Paullones Are Potent inhibitors of Glycogen Synthase Kinase-3B and Cyclin-dependent Kinase 5/p25, Eur. J. Biochem. (2000), 267, 5983-5994, PCT Publication Nos. WO 99/65910 and WO09/010298, and U.S. Pat. Nos. 7,232,814 and 7,393,953 are also contemplated as suitable additional agents for inhibiting a biological pathway or target, e.g., GSK-3. The disclosures of each of the foregoing are incorporated by reference herein.

In some embodiments, the additional agent for modulating a biological pathway or target is a GSK-3β inhibitor. Exemplary GSK-3β inhibitors include, but are not limited to,

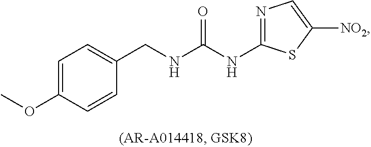 [SMILES: COc1ccc(CNC(=O)Nc2ncc([N+](=O)[O-])s2)cc1]

N-Butyl-N′-(5-nitro-1,3-thiazol-2-yl) urea; N-(5-Nitro-1,3-thiazol-2-yl)pentanamide; 1-{4-Amino-2-[(4-methoxyphenyl)amino]-1,3-thiazol-5-yl}ethanone; N-Benzyl-N′-(5-nitro-1,3-thiazol-2-yl) urea; N-(4-methoxybenzyl)-N′-(5-nitro-1,3-thaizol-2-yl) urea; 3-(4-Methoxyphenyl)-N-(5-nitro-1,3-thiazol-2-yl) propanamide; 4-(4-Methoxyphenyl)-N-(5-nitro-1,3-thiazol-2-yl) butanamide; 2-(3-Methoxyphenyl)-N-(5-nitro-1,3-thiazol-2yl) acetamide; 2-(4-Fluorophenyl)-N-(5-nitro-1,3-thiazol-2-yl) propanamide; 2-(3-Methylphenyl)-N-(5-nitro-1,3-thiazol-2-yl) acetamide; 1-benzyl-3-naphthalen-1-yl-urea or 1-benzyl[1,3]dioxol-5-yl-3-benzyl-urea. Other GSK-3β inhibitors amenable to the invention are described in U.S. Pat. No. 7,056,939 and PCT Publication Nos. WO07/017145 and WO03/089419, content of both of which is incorporated herein by reference.

In some embodiments, the additional agent for modulating a biological pathway or target is a GSK/CDK inhibitor. Exemplary GSK/CDK inhibitors include, but are not limited to,

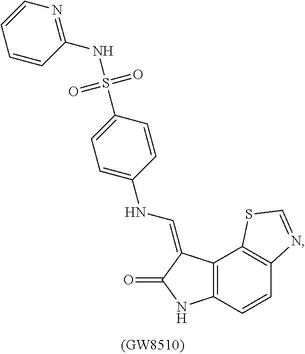 [SMILES: O=C1Nc2ccc3ncsc3c2/C1=C/Nc1ccc(S(=O)(=O)Nc2ccccn2)cc1]

and derivatives, isomers, and pharmaceutically acceptable salts thereof.

In some embodiments, the additional agent for modulating a biological pathway or target is an IKK-2 inhibitor. Exemplary IKK-2 Inhibitors include, but are not limited to,

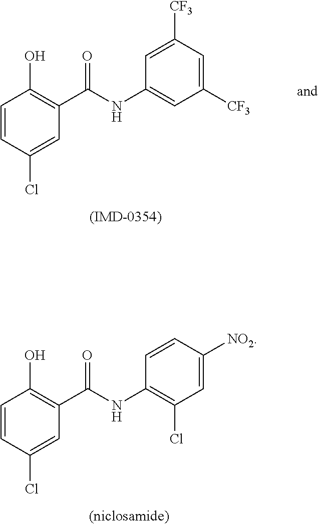 [SMILES: O=C(Nc1cc(C(F)(F)F)cc(C(F)(F)F)c1)c1cc(Cl)ccc1O ; O=C(Nc1ccc([N+](=O)[O-])cc1Cl)c1cc(Cl)ccc1O]

In some embodiments, the additional agent for modulating a biological pathway or target is an inhibitor of PKR pathway and inhibits protein kinase R (PKR). PKR inhibitors include, but are not limited to, 3-[1-(3H-imidazol-4-yl)-meth-(Z)-ylidene]-5-methoxy-1,3-dihydroindol-2-one (SU9516), 2-aminopurine, 9-(4-bromo-3,5-dimethylpyridin-2-yl)-6-chloro-9H-purin-2-ylamine, 9-(4-bromo-3,5-dimethyl-pyridin-2-ylmethyl)-6-chloro-9H-purin-2ylamine, phosphate salt, 9-(4-bromo-3,5-dimethyl-pyridin2-ylmethyl)-6-chloro-9H-purin-2-ylamine, hydrochloric acid salt, 6-bromo-9-(4-bromo-3,5-dimethyl-pyridin-2-ylmethyl)-9H-purin-2-ylamine, 6-bromo-9-(4-bromo-3,5-dimethyl-1-oxy-pyridin-2-yl methyl)-9H-purin-2-ylamine, 2-(2amino-6-chloro-purin-9-ylmethyl)-3,5-dimethyl-pyridin-4ol, 9-(4-allyloxy-3,5-dimethyl-pyridin-2-ylmethyl)-6-chloro-9H-purin-2-ylamine, 6-chloro-9-[4-(2-ethoxyethoxy)-3,5-dimethyl-pyridin-2-ylmethyl]-9H-purin-2ylamine, 6-chloro-9-(4-cyclopropylmethoxy-3,5-dimethylpyridin-2-ylmethyl)-9H-purin-2-ylamine, 6-chloro-9-(4isobutoxy-3,5-dimethyl-pyridin-2-ylmethyl)-9H-purin-2ylamine, 6-chloro-9-(4-chloro-3,5-dimethyl-pyridin-2-ylmethyl)-9H-purin-2-ylamine, 6-chloro-9-(3,5-dimethylpyridin-2-yl methyl)-9H-purin-2-ylamine, and 6-bromo-9(4-methoxy-3,5-dimethyl-pyridin-2-ylmethyl)-9H-purin-2ylamine, 3-[3,5-dimethyl-4-(4-methylpiperazine-1-carbonyl)-1H-pyrrol-2ylmethylene)-4-pyridin-4-yl-1,3-dihydroindol-2-one, 3-[3-methyl-4-(piperidine-1-carbonyl)-1H-pyrrol-2-ylmethylene]-4-pyridin-4-yl1,3-dihydroindol-2-one, 3-(3,5-dimethyl-1H-pyrrol-2-ylmethylene)-4-pyridin-4-yl-1,3-dihydroindol-2-one, 3-[2(2-oxo-4-pyridin-4-yl-1,2-dihydroindol-3-ylidenemethyl-4,5,6,7-tetrahydro-1H-indol-3-yl]-propionic acid, 3-[5-ethyl2-(2-oxo-4pyridin-4-yl-1,2-dihydroindol-3ylidenemethyl-1H-pyrrol 3-yl]-propionic acid, 4-(2carboxyethyl)-2-methyl-5-(2-oxo-4-pyridin-4-yl-1,2dihydro-indol-3-ylidenemethyl)-1H-pyrrole-3-carboxylic acid ethyl ester, 3-[2,4-dimethyl-5-(2-oxo-4-pyridin-4-yl-1,2-dihydroindol-ylidenemethyl)-1H-pyrrol-3-yl]-propionic acid, 4-pyridin-4-yl-3-(4,5,6,7-tetrahydro-1H-indol-2-ylmethylene), 5-methyl-2-(2-oxo-4-pyridin-4-yl-1,2-dihydroindol-ylidenemethyl)-1H-pyrrole-3-carboxylic acid, 3-[3-(3morpholin-4-yl-propyl)-4,5,6,7-tetrahydro-1H-indol-2 ylmethylene]-4-pyridin-4-yl-1,3-dihydroindol-2-one, 3-[3methyl-5-(4-methylpiperazine-1-carbonyl)-1H-pyrrol-ylmethylene]-4-pyridin-4-yl-1,3-dihydroindol-2-one, 3-(5-methylthiophen-2-ylmethylene)-4-pyridin-4-yl-1,3-dihydroindol-2-one, 4-[4-(2-oxo-4-pyridin-4-yl-1,2-dihydroindol3-ylidenemethy)phenyl]-piperazine-1-carbaldehyde, 4-(2hydroxyethyl)-5-2-oxo-4-pyridin-4-yl-1,2-dihydroindol-3ylindenemethyl)-1H-pyrrole-3-carboxylic acid, [3-methyl-4(piperidine-1-carbonyl)-1H-pyrrol-2 ylmethylene]-4-piperidin-4-yl-1,3-dihydro-indol-2-one, 3-[3-methyl-4(morpholine-4-carbonyl)-1H-pyrrolylmethylene]-4piperidin-4-yl-1,3-dihydroindol-2-one, 3-(3,5-dibromo-4hydroxy-benzylidine-2-oxo-2,3-dihydro-1H-indole-5carbonitrile, 3-(3,5-dibromo-4-hydroxy-benzylidene)-5-(2methyl-thiazol-4-yl)-1,3-dihydro-indol-2-one, 3-(3-bromo5-ethoxy-4-hydroxy-benzylidene)-5-(2-methyl-thiazol-4yl)-1,3-dihydro-indol-2-one, 3-(3,5-dichloro-4-hydroxybenzylidene-5-(2-methyl-thiazol-4-yl)-1,3-dihydro-indol-2one, (butanoyl)-1,3-dihydro-indol-2-one, 3-(3,5-dibromo-4hydroxy-benzylidene-5-(3-methy-butanoyl-1,3-dihydroindol-2-one, 5-benzoyl-3-(3,5-dibromo-4-hydroxybenzylidene)-1,3-dihydro-indol-2-one, 5-benzoyl-3-(3,5dichloro-4-hydroxy-benzylidene)-1,3-dihydro-indol-2-one, 5-benzoyl-3-(3-bromo-5-ethoxy-4-hydroxy-benzylidene)-1,3-dihydro-indol-2-one, 3-(3,5-dichloro-4-hydroxy-benzylidene-5-(3-methyl-butanoyl)-1,3-dihydro-indol-2-one, 3-(3-bromo-5-ethoxy-4-hydroxy-benzylidene)-5-(3-methylbutanoyl)-1,3-dihydro-indol-2-one, 3-(3,5-dibromo-4-hydroxy-benzylidene-5-(pyridine-3-carbonyl)-1,3-dihydro-indol-2-one, 3-(3,5-dichloro-4-hydroxy-benzylidene)-5(pyridine-3-carbonyl)-1,3-dihydro-indol-2-one, 3-(3,5dibromo-4-hydroxy-benzylidene)-5-(pyridine-4-carbonyl)1,3-dihydro-indol-2-one, 3-(3-bromo-5-ethoxy-4-hydroxybenzylidene)-5-(pyridine-4-carbonyl)-1,3-dihydro-indol-2one, 3-(3,5-dichloro-4-hydroxy-benzylidene)-5-(pyridine-4carbonyl)-1,3-dihydro-indol-2-one, 3-(3-bromo-5-ethoxy-4hydroxy-benzylidene)-5-(pyridine-3-carbonyl)-1,3dihydro-indol-2-one, 3-(3,5-dichloro-4-hydroxybenzylidene-5-(oxazol-5-yl)-1,3-dihydro-indol-2-one, 3-(3,4-dibromo-4-hydroxy-benzylidene)-5-(oxazol-5-yl)-1,3dihydro-indol-2-one, 3-(3,5-dibromo-4-hydroxybenzylidene)-5-(2-ethyl-thiazo-4-yl)-1,3-dihydro-indol-2one, 3-(3,5-dibromo-4-hydroxy-benzylidene)-2-oxo-2,3dihydro-1H-indol-5-carboxylic acid methyl ester, 3-(3,5dibromo-4-hydroxy-benzylidene-5-(furan-2-carbonyl)-1,3dihydro-indol-2-one, 3-(3,5-dichloro-4-hydroxybenzylidene-5-(furan-2-carbonyl)-1,3-dihydro-indol-2-one, 3-(3-bromo-5-ethoxy-4-hydroxy-benzylidene-5-(furan-2carbonyl)-1,3-dihydro-indol-2-one, 5-cyclopropanecarbonyl-3-(3,5-dibromo-4-hydroxy-benzylidene)-1,3-dihydroindol-2-one, 5-aminomethyl-3-(3,5-dibromo-4-hydroxybenzylidene)-1,3-dihydro-indol-2-one, 5-cyclopentanecarbonyl-3-(3,5-dibromo-4-hydroxy-benzylidene)-1,3-dihydro-indol-2-one, 3-(3,5-dichloro-4-hydroxy-benzylidene)-2-oxo-2,3-dihydro-1H-indole-5-carboxylic acid methyl ester, 3-(3,5-dibromo-4-hydroxybenzylidene)-1,3-dihydro-indol-2-one, 3-(3,5-dibromo-4hydroxy-benzylidene)-5-(thiophene-2-carbonyl)-1,3dihydro-indol-2-one, 5-(2-amino-thiazol-4-yl)-3-(3,5-dibromo-4-hydroxy-benzylidene)-1,3dihydro-indo-2-one, 3-(3,5-dibromo-4-hydroxy-benzylidene)-5-(imidazo[1,2a]pyridin-2-yl)-1,3-dihydro-indol-2-one, 3-(3,5-dibromo-4hydroxy-benzylidene)-5-propionyl-1,3-dihydro-indol-2one, 3-(3,5-dibromo-4-hydroxy-benzylidene)-2-oxo-2,3dihydro-1H-indole-5-sulfonic acid amide, 3-(3,5-dibromo4-hydroxy-benzylidene)-2-oxo-2,3 dihydro-1H-indole-5sulfonic acid N,N-diethylamide, 3-(3,5-dibromo-4-hydroxybenzylidene)-5-(pyrrolidine-1-sulfonyl)-1,3-dihydro-indol2-one, 3-(3,5-dibromo-4-hydroxy-benzylidene)-2-oxo-2,3dihydro-1H-indol-5-sulfonic acid (N-2-dimethylaminoethyl)-N-methyl-amide, 3-(3,5-dibromo-4-hydroxybenzylidene)-5-(isoxazole-5-carbonyl)-1,3-dihydro-indol2-one, 5-chloro-3-(3,5-dibromo-4-hydroxy-benzylidene)-1,3-dihydro-indol-2-one, 5-chloro-3-(3,5-dichloro-4-hydroxy-benzylidene)-1,3-dihydro-indol-2-one, 3-(3,5dibromo-4-hydroxy-benzylidene)-5-trifluoromethoxy-1,3dihydro-indol-2-one, 5-bromo-3-(3,5-dichloro-4-hydroxybenzylidene)-1,3-dihydro-indol-2-one, 3-(3-bromo-5-ethoxy-4-hydroxy-benzylidene)-5-iodo-1,3-dihydro-indol2-one, 3-(3-bromo-4-hydroxy-5-methoxy-benzylidene)-5 iodo-1,3-dihydro-indol-2-one, 5-bromo-3-(3,5-diiodo-4hydroxy-benzylidene)-1,3-dihydro-indol-2-one, 3-(3,5dihydro-4-hydroxy-benzylidene)-5-trifluoromethoxy-1,3dihydro-indol-2-one, 3-(3-bromo-4-hydroxy-5-methoxybenzylidene)-1,3-dihydro-indol-2-one, 3-(3,5-dinitro-4hydroxy-benzylidene)-1,3-dihydro-indol-2-one, 3-(3,5dichloro-4-hydroxy-benzylidene)-1,3-dihydro-indol-2-one, 3-(3-chloro-4-hydroxy-5-methoxy-benzylidene)-19,3-dihydro-indol-2-one, 3-(3,5-diiodo-4-hydroxy-benzylidene)-1,3dihydro-indol-2-one, 3-(3-bromo-4-hydroxy-5-methoxybenzylidene)-5-chloro-1,3-dihydro-indol-2-one, 5-chloro-3-(3,5-dinitro-4-hydroxy-benzylidene)-1,3-dihydro-indol-2one, 5-chloro-3-(4-hydroxy-3-methoxy-5-nitrobenzylidene)-1,3-dihydro-indol-2-one, 5-chloro-3-(3-chloro-4-hydroxy-5-methoxy-benzylidene)-1,3-dihydroindol-2-one, 5-chloro-3-(3,5-diiodo-4-hydroxybenzylidene)-1,3-dihydro-indol-2-one, 5-bromo-3-(3-bromo-5-ethoxy-4-hydroxy-benzylidene)-1,3-dihydroindol-2-one, 5-bromo-3-(3-bromo-4-hydroxy-5-methoxybenzylidene)-1,3-dihydro-indol-2-one, 3-(3-bromo-4-hydroxy-5-methoxy-benzylidene)-5,6-difluoro-1,3-dihydroindol-2-one, 3-(3-bromo-5-ethoxy-4-hydroxy-benzylidene)5-trifluoromethoxy-1,3-dihydro-indol-2-one, 3-(3,5-dichloro-4-hydroxy-benzylidene)-5-trifluoromethoxy-1,3dihydro-indol-2-one, 3-(3-bromo-4-hydroxy-5-methoxybenzylidene)-5-trifluoromethoxy-1,3-dihydro-indol-2-one, 3-(3,5-dibromo-4-hydroxy-benzylidene)-5,7-dinitro-1,3-dihydro-indol-2-one, 3-(3,5-dibromo-4-hydroxybenzylidene)-5-nitro-1,3-dihydro-indol-2-one, 3-(3,5-dibromo4-hydroxy-benzylidene)-7-iodo-1,3-dihydro-indol-2-one, 3-(3,5-dichloro-4-hydroxy-benzylidene)-5-nitro-1,3-dihydro-indol-2-one, 3-(3,5-dibromo-4-hydroxy-benzylidene)7-iodo-1,3-dihydro-indol-2-one, 7-bromo-3-(3,5-dichloro4-hydroxy-benzylidene)-1,3-dihydro-indol-2-one, 3-(3bromo-5-ethoxy-4-hydroxy-benzylidene)-5-nitro-1,3dihydro-indol-2-one, and 2-(N-{3-[3-(3,5-dibromo-4hydroxy-benzylidene)-2-oxo-2,3-dihydro-1H-indol-5-yl]-2oxo-ethyl}-N-methyl-amino)-acetamide. Analogs of SU9516 and PKR inhibitor are described in Yu et al., Biochem. Pharmacol. 64:10911100, 2002, Lane et al., Cancer Res. 61:6170-6177, 2001, Jammi et al., Biochem. Biophys. Res. Commun. 308:50-57, 2003, Shimazawa et al, Neurosci. Lett. 409:192-195, 2006, Peel, J. Neuropathol. Exp. Neurol. 63:97-105, 2004, Bando et al, Neurochem. Int. 46:11-18, 2005, Peel et al. Hum. Mol. Genet. 10:1531-1538, 2001, and Chang et al, J. Neurochem. 83:1215-1225, 2002, the contents of which each are incorporated herein by reference in their entirety.

In some embodiments, the additional agent for modulating a biological pathway or target is a CDK inhibitor. Exemplary CDK inhibitors include, but are not limited to, 2-(3-Hydroxypropylamino)-6(O-hydroxybenzylamino)-9-isopropylpurine, 2-bromo-12,13-dihydro-5H-indolo[2,3-a]pyrrolo[3,4-c]carbazole-5,7(6H)-dione, 3-(2-Chloro-3-indolylmethylene)-1,3-dihydroindol-2-one, 2(bis-(Hydroxyethyl)amino)-6-(4methoxybenzylamino)-9-isopropyl-purine, 3-Amino-1Hpyrazolo[3,4-b]quinoxaline, 5-amino-3-((4-(aminosulfonyl)phenyl)amino)-N-(2,6-difluorophenyl)-1H1,2,4-triazole-1-carbothioamide, aloisine A, aloisine RP106, alsterpaullone 2-cyanoethyl, alvocidib, aminopurvalanol A, bohemine, CGP74514A, ethyl-(6-hydroxy-4-phenylbenzo[4,5]furo[2,3-b])pyridine-3-carboxylate, fisetin, N4-(6-Aminopyrimidin-4-yl)-sulfanilamide, flavopiridol, kenpaullone, NSC 625987, NU6102, NU6140, olomoucine, olomoucine 11, roscovitine, SU9516, WR 216174, p21Cip1 (CDKNIA), p27Kip1, (CDKN1B), and analogs thereof. Additional CDK inhibitors are described in U.S. Pat. Nos. 7,084,271, 7,078,591, 7,078,525, 7,074,924, 7,067,661, 6,992,080, 6,939,1172, 6,919,341, 6,710,227, 6,683,095, 6,677,345, 6,610,684, 6,593,356, 6,569,878, 6,559,152, 6,531,477, 6,500,846, 6,448,264, and 6,107,305, the contents of which each are incorporated herein by reference in their entirety.

In some embodiments, the additional agent for modulating a biological pathway or target is a HDAC inhibitor. Inhibitors of HDAC include small molecular weight carboxylates (e.g., less than about 250 amu), hydroxamic acids, benzamides, epoxyketones, cyclic peptides, and hybrid molecules. (See, for example, Drummond D. C., at al. Annu. Rev. Pharmacol. Toxicol. (2005) 45: 495-528, (including specific examples therein) which is hereby incorporated by reference in its entirety). Non-limiting examples HDAC inhibitors include, but are not limited to, Suberoylanilide Hydroxamic Acid (SAHA (e.g., MK0613, vorinoatat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (i.e., N-acetyl dinaline) and MS-27-275), MGCDO103, NVP-LAQ-824, CBHA (m-carboxycinnaminie acid bishydroxamic acid), JNJ16241199, Tubacin, A-161906, proxamide, oxamflatin, 3-Cl-UCHA (i.e., 6-(3-chlorophenylurcido)caproic hydroxamic acid), AOE (2-amino-8-oxo-9,10-epoxydecanoic acid), CHAP31 and CHAP 50. Other inhibitors include, for example, dominant negative forms of the HDACs (e.g., catalytically inactive forms) siRNA inhibitors of the HDACs, and antibodies that specifically bind to the HDACs. HDAC inhibitors are commercially available, e.g., from BIOMOL International, Fukasawa, Merck Biosciences, Novartis, Gloucester Phanmaceuticals, Atom Pharma, Titan Pharmaceuticals, Schering A G, Pharmion, MethylGene, and Sigma Aldrich. Further HDAC ihibitors amenable to the invention include, but are not limited to, those that are described in U.S. Pat. Nos. 7,183,298; 6,512,123; 6,541,661; 6,531472; 6,960,685; 6,897,220; 6,905,669; 6,888,207; 6,800,638 and 7,169,801, and U.S. patent application Ser. Nos. 10/811,332; 12/286,769; 11/365,268; 11/581,570; 10/509,732; 10/546,153; 10/381,791 and 11/516,620, the contents of which each are incorporated herein by reference in their entirety.

In some embodiments, the additional agent for modulating a biological pathway or target is a proteasome inhibitor. Exemplary proteasome inhibitors amenable to the invention include, but are not limited to those that are described in U.S. Pat. Nos. 5,693,617; 5,780,454; 5,834,487; 6,465,433; 6,794.516; 6,747,150; 6,117,887; 6,133,308; 6,6617,317; 6,294,560; 6,849,743; 6,310.057; 6,566,553; 6,07,5150; 6,083,903; 6,066,730; 6,297,217 and 6,462,019, the contents of which each are incorporated herein by reference in their entirety. In one embodiment, the proteasome inhibitor is not lacacystin or those described in U.S. Pat. Publication No. 2007/0207144.

In some embodiments, the additional agent for modulating a biological pathway or target is a dopamine receptor ligand. Exemplary dopamine receptor ligands amenable to the invention include, but are not limited to those that are described in U.S. Pat. Nos. 6,469,141; 5,998,414; 6,107,313; 5,849,765; 5,861,407; 5,798,350; 6,103,715; 5,576,314; 5,538,965; 5,968,478; 5,700,445; 5,407,823 and 5,602,121, the contents of which each are incorporated herein by reference in their entirety.

In some embodiments, the additional agent for modulating a biological pathway or target is a cannabinoid (CB) receptor agonist. In one embodiment, the cannabinoid receptor agonist is WIN55,212-2 or anandamide. Other exemplary cannabinoid receptor agonists amenable to the invention include, but are not limited to those that are described in U.S. Pat. Nos. 5,607,933; 5,324,737; 5,013,837; 5,081,122; 6,825,209; 5,817,651; 7,057,076; 5,068,234; 5,605,906; 6,995,187; 6,166,066; 6,509,367; 6,100,259; 7,119,108; 4,973,587; 5,112,820; 7,037,910; 5,948,777; 5,925,768; 6,013,648; 6,864291; 6,903,137; 6,943,266 and 5,596,106, the contents of which each are incorporated herein by reference in their entirety.

In some embodiments, the additional agent for modulating a biological pathway or target is FGF, EGF, NGF, TGF, PDGF, PDGF-BB or insulin, which compound activates PI-3K signaling pathway.

In some embodiments, the additional agent for modulating a biological pathway or target is an activator of PI-3K pathway, which compound activates PI3K. PDK or PKB.

In some embodiments, the additional agent for modulating a biological pathway or target is inhibits IκB kinase 2 (IKK-2). Exemplary IKK-2 inhibitors include, but are not limited to SC-514, SPC-839, IKK-2 inhibitor IV (CAS: 507475-17-4) and IKK-2 inhibitor VI. Other IKK-2 inhibitors amenable to the present invention include those described in U.S. Pat. Nos. 7,122,544; 6,462,036; and 7,125,896, and U.S. patent application Ser. Nos. 11/271,591; 11/211,383; 10/542,044; 11/272,401; 11/430,215; 11/346,986; and 10/542,326, the contents of which each are incorporated herein by reference in their entirety. Further IKK-2 inhibitors are described in Bingham, A. H., et al., Bioorg. Med. Chem. (2003), 14, 409-412 and Liddle, J., et al., Bioorg. Med. Chem. Lett (2009), 19, 2504-2508, the contents of which each are incorporated herein by reference in their entirety.

In some embodiments, the additional agent for modulating a biological pathway or target is a modulator of TGF-β signaling. Exemplary modulators of TGF-β signaling include, but are not limited to, AP-12009 (TGF-β Receptor type U antisense oligonucelotide), Lerdelimumab (CAT 152, antibody a TGF-β Receptor type II) GC-1008 (antibody to all isoforms of human TGF-β), ID11 (antibody to all isoforms of murine TGF-β), soluble TGF-β, soluble TGF-β Receptor type II, dihydropyrroloimidazole analogs (e.g., SKF-104365), triarylimidazole analogs (e.g., SB-202620 (4-(4-(4-fluorophenyl)-5-(pyridin-4-yl)-1H-imidazol-2-yl)benzoic acid) and SB-203580 (4-(4-Fluorophenyl)-2-(4-methylsulfinyl phenyl)-5-(4-pyridyl)-1H-imidazole)), RL-0061425, 1,5-naphthyridine aminothiazole and pyrazole derivatives (e.g. 4-(6-methyl-pyridin-2-yl)-5-(1,5-naphthyridin-2-yl)-1,3-thiazole-2-amine and 2-[3-(6-methyl-pyridin-2-yl)-1H-pyrazole-4-yl]-1,5-naphthyridine), SB-431542 (4-(5-Benzol[1,3]dioxol-5-yl-4-pyridin-2-yl-1H-imidazol-2-yl)-benzamide), GW78838 (4-(4-(3-(pyridin-2-yl)-1H-pyrazol-4-yl)pyridin-2-yl)-N-(tetrahydro-2H-pyran-4-yl)benzamide), A-83-01 (3-(6-Methyl-2-pyridinyl)-N-phenyl-4-(4-quinolinyl)-1H-pyrazole-1-carbothioamide), Decorin, Lefty 1, Lefty 2, Follistatin, Noggin, Chordin, Cerberus, Gremlin, Inhibin, BIO (6-brmo-indirubin-3′-oxime), Smad proteins (e.g., Smad6, Smad7), Cystatin C, soluble TGF-β Receptor type I, AP-11014 (TGF-β Receptor type I antisense oligonucleotide), Metelimumab (CAT 152, TGF-β Receptor type I antibody), LY550410, LY580276 (3-(4-fluorophenyl)-5,6-dihydro-2-(6-methylpyridin-2-yl)-4H-pyrrolo[1,2-b]pyrazole), LY364947 (4-[3-(2-Pyridinyl)-1H-pyrazol-4-yl]-quinoline), LY2109761. LY573636 (N-((5-bromo-2-thienyl)-2,4-dichlorobenzamide), SB-505124 (2-(5-Benzo[1,3]dioxol-5-yl-2-tert-butyl-3H-imidazol-4-yl)-6-methylpyridine), SD-208 (2-(5-Chloro-2-fluorophenyl)-4-[(4-pyridyl)amino]pteridine), SD-093. K12689, SM16, FKBP12 protein, 3-(4-(2-(6-methylpyridin-2-yl)H-imidazo[1,2-a]pyridin-3-yl)quinolin-7-yloxy)-N,N-dimethylpropen-1-amine, and analogs thereof. Other modulators of TGF-β signaling amenable to the invention are described in Callahan, J. F. et al., J. Med. Chem. 45, 999-1001 (2002); Sawyer, J. S. et al., J. Med. Chem. 46, 3953-3956 (2003); Sawyer J. S. et al., Bioorg. Med. Chem. Lett. 14, 3581-3584 (2004); Gellibert, F. et al., J. Med. Chem. 47, 4494-4506 (2004); Yingling, J. M. et al., Nature Rev. Drug Disc. 3, 1011-1022 (2004): Tojo, M. et al., Cancer Sci. 96: 791-800 (2005); Valdimarsdottir, G. et al., APMIS 113, 773-389 (2005); Petersen et al. Kidney International 73, 705-715 (2008); Yingling, J. M. et al., Nature Rev. Drug Disc. 3, 1011-1022 (2004); Byfield, S. D. et al., Mol. Pharmacol., 65, 744-752 (2004); Byfield, S. D., and Roberts. A. B., Trends Cell Biol. 14, 107-111 (2004); Dumont, N, et al., Cancer Cell 3, 531-536 (2003); WO Publication No. 2002/094833; WO Publication No. 2004/026865; WO Publication No. 2004/067530; WO Publication No. 2091032667; WO Publication No. 2004/013135; WO Publication No. 2003/097639; WO Publication No. 2007/048857; WO Publication No. 2007/018818; WO Publication No. 2006/018967; WO Publication No. 2005/039570; WO Publication No. 2000/031135; WO Publication No. 1999/058128; WO Publication No. 2004/026871; WO Publication No. 2004/021989; WO Publication No. 2004/026307; WO Publication No. 2000/012497; U.S. Pat. No. 6,509,318; U.S. Pat. No. 6,090,383; U.S. Pat. No. 6,419,928; U.S. Pat. No. 9,927,738; U.S. Pat. No. 7,223,766; U.S. Pat. No. 6,476,031; U.S. Pat. No. 6,419,928; U.S. Pat. No. 7,030,125; U.S. Pat. No. 6,943,191; U.S. Pat. No. 5,731,144; U.S. Pat. No. 7,151,169; U.S. Publication No. 2005/0245520; U.S. Publication No. 2004/0147574; U.S. Publication No. 2007/0066632; U.S. Publication No. 2003/0028905; U.S. Publication No. 2005/0032835; U.S. Publication No. 2008/0108656; U.S. Publication No. 2004/015781; U.S. Publication No. 2004/0204431; U.S. Publication No. 2006/0003929; U.S. Publication No. 2007/0155722; U.S. Publication No. 2004/0038856; U.S. Publication No. 2005/0245508; U.S. Publication No. 2004/01381188 and U.S. Publication No. 2009/0036382, contents of all of which are herein incorporated in their entireties. Oligonucleotide based modulators of TGF-0 signaling, such as siRNAs and antisense oligonucleotides, are described in U.S. Pat. No. 5,731,424; U.S. Pat. No. 6,124,449; U.S. Publication Nos. 2008/0015161; 2006/0229266; 2004/0006030; 2005/0227936 and 2005/0287128, contents of all of which are herein incorporated in their entireties. Other antisense nucleic acids and siRNAs can be obtained by methods known to one of ordinary skill in the art.

In some embodiments. BMP/TGFβ ligand is BMP4 (bone morphogenetic protein 4). Other exemplary BMP/TGF modulators are described in U.S. Pat. Nos. 7,223,766 and 7,354,722, the contents of which each are incorporated herein by reference in their entirety. In some embodiments, the additional agent for modulating a biological pathway or target is a Na⁺, K⁺ and/or Ca²⁺ ion channel modulator. Exemplary ion channel modulators are described in U.S. Pat. Nos. 6,184,231; 6,479,498; 6,646,012; 5,565,483; 5,871,940; 6,172,085; 5,242,947; 7,132,422; 6,756,400; 7,183,323; 7,226,950 and 6,872,741, and U.S. Ser. Nos. 11/434,920; 11/546,669; 10/514,150; 11/450,695; 11/216,376; 10/743,280; 11/434,627; 10/977,609; 11/811,909; 11/418,163; 11/556,354; 11/432,997; 11/216,899; 11/517,754; 11/418,278; 10/792,688; 11/574,751; 11/643,622; 11/266,142; 10/935,008; 12/158,491; 10/450,215; 10/427,847 and 10/838,087, the contents of which each are incorporated herein by reference in their entirety. In some embodiments, Na⁺/K⁺ channel modulator is a cardiac glycoside selected from the group consisting of Ouabain, Digoxin, Dititoxin, Lanatoside C, and combinations thereof. In some embodiments, Ca²⁺ channel modulator is Thapsigargin, ionomycin or Caklcimycin. In some embodiments, K⁺ channel modulator is Veratridine, Monensin NA or Valinomycin.

In some embodiments, MAPK activator is Anysomycin or Coumermycin.

Exemplary inhibitors of PDE5 are described in U.S. Pat. Nos. 6,869,974; 6,680,047; 6,635,274; 6,555,663 and 6,472,425, the contents of which each are incorporated herein by reference in their entirety. In some embodiments, PDE5 inhibitor is selected from the group consisting of MBCQ, Dipyridamole, spironolactone, bucladesine, and combinations thereof.

In some embodiments, the additional agent for modulating a biological pathway or target is an activator of RTK signaling. Exemplary modulators of RTK signaling are described in U.S. Pat. Nos. 5,196,446; 5,374,652; 6,316,635; 7,214,700; 6,569,868; 5,302,606; and 6,849,641, the contents of which each are incorporated herein by reference in their entirety. In some embodiments, RTK activator is PDGF-BB.

In some embodiments, the additional agent for modulating a biological pathway or target is a growth factor.

In some embodiments, the additional agent for modulating a biological pathway or target is a nucleic acid. Nucleic acid modulators of biological pathways and targets include, but are not limited to, antisense oligonucleotide, siRNA, shRNA, ribozyme, aptamers, antisense RNA, and decoy oligonucleotides. Methods of preparing such nucleic acids are known in the art and easily available to those skilled in the art.

In some embodiments, amino acid based molecules, such a peptides, oligopeptides and proteins, can be used as additional agents to modulate the biological pathways or targets described herein.

In some embodiments, antibodies can be used as additional agents to modulate the biological pathways or targets described herein. As used herein, the term “antibody” includes complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)₂, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, singlo-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred. Two single chain antibodies can be combined to form a diabody, also known as a bivalent dimer. Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites. Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific. Thus, antibodies useful in the methods described herein include, but are not limited to, naturally occurring antibodies, bivalent fragments such as (Fab), monovalent fragments such as Fab, single chain antibodies, single chain Fv (scFv), single domain antibodies, multivalent single chain antibodies, diabodies, triabodies, and the like that bind specifically with an antigen. While both polyclonal and monoclonal antibodies can be used in the methods described herein, it is preferred that a monoclonal antibody is used where conditions require increased specificity for a particular protein. Antibodies can be raised against a biological pathway component or target by methods known to those skilled in the art. Such methods are described in detail, for example, in Harlow et al., 1988 in: Antibodies, A Laboratory Manual, Cold Spring Harbor, N.Y.

Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikahank et al. ((1997) J. Acquired Immune Deficiency Syndromes and Human Retrovirology 14:193).

In some embodiments, a compound, agent, or method described herein promotes motor neuron survival by increasing SMN protein levels n motor neuron. In some embodiments, the SMN protein levels are increased by about at least 5%, 10%, 20%, 30%, 40%, 50%, 60%, 70%, 80%, 90%. 95%, 100%, 0.1.1-fold, 1.25-fold, 1.5-fold, 1.75-fold, 2-fold, 3-fold, 4-fold, 5-fold, 10-fold or more relative to a control.

Without wishing to be bound by theory, some of the methods, agents, and compounds described herein can promote motor neuron survival without increasing SMN protein levels and/or GEMs in the motor neurons. In some embodiments, a compound, agent, or method described herein promotes motor neuron survival without increasing SMN protein levels in motor neuron. For a non-limiting example, kenpaullone and compounds GSK1 and GSK13 promote motor neuron survival without increasing SMN protein levels in the motor neuron.

### Compositions

The work described herein demonstrates, inter alia that HGK inhibition can promote motor neuron survival. Accordingly, in one aspect, disclosed herein is a composition comprising an HGK inhibitor.

The present invention relates to the discovery that kenpaullone, a known inhibitor of GSK-3, inhibits HGK and promotes motor neuron survival. In another aspect, disclosed herein is a composition, the composition comprising an effective amount of an HGK inhibitor and an effective amount of a GSK-3 inhibitor. It should be appreciated that the HGK inhibitor and the GSK-3 inhibitor can be any of the inhibitors described herein or otherwise apparent to those skilled in the art. It should also be appreciated that the HGK inhibitor and the GSK-3 inhibitor can be the same agent (e.g., kenpaullone) or different agents. 11001921 In some embodiments, the HGK inhibitor and the GSK-3 inhibitor work synergistically to increase motor neuron survival such that the increase in motor neuron survival is higher than the cumulative effect of the HGK inhibitor and the GSK-3 inhibitor alone. In some embodiments, the GSK-3 inhibitor modulates a pathway that is different from the pathway or target that is modulated (e.g., inhibited) by the HGK inhibitor.

Without limitations, the HGK inhibitor and the GSK-3 inhibitor can be present in any ratio by weight or by moles. For example, the HGK inhibitor and the GSK-3 inhibitor can be present in a ratio of 50:1, 40:1, 30:1,25:1,20:1, 15:1, 10:1, 5:1, 3:1, 2:1, 1:1.75, 1.5:1, or 1.25:1 to 1:1.25, 1:1.5, 1.75, 1:2, 1:3, 1:4, 1:5, 1:10, 1:15, 1:20, 1:20, 1:30, 1:40, or 1:50.

The work described herein also demonstrates, inter alia, that reduced activation of the HGK-Tak1-MKK4-JNK-c-Jun cell death signaling cascade promotes motor neuron survival. Accordingly, in one aspect, disclosed herein is a composition, the composition comprising an effective amount of an agent that decreases activation of the HGK-Tak1-MKK4-JNK-c-Jun cell death signaling cascade.

In some embodiments, the composition comprises an effective amount of an agent that decreases activation of the HGK-Tak1-MKK4-JNK-c-Jun cell death signaling cascade and an effective amount of a GSK-3 inhibitor. It should be appreciated that the agent that decreases activation of the HGK-Tak1-MKK4-JNK-c-Jun cell death signaling cascade and the GSK-3 inhibitor can be any of the inhibitors described herein or otherwise apparent to those skilled in the art. It should also be appreciated that the agent that decreases activation of the HGK-Tak1-MKK4-JNK-c-Jun cell death signaling cascade and the GSK-3 inhibitor can be the same agent (e.g., kenpaullone) or different agents.

In some embodiments, the agent that decreases activation of the HGK-Tak1-MKK4-JNK-c-Jun cell death signaling cascade and the GSK-3 inhibitor work synergistically to increase motor neuron survival such that the increase in motor neuron survival is higher than the cumulative effect of the agent that decreases activation of the HGK-Tak1-MKK4-JNK-c-Jun cell death signaling cascade and the GSK-3 inhibitor alone. In some embodiments, the GSK-3 inhibitor modulates a pathway that is different from the pathway or target that is modulated (e.g., inhibited) by the agent that decreases activation of the HGK-Tak1-MKK4-JNK-c-Jun cell death signaling cascade.

Without limitations, the agent that decreases activation of the HGK-Tak1-MKK4-JNK-c-Jun cell death signaling cascade and the GSK-3 inhibitor can be present in any ratio by weight or by moles. For example, the agent that decreases activation of the HGK-Tak1-MKK4-JNK-c-Jun cell death signaling cascade and the GSK-3 inhibitor can be present in a ratio of 50:1, 40:1, 30:1, 25:1, 20:1, 15:1, 10:1, 5:1, 3:1, 2:1, 1:1.75, 1.5:1, or 1.25:1 to 1:1.25, 1:1.5, 1.75, 1:2, 1:3, 1:4, 1:5, 1:10, 1:15, 1:20, 1:20, 1:30, 1:40, or 1:50.

As discussed herein, the inventors have discovered, inter alia, that while a compound of formula (I) generally inhibits motor neuron survival when used alone, unexpectedly, a compound of formula (I) can promote motor neuron survival in a synergistic manner when used together with an additional agent that modulates a biological pathway or target described herein. In other words, the compound of formula (I) and the additional agent work synergistically to increase motor neuron survival. The increase in motor neuron survival is higher than the cumulative effect of the compound of formula (I) and the additional agent used alone.

The additional agent can modulate a pathway that is different from the pathway or target that is modulated, e.g., inhibited by the compound of formula (I) described herein. In some embodiments, the additional agent modulates a non-CDK biological pathway, a non-CDK target, a non-PAK biological pathway or a non-PAK target. Accordingly, provided herein is also a synergistic composition comprising a PAK inhibitor and an additional agent, wherein the additional agent modulates a non-PAK biological pathway or a non-PAK target, e.g., a biological pathway or a target described herein. Without wishing to be bound by a theory, the PAK inhibitor and the additional agent in the synergistic composition can work together in a synergistic manner to increase motor neuron survival.

Without limitations, the compound of formula (I), e.g., a PAK or CKD inhibitor, and the additional agent can be present in any ratio by weight or by moles. For example, the PAK inhibitor and the additional agent can be present in a ratio of 50:1, 40:1, 30:1, 25:1, 20:1,15:1, 10:1,5:1, 3:1, 2:1, 1:1.75, 1.5:1, or 1.25:1 to 1:1.25, 1:1.5, 1.75, 1:2, 1:3, 1:4, 1:5, 1:10, 1:15, 1:20, 1:20, 1:30, 1:40, or 1:50.

### Diagnostic Tests

The disclosure also contemplates diagnostic tests and methods of diagnosing neurodegenerative disorders and/or disorders characterized by neuronal cell death.

In one aspect, a method of diagnosing a neurodegenerative disorder in a subject, comprises: (a) obtaining a biological sample from the subject comprising neuronal cells; (b) conducting at least one binding assay on the neuronal cells to detect the level or activity of HGK in the neuronal cells; and (c) diagnosing the subject as having the neurodegenerative disorder if the level or activity of HGK in the neuronal cells is increased relative to level or activity of HGK in a control.

In another aspect, a method of diagnosing disorder characterized by neuronal cell death in a subject, comprises: (a) obtaining a biological sample from the subject comprising neuronal cells; (b) conducting at least one binding assay on the neuronal cells to detect the level or activity of HGK in the neuronal cells; and (c) diagnosing the subject as having the neurodegenerative disorder if the level or activity of HGK in the neuronal cells is increased relative to level or activity of HGK in a control.

Any suitable control can be used. In some embodiments, the control is a subject that does not have the neurodegenerative disorder. In some embodiments, the control is a reference standard or level indicative of a subject that does not have the neurodegenerative disorder.

The disclosure contemplates using any binding assay that is capable of detecting the level or activity of HGK in a cell. In some embodiments, the at least one binding assay comprises a protein kinase assay to detect the phosphorylation activity of HGK. For example, a protein kinase assay can be used to detect HGK phosphorylation of Tak1 or a protein that interacts with Tak1. Those skilled in the art will appreciate how to conduct kinase assays suitable for detecting the level or activity of HGK in a cell.

In some embodiments, the at least one binding assay comprises an assay that measures a level of HGK mRNA or protein in the neuronal cell. Any assays that are capable of measuring mRNA or protein in a cell can be used (e.g., hybridization assays (e.g., microarrays, qRT-PCR, etc.), and immunological based assays (e.g., Western blotting, immunohistochemistry, etc.) It should be appreciated by the skilled artisan that increased levels of HGK mRNA and/or protein in a neuronal cell relative to a control neuronal cell obtained from a subject that does not have the neurodegenerative disorder or a reference standard or level is indicative that the subject has or is at risk for developing the neurodegenerative disorder and/or a disorder characterized by neuronal cell death.

In some embodiments, the at least one binding assay comprises a protein kinase assay to detect the level of phosphorylation of a protein kinase downstream to HGK in the HGK-Tak1-MKK4-JNK-c-Jun cell death signaling cascade. Examples of such kinases include Tak1, MKK4, and JNK.

In still another aspect, a method of diagnosing a neurodegenerative disorder in a subject comprises (a) obtaining a biological sample from the subject comprising neuronal cells; (b) conducting at least one kinase assay on the neuronal cells to detect activation of the HGK-Tak1-MKK4-JNK-c-Jun cell death signaling cascade in the neuronal cells; and (c) diagnosing the subject as having the neurodegenerative disorder if the HGK-Tak1-MKK4-JNK-c-Jun cell death is activated in the neuronal cells.

In some embodiments, the HGK-Tak1-MKK4-JNK-c-Jun cell death signaling cascade in the neuronal cells is activated if the phosphorylation level or activity of a protein kinase in the HGK-Tak1-MKK4-JNK-c-Jun cell death signaling cascade is increased relative to the phosphorylation level or activity of a protein kinase in the HGK-Tak1-MKK4-JNK-c-Jun cell death signaling cascade in a normal subject that does not have the neurodegenerative disorder.

In some embodiments, the diagnostic methods further comprise selecting a subject suspected of having a neurodegenerative disorder. In some embodiments, the diagnostic methods further comprise selecting a subject suspected of having a disorder characterized by neuronal cell death.

In some embodiments, the neuronal cells comprise motor neurons. In some embodiments, the neuronal cells comprise sensory neurons. In some embodiments, the neurodegenerative disorder is a neurodegenerative disorder described herein. In some embodiments, the disorder characterized by neuronal cell death is such a disorder described herein. In some embodiments, the neurodegenerative disorder is ALS.

In one aspect, a method of diagnosing amyotrophic lateral sclerosis (ALS) in a subject comprises: (a) obtaining a biological sample from the subject comprising neuronal cells; (b) conducting at least one binding assay on the neuronal cells to detect the level or activity of HGK in the neuronal cells; and (c) diagnosing the subject as having ALS if the level or activity of HGK in the neuronal cells is increased relative to level or activity of HGK in a control.

In one aspect, a method of diagnosing amyotrophic lateral sclerosis (ALS) in a subject comprises: (a) obtaining a biological sample from the subject comprising neuronal cells; (b) conducting at least one kinase assay on the neuronal cells to detect activation of the HGK-Tak1-MKK4-JNK-c-Jun cell death signaling cascade in the neuronal cells; and (c) diagnosing the subject as having ALS if the HGK-Tak1-MKK4-JNK-c-Jun cell death is activated in the neuronal cells.

In some embodiments, the HGK-Tak1-MKK4-JNK-c-Jun cell death signaling cascade in the neuronal cells is activated if the phosphorylation level or activity of a protein kinase in the HGK-Tak1-MKK4-JNK-c-Jun cell death signaling cascade is increased relative to the phosphorylation level or activity of a protein kinase in the HGK-Tak1-MKK4-JNK-c-Jun cell death signaling cascade in a control.

Any suitable control can be used. In some embodiments, the control is a subject that does not have ALS. In some embodiments, the control is a reference standard or level indicative of a lack of ALS. In some embodiments, the subject suspected of having ALS. In some embodiments, the neuronal cells comprise motor neurons.

### Motor Neurons

In some embodiments, the motor neurons comprise a mutation in a gene associated with a neurodegenerative disorder. One non limiting example of a gene associated with a neurodegenerative disorder is SMN1. Another non limiting example of a gene associated with a neurodegenerative disorder is SOD1. A variety of SOD1 mutant alleles are known to be associated with SMA and/or ALS, including without limitation. SOD1A4V, SOD1G85R, and SOD1G93A.

In some embodiments, methods of the invention employ cells that are not motor neurons, wherein the cells can comprise a mutation in a gene associated with a neurodegenerative disorder. In one non-limiting example, some methods the present invention employ fibroblasts comprising a mutation in a gene associated with a neurodegenerative disorder. In some embodiments, methods of the invention employ fibroblasts comprising a mutation in a SOD1 gene, such as, without limitation, SOD1A4V, SOD1G8SR, and SOD1G93A.

As used herein, the term “SOD1” refers to either the gene encoding superoxide dismutase 1 or the enzyme encoded by this gene. The SOD1 gene or gene product Is known by other names in the art including, but not limited to, ALS1, Cu/Zn superoxide dismutase, indophenoloxidase A, IPOA, and SODC_HUMAN. Those of ordinary skill in the art will be aware of other synonymous names that refer to the SOD1 gene or gene product. The SOD1 enzyme neutralizes supercharged oxygen molecules (called superoxide radicals), which can damage cells if their levels are not controlled. The human SOD1 gene maps to cytogenetic location 21q22.1. Certain mutations in SOD1 are associated with ALS in humans including, but not limited to, Ala4Val, Gly37Arg. G85R and Gly93Ala, and more than one hundred others. Those of ordinary skill in the art will be aware of these and other human mutations associated with ALS. Certain compositions and methods of the present invention comprise or employ cells comprising a SOD1 mutation.

“SOD1 mutations” refer to mutations in the SOD1 gene (NC 000021.8; NT_011512.11; AC_000064.1; NW_927384.1; AC_000153.1; NW_001838706.1 NM_000454.4; NP_000445.1 and NCBI Entrez GeneID: 6647) including but are not limited to Ala4Val, Cys6Gly, Va17Glu, Leu8Val, Gly10Val, Gly12Arg, Val14Met, Gly16Ala, Asn19Ser, Phe20Cys, Glu21Lys, Gln22Leu, Gly37Arg. Leu38Arg. Gly41Ser, His43Arg. Phe45Cys, His46Arg, Va147Phe, His48Gln, Glu49Lys, Thr54Arg, Ser59Ile, Asn65Ser, Leu67Arg, Gly72Ser, Asp76 Val, His80Arg, Leu84Phe, Gly85Arg. Asn86Asp, Va187Ala, Ala89Val, Asp90Ala, Gly93Ala, Ala95Thr, Asp96Asn, Val97Met, Glu100Gly, Asp10Asn, Ile104Phe, Ser105Leu, Leu106Val, Gly108Val, Ile12Thr, Ile13Phe, Gly114Ala, Arg115Gly, Val118Leu, Ala140Gly, Ala145Gly, Asp124Val, Asp124Gly, Asp125His, Leu126Ser, Ser134Asn, Asn 139His, Asn139Lys, Gly141Glu, Leu144Phe, Leu144Ser, Cys146Arg, Ala145Thr, Gly147Arg, Val148Gly, Va1148Ile, Ile149Thr, Ile151Thr, and Ile151Ser. SOD1 is also known as ALS, SOD, ALS1, IPOA, homodimer SOD1. “SOD1 mutation” databases can be found at Dr. Andrew C. R. Martin website at the University College of London (www.bioinfo.org.uk), the ALS/SOD1 consortium website (www.alsod.org) and the human gene mutation database (HGMD®) at the Institute of Medical Genetics at Cardiff, United Kingdom.

Contacting of Motor Neurons with Compounds

Motor neurons can be contacted with the compounds and/or agents described herein in a cell culture e.g., in vitro or ex vivo, or administrated to a subject, e.g., In vivo. In some embodiments of the invention, a compound and/or agent described herein can be administrated to a subject to treat, prevent, and/or diagnose neurodegenerative disorders, including those described herein. In some embodiments, a compound and/or agent described herein can be administered to a subject to treat, prevent, and/or diagnose ALS. In some embodiments, a compound and/or agent described herein can be administered to a subject to treat, prevent, and/or diagnose SMA.

The term “contacting” or “contact” as used herein in connection with contacting a motor neuror cell includes subjecting the cell to an appropriate culture media which comprises the indicated compound and/or agent Where the motor neuron is in vivo, “contacting” or “contact” includes administering the compound and/or agent in a pharmaceutical composition to a subject via an appropriate administration route such that the compound and/or agent contacts the motor neuron in vivo. Measurement of cell survival can be based on the number of viable cells after period of time has elapsed after contacting of cells with a compound or agent. For example, number of viable cells can be counted after about at least 5 minutes, 10 minutes, 20 minutes, 30 minutes, 40 minutes, 50 minutes, 1 hour, 2 hours, 4 hours, 6 hours, 12 hours, 24 hours, 2 days, 3 days or more and compared to number of viable cells in a non treated control.

For in vitro methods, motor neurons can be obtained from different sources. For example, motor neurons can be obtained from a subject, or derived from non motor neuron cells from a subject. In some embodiments, motor neuron is a whole cell. In some embodiments, the subject is suffering from a neurodegenerative disorder. In some embodiments, the subject is at risk of developing a neurodegenerative disorder. In some embodiments, the subject is suspected of having a neurodegenerative disorder. In some embodiments, the subject is at risk of developing a disorder characterized by neuronal cell death. In some embodiments, the subject is suspected of suffering from a disorder characterized by neuronal cell death. In some embodiments, the subject is suffering from neuronal cell death. In some embodiments, the subject is suffering from SMA. In some embodiments, the subject is suffering from ALS. In some embodiments, the subject is suffering from multiple sclerosis. In some embodiments, the subject is suffering from Parkinson's disease. In some embodiments, the subject is suffering from Huntington's disease. In some embodiments, the subject is a carrier e.g., a symptom-free carrier. In some embodiments, motor neuron cells are derived from a subject's embryonic stem cells (ESCs). In some embodiments, the subject is human. In some embodiments, the subject is mouse. In some embodiments, mouse is a transgenic mouse. Methods of inducing motor neuron differentiation from embryonic stem cells are known in the art, for example as described in Di Giorgio ea al., Nature Neuroscience (2007), published online 15 Apr. 2007; doi: 10.1038/nn1885 and Wichterle et al., Cell (2002) 110:385-397. In some instances induced pluripotent stem cells can be generated from a subject and then differentiated into motor neurosn. One exemplary method of deriving motor neurons from a subject is described in Dimos, J. T., et al. Science (2008) 321, 1218-122 (Epub Jul. 31, 2008).

For in vivo methods, a therapeutically effective amount of a compound described herein can be administered to a subject. Methods of administering compounds to a subject are known in the art and easily available to one of skill in the art.

As one of skill in the art is aware, promoting survival of motor neuron cells in a subject can lead to treatment, prevention or amelioration of a number of neurodegenerative disorders. By “neurodegenerative disorder” is meant any disease or disorder caused by or associated with the deterioration of cells or tissues of the nervous system. In some instances, the neurodegenerative disorder is characterized by neuronal cell death (e.g., motor neurons and/or sensory neurons). Exemplary neurodegenerative disorders are polyglutamine expansion disorders (e.g., HD, dentalorubropallidoluysian atrophy, Kennedy's disease (also referred to as spinobulber muscular atrophy), and spinocerebellar ataxia (e.g., type 1, type 2, type 3 (also referred to as Machado-Joseph disease), type 6, type 7, and type 17)), other trinucleotide repeat expansion disorders (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer disease, amyotrophic lateral sclerosis (ALS), ataxia telanglectasia, Batten disease (also referred to as Spielmeyer-Vogt-Sjogren-Batten disease), Canavan disease, Cockayne syndrome, corticobasal degeneration, Creutzfcldt-Jakob disease, ischemia stroke, Krabbe disease, Lewy body dementia, multiple sclerosis, multiple system atrophy, Parkinson's disease, Pelizacus-Merzbacher disease, Pick's disease, primary lateral sclerosis, Refsum's disease, Sandhoff disease, Schildets disease, spinal cord injury, spinal muscular atrophy (SMA), SteeleRichardson-Olszewski disease, and Tabes dorsalis.

Those skilled in the art will also appreciate that the compounds and/or agents disclosed herein can be used for promoting neuronal cell survival (e.g., motor neurons and/or sensory neurons). Promoting neuronal cell survival in a subject can lead to treatment, prevention or amelioration of a number of disorders characterized by neuronal cell death. Examples of neuronal cell death-related disorders or conditions that can be treated or can be prevented include, but are not limited to various neurodegenerative disorders (e.g. Alzheimer's disease, Huntington's Disease, prion diseases, Parkinson's Disease, amyotrophic lateral sclerosis, ataxia telangiectasia, spinobulbar atrophy, age-related reduction in number or in function, macular degeneration, retinal degeneration, dominant optic atrophy and Leber's hereditary optic neuropathy), diseases and conditions induced under various conditions of ischemia and/or excitotoxicity (e.g. ischemic stroke, hemorrhagic stroke and ischemic optic neuropathy), diseases due to nervous system trauma (e.g. spinal cord injury or traumatic optic neuropathy, or brain injury associated with physiological trauma), diseases due to inflammation (e.g. optic neuritis or multiple sclerosis), diseases due to infection (e.g. meningitis and toxoplasmosis optic neuropathy), diseases and conditions induced by certain medications or irrigating solutions (e.g. optic neuropathy induced by ethambutol or methanol), and diseases due to other etiologies (e.g. glaucoma).

The motor neuron diseases (MND) are a group of neurodegenerative disorders that selectively affect motor neurons, the nerve cells that control voluntary muscle activity including speaking, walking, breathing, swallowing and general movement of the body. Skeletal muscles are innervated by a group of neurons (lower motor neurons) located in the ventral horns of the spinal cord which project out the ventral roots to the muscle cells. These nerve cells are themselves innervated by the corticospinal tract or upper motor neurons that project from the motor cortex of the brain. On macroscopic pathology, there is a degeneration of the ventral horns of the spinal cord, as well as atrophy of the ventral roots. In the brain, atrophy may be present in the frontal and temporal lobes. On microscopic examination, neurons may show spongiosis, the presence of astrocytes, and a number of inclusions including characteristic “skein-like” inclusions, bunina bodies, and vacuolisation. Motor neuron diseases are varied and destructive in their effect They commonly have distinctive differences in their origin and causation, but a similar result in their outcome for the patient: severe muscle weakness. Amyotrophic lateral sclerosis (ALS), primary lateral sclerosis (PLS), progressive muscular atrophy (PMA), pseudobulbar palsy, progressive bulbar palsy, spinal muscular atrophy (SMA) and post-polio syndrome are all examples of MND. The major site of motor neuron degeneration classifies the disorders. As used herein, the phrase “motor neuron degeneration” or “degeneration of motor neuron” means a condition of deterioration of motor neurons, wherein the neurons die or change to a lower or less functionally-active form.

Common MNDs include amyotrophic lateral sclerosis, which affects both upper and lower motor neurons. Progressive bulbar palsy affects the lower motor neurons of the brain stem, causing slurred speech and difficulty chewing and swallowing. Individuals with these disorders almost always have abnormal signs in the arms and legs. Primary lateral sclerosis is a disease of the upper motor neurons, while progressive muscular atrophy affects only lower motor neurons in the spinal cord. Means for diagnosing MND are well known to those skilled in the art. Non limiting examples of symptoms are described below.

### Amyotrophic Lateral Sclerosis (ALS)

Amyotrophic lateral sclerosis (ALS), also called Lou Gehrig's disease or classical motor neuron disease, is a progressive, ultimately fatal disorder that eventually disrupts signals to all voluntary muscles. In the United States, doctors use the terms motor neuron disease and ALS interchangeably. Both upper and lower motor neurons are affected. Approximately 75 percent of people with classic ALS will also develop weakness and wasting of the bulbar muscles (muscles that control speech, swallowing, and chewing). Symptoms are usually noticed first in the arms and hands, legs, or swallowing muscles. Muscle weakness and atrophy occur disproportionately on both sides of the body. Affected individuals lose strength and the ability to move their arms, legs, and body. Other symptoms include spasticity, exaggerated reflexes, muscle cramps, fasciculations, and increased problems with swallowing and forming words. Speech can become slurred or nasal. When muscles of the diaphragm and chest wall fail to function properly, individuals lose the ability to breathe without mechanical support. Although the disease does not usually impair a person's mind or personality, several recent studies suggest that some people with ALS may have alterations in cognitive functions such as problems with decision-making and memory. ALS most commonly strikes people between 40 and 60 years of age, but younger and older people also can develop the disease. Men are affected more often than women. Most cases of ALS occur sporadically, and family members of those individuals are not considered to be at increased risk for developing the disease. However, there is a familial form of ALS in adults, which often results from mutation of the superoxide dismutase gone, or SOD, located on chromosome 21. In addition, a rare juvenile-onset form of ALS is genetic. Most individuals with ALS die from respiratory failure, usually within 3 to 5 years from the onset of symptoms. However, about 10 percent of affected individuals survive for 10 or more years.

### Spinal Muscular Atrophy (SMA)

Spinal muscular atrophy (SMA) refers to a number of different disorders, all having in common a genetic cause and the manifestation of weakness due to loss of the motor neurons of the spinal cord and brainstem. Weakness and wasting of the skeletal muscles is caused by progressive degeneration of the anterior horn cells of the spinal cord. This weakness is often more severe in the legs than in the arms. SMA has various forms, with different ages of onset, patterns of inheritance, and severity and progression of symptoms. Some of the more common SMAs are described below.

Defects in SMN gene products are considered as the major cause of SMA and SMN protein levels correlate with survival of subject suffering from SMA. The most common form of SMA is caused by mutation of the SMN gene. The region of chromosome 5 that contains the SMN (survival motor neuron) gene has a large duplication. A large sequence that contains several genes occurs twice in adjacent segments. There are thus two copies of the gene, SMN1 and SMN2. The SMN2 gene has an additional mutation that makes it less efficient at making protein, though it does so in a low level. SMA is caused by loss of the SMN1 gene from both chromosomes. The severity of SMA, ranging from SMA 1 to SMA 3, is partly related to how well the remaining SMN 2 genes can make up for the loss of SMN 1.

SMA type I, also called Werdnig-Hoffmann disease, is evident by the time a child is 6 months old. Symptoms may include hypotonia (severely reduced muscle tone), diminished limb movements, lack of tendon reflexes, fasciculations, tremors, swallowing and feeding difficulties, and impaired breathing Some children also develop scoliosis (curvature of the spine) or other skeletal abnormalities. Affected children never sit or stand and the vast majority usually die of respiratory failure before the age of 2.

Symptoms of SMA type II usually begin after the child is 6 months of age. Features may include inability to stand or walk, respiratory problems, hypotonia, decreased or absent tendon reflexes, and fasciculations. These children may learn to sit but do not stand. Life expectancy varies, and some individuals live into adolescence or later.

Symptoms of SMA type III (Kugelberg-Welander disease) appear between 2 and 17 years of age and include abnormal gait; difficulty running, climbing steps, or rising from a chair, and a fine tremor of the fingers. The lower extremities are most often affected. Complications include scoliosis and joint contractures—chronic shortening of muscles or tendons around joints, caused by abnormal muscle tone and weakness, which prevents the joints from moving freely.

Other forms of SMA include e.g., Hereditary Bulbo-Spinal SMA Kennedy's disease (X linked, Androgen receptor), SMA with Respiratory Distress (SMARD 1) (chromsome 11. IGHMBP2 gene), Distal SMA with upper limb predominance (chromosome 7, glycyl tRNA synthase), and X-Linked infantile SMA (gene UBE1).

Current treatment for SMA consists of prevention and management of the secondary effect of chronic motor unit loss. Some drugs under clinical investigation for the treatment of SMA include butyrates, Valproic acids, hydroxyurea and Riluzole.

Symptoms of Fazio-Londe disease appear between 1 and 12 years of age and may include facial weakness, dysphagia (difficulty swallowing), stridor (a high-pitched respiratory sound often associated with acute blockage of the larynx), difficulty speaking (dysauthria), and paralysis of the eye muscles. Most individuals with SMA type III die from breathing complications.

Kennedy disease, also known as progressive spinobulber muscular atrophy, is an X-linked recessive disease. Daughters of individuals with Kennedy disease are carriers and have a 50 percent chance of having a son affected with the disease. Onset occurs between 15 and 60 years of age. Symptoms include weakness of the facial and tongue muscles, hand tremor, muscle cramps, dysphagia, dysarthria, and excessive development of male breasts and mammary glands. Weakness usually begins in the pelvis before spreading to the limbs. Some individuals develop noninsulin-dependent diabetes mellitus.

The course of the disorder varies but is generally slowly progressive. Individuals tend to remain ambulatory until late in the disease. The life expectancy for individuals with Kennedy disease is usually normal.

Congenital SMA with arthrogryposis (persistent contracture of joints with fixed abnormal posture of the limb) is a rare disorder. Manifestations include severe contractures, scoliosis, chest deformity, respiratory problems, unusually small jaws, and drooping of the upper eyelids.

Progressive bulbar palsy, also called progressive bulbar atrophy, involves the bulb-shaped brain stem—the region that controls lower motor neurons needed for swallowing, speaking, chewing, and other functions. Symptoms include pharyngeal muscle weakness (involved with swallowing), weak jaw and facial muscles, progressive loss of speech, and tongue muscle atrophy. Limb weakness with both lower and upper motor neuron signs is almost always evident but less prominent. Affected persons have outbursts of laughing or crying (called emotional lability). Individuals eventually become unable to eat or speak and are at increased risk of choking and aspiration pneumonia, which is caused by the passage of liquids and food through the vocal folds and into the lower airways and lungs. Stroke and myasthenia gravis each have certain symptoms that are similar to those of progressive bulbar palsy and must be ruled out prior to diagnosing this disorder. In about 25 percent of ALS cases early symptoms begin with bulbar involvement Some 75 percent of individuals with classic ALS eventually show some bulbar involvement Many clinicians believe that progressive bulbar palsy by itself, without evidence of abnormalities in the arms or legs, is extremely rare.

Pseudobulbar palsy, which shares many symptoms of progressive bulbar palsy, is characterized by upper motor neuron degeneration and progressive loss of the ability to speak, chew, and swallow. Progressive weakness in facial muscles leads to an expressionless face. Individuals may develop a gravelly voice and an increased gag reflex. The tongue may become immobile and unable to protrude from the mouth. Individuals may also experience emotional lability.

Primary lateral sclerosis (PLS) affects only upper motor neurons and is nearly twice as common in men as in women. Onset generally occurs after age 50. The cause of PLS is unknown. It occurs when specific nerve cells in the cerebral cortex (the thin layer of cells covering the brain which is responsible for most higher level mental functions) that control voluntary movement gradually degenerate, causing the muscles under their control to weaken. The syndrome-which scientists believe is only rarely hereditary-progresses gradually over years or decades, leading to stiffness and clumsiness of the affected muscles. The disorder usually affects the legs first, followed by the body trunk, arms and hands, and, finally, the bulbar muscles. Symptoms may include difficulty with balance, weakness and stiffness in the legs, clumsiness, spasticity in the legs which produces slowness and stiffness of movement, dragging of the feet (leading to an inability to walk), and facial involvement resulting in dysarthria (poorly articulated speech). Major differences between ALS and PLS (considered a variant of ALS) are the motor neurons involved and the rate of disease progression. PLS may be mistaken for spastic paraplegia, a hereditary disorder of the upper motor neurons that causes spasticity in the legs and usually starts in adolescence. Most neurologists follow the affected individual's clinical course for at least 3 years before making a diagnosis of PLS. The disorder is not fatal but may affect quality of life. PLS often develops into ALS.

Progressive muscular atrophy (PMA) is marked by slow but progressive degeneration of only the lower motor neurons. It largely affects men, with onset earlier than in other MNDs. Weakness is typically seen first in the hands and then spreads into the lower body, where it can be severe. Other symptoms may include muscle wasting, clumsy hand movements, fasciculations, and muscle cramps. The trunk muscles and respiration may become affected. Exposure to cold can worsen symptoms. The disease develops into ALS in many instances.

Post-polio syndrome (PPS) is a condition that can strike polio survivors decades after their recovery from poliomyelitis. PPS is believed to occur when injury, illness (such as degenerative joint disease), weight gain, or the aging process damages or kills spinal cord motor neurons that remained functional after the initial polio attack. Many scientists believe PPS is latent weakness among muscles previously affected by poliomyelitis and not a new MND. Symptoms include fatigue, slowly progressive muscle weakness, muscle atrophy, fasciculations, cold intolerance, and muscle and joint pain. These symptoms appear most often among muscle groups affected by the initial disease. Other symptoms include skeletal deformities such as scoliosis and difficulty breathing, swallowing, or sleeping. Symptoms are more frequent among older people and those individuals most severely affected by the earlier disease. Some individuals experience only minor symptoms, while others develop SMA and, rarely, what appears to be, but is not, a form of ALS. PPS is not usually life threatening. Doctors estimate the incidence of PPS at about 25 to 50 percent of survivors of paralytic poliomyelitis.

In some embodiments, neurodegenerative disorder can be SMA or ALS.

By “treatment, prevention or amelioration of neurodegenerative disorder” is meant delaying or preventing the onset of such a disorder (e.g. death of motor neurons), at reversing, alleviating, ameliorating, inhibiting, slowing down or stopping the progression, aggravation or deterioration the progression or severity of such a condition. In one embodiment, the symptom of a neurodegenerative disorder is alleviated by at least 20%, at least 30%, at least 40%, or at least 50%. In one embodiment, the symptom of a neurodegenerative disorder is alleviated by more that 50%. In one embodiment, the symptom of a neurodegenerative disorder is alleviated by 80%, 90%, or greater. Treatment also includes improvements in neuromuscular function. In some embodiments, neuromuscular function improves by at least about 10%, 20%, 30%, 40%, 50% or more.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. In some embodiments of the invention, the subject suffers from a neurodegenerative disorder.

In some embodiments, the methods described herein further comprise selecting a subject diagnosed with a neurodegenerative disorder. A subject suffering from a neurodegenerative disorder can be selected based on the symptoms presented. For example a subject suffering from SMA may show symptoms of hypotonia, diminished limb movements, lack of tendon reflexes, fasciculations, tremors, swallowing, feeding difficulties, impaired breathing, scoliosis or other skeletal abnormalities, inability to stand or walk, abnormal gait, difficulty running difficulty climbing steps, difficulty rising from a chair, and/or fine tremor of the fingers.

In some embodiments, the methods described herein further comprise selecting a subject at risk of developing a neurodegenerative disorder. A subject at risk of developing a neurodegenerative disorder can be selected based on a genetic diagnostic test (e.g., for a mutation in a gene associated with a neurodegenerative disorder (e.g., a mutation in the SOD1 gene, or in an SMN gone)) or based on the symptoms presented. For example a subject suffering from SMA may show symptoms of hypotonia, diminished limb movements, lack of tendon reflexes, fasciculations, tremors, swallowing feeding difficulties, impaired breathing, scoliosis or other skeletal abnormalities, inability to stand or walk, abnormal gait, difficulty running, difficulty climbing steps, difficulty rising from a chair, and/or fine tremor of the fingers.

In some embodiments, the methods described herein further comprise selecting a subject suspected of having a neurodegenerative disorder. A subject suspected of having a neurodegenerative disorder can be selected based on a genetic diagnostic test (e.g., for a mutation in a gene associated with a neurodegenerative disorder (e.g., a mutation in the SOD1 gene, or in an SMN gene)) or based on the symptoms presented or a combination thereof. For example a subject suffering from SMA may show symptoms of hypotonia, diminished limb movements, lack of tendon reflexes, fasciculations, tremors, swallowing, feeding difficulties, impaired breathing, scoliosis or other skeletal abnormalities, inability to stand or walk, abnormal pit, difficulty running, difficulty climbing steps, difficulty rising from a chair, and/or fine tremor of the fingers.

In another aspect of the invention, the compounds and/or agents described herein inhibit the activity of at least one kinase. In some embodiments of this and other aspects described herein, the kinase is selected from the group consisting of HGK. Tak1, MKK4, JNK, phosphoinositide 3-kinases (PI-3 kinases), phosphoinositide dependant kinase 1 (PDK1), SOK, glycogen synthase kinase 3 (GSK-3), inhibitor of IκB kinase 2 (IKK2), cyclin dependant kinase 2 (CDK2), and RNA dependant protein kinase.

In some embodiments of this and other aspects described herein, the compounds and/or agents described herein inhibit the activity of at least two different kinases. For example, a compound and/or agent described herein can inhibit the activity of GSK-3 and a second kinase. In some embodiments, the second kinase is CDK. As another example, a compound and/or agent described herein can inhibit the activity of HGK and a second kinase (e.g., GSK-3). In some embodiments, a compound and/or agent described herein can inhibit the activity of GSK-3, HGK, and a PAK.

### Screening Assay

The present disclosure contemplates various assays for identifying agents that can increase motor neuron survival, as well as agents that can be used for treating neurodegenerative disorders (e.g., ALS or SMA).

In one aspect, a method of identifying a candidate agent that promotes motor neuron survival comprises (a) contacting a motor neuron with a test agent, (b) measuring (i) the level or activity of HGK or (ii) activation of the HGK-Tak1-MKK4-JNK-c-Jun cell death signaling cascade, in the presence of the test agent, and (c) identifying the candidate agent that promotes motor neuron survival, wherein the test agent is a candidate agent for promoting motor neuron survival if the test agent (i) decreases the level or activity of HGK or (ii) decreases activation of the HGK-Tak1-MKK4-JNK-c-Jun cell death signaling cascade, in the presence of the test agent.

In another aspect, a method of identifying a candidate agent for treating or preventing a neurodegenerative disorder comprises (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent for treating a neurodegenerative disorder, wherein the test agent is a candidate agent for treating a neurodegenerative disorder if the test agent (i) decreases the level or activity of HGK or (ii) decreases activation of the HGK-Tak1-MKK4-JNK-c-Jun cell death signaling cascade, in the presence of the test agent.

In yet another aspect, a method of identifying a candidate agent for treating or preventing ALS, comprises (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent for treating ALS, wherein the test agent is a candidate agent for treating ALS if the test agent (i) decreases the level or activity of HGK or (ii) decreases activation of the HGK-Tak1-MKK4-JNK-c-Jun cell death signaling cascade, in the presence of the test agent.

In still another aspect, a method of identifying a candidate agent for treating or preventing SMA comprises (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent for treating SMA, wherein the test agent is a candidate agent for treating SMA if the test agent (i) decreases the level or activity of HGK or (ii) decreases activation of the HGK-Tak1-MKK4-JNK-c-Jun cell death signaling cascade, in the presence of the test agent.

In some embodiments, the contacting is performed in the absence of trophic factors. In some embodiments, the motor neuron comprises an in vitro-differentiated motor neuron as described herein.

In yet another aspect, disclosed herein are methods for identifying agents that can increase motor neuron survival. In an exemplary embodiment, a method for identifying an agent that can increase motor neuron survival comprises determining the effect of a test agent on a motor neuron, wherein motor neuron is cultured under conditions which minimize survival of motor neurons, e.g., withdrawal of one or more trophic factors, and wherein a higher number of motor neurons in the presence of a test agent relative to a control indicate that the test agent can promote motor neuron survival.

As used herein a “trophic factor” is a molecule that directly or indirectly affests the survival or function of a trophic factor responsive cell. Exemplary trophic factors include Ciliary Neurotrophic Factor (CNTF), basic Fibroblast Growth Factor (bFGF), insulin and insulin-like growth factors (e.g., IGF-I, IGF-H, IGF-IH), inteferons, interleukins, cytokines, and the neurotrophins, including Nerve Growth Factor (NGF), Neurotrophin-3 (NT-3), Neurotrophin-4/5 (NT-4/5) and Brain Derived Neurotrophic Factor (BDNF).

A “trophic factor-responsive cell” is a cell which includes a receptor to which a trophic factor can specifically bind; examples include neurons (e.g., motor neurons) and non-neuronal cells (e.g., monocytes and neoplastic cells).

For the assay, motor neurons are optionally allowed to grow for a period time and trophic factors removed to induce cell death. Period of cell growth can be optimized depending on the assay format, initial plating density of the cells. In some embodiments, a practioner can obtain cells that are already planted in the appropriate vessel and allowed to grow for a period of time. In other embodiments, the pactioner plates the cell in the appropriate vessel and allow the cells to grow for a period time, e.g., at least one day, at least two days, at least three days, at last four days, at least five days, at least six days, at least seven days or more before withdrawal of at least one trophic factor. In one embodiment, cells are grown for four days before withdrawal of at least one trophic factor.

After withdrawal of at least one trophic factor, the test agent is contacted or incubated with the motor neurons. After a sufficient period of time, motor neurons that have survived are counted and their number compared to a control. It has been shown that, while the expression of mutant SOD1 doesn't interfere with the specification and differentiation of MNs, MNs derived from mutant SOD1 ES cells develop more inclusions, have elevated levels of ubiquitinated proteins, and have a later-onset, long-term survival disadvantage when compared to wild-type SOD1 MNs. Now the inventors have discovered that, while there is a survival defect as a consequence of the SOD1^{G93A} genotype at a later stage of MN cultures, there is no significant difference in the basal MN survival between Hb9 and G93A cultures in the first week. Generally the survival rate of G93A MNs is more variable from batch to batch, indicating that the mutant genotype renders the cells more susceptible to mechanical or environmental perturbations (e.g. EB dissociation). Accordingly, for the screening assay, the inventors induced MN death within the first week using trophic factor withdrawal that is independent of the genotype of the cells. This allows implementing the assay in a straightforward and efficient screening (HTS) platform.

Generally, most motor neurons can survive well after two days in the absence of trophic supply (BDNF, GDNF and CNTF). A large proportion of MNs dies after three days in absence of trophic factors, and almost all MNs are killed after four days of starvation. Accordingly, motor neurons are allowed to grow for 2, 3, 4, 5, 6, or 7 days after withdrawal of the trophic factors before counting the motor neurons that survived after trophic factor withdrawal. In one embodiment, cell counting is three days after trophic factor withdrawal. Trophic factors are also referred to a neurotrophic factors or growth factors in the art.

A control can be a sample that is that is not contacted with a compound. A control can be a sample that is treated with a known promoter of motor neuron survival. This can serve as a positive control. A control can be a sample that is treated with a known inhibitor of motor neuron survival.

Some exemplary promoters of motor neuron survival include, but are not limited to, kenpaullone, alsterpaullone, cycloheximide (CHX), and derivatives thereof. Additional promoters of motor neuron survival include those described, for example, in PCT/US2009/061468, filed Oct. 21, 2009, content of which is incorporated herein by reference in its entirety.

As used herein, the term “test agent” refers to agents and/or compositions that are to be screened for their ability to stimulate and/or increase and/or promote motor neuron survival. The test agents can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; nucleic acids; nucleic acid analogs and derivatives; an extract made from biological materials such as bacteria, plants, fungi, or animal cells; animal tissues; naturally occurring or synthetic compositions; and any combinations thereof. In some embodiments, the test agent is a small molecule.

The number of possible test agents runs into millions. Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156(2001). Commercially available compound libraries can be obtained from, e.g., ArQule, Pharmacopia, graffinity, Panvera, Vitas-M Lab, Biomol International and Oxford. These libraries can be screened using the screening devices and methods described herein. Chemical compound libraries such as those from NIH Roadmap, Molecular Libraries Screening Centers Network (MLSCN) can also be used. A comprehensive list of compound libraries can be found at www.broad.harvard.edu/chembio/platform/screening/compoud_libraries/index.htm. A chemical library or compound library is a collection of stored chemicals usually used ultimately in high-throughput screening or industrial manufacture. The chemical library can consist in simple terms of a series of stored chemicals. Each chemical has associated information stored in some kind of database with information such a the chemical structure, purity, quantity, and physiochemical characteristics of the compound.

Depending upon the particular embodiment being practiced, the test agents can be provided free in solution, or may be attached to a carrier, or a solid support, e.g., beads. A number of suitable solid supports may be employed for Immobilization of the test agents. Examples of suitable solid supports include agarose, cellulose, dextran (commercially available as, i.e., Sephadex, Sepharose) carboxymethyl cellulose, polystyrene, polyethylene glycol (PEG), filter paper, nitrocellulose, ion exchange resins, plastic films, polyaminomethylvinylether maleic acid copolymer, glass beads, amino acid copolymer, ethylene-maleic acid copolymer, nylon, silk, etc. Additionally, for the methods described herein, test agents may be screened individually, or in groups. Group screening is particularly useful where hit rates for effective test agents are expected to be low such that one would not expect more than one positive result for a given group.

Without limitations, motor neurons can be plated at any density that provides a optimal signal-to-noise ratio. For example, motor neurons can be plated at a density of 1,000 to 20,000 cells/well in a 384-well plate. In some embodiments, motor neurons are plated at density of 1,000; 2,000; 4,000; 8,000; 12,000; 16,000; or 20,000 cells/well in a 384-well plate. In one embodiment, motor neurons are plated at a density of 8,000 cells/well in a 384-well plate. Based foregoing, one of ordinary skill can adjust the plating density for other cell culturing vessels. For example one can calculate the dimensions of a well in the 384-well plate and the vessels to be used and scale the number of cells to be plated based on volume or surface area ratio between a well from the 384-well plate and the vessel to be used.

In some embodiments, the step of assessing motor neuron survival comprises detecting a motor neuron marker and a cell-replication marker. A selected test agent can be further limited to the agent where the motor neuron marker and the cell-replication marker co-localize in the same cell.

Any available method for identifying and counting motor neurons in a culture can be employed. For example, a motor neuron can comprise a detectable label for identification or counting. As used herein, the term “detectable label” refers to a molecule or an element or functional group in a molecule that allows for the detection, imaging, and/or monitoring of the presence the molecule. Without limitations, a detectable label can be an echogenic substance (either liquid or gas), non-metallic isotope, an optical reporter, a boron neutron absorber, a pramagnetic metal ion, a ferromagnetic metal, a gamma-emitting radioisotope, a positron-emitting radioisotope, or an x-ray absorber.

A detectable response generally refers to a change in, or occurrence of, a signal that is detectable either by observation or instrumentally. In certain instances, the detectable response is fluorescence or a change in fluorescence, e.g., a change in fluorescence intensity, fluorescence excitation or emission wavelength distribution, fluorescence lifetime, and/or fluorescence polarization.

Suitable optical reporters include, but are not limited to, fluorescent reporters and chemiluminescent groups. A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benothiazole, cyanine, carbocyaninc, salicylate, anthranilate, coumarin, fluorescein, rhodamine or other like compound. Suitable fluorescent reporters include xanthene dyes, such as fluorescein or rhodamine dyes, including, but not limited to, Alexa Fluor® dyes (InvitrogenCorp.; Carlsbad, Calif.), fluorescein, fluorescein isothiocyanate (FITC), Oregon GreenTM, rhodamine, Texas red, tetrarhodamine isothiocynate (TRITC), 5-carboxyfluorescein (FAM), 2′7-dimethoxy-4′5′-dichloro-6-carboxyfluorescein (JOE), tetrachlorofluorescein (TET), 6-carboxyrhodamine (R6G), N,N,N,N′-tetramethyl-6-carboxyrhodamine (TAMRA), 6-carboxy-X-rhodamine (ROX). Suitable fluorescent reporters also include the naphthylamine dyes that have an amino group in the alpha or beta position. For example, naphthylamino compounds include 1-dimethylamino-naphthyl-5-sulfonate, 1-anilino-8-naphthalene sulfonate, 2-p-toluidinyl-6-naphthalene sulfonate, and 5-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p(2-benzoxazolyl)phenyl)maleimide; cyanines, such as Cy2, indodicarbocyanine 3 (Cy3), indodicarbocyanine 5 (Cy5), indodicarbocyanine 5.5 (Cy5.5), 3-(-carboxy-pentyl)-3′ethyl-5,5′-dimethyloxacarbocyanine (CyA); 1H,5H, 11H, 15H-Xantheno[2,3,4-ij:5,6,7-i′j′]diquinolizin-18-ium, 9-[2(or 4)-[[[6-[2,5-dioxo-1-pyrrolidinyl)oxy]-6-oxohexyl]amino]sulfonyl]-4(or 2)-sulfophenyl]-2,3,6,7,12,13,16,17octahydro-inner salt (TR or Texas Red); BODIPY™ dyes; benzoxedilaoles; stilbenes; pyrenes; and the like. Many suitable forms of these fluorescent compounds are available and can be used.

In some embodiments, the motor neurons express a fluorescent protein. Examples of fluorescent proteins suitable for use as detectable label include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al, Mol. Microblol, 55:1767-1781 (2005), the GFP variant described in Crameri et al, Nat. Bioechnol., 14:315319 (1996), the cerulean fluorescent proteins described in Rizzo et al, Nat. Biotechnal 22:445 (2004) and Tsien, Annu. Rev. Biochem., 67:509 (1998), and the yellow fluorescent protein described in Nagal et al, Nat. Biotechnol., 20:87-90 (2002). DsRed variants are described in, e.g., Shaner et al, Nat. Biolechnol., 22:1567-1572 (2004), and include mStrawberry, mCherry, morange, mBanana, mHoneydew, and mTangerine.

Additional DsRed variants are described in, e.g., Wang at al, Proc. Natl. Acad Sci U.S.A. 101:16745-16749 (2004) and include mRaspberry and mPlum. Further examples of DsRed variants include mRFPmars described in Fischer et al, FEBS Lett., 577:227-232 (2004) and mRFPruby described in Fischer et al, FEBS Lett, 580:2495-2502 (2006).

A non-limiting list of fluorescent proteins incudes AcceGFP, AcGFPI, AmCyan, AQ143, AsRed2, Azami-Green (mAG), Cerulean, Cerulean, Citrine, cGFP, CopGFP, Cyan, CyPet, Dronpa, DsRed/DsRed2/DsRed-Express, DsRed-Monomer, EBFP, ECFP, EGFP, Emerald, eqFP611, EYFP, GFPs, HcRed1, HcRed-tandem, J-Red, Kaede, KFP, KikOR, mBanana, mCFP, mChenry, mCitrine, mEosEP, mHoneydew, MiCy, mKO, mOrange, mPlum, mRaspberry, mRFPI, mStrawberry, mTangerine, mYFP, mYFP, mYFP, PA-GFP, PA-mRFP, PhiYFP, PS-CFP-2, Renilla, tdEosFP, tdTomato, T-Sapphire, TurboGFP, UV-T-Sapphire, Venus, YPet, ZsYellowl, and derivatives and analogs thereof. In one embodiment, the fluorescent protein is Green Fluorescent Protein (GFP).

Specific devices or methods known in the art for the detection of fluorescence, e.g., from fluorophores or fluorescent proteins, include, but are not limited to, in vivo near-infrared fluorescence (see, e.g., Frangioni, Curr Opin. Chem. Biol, 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramantiam et al, IEEE Transactions on Biomedical Engineering, 48:1034-1041 (2001), and the like. Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

A fluorescent protein can be expressed from a transgentic reporter gene in the motor neuron. Expression of the fluorescent protein from the transgenic reporter gene can be operably linked to expression of a motor neuron specific gene or can be under the control of a motor neuron specific promoter. Accordingly, in some embodiments, the sequence encoding the fluorescent protein is operably linked to a promoter element for a gene specific for motor neurons. In one embodiment, the sequence encoding the fluorescent protein is operably linked to one or more promoter elements from HB9 gene. Thus, in one embodiment, the motor neuron comprises a transgenic reporter gene comprising a fluorescent protein operably linked to one or more promoter elements from the HB9 gene.

In some embodiments, motor neurons are counted by an image-based method. Presence of a detectable label makes image-based method more amenable to autonmation. When the motor neurons express a fluorescent protein, surviving motor neurons can be those that are expressing the fluorescent protein when the counting is performed. In some embodiments, the number of motor neuron surviving after incubation with the test agent is at least about at least about 5%, 10%, 15%, 20%,25%, 30%, 35%, 40%, 45%,50%, 55%, 60%, 65%, 70%, 75%, 80%, 85%, 90%, 95%, 100%, 1.1-fold, 1.25-fold, 1.5-fold, 1.75-fold, 2-fold, 3-fold, 4-fold, 5-fold, 10-fold or higher than a control.

In some embodiments, number of motor neurons in a sample is assessed via automated image acquisition and analysis using a Celiomics ArrayScan VTI. The acquisition thresholds/parameters are established such that the computer-based calls of number of motor neurons are consistent with human-based calls. Such automated image acquisition and analysis allows for high-throughput screening of compounds.

Number of motor neurons can be assessed by: (i) increased total number of cells in the culture, as compared to an untreated control; (ii) increased total number of cells expressing a m detectable label in the test culture, as compared to an untreated control; (iii) increased ratio of cells expressing a detectable label to the total number of cells in the culture, as compared to an untreated control; or (iv) a combination thereof.

In some embodiments of this and other aspects described herein, the motor neurons can be cultured in the presence of non-neuronal cells. Without wishing to be bound by a theory, culturing in the presence of non-neuronal cells can identify compounds that do not act directly on motor neurons. In some embodiments, non-neuronal cells include glial cells.

The assay can be performed any suitable container or apparatus available to one of skill in the art for cell culturing. For example, the assay can be performed in 24-, 96-, or 384-well plates. In one embodiment, the assay is performed in a 384-well plate.

Motor neurons for the aspects disclosed heroin can be obtained from any source available to one of skill in the art. Additionally, motor neuron can be of any origin. Accordingly, in some embodiments, the motor neuron is a mammalian motor neuron. In one embodiment, the motor neuron is a human motor neuron or a mouse motor neuron. In one embodiment, motor neuron is a mouse ES cell-derived motor neuron.

In some embodiments, the motor neuron is from a subject, e.g., a patient. In some embodiments, the subject. e.g., a patient, is suffering from a neurodegenerative disorder. In some embodiments, the neurodegenerative disorder is ALS or SMA. In an embodiment the motor neuron is from a carrier, e.g., a symptom-free carrier.

In some embodiments, the screening method is a high-throughput screening. High-throughput screening (HTS) is a method for scientific experimentation that uses robotics, data processing and control software, liquid handling devices, and sensitive detectors. High-Throughput Screening or HTS allows a researcher to quickly conduct millions of biochemical, genetic or pharmacological tests. High-Throughput Screening are well known to one skilled in the art, for example, those described in U.S. Pat. Nos. 5,976,813; 6,472,144; 6,692,856; 6,824,982; and 7,091,048, and contents of each of which is herein incorporated by reference in its entirety.

HTS uses automation to run a screen of an assay against a library of candidate compounds. An assay is a test for specific activity: usually inhibition or stimulation of a biochemical or biological mechanism. Typical HTS screening libraries or “decks” can contain from 100,000 to more than 2,000.000 compounds.

The key labware or testing vessel of HTS is the microtiter plate: a small container, usually disposable and made of plastic that features a grid of small, open divots called wells. Modern microplates for HTS generally have either 384, 1536, or 3456 wells. These are all multiples of 96, reflecting the original 96 well microplate with 8×12 9 mm spaced wells.

To prepare for an assay, the researcher fills each well of the plate with the appropriate reagents that he or she wishes to conduct the experiment with, such as a motor neuron cell population. After some incubation time has passed to allow the reagent to absorb, bind to, or otherwise react (or fail to react) with the compounds in the wells, measurements are taken across all the plate's wells, either manually or by a machine. Manual measurements are often necessary when the researcher is using microscopy to (for example) seek changes that a computer could not easily determine by itself. Otherwise, a specialized automated analysis machine can run a number of experiments on the wells such as colorimetric measurements, radioactivity counting, etc. In this case, the machine output the result of each experiment as a grid of numeric values, with each number mapping to the value obtained from a single well. A high-capacity analysis machine can measure dozens of plates in the space of a few minutes like this, generating thousands of experimental data points very quickly.

In another aspect, the invention provides a compound or agent selected by the screening assay described herein. It is to be understood that analogs, derivatives, isomers, and pharmaceutically acceptable salts of the compounds selected by the screening assays described herein are also claimed herein.

In yet another aspect, disclosed herein is a method for identifying a biological pathway that regulates or promotes motor neuron survival, the method comprising identifying a compound that promotes motor neuron survival using a method described herein, and establishing the cellular target of the compound, thereby determining whether the biological pathway comprising the cellular target regulates or promotes motor neuron survival.

In some embodiments, the test agent has known biological activity and/or cellular target(s). In some embodiments, the test agent is known to modulate a biological pathway.

### Methods of Treatment

In still yet another aspect, disclosed herein are methods for treating neurodegenerative disorders in a subject.

In one aspect, a method of treating or preventing a neurodegenerative disorder in a subject in a subject in need thereof is disclosed. An exemplary method of treating or preventing a neurodegenerative disorder in a subject in need thereof comprises administering an effective amount of an agent that inhibits HGK to the subject Another exemplary method of treating or preventing a neurodegenerative disorder in a subject in need thereof comprises administering an effective amount of an agent that decreases activation of the HGK-Tak1-MKK4-JNK-c-Jun cell death signaling cascade to the subject.

In another aspect, a method of treating or preventing a disorder characterized by neuronal cell death in a subject in need thereof is disclosed. An exemplary method of treating or preventing a disorder characterized by neuronal cell death in a subject in need thereof comprises administering an effective amount of an agent that inhibits HGK to the subject. Another exemplary method of treating or preventing a disorder characterized by neuronal cell death in a subject in need thereof comprises administering an effective amount of an agent that decreases activation of the HGK-Tak1-MKK4-JNK-c-Jun cell death signaling cascade to the subject.

In another aspect, a method of treating or preventing ALS in a subject in need thereof is disclosed. An exemplary method of treating or preventing ALS in a subject in need thereof comprises administering an effective amount of an agent that inhibits HGK to the subject. Another exemplary method of treating or preventing ALS in a subject in need thereof comprises administering an effective amount of an agent that decreases activation of the HGK-Tak1-MKK4-JNK-c-Jun cell death signaling cascade to the subject.

In yet another aspect, a method of treating or preventing SMA in a subject in need thereof is disclosed. An exemplary method of treating or preventing SMA in a subject in need thereof comprises administering an effective amount of an agent that inhibits HGK to the subject Another exemplary method of treating or preventing SMA in a subject in need thereof comprises administering an effective amount of an agent that decreases activation of the HGK-Tak1-MKK4-JNK-c-Jun cell death signaling cascade to the subject.

In some embodiments, the agent inhibits HGK and promotes motor neuron survival in the subject. In some embodiments, the agent inhibits HGK and ameliorates at least one symptom associated with the neurodegenerative disorder in the subject. In some embodiments, the agent inhibits HGK and treats the subject's neurodegenerative disorder. In some embodiments, the agent inhibits HGK and prevents the subject from developing a neurodegenerative disorder. In some embodiments, the agent inhibits HGK and prevents the subject's neurodegenerative disorder from progressing.

In some embodiments, the agent inhibits HGK and ameliorates at least one symptom associated with ALS in the subject. In some embodiments, the agent inhibits HGK and treats the subject's ALS. In some embodiments, the agent inhibits HGK and prevents the subject from developing ALS. In some embodiments, the agent inhibits HGK and prevents the subject's ALS from progressing.

In some embodiments, the agent inhibits HGK and ameliorates at least one symptom associated with SMA in the subject. In some embodiments, the agent inhibits HGK and treats the subject's SMA. In some embodiments, the agent inhibits HGK and prevents the subject from developing SMA. In some embodiments, the agent inhibits HGK and prevents the subject's SMA from progressing.

In some embodiments, the agent decreases activation of the HGK-Tak1-MKK4-JNK-c-Jun cell death signaling cascade and promotes motor neuron survival in the subject. In some embodiments, the agent decreases activation of the HGK-Tak1-MKK4-JNK-c-Jun cell death signaling cascade and ameliorates at least one symptom associated with the neurodegenerative disorder in the subject. In some embodiments, the agent decreases activation of the HGK-Tak1-MKK4-JNK-c-Jun cell death signaling cascade and treats the subject's neurodegenerative disorder. In some embodiments, the agent decreases activation of the HGK-Tak1-MKK4-JNK-c-Jun cell death signaling cascade and prevents the subject from developing a neurodegenerative disorder. In some embodiments, the agent decreases activation of the HGK-Tak1-MKK4-JNK-c-Jun cell death signaling cascade and prevents the subject's neurodegenerative disorder from progressing.

In some embodiments, the agent decreases activation of the HGK-Tak1-MKK4-JNK-c-Jun cell death signaling cascade and ameliorates at least one symptom associated with ALS in the subject. In some embodiments, the agent decreases activation of the HGK-Tak1-MKK4-JNK-c-Jun cell death signaling cascade and treats the subject's ALS. In some embodiments, the agent decreases activation of the HGK-Tak1-MKK4-JNK-c-Jun cell death signaling cascade and prevents the subject from developing ALS. In some embodiments, the agent decreases activation of the HGK-Tak1-MKK4-JNK-c-Jun cell death signaling cascade and prevents the subject's ALS from progressing.

In some embodiments, the agent decreases activation of the HGK-Tak1-MKK4-JNK-c-Jun cell death signaling cascade and ameliorates at least one symptom associated with SMA in the subject. In some embodiments, the agent decreases activation of the HGK-Tak1-MKK4-JNK-c-Jun cell death signaling cascade and treats the subject's SMA. In some embodiments, the agent decreases activation of the HGK-Tak1-MKK4-JNK-c-Jun cell death signaling cascade and prevents the subject from developing SMA. In some embodiments, the agent decreases activation of the HGK-Tak1-MKK4-JNK-c-Jun cell death signaling cascade and prevents the subject's SMA from progressing.

In some embodiments, a method of treating or preventing a neurodegenerative disorder in a subject (e.g., ALS or SMA) encompasses co-administering an HGK inhibitor and an additional agent to a subject in need thereof.

In some embodiments, a method of treating or preventing a neurodegenerative disorder in a subject (e.g., ALS or SMA) encompasses co-administering an agent that decreases activation of the HGK-Tak1-MKK4-JNK-c-Jun cell death signaling cascade and an additional agent to a subject in need thereof.

In some embodiments, the method encompasses co-administering a PAK inhibitor and an additional agent to a subject in need thereof. The additional agent can modulate a biological pathway or a target described herein. Generally, the biological pathway or a target is a non-PAK biological pathway or a non-PAK target.

The term “co-administering,” “co-administration,” or “co-administer” refers to the administration of a compound (e.g., a PAK inhibitor) or an agent (e.g., an HGK inhibitor) and an additional agent (e.g., a GSK inhibitor), wherein the compound or the agent and the additional agent can be administered simultaneously, or at different times, as long as they work together (e.g., synergistically) to increase motor neuron survival.

Without limitations, the PAK inhibitor and the additional agent can be administered in the same formulation or in separate formulations. When administered in separate formulations, the PAK inhibitor and the additional agent can be administered within any time of each other. For example, the PAK inhibitor and the additional agent can be administered within 24 hours, 12 hours. 6 hours. 5 hours, 4 hours, 3 hours, 2 hours. 1 hours, 45 minutes, 30 minute, 25 minutes, 20 minutes, 15 minutes, 10 minutes, 5 minutes or less of each other.

When administered in separate formulations, the PAK inhibitor or the additional agent can be administered first Thus, in some embodiments, the PAK inhibitor can be administered first. In some other embodiments, the additional agent is administered first.

Additionally, co-administration does not require the PAK inhibitor and the additional agent to be administered by the same route. As such, each can be administered independently or as a common dosage form.

The PAK inhibitor and the additional agent can be administered in any ratio to each other by weight or moles. For example, the PAK inhibitor and the additional agent can be administered in a ratio of from about 50:1, 40:1, 30:1, 25:1, 20:1, 15:1, 10:1, 5:1, 3:1, 2:1, 1:1.75, 1.5:1, or 1.25:1 to 1:1.25, 1:1.5, 1.75, 1:2, 1:3, 1:4, 1:5, 1:10, 1:15, 1:20, 1:20, 1:30, 1:40, or 1:50. The ratio can be based on the effective amount of the PAK inhibitor or the additional agent.

Without limitations, the HGK inhibitor and the additional agent can be administered in the same formulation or in separate formulations. When administered in separate formulations, the HGK inhibitor and the additional agent can be administered within any time of each other. For example, the HGK inhibitor and the additional agent can be administered within 24 hours, 12 hours, 6 hours, 5 hours, 4 hours, 3 hours, 2 hours, 1 hours, 45 minutes, 30 minute. 25 minutes, 20 minutes, IS minutes, 10 minutes, 5 minutes or less of each other.

When administered in separate formulations, the HGK inhibitor or the additional agent can be administered first. Thus, in some embodiments, the HGK inhibitor can be administered first. In some other embodiments, the additional agent is administered first.

Additionally, co-administration does not require the HGK inhibitor and the additional agent to be administered by the same route. As such, each can be administered independently or as a common dosage form.

The HGK inhibitor and the additional agent can be administered in any ratio to each other by weight or moles. For example, the HGK inhibitor and the additional agent can be administered in a ratio of from about 50:1, 40:1, 30:1, 25:1, 20:1, 15:1, 10:1, 5:1, 3:1, 2:1, 1:1.75, 1.5:1, or 1.25:1 to 1:1.25, 1:1.5, 1.75, 1:2, 1:3, 1:4, 1:5, 1:10, 1:15, 1:20, 1:20, 1:30, 1:40, or 1:50. The ratio can be based on the effective amount of the HGK inhibitor or the additional agent.

The present invention also contemplates co-administering a PAK inhibitor, an HGK inhibitor, and an additional agent (e.g., an agent that modulates a biological pathway or target described herein, e.g., a GSK inhibitor), as well as contacting motor neurons with a PAK inhibitor, an HGK inhibitor, and an additional agent (e.g., an agent that modulates a biological pathway or target described herein, e.g., a GSK inhibitor).

In some embodiments, the method further comprises identifying the compound that promotes motor neuron survival using a method described herein.

In some embodiments, the subject is a human. In some embodiments, the neurodegenerative disorder is ALS or SMA.

In some embodiments, another therapeutic agent is also administered to the subject. Such a therapeutic agent can be administered in the same formulation or in separate formulations Ex e.g., Butyrates, Valproic acid, Hydroxyurea or Riluzole.

In one aspect, the invention described herein features a method of treating ALS or SMA in a subject, the method comprising: identifying a subject in need of modulation of the level of SMN on the basis of modulation of a biological pathway or target described herein, and treating the subject with a compound that modulates the level of SMN.

In some embodiments, the subject is treated on the basis of identifying that the subject is in need of modulation of the level of SMN.

In some embodiments, the method further comprises identifying a compound that elevates the SMN level in a cell using a method described herein.

In some embodiments, the subject is a human. In some embodiments, the subject is suffering from a neurodegenerative disorder. In some embodiments, the neurodegenerative disorder is SMA.

In some embodiments, the compound is an additional agent for modulating a biological pathway or target described herein. In some embodiments, the compound is a Na+/K+ channel modulator e.g., cardiac glycosides (e.g., Ouabain, Digoxin, Dititoxin or Lanatoside C). In some embodiments, the compound is an activator of MAPK (e.g., Anysomycin or Coumermycin). In some embodiments, the compound is a cannabinoid receptor or GPCR agonist (e.g., WIN 55,212-2 or Anandamide). In some embodiments, the compound is a Ca2+ channel modulator (e.g., Thapsigargin, tonomycin or Calcimycin). In some embodiments, the compound is a K+ channel modulator (e.g., Veratridine, Monensin Na or Valinomycin). In some embodiments, the compound is a PDE5 Inhibitor (e.g., MBCQ or Dipyridamole). In some embodiments, the compound is a kinase inhibitor. In some embodiments, the kinase inhibitor is an inhibitor of GSK/CDK e.g., Alsterpaullone or its structural analogs (e.g., 1-aza-alsterpaullone or 2-cyanoethyl-alsterpaullone). In some embodiments, the kinase inhibitor is an inhibitor of GSK e.g., AR-A014418, CHIR98014 or CHIR99021. In some embodiments, the GSK inhibitor increases motor neuron survival.

In some embodiments, the kinase inhibitor is an inhibitor of PKR. In some embodiments, the kinase inhibitor is an inhibitor of CDK2 e.g., GW8510. In some embodiments, the kinase inhibitor is an inhibitor of IKK-2 e.g., IMD-0354 or its structural analog Niclosamide. In some embodiments, the compound is an HDAC inhibitor e.g., trichostatin. In some embodiments, the compound is a proteasome inhibitor. In some embodiments, the compound is a BMP/TGFβ ligand e.g., BMP4. In some embodiments, the compound is a Dopamine receptor ligand In some embodiments, the compounds and/or agents described herein are used in combination with another therapeutic agent e.g., Butyrates, Valproic acid, Hydroxyurea or Riluzole.

In some embodiments, the compounds and/or agents described herein are used in combination with another therapeutic agent suitable for use in treating one or more symptoms of ALS, including, but not limited to, one or more of (i) hydrogenated pyrido[4,3-b]indoles or pharmaceutically acceptable salts thereof and (ii) agents that promote or increase the supply of energy to muscle cells, COX-2 inhibitors, poly(ADP-ribose)polymerase-1 (PARP-1) inhibitors, 30S ribosomal protein inhibitors, NMDA antagonists, NMDA receptor antagonists, sodium channel blockers, glutamate release inhibitors, K(V) 4.3 channel blockers, anti-inflammatory agents, 5-HT1A receptor agonists, neurotrophic factor enhancers, agents that promote motoneuron phenotypic survival and/or neuritogenesis, agents that protect the blood brain barrier from disruption, inhibitors of the production or activity of one or more proinflammatory cytokines, immunomodulators, neuroprotectants, modulators of the function of astrocytes, antioxidants (such as small molecule catalytic antioxidants), free radical scavengers, agents that decrease the amount of one or more reactive oxygen species, agents that inhibit the decrease of non-protein thiol content, stimulators of a normal cellular protein repair pathway (such as agents that activate molecular chaperones), neurotrophic agents, inhibitors of nerve cell death, stimulators of neurite growth, agents that prevent the death of nerve cells and/or promote regeneration of damaged brain tissue, cytokine modulators, agents that reduce the level of activation of microglial cells, cannabinoid CB1 receptor ligands, nonsteroidal anti-inflammatory drugs, cannabinoid CB2 receptor ligand, creatine, creatine derivatives, stereoisomers of a dopamine receptor agonist such as pramipexole hydrochloride, ciliary neurotrophic factors, agents that encode a ciliary neurotrophic factor, glial derived neurotrophic factors, agents that encode a glial derived neurotrophic factor, neurotrophin 3, agents that encode neurotrophin 3, or any combination thereof.

In some embodiments, the compounds and/or agents described herein are used in combination with another therapeutic agent suitable for use in treating one or more symptoms of SMA, including, but not limited to, one or more of antibiotics (e.g., Aminoglycosides, Cephalosporins, Chloramphenicol, Clindamycin, Erythromycins, Fluoroquinolones, Macrolides, Azolides, Metronidazole, Penicillins, Tetracyclines, Trimethoprim-sulfamethoxazole, Vancomycin), steroids (e.g., Andranes (e.g., Testosterone), Cholestanes (e.g., Cholesterol), Cholic acids (e.g., Cholic acid), Corticosteroids (e.g., Dexamethasone), Eatraenes (e.g., Estradiol), Pregnanes (e.g., Progesterone), narcotic and non-narcotic analgesics (e.g., Morphine, Codeine, Heroin, Hydromorphone, Levorphanol, Meperidine, Methadone, Oxydone, Propoxyphene, Fentanyl, Methadone, Naloxone, Buprenorphine, Butorphanol, Nalbuphine, Pentazocine), anti-inflammatory agents (e.g., Alclofenac, Alclometasone Dipropionate, Algestone Acetonide, alpha Amylase, Amcinafal, Amcinafide, Amfenac Sodium, Amiprilose Hydrochloride, Anakinra, Anirolac, Anitrazafen, Apazone, Balsalazide Disodium, Bendazac, Benoxaprofen, Benzydamine Hydrochloride, Bromelains, Broperamole, Budesonide, Carprofen, Cicloprofen, Cintazone, Cliprofen, Clobetasol Propionate, Clobetasone Butyrate, Clopirac, Cloticasone Propionate, Cormethasone Acetate, Cortodoxone, Decanoate, Deflazacort, Delatestryl, Depo-Testosterone, Desonide, Desoximetasone, Dexamethasone Dipropionate, Diclofenac Potassium, Diclofenec Sodium, Diflorasone Diacetate; Diflumidone Sodium, Diflunisal, Difluprednate, Diftalone, Dimethyl Sulfoxide, Drocinonide, Endrysone, Enlimomab, Enolicam Sodium, Epirizole, Etodolac, Etofenamate, Felbinac, Fenamole, Fenbufen, Fenclofenac, Fenclorac, Fendosal, Fenpipalone, Fentiazac, Flazalone, Fluazacort, Flufenamic Acid, Flumizole, Flunisolide Acetate, Flunixin, Flunixin Meglumine, Fluocortin Butyl, Fluorometholone Acetate, Fluquazone, Flurblprofbn, Fluretofen, Fluticasone Propionate, Furaprofen, Furobufen, Halcinonide, Halobetasol Propionate, Halopredone Acetate, Ibufenac, Ibuprofen, Ibuprofen Aluminum, Ibuprofen Piconol, Ilonidap, Indomethacin, Indomethacin Sodium, Indoprofen, Indoxole, Intrazole, Isoflupredone Acetate, Isoxepac, Isoxicam, Ketoprofen. Lofemizole Hydrochloride, Lomoxicam, Loteprednol Etabonate, Meclofenamate Sodium, Meclofenamic Acid, Meclorisone Dibutyrate, Mefenamic Acid, Mesalamine, Meseclazone, Mesterolone, Methandrostenolone, Methenolone, Methenolone Acetate, Methylprednisolone Suleptanate, Momiflumate, Nabumetone, Nandrolone, Naproxen, Naproxen Sodium, Naproxol, Nimazone, Olsalazine Sodium, Orgotein, Orpanoxin, Oxandrolane, Oxaprozin, Oxyphenbutazone, Oxymetholone. Paranyline Hydrochloride, Pentosan Polysulfate Sodium, Phenbutazone Sodium Glycerate, Pirfenidone, Piroxicam, Piroxicam Cinnamate, Piroxicam Olamine, Pirprofen, Prednazate, Prifelone, Prodolie Acid, Proquazone, Proxazole, Proxazole Citrate, Rimexolone, Romazarit, Salcolex, Salnacedin, Salsalate, Sanguinarium Chloride, Seclazone, Sermetacin, Stanozolol, Sudoxicam, Sulindac, Suprofen, Talmetacin, Talniflumate, Talosalate, Tebufelone, Tenidap, Tenidap Sodium, Tenoxicam, Tesicam, Tesimide, Testosterone, Testosterone Blends, Tetrydamine, Tiopinac, Tixocortol Pivalate, Tohmetin, Tolmetin Sodium, Triclonide, Triflumidate, Zidometacin, Zomepirac Sodium), or manti-histaminic agents (e.g., Ethanolamines (like diphenhydrmine carbinoxamine), Ethylenediamine (like tripelennamine pyrilamine), Alkylamine (like chlorpheniramine, dexchlorpheniramine, brompheniramine, triprolidine), other anti-histamines like astemizole, loratadine, fexofenadine, Bropheniramine, Clemastine, Acetaminophen, Pseudoephedrine, Triprolidine). In some embodiments, the method further comprises identifying a biological pathway that regulates the SMN level in a cell using a method described herein.

In some embodiments, the neurodegenerative disorder is SMA. In an embodiment, the biological pathway is PI-3/AKT/GSK pathway. In some embodiments, the biological pathway is PI-3K signaling pathway. In some embodiments, the biological pathway is Akt signaling pathway. In some embodiments, the biological pathway is PDGF pathway. In some embodiments, the biological pathway is PKR pathway. In some embodiments, the biological pathway is Insulin Receptor pathway. In some embodiments, the biological pathway is MAPK signaling pathway. In some embodiments, the biological pathway is Ras pathway. In some embodiments, the biological pathway is eIF2 pathway. In some embodiments, the biological pathway is mTOR pathway. In some embodiments, the biological pathway is NGF signaling pathway. In some embodiments, the biological pathway is EGF pathway. In some embodiments, the biological pathway is FGF pathway. In some embodiments, the biological pathway is TGF pathway. In some embodiments, the biological pathway is GSK signaling pathway. In some embodiments, the biological pathway is BMP pathway. In some embodiments, the agonist or antagonist of the biological pathway is a small molecule, an antibody, or a nuclear acid. In some embodiments, the agonist or antagonist of the biological pathway binds to at least one component in the pathway. In some embodiments, the agonist of the PI-3K signaling pathway is PDGF, PDGP-BB or insulin.

In some embodiments, the agonist of the PI-3K signaling pathway is FGF, EGF, NGF or TGF. In some embodiments, the agonist of the PI-3K signaling pathway activates PI3K, PDK or PKB. In some embodiments, the agonist of the PI-3K signaling or GSK-3 signaling pathway is a GSK inhibitor. In some embodiments, the GSK inhibitor is Alsterpaullone or its structural analogs (e.g., 1-aza-alsterpaullone or 2-cyanoethyl-alsterpaullone), AR-A014418, CHIR98014 or CHIR99021. In some embodiments, the GSK inhibitor increases motor neuron survival. In some embodiments, the antagonist of PKR pathway inhibits PKR. In some embodiments, the agonist or antagonist elevates the SMN level by activation of protein synthesis e.g., translation. In some embodiments, the pathway comprises GSK-3b, CDK2, CDKS, PKR or IKK-2b.

In some embodiments, the antagonist of a biological pathway is a compound that inhibits cyclin-dependent kinase (CDK) or glycogen synthase kinase (GSK).

In some embodiments, the antagonist of a biological pathway is a compound that inhibits protein kinase R.

In some embodiments, the compound is used in combination with another therapeutic agent e.g., Butyrates, Valproic acid, Hydroxyurea or Riluzole.

In one aspect, the invention described herein features a method of treating SMA in a subject, the method comprising: selecting a compound on the basis that the compound modulates the level of SMN by modulating of a biological pathway or target described herein, and administering to the subject the selected compound.

In some embodiments, the method further comprises identifying a compound that elevates the SMN level in a cell using a method described herein.

In some embodiments, the subject is a human. In some embodiments, the subject is suffering from a neurodegenerative disorder. In some embodiments, the neurodegenerative disorder is SMA.

In some embodiments, the compound is a compound or agent described herein. In some embodiments, the compound is of formula (I), (II), (III), (IV), (V), or (VI) as described herein. In some embodiments, the compound is a Na+/K+ channel modulator e.g., cardiac glocosides (e.g., Ouabain, Digoxin, Dititoxin or Lanatoside C). In some embodiments, the compound is an activator of MAPK (e.g., Anysomycin or Coumermycin). In some embodiments, the compound is a cannabinoid receptor or GPCR agonist (e.g., WIN 55,212-2 or Anandamide). In some embodiments, the compound is Ca2+ channel modulator (e.g., Thapsigargin, lonomycin or Calcimycin). In some embodiments, the compound is a K+ channel modulator (e.g., Veratridine, Monensin Na or Valinomycin). In some embodiments, the compound is a PDE5 inhibitor (e.g., MBCQ or Dipyridamole). In some embodiments, the compound is a kinase inhibitor. In some embodiments, the kinase inhibitor is an inhibitor of GSK/CDK e.g., Alsterpaullone or its structural analogs (e.g., l-aza-alsterpaullone or 2-cyanoethyl-alsterpaullone). In some embodiments, the kinase inhibitor is an inhibitor of GSK e.g., AR-A014418, CHIR98014 or CHIR99021. In some embodiments, the GSK inhibitor increases motor neuron survival.

In some embodiments, the kinase inhibitor is an inhibitor of PKR. In some embodiments, the kinase inhibitor is an inhibitor of CDK2 e.g., GW8510. In some embodiments, the kinase inhibitor is an inhibitor of IKK-2 e.g., IMD-0354 or its structural analog Niclosamide. In some embodiments, the compound is an HDAC inhibitor. In some embodiments, the compound is a proteasome inhibitor. In some embodiments, the compound is a BMP/TGFβ ligand. In some embodiments, the compound is a Dopamine receptor ligand.

In some embodiments, the compound is used in combination with another therapeutic agent e.g., Butyrates, Valproic acid, Hydroxyurea or Riluole.

In some embodiments, the method further comprises identifying a biological pathway that regulates the SMN level in a cell using a method described herein.

In some embodiments, the neurodegenerative disorder is SMA. In an embodiment, the biological pathway is PI-3/AKT/GSK pathway. In some embodiments, the biological pathway is PI-3K signaling pathway. In some embodiments, the biological pathway is Akt signaling pathway. In some embodiments, the biological pathway is PDGF pathway. In some embodiments, the biological pathway is PKR pathway. In some embodiments, the biological pathway is Insulin Receptor pathway. In some embodiments, the biological pathway is MAPK signaling pathway. In some embodiments, the biological pathway is Ras pathway. In some embodiments, the biological pathway is eIF2 pathway. In some embodiments, the biological pathway is mTOR pathway. In some embodiments, the biological pathway is NGF pathway. In some embodiments, the biological pathway is EGF pathway. In some embodiments, the biological pathway is FGF pathway. In some embodiments, the biological pathway is TGF pathway. In some embodiments, the biological pathway is GSK pathway. In some embodiments, the biological pathway is BMP pathway. In some embodiments, the agonist or antagonist of the biological pathway is a small molecule, an antibody, or a nuclear acid. In some embodiments, the agonist or antagonist of the biological pathway binds to at least one component in the pathway. In some embodiments, the agonist of the PI-3K signaling pathway is PDGF, PDGF-BB or insulin. In some embodiments, the agonist of the PI-3K signaling pathway is FGF, EGF, NGF or TGF. In some embodiments, the agonist of the PI-3K signaling pathway activates PI3K, PDK or PKB.

In some embodiments, the agonist of the PI-3K signaling or GSK-3 signaling pathway is a GSK inhibitor. In some embodiments, the GSK inhibitor is Alsterpaullone or its structural analogs (e.g., 1-aza-alsterpaullone or 2-cyanoethyl-alsterpaullone) or AR-A014418. In some embodiments, the GSK inhibitor increases motor neuron survival. In some embodiments, the antagonist of PKR pathway inhibits PKR. In some embodiments, the agonist or antagonist elevates the SMN level by activation of protein synthesis e.g., translation. In some embodiments, the pathway comprises GSK-3b, CDK2, CDKS, PKR or IKK-2b.

In some embodiments, the antagonist of a biological pathway is a compound that inhibits cyclin-dependent kinase (CDK) or glycogen synthase kinase (GSK).

In one aspect, the invention teaches a method for treating a neurological disorder such as SMA in a subject using a compound that increases motor neuron survival and/or improves neuromuscular function e.g., by modulating the level of SMN e.g., by modulating a biological pathway or target described herein, and administering to the subject the selected compound.

In one aspect, the invention features a kit comprising an agent identified by the method described herein, and instructions to treat a neurodegenerative disorder e.g. ALS or SMA using a method described herein.

### Formulations and Administration

For administration to a subject, the inhibitors, modulators, or other compounds and/or agents described herein can be administered orally, parenterally, for example, subcutaneously, intravenously, intramuscularly, intraperitoneally, by intranasal instillation, or by application to mucous membranes, such as, that of the nose, throat, and bronchial tubes. One method for targeting the nervous system, such as spinal cord glia, is by intrathecal delivery. The targeted compound and/or agent is released into the surrounding CSF and/or tissues and the released compound can penetrate into the spinal cord parenchyma, just after acute intrathecal injections. For a comprehensive review on drug delivery strategies including CNS delivery, see Ho et al., Curr. Opin. Mol. Ther. (1999). 1:336-3443; Groothuis et al., J. Neuro Virol. (1997), 3:387-400; and Jan, Drug Delivery Systems: Technologies and Commercial Opportunities, Decision Resources, 1998, content of all which is incorporate herein by reference.

They can be administered alone or with suitable pharmaceutical carriers, and can be in solid or liquid form such as, tablets, capsules, powders, solutions, suspensions, or emulsions.

As used herein, the term “administered” refers to the placement of a compound or agent described herein, into a subject by a method or route which results in at least partial localization of the compound at a desired site. A compound described herein can be administered by any appropriate route which results in effective treatment in the subject, i.e. administration results in delivery to a desired location in the subject where at least a portion of the composition delivered. Exemplary modes of administration include, but are not limited to, injection, infusion, instillation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The compounds and/or agents can be formulated in pharmaceutically acceptable compositions which comprise a therapeutically-effective amount of the compound, formulated together with one or more pharmaceutically acceptable carriers (additives) and/or diluents. The compounds and/or agents can be specially formulated for administration in solid or liquid form, including those adapted for the following: (I) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds and/or agents can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960.

As used here, the term “pharmaceutically acceptable” refers to those compounds, agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes: (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, costing agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

Pharmaceutically-acceptable antioxidants include, but are not limited to, (I) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lectithin, propyl gallate, alpha-tocopherol, and the like; and (3) metal chelating agents, such as citric acid, ethylenediamine tetraacetic acid (EDTA), sorbitol, tartaric acid, phosphoric acids, and the like.

“PEG” means an ethylene glycol polymer that contains about 20 to about 2000000 linked monomers, typically about 50-1000 linked monomers, usually about 100-300. Polyethylene glycols include PEGs containing various numbers of linked monomers, e.g., PEG20, PEG30, PEG40, PEG60, PEG80, PEG100, PEG115, PEG200, PEG 300, PEG400, PEG500, PEG600, PEG1000, PEG1500, PEG2000, PEG3350, PEG4000, PEG4600, PEG5000, PEG6000, PEG8000, PEG11000, PEG12000, PEG2000000 and any mixtures thereof.

The compounds and/or agents can be formulated in a gelatin capsule, in tablet form, dragee, syrup, suspension, topical cream, suppository, injectable solution, or kits for the preparation of syrups, suspension, topical cream, suppository or injectable solution just prior to use. Also, compounds and/or agents can be included in composites, which facilitate its slow release into the blood stream, e.g., silicon disc, polymer beads.

The formulations can conveniently be presented in unit dosage form and may be prepared by any of the methods well known in the art of pharmacy. Techniques, excipients and formulations generally are found in, e.g., Remington's Pharmaceutical Sciences. Mack Publishing Co., Easton, Pa. 1985, 17th edition, Nema et al., PDA J. Pharm. Sci. Tech 1997 51:166-171. Methods to make invention formulations include the step of bringing into association or contacting an ActRIIB compound with one or more excipients or carriers. In general, the formulations are prepared by uniformly and intimately bringing into association one or more compounds and/or agents with liquid excipients or finely divided solid excipients or both, and then, if appropriate, shaping the product.

The preparative procedure may include the sterilization of the pharmaceutical preparations. The compounds and/or agents may be mixed with auxiliary agents such as lubricants, preservatives, stabilizers, salts for influencing osmotic pressure, etc., which do not react deleteriously with the compounds and/or agents.

Examples of injectable form include solutions, suspensions and emulsions. Injectable forms also include sterile powders for extemporaneous preparation of injectable solutions, suspensions or emulsions. The compounds and/or agents of the present invention can be injected in association with a pharmaceutical carrier such as normal saline, physiological saline, bacteriostatic water, Cremophor™ EL (BASF, Parsippany, N.J.), phosphate buffered saline (PBS), Ringer's solution, dextrose solution, ethanol, polyol (e.g., glycerol, propylene glycol, and liquid polyethylene glycol), vegetable oils, and suitable mixtures thereof, and other aqueous carriers known in the art. Appropriate non-aqueous carriers may also be used and examples include fixed oils and ethyl oleate. In all cases, the composition must be sterile and should be fluid to the extent that easy syringability exists. It must be stable under the conditions of manufacture and storage and must be preserved against the contaminating action of microorganisms such as bacteria and fungi. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by Including in the composition an agent which delays absorption, for example, aluminum monostearate and gelatinA suitable carrier is 5% dextrose in saline. Frequently, it is desirable to include additives in the carrier such as buffers and preservatives or other substances to enhance isotonicity and chemical stability.

In some embodiments, compounds and/or agents described herein can be administrated encapsulated within liposomes. The manufacture of such liposomes and insertion of molecules into such liposomes being well known in the art, for example, as described in U.S. Pat. No. 4,522,811. Liposomal suspensions (including liposomes targeted to particular cells, e.g., a pituitary cell) can also be used as pharmaceutically acceptable carriers.

In one embodiment, the compounds and/or agents are prepared with carriers that will protect the compound and/or agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In the case of oral ingestion, excipients useful for solid preparations for oral administration are those generally used in the art, and the useful examples are excipients such as lactose, sucrose, sodium chloride, starches, calcium carbonate, kaolin, crystalline cellulose, methyl cellulose, glycerin, sodium alginate, gum arabic and the like, binders such as polyvinyl alcohol, polyvinyl ether, polyvinyl pyrrolidone, ethyl cellulose, gum arabic, shellac, sucrose, water, ethanol, propanol, carboxymethyl cellulose, potassium phosphate and the like, lubricants such as magnesium stearate, talc and the like, and further include additives such as usual known coloring agents, disintegrators such as alginic acid and PRIMOGEL™ and the like.

The compounds and/or agents can be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compounds and/or agents may be incorporated with excipients and used in the form of tablets, capsules, elixirs, suspensions, syrups, and the like. Such compositions and preparations should contain at least 0.1% of compound and/or agent. The percentage of the agent in these compositions may, of course, be varied and may conveniently be between about 2% to about 60% of the weight of the unit. The amount of compound and/or agent in such therapeutically useful compositions is such that a suitable dosage will be obtained. Preferred compositions according to the present invention are prepared so that an oral dosage unit contains between about 100 and 2000 mg of compound and/or agent.

Examples of bases useful for the formulation of suppositories are oleaginous bases such as cacao butter, polyethylene glycol, lanolin, fatty acid triglycerides, witepsol (trademark. Dynamite Nobel Co. Ltd.) and the like. Liquid preparations may be in the form of aqueous or oleaginous suspension, solution, syrup, elixir and the like, which can be prepared by a conventional way using additives.

The compositions can be given as a bolus dose, to maximize the circulating levels for the greatest length of time after the dose. Continuous infusion may also be used after the bolus dose.

The compounds and/or agents can also be administrated directly to the airways in the form of an acrosol. For administration by inhalation, the compounds and/or agents in solution or suspension can be delivered in the form of an aerosol spray from pressured container or dispenser which contains a suitable propellant, e.g., a gas such as carbon dioxide, or hydrocarbon propellant like propane, butane or isobutene. The compounds and/or agents can also be administrated in a no-pressurized form such as in an atomizer or nebulizer.

The compounds and/or agents can also be administered parenterally. Solutions or suspensions of these compounds and/or agents can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

It may be advantageous to formulate oral or parenteral compositions in dosage unit form for ease of administration and uniformity of dosage. As used herein, “dosage unit” refers to physically discrete units suited as unitary dosages for the subject to be treated; each unit containing a predetermined quantity of compound and/or agent calculated to produce the desired therapeutic effect in association with the required pharmaceutical carrier.

Administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fisidic acid derivatives. Transmucosal administration can be accomplished through the use of nasal sprays or suppositories. For transdermal administration, the compounds and/or agents are formulated into ointments, salves, gels, or creams as generally known in the art.

The compounds and/or agents can be administrated to a subject in combination with other pharmaceutically active agents. Exemplary pharmaceutically active compounds and/or agents include, but are not limited to, those found in Harrison's Princples of Internal Medicine, 13a Edition, Eds. T. R. Harrison et al McGraw-Hill N.Y., NY; Physicians Desk Reference, 50^{th} Edition, 1997, Oradell N.J., Medical Economics Co.; Pharmacological Basis of Therapeutics, 8^{th} Edition, Goodman and Gilman, 1990; United States Pharmacopeia, The National Formulary, USP XII NF XVII, 1990, the complete contents of all of which are incorporated herein by reference. In some embodiments, the pharmaceutically active agent is selected from the group consisting of butyrates, valproic acid, hydroxyuirae and Riluzole.

The compounds and/or agents and the other pharmaceutically active agent can be administrated to the subject in the same pharmaceutical composition or in different pharmaceutical compositions (at the same time or at different times). For example, a PKA inhibitor and an additional agent for modulating a biological pathway or target can be administrated to the subject in the same pharmaceutical composition or in different pharmaceutical compositions (at the same time or at different times). As an additional example, a HGK inhibitor and an additional agent described herein can be administered to the subject in the same pharmaceutical composition or in different pharmaceutical compositions (at the same or at different times).

The amount of compound and/or agent which can be combined with a carrier material to produce a single dosage form will generally be that amount of the compound and/or agent which produces a therapeutic effect. Generally out of one hundred percent, this amount will range from about 0.1% to 99% of compound, preferably from about 5% to about 70%, most preferably from 10% to about 30%.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearte; and a sweetening agent such as sucrose, lactose, or saccharin. When the dosage unit form is a capsule, it may contain, in addition to materials of the above type, a liquid carrier, such as a fatty oil.

Various other materials may be present as coatings or to modify the physical form of the dosage unit. For instance, tablets may be coated with shellac, sugar, or both. A syrup may contain, in addition to the active ingredient, sucrose as a sweetening agent, methyl and propylparabens as preservatives, a dye, and flavoring such as cherry or orange flavor.

The pharmaceutical compositions can be included in a container, pack, or dispenser together with instructions for administration.

As used herein, the term “therapeutically effective amount” means an amount of the compound and/or agent which is effective to promote the survival of motor neuron cells or to prevent or slow the death of such cells. Determination of a therapeutically effective amount is well within the capability of those skilled in the art. Generally, a therapeutically effective amount can vary with the subject's history, age, condition, sex, as well as the severity and type of the medical condition in the subject, and administration of other agents that inhibit pathological processes in neurodegenerative disorders.

Guidance regarding the efficacy and dosage which will deliver a therapeutically effective amount of a compound and/or agent to treat ALS or SMA can be obtained from animal models of ALS or SMA, see e.g., those described in Hsieh-Li et al. Nature Genetics. 2000; 24:66-70 and references cited therein.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds and/or agents lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay. Examples of suitable bioassays include DNA replication assays, transcription based assays, GDF-8 binding assays, and immunological assays.

The dosage may be determined by a physician and adjusted, as necessary, to suit observed effects of the treatment Generally, the compositions are administered so that the compound and/or agent is given at a dose from 1 μg/kg to 100 mg/kg. 1 μg/kg to 50 mg/kg 1 μg/kg to 20 mg/kg, 1 μg/kg to 10 mg/kg, 1 μg/kg to 1 mg/kg, 100 μg/kg to 100 mg/kg, 100 μg/kg to 50 mg/kg, 100 μg/kg to 20 mg/kg, 100 μg/kg to 10 mg/kg. 100 μg/kg to 1 mg/kg, 1 mg/kg to 100 mg/kg, 1 mg/kg, 50 mg/kg, 1 mg/kg to 20 mg/kg, 1 mg/kg to 10 mg/kg, 10 mg/kg to 100 mg/kg, 10 mg/kg to 50 mg/kg, or 10 mg/kg to 20 mg/kg. For antibody compounds and/or agents, one preferred dosage is 0.1 mg/kg of body weight (generally 10 mg/kg to 20 mg/kg). If the antibody is to act in the brain, a dosage of 50 mg/kg to 100 mg/kg is usually appropriate.

With respect to duration and frequency of treatment, it is typical for skilled clinicians to monitor subjects in order to determine when the treatment is providing therapeutic benefit, and to determine whether to increase or decrease dosage, Increase or decrease administration frequency, discontinue treatment, resume treatment or make other alteration to treatment regimen. The dosing schedule can vary from once a week to daily depending on a number of clinical factors, such as the subjects sensitivity to the polypeptides. The desired dos can be administered at one time or divided into subdoses, e.g., 2-4 subdoses and administered over a period of time, e.g., at appropriate intervals through the day or other appropriate schedule. Such sub-doses can be administered as unit dosage forms. Examples of dosing schedules are administration once a week, twice a week, three times a week, daily, twice daily, three times daily or four or more times daily.

### Biological Pathways and Targets

Non-limiting examples of the biological pathways that are amenable to the present disclosure include e.g. HGK-Tak1-MKK4-JNK-c-Jun cell death signaling cascade, PI3K signaling pathway, Akt signaling pathway, MAPK signaling pathway, PDGF pathway, RAS pathway, eIF2 pathway, GSK pathway, PKR pathway, Insulin Receptor Pathway, mTOR pathway, EGF pathway, NGF pathway, FGF pathway and BMP/TGFβ pathway. Non-limiting examples of the targets that modulate levels of SMN include e.g. components of the biological pathways described herein. In some preferred embodiments, the targets that modulate levels of SMN include e.g., Na+/K+ channel, MAPK, cannobinoid receptor, GPCR, Ca2+ channel, K+ channel, PDE5, GSK/CDK. GSK. PKR, CDK2, IKK-2, HDAC, proteasome, BMP/TGFβ receptor and Dopamine receptor.

### PI3K Signaling Pathway

The definition and details of the PI3K signaling pathway are disclosed in the art e.g., Abell K. and Watson, C. J. Cell Cycle. 4, 897-900 (2005): Brachmann, S. M. et al., Mol. Cell Biol. 25, 2593-2606 (2005); Katso R. et al., Annu. Rev. Cell Dev. Bio. 17, 615-675 (2001); and Vanhaesebroeck B. and Waterfield M. D. Exp. Cell Res. 253, 239-254 (1999).

### Akt Signaling Pathway

The definition and details of the PI3K signaling pathway are disclosed in the art e.g., Downward, J. Curr. Opin. Cell Biol. 10, 262-267(1988); Jimenez, C. et al., J. Biol. Chem. 277(44):41556-41562 (2002); Kitamura, T. et al., Mol. Cell Biol. 19, 6286-6296 (1999); Ruggero D. and Sonenberg N. Oncogene. 24, 7426-34 (2005); Testa J. R. and Tsichlis P. N. Oncogene. 7391-7393 (2005); and Zhou X. M. et al., J. Biol. Chem. 275, 25046-25051 (2000).

### MAPK Signaling Pathway

The definition and details of the MAPK signaling pathway are disclosed in the art e.g., Ichio H. et al., Science 275, 90-94 (1997); Qiu M. S. and Green S. H. Neuron 7, 937-946 (1991); Rubinfeld H. and Seger R. Mol. Biotechnol. 31, 151-174 (2005); and Yoon S. and Seger R. Growth Factors. 24, 21-44 (2006).

### PDGF Pathway

The definition and details of the PDGF pathway are disclosed in the art e.g., Fredriksson L. et al., J. Biol. Chem. 280, 26856-26862 (2005); Hauck C. R. et al., J. Biol Chem. 275, 41092-41099 (2000); Hoch R. V. and Soriano P. Development 130, 4769-4784 (2003); Jiang B. et al., Surgery. 120, 427-431(1996); and Reigstad L. J. et al., FEBS J. 272, 5723-5741 (2005).

### RAS Pathway

The definition and details of the RAS pathway are disclosed in the art e.g., Ada-Nguema A. S. et. al. J. Cell Sc. 119, 1307-1319 (2006); Hofer F. et al., Pro. Natl. Acad Sci. 91, 11089-11093 (1994); Kikuchi A. et. al. Mol. Cell. Biol. 14, 7483-7491 (1994); Rodriguez-Viciana, P. et al., Nature 370, 527-532; and Rubio, I. et al., Biochem. J. 326, 891-895 (1997).

eIF2 Pathway

The definition and details of the eIF2 pathway are disclosed in the art e.g., Clemens M. J. Prog. Mol. Subcell Biol. 27, 57-89 (2001); Proud C. G. Semin. Cell Dev. Biol. 16, 3-12 (2005); and Wek R. C. et al., Biochem. Soc. Trans. 34, 7-11 (2006).

### GSK-3 Pathway

The definition and details of the GSK-3 pathway are disclosed in the art e.g., Biondi R. M. and Nebreda A. R. Biochem J. 372, 1-13 (2003); Jope R. S. and Johnson G. V. Trends Biochem Sci. 29, 95-102 (2004); and Polakis P. Curr. Biol. 12, R499-R501 (2002).

### PKR Pathway

The definition and details of the PKR pathway are disclosed in the art e.g., Bennett R. L. et al., Blood. 106, 821-829 (2006); Donze O. et al., EMBO J. 23, 564-571 (2004); Guerra S. et al., J. Biol Chem. 281, 18734-18745 (2006); and Li S. et al., Proc. Natl. Acad Sci. USA. 103, 10005-10010 (2006).

### Insulin Receptor Pathway

The definition and details of the Insulin Receptor pathway are disclosed in the art e.g., Dudek H. et al., Science. 275, 661-665 (1997); Pandini G. et al., J. Biol Chem. 277, 39684-39695 (2002); and White M. F. and Myers M. G. In Endocrinology (DeGroot, L. J., and Jameson, J. L., eds), W. B. Saunders Co., Philadelphia (2001).

mTOR Pathway

The definition and details of the mTOR pathway are disclosed in the art e.g., Gingras A. C. et al., Genes Dev. 15, 807-826 (2001); Hamnan K. M. et al., Mol. Cell Biol. 23, 8862-8877 (2003); Kim D. H. et al., Cell 110, 163-175 (2002); Kumar V. et al., J. Biol. Chem. 275, 10779-10787 (2000); and Raught B. et al., Proc. Natl. Acad. Sci. USA 98, 7037-7044 (2001).

### EGF Pathway

The definition and details of the EGF pathway are disclosed in the art e.g., Carpenter G. and Ji Q. Exp. Cell Res. 253, 15-24 (1999); Garcia R. et al., Oncogene 20, 2499-2513 (2001): Henson E. S. and Gibson S. B. Cell Signal. (2006); Olayioye M. A. et al., J. Biol. Chem. 274, 17209-17218 (1999); Guren T. K. et al. J. Cell Physiol. 196, 113-123 (2003); and Salo K. et al., J. Biol. Chem. 277, 29568-29576 (2002).

### NGF Pathway

The definition and details of the EGF pathway are disclosed in the art e.g., Coulson E. J. Prog. Brain Res. 146, 41-62 (2004); Huang E. J. and Reichardt L. F. Annu. Rev. Biochem. 72, 609-642 (2003); Miller F. D. and Kaplan D. R. Cell Mol. Life Sci 58, 1045-1053 (2001); and Rabiadeh S. and Bredesen D. E. Cytokine Growth Factor Rev. 14, 225-239 (2003).

### FGF Pathway

The definition and details of the FGF pathway are disclosed in the art e.g., Lee P. L. et al., Science. 245, 57-60 (1989); Mignatti P. et al., J. Cell Physiol. 151, 81-93 (1992); Miki T. et al., Proc. Natl. Acad. Sci. USA. 89, 246-250 (1992); Gringel S. et al., J. Biol. Chem. 385, 1203-1208 (2004); and Ornitz D. M. and Itoh, N. Genome Biol. 2, 1-12 (2001); Sorensen V. et al., Bioessays. 28, 504-514 (2006).

### BMP/TGFβ Pathway

The definition and details of the BMP/TGFβ pathway are disclosed in the art e.g., Kawabata M. and Miyazono K., J. Biochem. (Tokyo), 125, 9-16 (1999); Wrana J. L. Miner. Electrolyte Metab., 24, 120-130 (1998); and Markowitz S. D., and Roberts A. B., Cytokine Growth Factor Rev., 7, 93-102 (1996).

### Phosphoinositide 3-Kinases

Phosphoinositide 3-kinases (PI 3-kinases or PI3Ks) are a family of related enzymes that are capable of phosphorylating the 3 position hydroxyl group of the inositol ring of phosphatidylinositol (PtdIns). http//en.wikipedia.org wiki/Phosphoinositide_3-kinase-cite_note-0. They are also known as phosphatidylinositol-3-kinases.

PI3Ks interact with the IRS (Insulin receptor substrate) in order to regulate glucose uptake through a series of phosphorylation events. The phophoinositol-3-kinase family is composed of Class I, II and Class III, with Class I the only ones able to convert PI(4,5)P2 to PI(3,4,5)P3 on the inner leaflet of the plasma membrane.

Class I PI3K are heterodimeric molecules composed of a regulatory and a catalytic subunit they are further divided between IA and IB subsets on sequence similarity. Class IA PI3K are composed of one of five regulatory p85α, p55α, p50α, p85β or p55γ subunit attached to a p110α, β or δ catalytic subunit. The first three regulatory subunits are all splice variants of the same gene (Pik3r1), the other two being expressed by other genes (Pik3r2 and Pik3r3, p85β and p55γ, respectively). The most highly expressed regulatory subunit is p85α, all three catalytic subunits are expressed by separate genes (Pik3ca, PIk3cb and Pik3cd for pi10α, pi10β and p110δ, respectively). The first two p110 isoforms (α and β) are expressed in all cells, but p110δ is primarily expressed in leukocytes and it has been suggested it evolved in parallel with the adaptive immune system. The regulatory p101 and catalytic p110γ subunits comprise the type IB PI3K and are encoded by a single gene each.

Class II comprises three catalytic isoforms (C2α, C2β, and C2γ), but unlike Classes I and III, no regulatory proteins. These enzymes catalyse the production of PI(3)P from PI (may also produce PI(3,4)P2 from PI(4)P). C2α and C2β are expressed throughout the body, however expression of C2γ is limited to hepatocytes. The distinct feature of Class II PI3Ks is the C-terminal C2 domain. This domain lacks critical Asp residues to coordinate binding of Ca²⁺, which suggests class II PI3Ks bind lipids in a Ca²⁺ independent manner.

Class III are similar to II in that they bias the production of PI(3)P from PI, but are more similar to Class I in structure, as they exist as a heterodimers of a catalytic (Vps34) and a regulatory (p150) subunits. Class III seems to be primarily involved in the trafficking of proteins and vesicles.

All PI 3-kinases are inhibited by the drugs wortmamin and LY294002, although certain member of the class II PI 3-kinase family show decreased sensitivity.

PI 3-kinases have been linked to an extraordinarily diverse group of cellular functions, including cell growth, proliferation, differentiation, motility, survival and intracellular trafficking. Many of these functions relate to the ability of class I PI 3-kinases to activate protein kinase B (PKB, aka Akt). The class IA PI 3-kinase p110α is mutated in many cancers. The PtdIns(3,4,5)P₃ phosphalase PTEN which antagonises PI 3-kinase signalling is absent from many tumors. Hence, PI 3-kinase activity contributes significantly to cellular transformation and the development of cancer. The p110δ and p110γ isoforms regulate different aspects of immune responses. PI 3-kinases are also a key component of the insulin signaling pathway.

AKT is activated as a result of PI3-kinase activity, because AKT requires the formation of the PtdIns(3,4,5)P3 (or “PIP3”) molecule in order to be translocated to the cell membrane. At PIP3, AKT is then phosphorylated by phosphoinositide dependent kinase 1 (PDK1), and is thereby activated. The “PI3-k/AKT” signaling pathway has been shown to be required for an extremely diverse array of cellular activities such as cellular proliferation and survival.

In addition to AKT and PDK1, one other related serine threonine kinase is bound at the PIP3 molecule created as a result of PI3-kinase activity, SGK.

PI3K has also been implicated in Long term potentiation (LTP). The PI3K pathway also recruits many other proteins downstream, including mTOR, GSK-3β, and PSD-95. The PI3K-mTOR pathway leads to the phosphorylation of p70S6K, a kinase which facilitates translational activity.

### Glycogen Synthase Kinase 3 (GSK-3)

Glycogen synthase kinase 3 (GSK-3) Is a serine/threonine protein kinase. In mammals GSK-3 is encoded by two known genes GSK-3α, http//en.wikipedia.org/wiki/GSK-3A and β.

The nucleotide and amino acid sequences of human GSK-3α are disclosed in the art e.g., Hoshino T. et al., “Isolation of cDNA clones for human glycogen synthase kinase 3alpha.”, Submitted (November-1997) to the EMBL/GenBank/DDBJ databases; Grimwood J., et al., Nature 428:529-535(2004); and The MOC Project Team, Genome Res. 14:2121-2127(2004). The nucleotide and amino acid sequences of human GSK-3β are disclosed in the art e.g., Stambolic V. and Woodgett J. R. Biochem. J. 303:701-704(1994); The MGC Project Team, Genome Res. 14:2121-2127(2004); Rhoads A. R. et al., Mol. Psychiatry 4:437-442(1999); and Lau K. F. et al., Genomics 60:121-128(1999).

GSK-3α is implicated in the hormonal control of several regulatory proteins including glycogen synthase, MYB and the transcription factor JUN. GSK-3 participates in the Wnt signaling pathway. It is implicated in the hormonal control of several regulatory proteins including glycogen synthase, MYB and the transcription factor JUN. It also phosphorylates JUN at sites proximal to its DNA-binding domain, thereby reducing its affinity for DNA. It phosphorylates MUC1 in breast cancer cells, and decreases the interaction of MUC1 with CTNNB1/beta-catenin. GSK-3β is inhibited when phosphorylated by AKT1.

GSK-3β is expressed in testis, thymus, prostate and ovary and weakly expressed in lung, brain and kidney.

The unprocessed human GSK-3α protein is 483 amino acid in length with a molecular weight of about 51 kDa. The unprocessed human GSK-30 protein is 420 amino acid in length with a molecular weight of about 47 kDa.

### Ca²⁺ Channel

A Calcium channel is an ion channel which displays selective permeability to calcium ions. It is also called as voltage-dependent calcium channel, although there are also ligand-gated calcium channels.

Calcium channel blockers are a class of drugs and natural substances with effects on many excitable cells of the body such as the muscle of the heart, smooth muscles of the vessels or neuron cells. Classes of calcium channel blockers include e.g., Dihydropyridine, Phenylalkylamine, Benzothiazepine.

cGMP-Specific 3′,5′-Cyclic Phosphodiesterase (PDE5)

PDE5 refers to a cGMP-binding. cGMP-specific phosphodiesterase, a member of the cyclic nucleotide phosphodiesterase family. This phosphodiesterase specifically hydrolyzes cGMP to 5′-GMP. It is involved in the regulation of intracellular concentrations of cyclic nucleotides and is important for smooth muscle relaxation in the cardiovascular system. Human PDE5 is expressed in aortic smooth muscle cells, heart, placenta, skeletal muscle and pancreas and, to a much lesser extent, in brain, liver and lung.

A PDE5 inhibitor, is a drug used to block the degradative action of PDE5 on cyclic GMP in the smooth muscle cells lining the blood vessels supplying the corpus cavemrnosum of the penis. These drugs are used in the treatment of erectile dysfunction, Because PDE5 is also present in the arterial wall smooth muscle within the lungs, PDE5 inhibitors have also been explored for the treatment of pulmonary hypertension, a disease in which blood vessels in the lungs become abnormally narrow.

### Camabinoid Receptors

The cannabinoid receptors refer to members of the family of guanine-nucleotide-binding protein (G-protein) coupled receptors which inhibit adenylate cyclase activity in a dose-dependent, stereoselective and pertussis toxin-sensitive manner. The cannabinoid receptors have been found to be involved in the cannabinoid-induced CNS effects (including alterations in mood and cognition) experienced by users of marijuana. Their ligands are known as cannabinoids or endocamabinoids.

### Histone Deacetylase (HDAC)

Histone deacetylases (HDAC) are a class of enzymes that remove acetyl groups from an ε-N-acetyl lysine amino acid on a histone. Exemplary HDACs include those Class I HDAC: HDAC1, HDAC2, HDAC3, HDAC8; and Class II HDACs: HDAC4, HDAC5, HDAC6, HDAC7A, HDAC9, HDAC10. Type I mammalian HDACs include: HDAC1, HDAC2, HDAC3, HDAC8, and HDAC11. Type II mammalian HDACs include: HDAC4, HDAC5, HDAC6, HDAC7, HDAC9, and HDAC1.

### Cardiac Glycosides

Cardiac glycosides are drugs used in the treatment of congestive heart failure and cardiac arrhythmia. Cardiac glycosides work by inhibiting the Na⁺/K⁺ pump. This causes an increase in the level of sodium ions in the myocytes, which then leads to a rise in the level of calcium ions. This inhibition increases the amount of Ca²⁺ ions available for contraction of the heart muscle, improves cardiac output and reduces distention of the heart.

### Inhibitor of IκB Kinase 2 (IKK2)

IKK2 is a protein which is a component of a cytokine-activated intracellular pathway involved in triggering immune responses. Activation of IKK2 leads to phosphorylation of the inhibitor of Nuclear Transcription factor kappa-B (IkB). Phosphorylation of IkB causes the degradation of the inhibitor IkB via the ubiquination pathway, thereby allowing the transcription factor NFkB to enter the cells nucleus and activate various genes involved in inflammation and other immune responses.

IKK2 plays a significant factor in the state of brain cells after a stroke. Experimental mice that had an overactive form of IKK2 experienced the loss of many more neurons than controls did after a stroke-simulating event.

### Cyclin Dependent Kinase 2 (CDK2)

The protein encoded by this gene is a member of the cyclin-dependent kinase family of Ser/Thr protein kinases. This protein kinase is highly similar to the gene products of S. cerevisiae cdc28, and S. pombe cdc2. It is a catalytic subunit of the cyclin-dependent kinase complex, whose activity is restricted to the G1-S phase of the cell cycle, and is essential for the G1/S transition. This protein associates with and regulated by the regulatory subunits of the complex including cyclin E or A. Cyclin E binds G1 phase Cdk2, which is required for the transition from G1 to S phase while binding with Cyclin A is required to progress through the S phase. Its activity is also regulated by phosphorylation. Two alternatively spliced variants and multiple transcription initiation sites of this gene have been reported. The role of this protein in G1-S transition has been recently questioned as cells lacking Cdk2 are reported to have no problem during this transition.

Known CDK inhibitors are p21Cip1 (CDKN1A) and p27Kip1 (CDKN1B). Drugs which inhibit Cdk2 and arrest the cell cycle may reduce the sensitivity of the epithelium to many cell cycle-active antitumor agents and therefore represent a strategy for prevention of chemotherapy-induced alopecia.

### Kits

A compound and/or agent described herein can be provided in a kit. The kit includes (a) the compound and/or agent, e.g., a composition that includes the compound and/or agent, and (b) informational material. The informational material can be descriptive, instructional, marketing or other material that relates to the methods described herein and/or the use of the compound and/or agent for the methods described herein. For example, the informational material describes methods for administering the compound and/or agent to promote motor neuron survival, treat or prevent a neurodegenerative disorder (e.g., ALS or SMA), or at least one symptom of disease neurodegenerative disorder, or a disease associated with dysfunctional or decreases motor neurons.

In one embodiment, the informational material can include instructions to administer the compound and/or agent in a suitable manner, e.g., in a suitable dose, dosage form, or mode of administration (e.g., a dose, dosage form, or mode of administration described herein). In another embodiment, the informational material can include instructions for identifying a suitable subject, e.g., a human, e.g., an adult human. The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is a link or contact information, e.g., a physical address, email address, hyperlink, website, or telephone number, where a user of the kit can obtain substantive information about the modulator and/or its use in the methods described herein. Of course, the informational material can also be provided in any combination of formats.

In addition to the compound and/or agent, the composition of the kit can include other ingredients, such as a solvent or buffer, a stabilizer or a preservative, and/or a second agent for treating a condition or disorder described herein, e.g. increased pancreatic islet mass. Alternatively, the other ingredients can be included in the kit, but in different compositions or containers than the compound and/or agent. In such embodiments, the kit can include instructions for admixing the compound and/or agent and the other ingredients, or for using the modulator together with the other ingredients.

The compound and/or agent can be provided in any form, e.g., liquid, dried or lyophilized form. It is preferred that the compound and/or agent be substntially pure and/or sterile. When the compound and/or agent is provided in a liquid solution, the liquid solution preferably is an aqueous solution, with a sterile aqueous solution being prefered. When the compound and/or agent is provided as a dried form, reconstitution generally is by the addition of a suitable solvent. The solvent, e.g., sterile water or buffer, can optionally be provided in the kit.

The kit can include one or more containers for the composition containing the compound and/or agent. In some embodiments, the kit contains separate containers, dividers or compartments for the compound and/or agent (e.g., in a composition) and informational material. For example, the compound and/or agent (e.g., in a composition) can be contained in a bottle, vial, or syringe, and the informational material can be contained in a plastic sleeve or packet. In other embodiments, the separate elements of the kit are contained within a single, undivided container. For example, the compound and/or agent (e.g., in a composition) is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the compound and/or agent (e.g., in a composition). For example, the kit includes a plurality of syringes, ampules, foil packets, or blister packs, each containing a single unit dose of the compound and/or agent. The containers of the kits can be air tight and/or waterproof.

The compound and/or agent (e.g., in a composition) can be administered to a subject, e.g., an adult subject, e.g., a subject in need of motor neurons. The method can include evaluating a subject, e.g., to evaluate motor neuron survival, and thereby identifying a subject as having decreased motor neurons or being predisposed to motor neuron death or dysfunction.

### SOME DEFINITIONS

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about” The term “about” when used in connection with percentages may mean±1%.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation. “e.g.” is derived from the Latin exempli gratia, and is used herein to indicate a non-limiting example. Thus, the abbreviation “e.g.” is synonymous with the term “for example.”

As used herein, the term “modulate” means to cause or facilitate a qualitative or quantitative change, alteration, or modification in a molecule, a process. pathway, or phenomenon of interest Without limitation, such change may be an increase, decrease, a change in binding characteristics, or change in relative strength or activity of different components or branches of the process, pathway, or phenomenon.

The term “modulator” refers to any molecule or compound that causes or facilitates a qualitative or quantitative change, alteration, or modification in a process, pathway, or phenomenon of interest. As used herein, the term “modulator” comprises both inhibitors and activators of a biological pathway or target.

As used herein, the phrase “modulation of a biological pathway” refers to modulation of activity of at least one component of the biological pathway. It is contemplated herein that modulator of the signaling pathway can be, for example, a receptor ligand (e.g., a small molecule, an antibody, an siRNA), a ligand sequestrant (e.g., an antibody, a binding protein), a modulator of phosphorylation of a pathway component or a combination of such modulators.

One of skill in the art can easily test a compound to determine if it modulates a signaling pathway by assessing, for example, phosphorylation status of the receptor or expression of downstream proteins controlled by the pathway in cultured cells and comparing the results to cells not treated with a modulator. A modulator is determined to be a signaling pathway modulator if the level of phosphorylation of the receptor or expression of downstream proteins in a culture of cells is reduced by at least 20% compared to the level of phosphorylation of the receptor or expression of downstream proteins in cells that are cultured in the absence of the modulator, preferably the level of phosphorylation is altered by at least 30%, at least 40%, at least 50%, at least 60%, at least 70%, at least 80%, at least 90%, at least 95%, or at least 99% in the presence of a pathway modulator.

The terms “decrease”, “reduced”, “reduction”, “decrease” or “inhibit” are all used herein generally to mean a decrease by a statistically significant amount. However, for avoidance of doubt, ““reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, where the decrease is less than 100%. In one embodiment, the decrease includes a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kD), preferably less than 3 kD, still more preferably less than 2 kD, and most preferably less than 1 kD. In some cases it is preferred that a small molecule have a molecular weight equal to or less than 700 Daltons.

As used herein, an “RNA interference molecule” refers to a compound which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), microRNA (miRNA) and small molecules which interfere with or inhibit expression of a target gene by RNA interference (RNAi).

The term “polynucleotide” is used herein interchangeably with “nucleic acid” to indicate a polymer of nucleosides. Typically a polynucleotide of this invention is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynuceotide sequence” as used herein can refer to the polynucleotide material itself and/or to the sequence information (e.g. The succession of letters used as abbreviations for bases) that biochemically characterizes a specific nucleic acid. A polynucleotide sequence presented herein is presented in a 5′ to 3′ direction unless otherwise indicated.

The nucleic acid molecules that modulate the biological pathways or targets described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. Proc. Natl. Acad. Sci. USA 91:3054-3057, 1994). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example. by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Exemplary modifications include glycosylation and palmitoylation. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (e.g., the succession of letters or three letter codes used as abbreviations for amino acid names) that biochemically characterizes a polypeptide. A polypeptide sequence presented herein is presented in an N-terminal to C-terminal direction unless otherwise indicated.

The term “identity” as used herein refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Ahtschul at al. (Altsechul, et al., J. Mol. Biol. 215:403-410, 1990). To obtain gapped alignments for comparison purposes, Gapped BLAST is utilized as described in Altachul et al. (Alschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL www.ncbi.nlm.nih.gov for these programs. In a specific embodiment, percent identity is calculated using BLAST2 with default parameters as provided by the NCBI.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms “alkoxy”, “alkylaunino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “halo” refers to any radical of fluorine, chlorine, bromine or iodine.

The term “acyl” refers to an alkylcarbonyl, cycloalkylcarbonyl, arycarbonyl, heterocyclykarbonyl, or heterorylcarbonyl substituent, any of which may be further substituted by substituents. Exemplary acyl groups include, but are not limited to, (C₁-C₆)alkanoyl (e.g., formyl, acetyl, propionyl, butyryl, valeryl, caproyl, t-butylacetyl, etc.), (C₃-C₅)cycloalkycarbonyl (e.g., cyclopropylcarbonyl, cyclobutylcarbonyl, cyclopentycarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions.

The term “alkyl” refers to saturated non-aromatic hydrocarbon chains that may be a straight chain or branched chain, containing the indicated number of carbon atoms (these include without limitation methyl, ethyl, propyl, allyl, or propargyl), which may be optionally inserted with N, O, S, SS, SO₂, C(O), C(O)O, OC(O), C(O)N or NC(O). For example, C1-Ca indicates that the group may have from 1 to 6 (inclusive) carbon atoms in it.

The term “alkenyl” refers to an alkyl that comprises at least one double bond. Exemplary alkenyl groups include, but are not limited to, for example, ethenyl, propenyl, butenyl, 1-methyl-2-buten-1-yl and the like.

The term “alkynyl” refers to an alkyl that comprises at least one triple bond.

The term “alkoxy” refers to an —O-alkyl radical.

The term “aminoalkyl” refers to an alkyl substituted with an amino.

The term “mercapto” refers to an —SH radical.

The term “thioalkoxy” refers to an —S-alkyl radical.

The term “aryl” refers to monocyclic, bicyclic, or tricyclic aromatic ring system wherein 0, 1, 2, 3, or 4 atoms of each ring may be substituted by a substituent. Examplary aryl groups include, but are not limited to, phenyl, naphthyl, anthracenyl, azulenyl, fluorenyl, indanyl, indenyl, naphthyl, phenyl, tetrahydronsphthyl, and the like.

The term “arylalkyl” refers to an alkyl substituted with an aryl.

The term “cyclyl”. “cyclic” or“cycloalkyl” refers to saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Exemplary cycloalkyl groups include, but are not limited to, cyclopropyl, cyclobutyl, cyclopentyl, cyclopentenyl, cyclohexyl, cyclohexenyl, cycloheptyl, cyclooctyl, and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms ofN, O, or S if monocyclic, bicyclic, or tricyclic, respectively), wherein 0, 1, 2, 3, or 4 atoms of each ring may be substituted by a substituent. Examplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, and the like.

The term “heteroarylalkyl” refers to an alkyl substituted with a heteroaryl.

The term “heterocyclyl”, “heterocycle” or “heterocyclic” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examplary heterocyclyl groups include, but are not limited to piperazinyl, pyrrolidinyl, dioxanyl, morpholinyl, tetrahydrofuranyl, and the like.

The term “haloalkyl” refers to an alkyl group having one, two, three or more halogen atoms attached thereto. Exemplary haloalkyl groups incude, but are not limited to chloromethyl, bromoethyl, trifluoromethyl, and the like.

The term “optionally substituted” means that the specified group or moiety, such as an alkyl, aryl group, heteroaryl group and the like, is unsubstituted or is substituted with one or more (typically 1-4 substituents) independently selected from the group of substituents listed below in the definition for “substituents” or otherwise specified.

The term “substituents” refers to a group “substituted” on an alkyl, alkenyl, alkynyl, cycloalkyl, aryl, heterocyclyl, heteroaryl, acyl, amino group at any atom of that group. Suitable substituents include, without limitation, halo, hydroxy, oxo, nitro, haloalkyl, alkyl, alkenyl, alkynyl, alkaryl, aryl, aralkyl, alkoxy, aryloxy, amino, acylamino, alkylcarbanoyl, arylcarhanoyl, aminoalkyl, alkoxycarbonyl, carboxy, hydroxyalkyl. alkylthio, CF₃, N-morphilino, phenylthio, alkanesulfonyl, arenesulfonyl, alkanesulfonamido, arenesulfonamido, aralkylsulfonamido, alkylcarbonyl, acyoxy, cyano or ureido. In some embodiments, substituent can itself be optionally substituted. In some cases, two substituents, together with the carbons to which they are attached to can form a ring.

The compounds described herein and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

As used herein, the terms “stereoisomer” or “optical isomer” mean a stable isomer that has at least one chiral atom or restricted rotation giving rise to perpendicular dissymmetric planes (e.g., certain biphenyls, allenes, and spiro compounds) and can rotate plane-polarized light Because asymmetric centers and other chemical structure exist in the compounds described herein as suitable for use in the present invention which may give rise to stereoisomerism, the invention contemplates stereoisomers and mixtures thereof. The term “enantiomers” means a pair of stereoisomers that are non-superimposable mirror images of each other. The term “diastereoisomers” or “diastereomers” mean optical isomers which are not mirror images of each other. The term “racemic mixture” or “racemate” mean a mixture containing equal parts of individual enantiomers. The term “non-racemic mixture” means a mixture containing unequal parts of individual enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

ee=100×(E¹−E²)(E¹+E²),
wherein E¹ is the amount of the first enantiomer and E² is the amount of the second enantiomer.

In some embodiments, compound described herein have an enantiomeric excess of at least 1%, 5%, 10%, 20%, 30%, 40%, 50%, 60%. 70%, 80%, 90%, 95% or more. Generally, an ee of greater than 90% is preferred, an ee of greater than 95% is most preferred and an ee of greater than 99% is most especially preferred.

Enantiomeric enrichment is readily determined by one of ordinary skill in the art using standard techniques and procedures, such as gas or high performance liquid chromatography with a chiral column, Choice of the appropriate chiral column, eluent and conditions necessary to effect separation of the enantiomeric pair is well within the knowledge of one of ordinary skill in the at. In addition, the enantiomers of compounds can be resolved by one of ordinary skill in the art using standard techniques well known in the art, such as those described by J. Jacques, et al., “Enantiomers, Racemates, and Resolutions”, John Wiley and Sons, Inc., 1911. Examples of resolutions include recrystallization techniques or chiral chromatography.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors an not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The following examples illustrate some embodiments and aspects of the invention. It will be apparent to those skilled in the relevant art that various modifications. additions, substitutions, and the like can be performed without altering the spirit or scope of the invention, and such modifications and variations are encompassed within the scope of the invention as defined in the claims which follow. The following examples do not in any way limit the invention.

### Examples

### Materials and Methods

Chemical:

Kenpaullone, LY294002 and PI-103 were purchased from Tocris Bioscience. Alsterpaullone, GSK-3 inhibitor VII and GSK-3 inhibitor IX were purchased from EMD Millipore. CHIR 99021 was purchased from Stemgent CHIR 98014 was from Axon Medchem. Ara-C and Purvalanol A were purchased from Sigma-Aldrich.

Mouse ESC Culture and MN Diferentiation:

Mouse ESCs were differentiated into MNs and cultured as described (Makhortova et al., 2011) and plated in polyornithine-coated 96-well plates or poly-D-lysin-coated 384-well plates. For some experiments, MNs were purified by FACS, using a MoFlo XDP (Beckman Coulter) flow cytometer to isolate GFP⁺ cells from freshly dissociated embryoid bodies (EBs) resuspended in MN medium containing DMEM-F12 (Life Technologies), 2% FBS (Life Technologies), 827 supplement (Life Technologies), glial cell-derived neurotrophic factor (GDNF), brain-derived neuotrophic factor (BDNF), ciliary neurotrophic factor (CNTF) (R&D Systems), insulin, progesterone, penicillin/streptomycin, BSA, selenite and apo-transferrin (Sigma-Aldrich). FACS purified MNs were plated on growth factor reduced Matrigel (BD Biosciences).

Mouse MN Survival Assay:

For the small molecule screen or for standard survival assays, freshly dissociated EBs were plated at a density of 8,000 GFP⁺ cells (384-well) or 30.000 GFP+ cells (96-well) per well. Four days later (day 4), trophic factors (BDNF, GDNF and CNTF) were removed, and individual compounds were added to the wells. For the primary screen each was tested at three concentrations (0.1 μM, 1 μM and 10 μM) in duplicate. After an additional 72 hours (day 7), cells were fixed in 4% paraformaldehyde, stained with Hoechst 33342 (Life Technologies) and scanned using an automated confocal microscope (PerkinElmer Opera) at 10× magnification. The number of MNs surviving was analyzed by counting the remaining GFP⁺ cells in the whole well. Total cell numbers were obtained by counting Hoechst-positive nuclei. A size threshold was set based on control cultures to eliminate apoptotic MNs Unless stated otherwise, survival is measured as fold-increase compared to cultures maintained without trophic factors.

For long-term MN survival assays, MN cultures were fed with fresh medium and compounds every two days until fixation and quantification as above. For gene-specific knockdown with shRNAs, plated MNs were infected with lentivirus carrying shRNAs on day 2, trophic factors were withdrawn on day 4, and cells were fixed on day 7.

Human FSC/PSC Cultures and MN Differentiation:

Human ESCs and iPSCs were maintained on a feeder layer of inactivated mouse embryo fibroblasts (GlobelStem) in human ESC medium [KO-DMEM (Life Technologies), 10% KO Serum Replacement, penicillin/streptomycin, 2 mM GlutaMAX (Life Technologies), 100 μM non-essential amino acids (Life Technologies), 55 μM beta-merceptoethanol. 10% Plasmanate (Bayer), and 10 ng/ml bFGF2 (R&D SYSTEMS)]. The cells were cultured at 37° C. and 5% CO₂. Medium was replaced daily for the duration of ESC and iPSC culturing, and the cells were passaged every 5-7 days using StemPro EZPassage Disposable Stem Cell Passaging Tool (Life Technologies).

To differentiate human ESCs/iPSCs into MNs, ESC or iPSC colonies were detached and dissociated using StemPro EZPassage Disposable Stem Cell Passaging Tool, centrifuged for 5 min at 800 rpm, and resuspended in human ESC medium containing 10 μM ROCK inhibitor (Y-37632; EMD Millipore), but without bFGF2. Cells were filtered through 40 μm strainers and plated in ultra low attachment plates (Thermo Fisher Scientific) at 400,000 cells/ml. After 24 hrs (day 1), the cells were filtered again through 40 μm strainers in the same medium. On day 3, the cells had aggregated to form EBs and the medium was changed to human EB medium [DMEM/F12 (Life Technologies), penicillin/streptomycin, 2 mM GlutaMAX (Life Technologies), B27 supplement (Life Technologies) and N2 supplement (Life Technologies)] containing 1 μM retinoic acid. On day 9, a human hedgehog (Shh) agonist was added to EBs at a priming dose of 100 nM. On day 11, EBs were switched to medium containing 1 μM agonist. The medium was changed twice a week. EBs were dissociated between days 28 and 32 using 0.05% trypsin. Cells were plated in human MN medium (DMEM/F12, penicillin/streptomycin, 2 mM GlutaMAX, 0.2 mM ascorbic acid, and 0.16% glucose) containing 10 ng/ml BDNF and 10 ng/ml GDNF. The average MN differentiation rate using this protocol ranged from 5% to 20% based on Hb9 expression, or 20% to 40% based on Islet/MAP2 double-positivity. The neurons were grown on a monolayer of primary mouse cortical astrocytes prepared according to Di Giorgio et al. (2008) to increase MN survival. Uridine and 5-FU (5 μM each) were present during the first four days to reduce the number of proliferating progenitors. For human MN survival assays, trophic factors were removed and compounds were added on day 4, and cells were fixed after a further 14-day incubation. MN survival was determined by counting Hb9::GFP⁺ cells (HuES-3/Hb9::GFP) or Isl⁺/MAP2⁺ cells (iPSCs).

Human MN Differentiation and Survival Assay:

Human MNs were derived from human ESCs and iPSCs as previously described (Ding et al., 2013). After 30 days of differentiation, the cultures were dissociated with papain solution (Worthington) and plated as single cells on 384-well plates (Greiner) pre-seeded with mouse astrocytes. The medium used was Neuobasal containing 2% B27 and 1% N2 (Life Technologies) supplemented with 20 ng/mL of BDNF, GDNF and CNTF (R&D Systems). For human MN survival assays. trophic factors were removed, compounds were added on day 6, and cells were fixed after a further 14-day incubation. Ara-C was present throughout the entire experiment to eliminate proliferating progenitors. MN survival was determined by counting Hb9::GFP+ cells (HUES-3/Hb9:GFP) and Isl+/MAP2+ or Isl+/Tuj1+ cells (human iPSCs).

Immunocytochemistry:

Immunostaining was carried out using standard protocols (Makhortova et al., 2011). The following primary antibodies were used: mouse anti-Islet (DSHB), mouse or rabbit anti-Tuj1 (Covance), rabbit anti-MAP2 (EMD Millipore), rabbit anti-synapsin (EMD Millipore), mouse anti-PSD95 (BD Biosciences), mouse anti-hSOD1 (Sigma-Aldrich), goat anti-mSOD1 (R&D SYSTEMS), rabbit anti-ubiquitin (DAKO), rabbit anti-Olig2 (EMD Millipore), and anti-BrdU-Alexa 555 (BD Biosciences). Secondary antibodies conjugated with Alexa 488, Alexa 546 or Alexa 647 were purchased from Life Technologies. Hoechst 33342 was used for nuclear staining.

Electrophyslology:

Whole-cell voltage-clamp and current-clamp recordings were performed as described (Son et al., 2011).

Small Molecule Libraries for Motor Neuron Survival Screens:

The small molecules we used in these screens were: LOPAC1280 Collection (Sigma-Aldrich), Spectrum Collection (Microsource Discovery Systems), and Prestwick Chemical Library. A selected additional set of individual kinase inhibitors and GPCR ligands was also included.

Hit Identification:

In the primary screen, the inventors selected the top ranked compounds in each plate that also scored greater than 3 standard deviations above the low ctrl in terms of the numbers of GFP⁺ MNs. Compounds that scored as hits but gave rise to abnormal MN morphology (for example, cells with markedly reduced soma size) were excluded. All primary hits were subsequently tested in a nine-point dose-response assay, using the same screening protocol.

Image Analyses for Quantification of Synapses and Neurite Arborization:

For synapse analysis, the inventors quantified imaging data using the PerkinElmer Acapella software spot detection algorithm. Synapses were defined as the overlap of presynaptic and postsynaptic spots on MNs. On each GFP⁺ cell, we first drew a circle around the cell body with a diameter of approximately two cell body diameters, similar to that described by Ullian et al. (Schwann cells and astrocytes induce synapse formation by spinal motor neurons in culture. Mol Cell Neurosci., 2004, 25: 241-251) as the area for spot detection. Within each circle, presynaptic sites were detected using a primary antibody against synapsin and a secondary antibody conjugated with Alexa-632. Candidate spots that met an average intensity threshold were accepted as legitimate presynaptic punctac. Then we expanded the radius of each presynaptic puncta by 1 pixel, since the pre- and post-synaptic spots often were slightly offset, especially at higher magnifications, and measured the intensity in the postsynaptic channel, using a PSD95 primary antibody and a secondary antibody conjugated with Alexa-546. Postsynaptic punctae that exceeded the threshold mean intensity threshold were accepted, permitting us to define “colocalized” spots, or synapses.

The neurite tracing method in Acapella software is based on methods developed at CSIRO at perkinelmer.com/pages/020/cellularimaging/assaysneuriteoutgrowth.xhtm. The cell bodies of MNs were located by expression of HB9:GFP, and the neurites were also detected by GFP fluorescence. The algorithm measured the lengths and fluorescence intensities of each neurite and the branching level (primary, secondary, etc). The total lengths of neurites and the numbers, lengths and fluorescent intensities of primary and secondary branches were measured based on selected threshold parameters.

Adjustment of Calculations for MN Survival Using GSK-3 Knockout Lines:

In these cultures, approximately 70% of Isl+/MAP2+ cells were GFP+(i.e., MNs). However, after trophic factor withdrawal, because MNs die selectively under these conditions, only about 50% of Isl+/MAP2+ cells were GFP+MNs. This means that counting Isl+/MAP2+ cells could overestimate the amount of survival after trophic factor withdrawal by approximately 40%. When deprived of neurotrophins, 35% of wild-type Isl+/MAP2+ cells survived, while 44%, 57% and 54% of Isl+/MAP2+ cells in GSK-3□-KO, GSK-3□-KO and 3/4-KO cultures survived. Using the correction factor for overcounting surviving MNs, this would mean that wild-type MN survival would be approximately 25%, while the KO MNs would have 31%, 41% and 39% survival, respectively.

Phospho-Kinase Antibody Array:

Freshly dissociated HB9::GFP EBs were plated at a density of 3 million GFP+ cells/10 cm dish and trophic factor withdrawal/compound addition was performed on day 4. Protein lysates were generated 6 and 24 h after treatment using RIPA buffer supplemented with protease and phosphatase inhibitors (Pierce Biotechnology). Equal amounts of total protein from treatment groups were added to Phospho-Kinase Antibody Arrays (R&D Systems, #ARY003). Arrays were prepared following the manufacturer's instructions and developed with SuperSignal West Pico Chemiluminescent Substrate (Pierce Biotechnology). Background signal was subtracted and duplicate spot pixel densities were averaged using QuantityOne software (BioRad).

JNK/c-Jun Signaling Pathway Analysis:

For JNK/c-Jun signaling pathway analysis, freshly dissociated SOD1G93A/HB9::GFP and HB9::GFP EBs were plated at a density of 30,000 GFP+ cells/well in 96-well plates and trophic factor withdrawal/compound addition was performed on day 4. Cells were fixed at 1, 6, 24 and 48 h after treatment, stained with appropriate antibodies, and imaged using an automated microscope (Perkin Elmer Operetta) at 20× magnification. GFP+ MNs were identified using Columbus analysis software (Perkin Elmer). Nuclear and cytoplasmic compartments were defined using Hoechst and GFP. Mean intensity of fluorescent staining across nuclear or cytoplasmic compartments was measured for each MN. Intensities were corrected for background staining levels (secondary antibody only) and the number of positive MNs per condition quantified.

Kinase Activity Assays:

For kinase activity assays, 0.4 mg of MLK3 and 0.1 mg of HGK active kinases (SignalChem) were incubated for 35 minutes at 30° C. with 0.5 mg of MBP (SignalChem) in the presence of [□-32] ATP and Kenpaullone/CHIR99021. The enzymatic reaction was stopped with Laemmli buffer and samples were separated in a polyacrylamide gel. Finally, the gel was exposed to a Storage Phosphor Screen (GE Healthcare) and scanned in a Typhoon 9400 scanner (GE Healthcare). Signal density was estimated with Quantity One software (Bio-Rad).

Results and Discussion

Assay Developer or Motor Neuron Survival Screens:

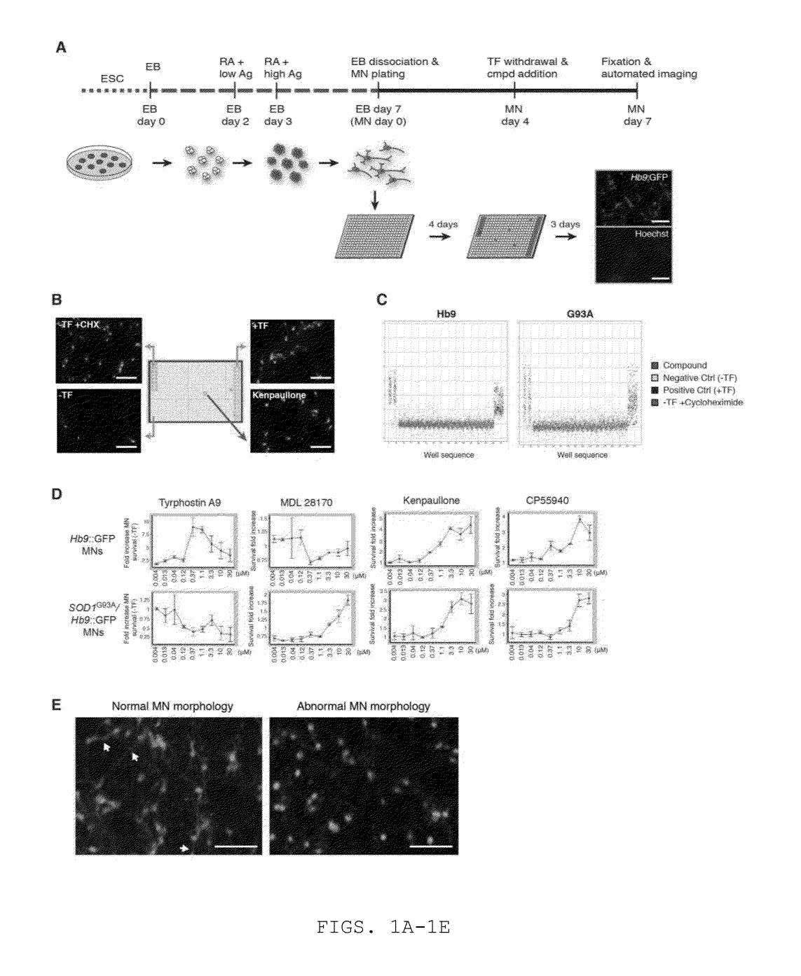

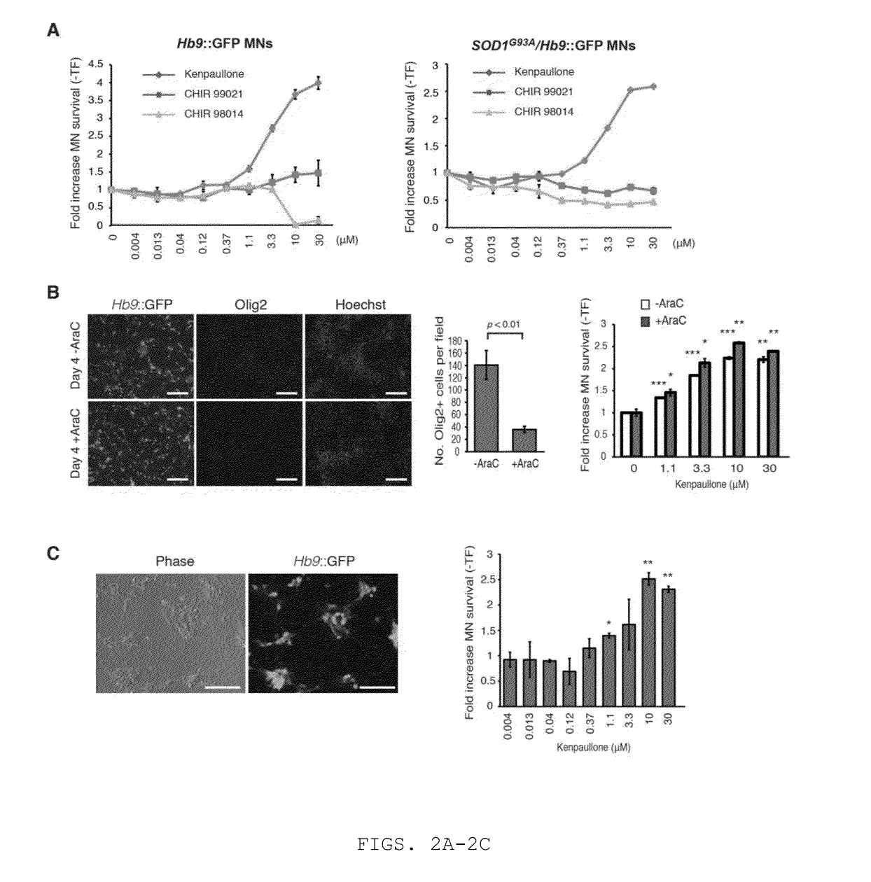

The inventors produced MNs from both wild-type (Hb9::GFP) and SOD1^{G93A} (SOD1^{G93A}/HB9::GFP) mouse ESCs using modifications of standard protocols (FIG. 1A and Methods and Materials section) and were able to generate cultures with 30%□50% MNs in both lines. Both ESC lines carry a transgenic reporter gene in which the expression of the green fluorescent protein (GFP) is driven by the promoter of the Hb9 gene, a transcription factor expressed in postmitotic MNs. As reported by Di Giorgio et al. (Di Giorgio et al., 2007), the inventors discovered that there was no significant difference in the basal survival between HB9::GFP and SOD1^{G93A}/HB9::GFP cultures in the first week, although the survival of SOD1^{G93A}/HB9::GFP MNs was somewhat more variable.

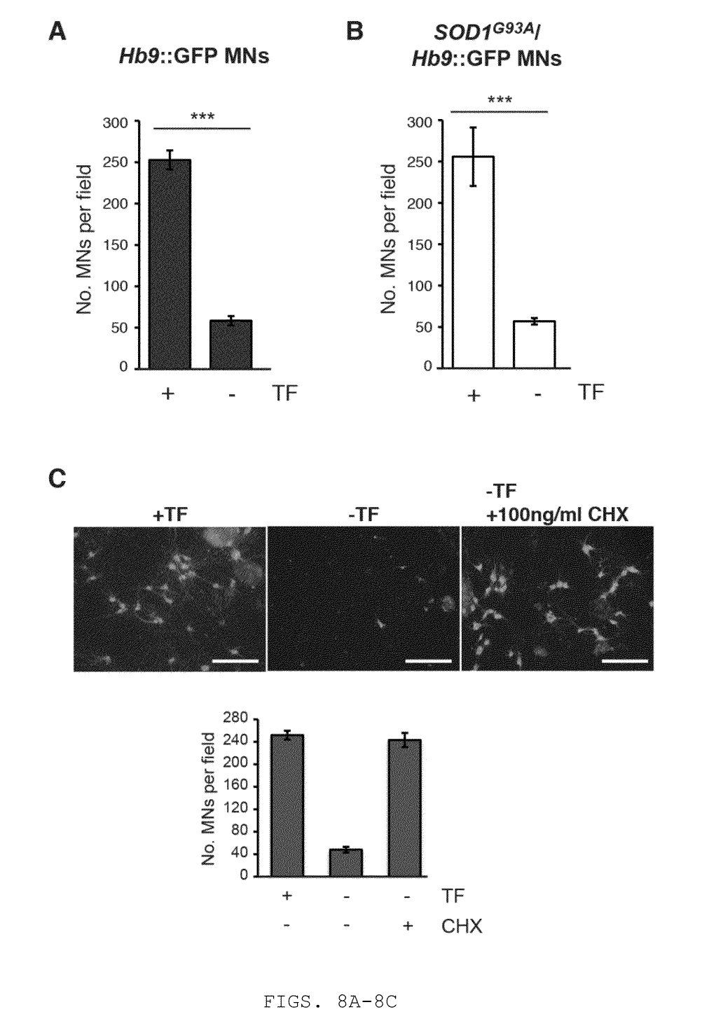

The inventors considered using co-culture with SOD1^{G93A} astrocytes to induce MN death, but were concerned that death occurs only over an extended period of time and that this assay may model only one form of the disease. Thus, the inventors chose to rely on a standard model of inducing death—trophic factor withdrawal—but applied it to both types of MNs. Various plating densities and times for initiation and duration of trophic factor withdrawal were optimized for a 384-well HTS format, and inventors discovered conditions such that about 80% of the MNs died in 3 days (FIG. 8). This trophic factor-induced death was relatively specific to MNs since the total numbers of non-MNs (determined by counting Hoechst⁺ nuclei) did not change after trophic factor withdrawal (data not shown). Cycloheximide (CHX), a protein synthesis inhibitor that blocks apoptosis (Mattson and Furukawa, 1997), was identified as a hit from a trial screen, was shown to be able to effectively rescue MNs from trophic factor-withdrawal-induced cell death (FIG. 8C, and was used as a positive control compound.

Identification of Compounds that Promote MN Survival:

A small molecule collection of approximately 5000 chemicals containing known drugs, bioactives and other annotated compounds was screened at three different concentrations (10 μM, 1 μM and 0.1 μM) in duplicate (FIG. 1). Hits were identified as those compounds that both robustly increased the number of MNs relative to wells without trophic factors and preserved cell morphology (size of soma, presence of neurites; FIG. 1F). Hit compounds from the primary screen were retested in dose-response survival assays. Confirmed hits were then categorized based on their annotated activities as summarized in Tables 3-5.

TABLE 3
A summary of hit classes fromHB9::GFP screen.

| Hit category | No. hits |
|---|---|
| Antimetabolite | 1 |
| Apoptosis | 2 |
| Cell Cycle | 7 |
| Cytoskeleton | 1 |
| DNA/Protein synthesis | 6 |
| Estrogen | 3 |
| GPCR | 2 |
| Ion channel | 1 |
| Kinase | 8 |
| Lipid Mediators | 1 |
| Metalloproteinase | 1 |
| Neurotransmission | 11 |
| NSAID | 1 |
| Phosphodiesterase | 1 |
| Retinoic Receptor | 1 |
| Unclassified | 7 |
| Total | 54 |

TABLE 4
A summary of hit classes fromSOD1^{G93A}/HB9::GFP screen.

| Hit category | No. hits |
|---|---|
| Apoptosis | 1 |
| Cell Cycle | 5 |
| DNA/protein Synthesis | 8 |
| Estrogen | 4 |
| GPCR | 1 |
| Ion channel | 2 |
| Kinase | 5 |
| Lipid Mediators | 1 |
| Metalloproteinase | 1 |
| Neurotransmission | 7 |
| Phosphodiesterase | 1 |
| Total | 36 |

TABLE 5
Of the 54 HB9::GFP hits and the 36 SOD1^{G93A}/HB9::GFP
hits, 22 overlapped between the two MN types.

| Hit category | No. hits |
|---|---|
| Apoptosis | 1 |
| Cell Cycle | 5 |
| DNA/protein Synthesis | 5 |
| Estrogen | 1 |
| GPCR | 1 |
| Ion channel | 1 |
| Kinase | 3 |
| Metalloproteinase | 1 |
| Neurotransmission | 3 |
| Phosphodiesterase | 1 |
| Total | 22 |

Screening on both HB9::GFP and SOD1^{G93A}/HB9::GFP cultures resulted in three sets of hits: compounds that scored only in HB9::GFP MNs, those that scored only in SOD1^{G93A}/HB9::GFP MNs, and those that scored in both (FIG. 1D and Tables 3-5). A significant number of hits or hit categories either act on targets previously shown to be involved in ALS or to be neuroprotective. Nine compounds that inhibit protein or DNA synthesis, including cycloheximide, the positive control for the assay, improved survival of one or both types of MNs. These types of compounds are well known to be anti-apoptotic (Mattson and Funrukawa, 1997; Martin, 2001). The matrix metalloproteinase (MMP) inhibitor, 1,10-phenanthroline monohydrate, promoted the survival of both types of MNs. MMPs have been implicated in MN degeneration in ALS, and MMP inhibitors have been shown to extend the survival of ALS mice (Lorenzl et al., 2006; Fang et al., 2009; Niebroj-Dobosz et al., 2010). CP55940 (a cannabinoid receptor agonist) promoted the survival of both HB9::GFP and SOD1^{G93A}/HB9::GFP MNs (FIG. 1E). Cannabanoid receptors have previously been shown to be elevated in spinal cords of symptomatic SOD1^{G93A} mice, and treatment with cannabinoid receptor agonists delayed disease onset and prolonged survival (Kim et al., 2006; Shoemaker et al., 2007). Additionally, the calpain inhibitor MDL 28170 promoted the survival of SOD1^{G93A}/HB9::GFP MNs (FIG. 1E), which agrees with the studies in which calpain inhibition prolonged the viability of SOD1^{G93A} MNs in culture and the lifespan of SOD1^{G93A} mice (Wootz et al., 2006; Tradewell and Durham, 2010). In addition, we observed that ligands for different neurotransmitter receptors (e.g. A 77636 hydrochloride and 3-Tropanylindole-3-carboxylate methiodide), and compounds targeting calcium channels (e.g. the calcium agonist FPL-64176) scored as hits in one or both types of MNs. This came as no surprise as neuronal activity and calcium flux are crucial regulators for neuronal survival, and ALS is known to involve the dysregulation of both (Bertel at al., 1991; Krieger et al., 1996; Sandyk, 2006; Grosskreutz et al., 2010). Interestingly, quinacrine dihydrochloride dihydrate (a multi-functional compound) has been shown to be neuroprotective via its prion-blocking activity (Fasano et al., 2008; Spilman at al., 2008), and deacetoxy-7-oxisogedunin (a tetranortriterpenoid compound) has been shown to reduce protein aggregation under some circumstances as well as to increase proteasome activity (Kioschis-Schneider and Hafner, 2010) Both compounds scored as hits in both types of MNs, potentially explaining part of their survival promoting activities although protein aggregation is not thought to occur at the time points of our assay (Ferraiuolo at al., 2011; Polymenidou and Cleveland, 2011). Finally, several kinase inhibitors were noted as hits. Tyrphostin A9 (a multi-kinase inhibitor) promoted the survival of HB9::GFP MNs alone, while Kenpaullone (annotated as a GSK-3 inhibitor) had strong effects on both types of MNs (FIG. 1E).

Kenpaullone and Some GSK-3 Inhibitors Promote Survival:

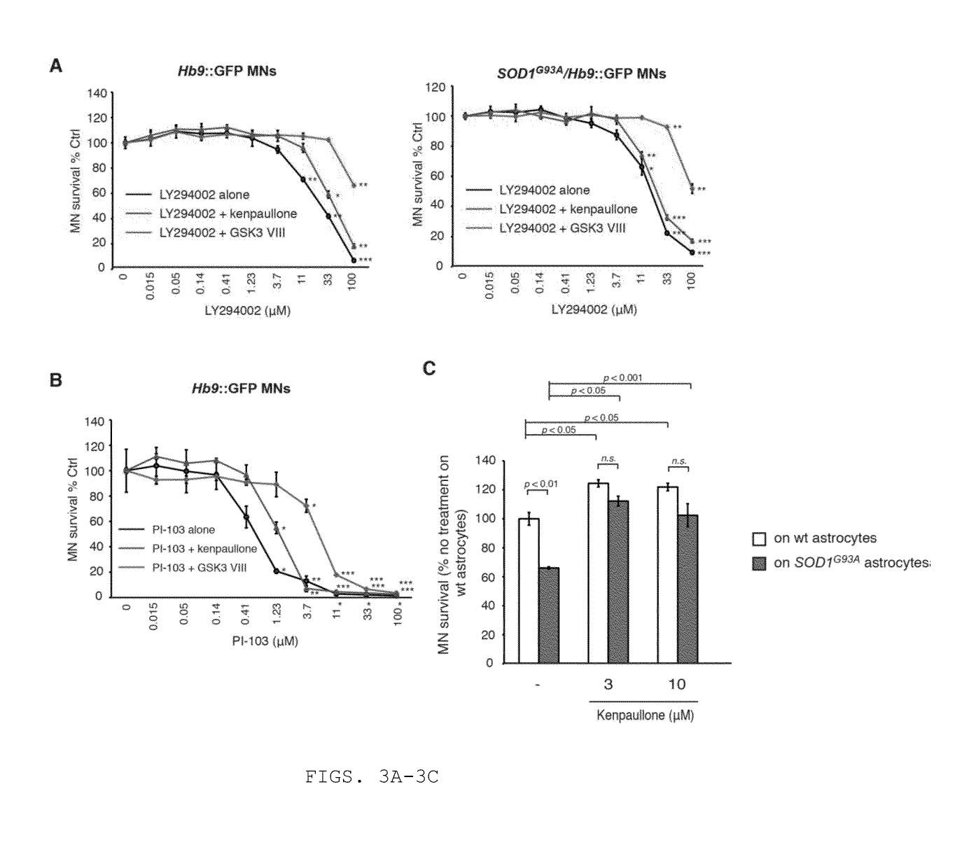

For follow-up studies the inventors focused on compounds that acted on more biologically interesting or pharmacologically “druggable” targets or pathways. The inventors also chose to pursue compounds that affected both MN types, thinking that they might have the best chance of working broadly across different ALS disease types. The inventors also tested additional commercially available compounds that act on similar targets to those affected by the best hit compounds. The inventors discovered that several putative GSK-3 inhibitors had some activity although kenpaullone, in particular, reproducibly and consistently showed potent survival promoting effects on both wild-type and mutant MNs (FIG. 2A). Surprisingly, another highly related GSK-3 inhibitor, alsterpaullone, showed much weaker and quite variable effects.

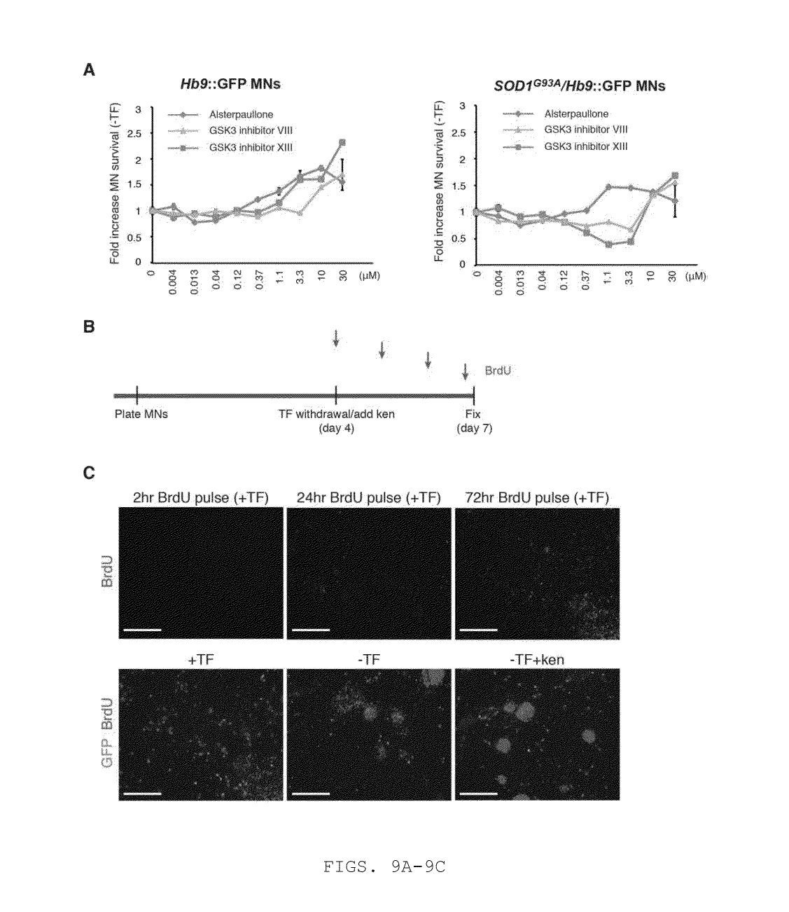

To expand on this finding, the inventors evaluated the activities of many GSK-3 inhibitors (FIGS. 2A and 9A). Two, GSK-3 inhibitor VIII (Anastassiadis et al., 2011) and GSK-3 inhibitor XIII, showed consistent, albeit significantly weaker, activity in the survival assays. However, again unexpectedly, some GSK-3 inhibitors, including those thought to be the most specific, did not promote survival at all (FIG. 9A). The varying ability of the different inhibitors to prolong survival seemed unlikely to be the result of incomplete GSK-3 inhibition, as kenpaullone, alsterpaullone and CHIR 99021 all should have blocked GSK-3 activity almost completely at the concentrations we used (Bain et al., 2007). These experiments suggested that, while GSK-3 inhibition might play a role in promoting survival, the particularly pronounced effect of kenpaullone could have resulted from additional molecular activities.

Kenpaullone Increases Survival, but not the Production of New MN:

Recent publications suggest that GSK-3 may be involved in regulating the proliferation and differentiation of neural progenitors (Hur and Zhou, 2010; Lange et al., 2010). To confirm that the increase in MN numbers produced by kenpaullone treatment was the result of an increase in survival of existing MNs, rather than in the number of newly born MNs derived from the progenitors in the cultures, the inventors treated the cultures with the antimitotic agent Ara-C to eliminate dividing progenitors before trophic factor withdrawal and treatment (FIG. 2B). While treatment with Ara-C significantly reduced the number of Olig2⁺ progenitor cells, it did not decrease the effect of kenpaullone. This demonstrates that the effect of kenpaullone was not the result of increased neuron generation.

The inventors also used BrdU to label proliferating cells and any MNa potentially derived from them (FIGS. 9B and 9C). During the trophic deprivation and kenpaullone incubation period, BrdU was added at different times (72 hrs, 48 hrs, 24 hrs and 2 hrs before fixation) and the number of BrdU⁺/GFP⁺ cells was measured. Interestingly, in none of the conditions examined did we find any BrdU⁺/GFP⁺ double-positive cells, suggesting that there was little production of new MNs during this period, strengthening the conclusion that there is a genuine survival promoting effect of kenpaullone.

The Effect of Kenpaullone is Cell Autonomous:

ESC-derived MN cultures are heterogeneous, with 50% to 70% of the cells being other types of neurons and non-neuronal cells. To determine if kenpaullone exerts its protective effect in a cell autonomous or non-cell autonomous way, the inventors used FACS to purify MNs based on their Hb9::GFP expression and established highly enriched cultures (FIG. 2C). In addition, we treated the cultures with Ara-C to ensure that there were a limited number of proliferating progenitors. When trophic factors were removed from these cultures, kenpaullone treatment was still able to promote survival, with a dose curve similar to that observed in mixed cultures. This suggests that kenpaullone acts on MNs themselves.

Kenpaullone Also Protect when Death is Initiated by Other Stimuli:

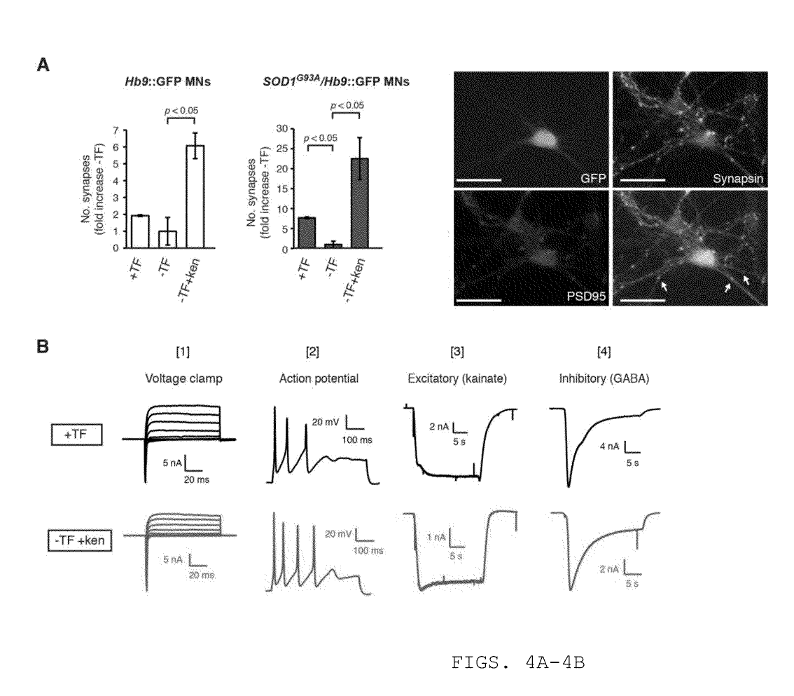

The PI3K/Akt pathway is well known to regulate neuronal survival including that of MNs (Brunet et al., 2001; Barthélémy et al., 2004). PI3K/Akt activity has been shown to be decreased in MNs of both sporadic and familial ALS patients, as well as in those in mutant SOD1 mice (Koh at al., 2004; Dewil at al., 2007). The inventors sought to examine if either kenpaullone or a more specific GSK-3 inhibitor (Inhibitor VIII) could protect MNs from death caused by inactivation of PI3K/AKT. As shown in FIG. 3A, the PI3K inhibitor LY294002 induced dose-dependent death in both types of cultures. However, when 10 μM kenpaullone was co-incubated with LY294002, MN death was significantly attenuated. On the other hand, Inhibitor VIII treatment produced only a very small increase in HB9::GFP MN survival and did not have any noticeable effect on SOD1^{G93A}/HB9::GFP MNs. Thus kenpaullone's effects are not restricted to protection after trophic factor withdrawal. These results again support the conclusion that kenpaullone may have activities beyond inhibiting GSK-3.

Several studies have shown that astrocytes derived from ALS patients or animal models, especially mutant SOD1-bearing astrocytes, contribute to MN death (Di Giorgio et al., 2007; 2008; Marchetto at al., 2008; Diaz-Amarilla at al., 2011; Haidet-Phillips et al., 2011). The inventors also observed that HB9: FP MNs that were plated on a monolayer of SOD1^{G93A} astrocytes survived less well than the same cells plated on wild-type astrocytes (FIG. 3B). To test if kenpaullone could reduce this type of death, the inventors treated the co-cultures with kenpaullone and found that it improved survival on both wild-type astrocytes and on SOD1^{G93A} astrocytes. However, the percentage increase was much greater on SOD1^{G93A} astrocytes, making survival on SOD1^{G93A} astrocytes almost indistinguishable from that on wild-type astrocytes. This result demonstrates that kenpaullone is able to act even when death is triggered by “toxic” glial factors.

Effect of Kenpaullone of MN Morphology and Function:

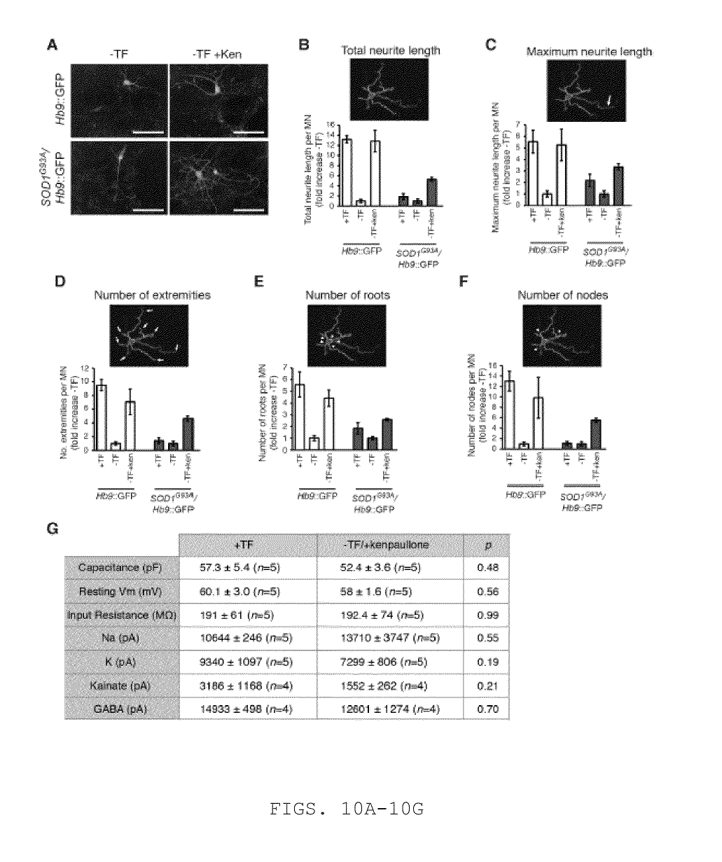

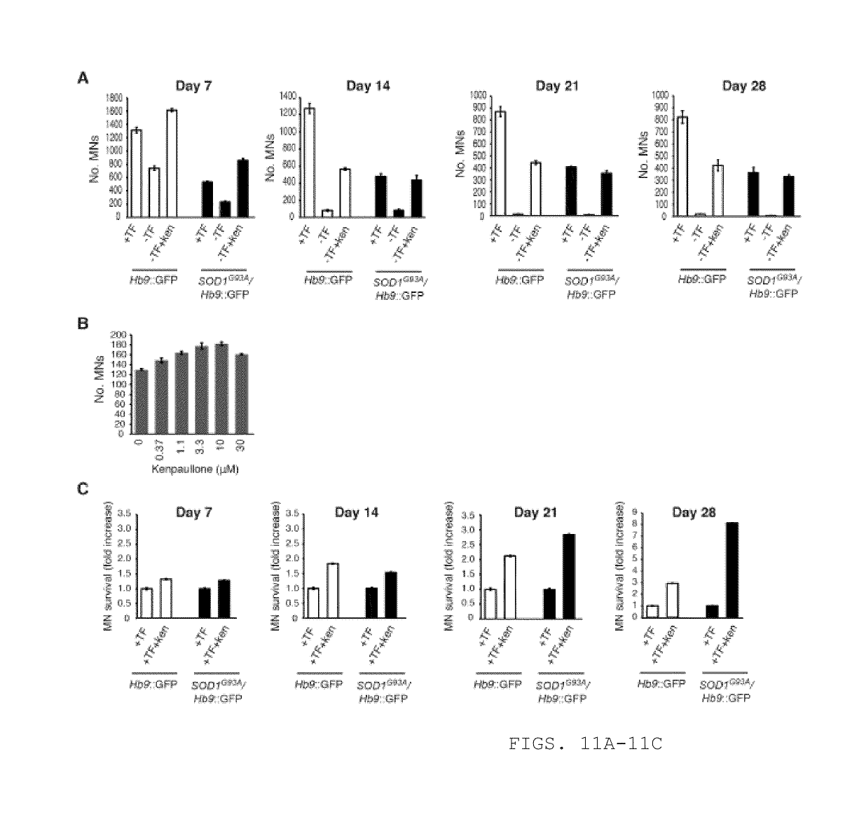

To determine whether MNs kept alive by kenpaullone in the absence of neurotrophic factors are intact, the inventors evaluated a variety of morphological and electrophysiological properties of the surviving cells. First, the Inventors discovered that kenpaullone substantially supported the structural integrity of MN processes even at extended time points (FIGS. 4A and 10A-10F). It is noteworthy that kenpaullone alone was able to maintain the neuritic network of these cells at levels seen in trophic factor supplemented cultures. Moreover, its effect on SOD1^{G93A}/HB9::GFP cultures was even more pronounced, allowing neurite growth beyond that seen in cells kept in trophic factors.

ESC-derived MNs and other spinal cord neurons in mixed cultures are able to form synapses (Palizvan et al., 2004; Bekkers, 2009). The inventors used an automated imager to count the number of synapses per MN (defined as overlapping regions that were positive for the presynaptic marker synapsin and the potsynaptic marker PSD95 on individual MNs; FIG. 4B). In Hb9::GFP cultures, MNs that survived trophic factor withdrawal exhibited a trend towards a decreased number of synapses per cell, but this was not statistically significant. However, kenpaullone treatment produced a dramatic increase in the number of synapses on individual MNs even in the prolonged absence of trophic support. In SOD1^{G93A}/HB9::GFP cultures minus trophic factors, surviving MNs (although there were few) had fewer synapses per cell. Kenpaullone addition not only increased the survival of these cells, but is supported their ability to maintain morphological synapses.

To examine whether kenpaullone could maintain neuronal health during trophic factor withdrawal, the inventors also compared the electrophysiological properties of MNs grown in complete medium (+TF) with those of cells in trophic factor-deficient (−TF) medium with or without kenpaullone supplementation (FIGS. 4C and 10G). First of all, when the inventors attempted to record from cells grown in medium lacking both trophic factors and kenpaullone, the inventors were unable to obtain stable recordings despite a significant number of patch attempts. However, in whole-cell voltage-clamp recordings from the other two groups of cells, depolarization elicited fast voltage-activated inward currents followed by slow outward currents, consistent with voltage-activated sodium and potassium currents, respectively. When the inventors compared peak sodium and steady-state potassium current amplitudes, the inventors found no difference between cells in those two groups (FIG. 10G). In current clamp mode, depolarizing stimuli elicited robust action potentials in both groups of cells. Two key measurements that reflect neuronal health are the resting membrane potential and input resistance, and we found that neither varied between the two groups (FIG. 10G). The inventors also recorded responses to excitatory neurotransmitters, such as kainate, and Inhibitory transmitters, such as GABA, in the two sets of MNs. Again, the inventors found no significant difference. Together, these data suggest that kenpaullone treatment preserves neuronal health when trophic factors are removed.

Kenpaullone Promotes Long-Term Survival of Both Types of MNs:

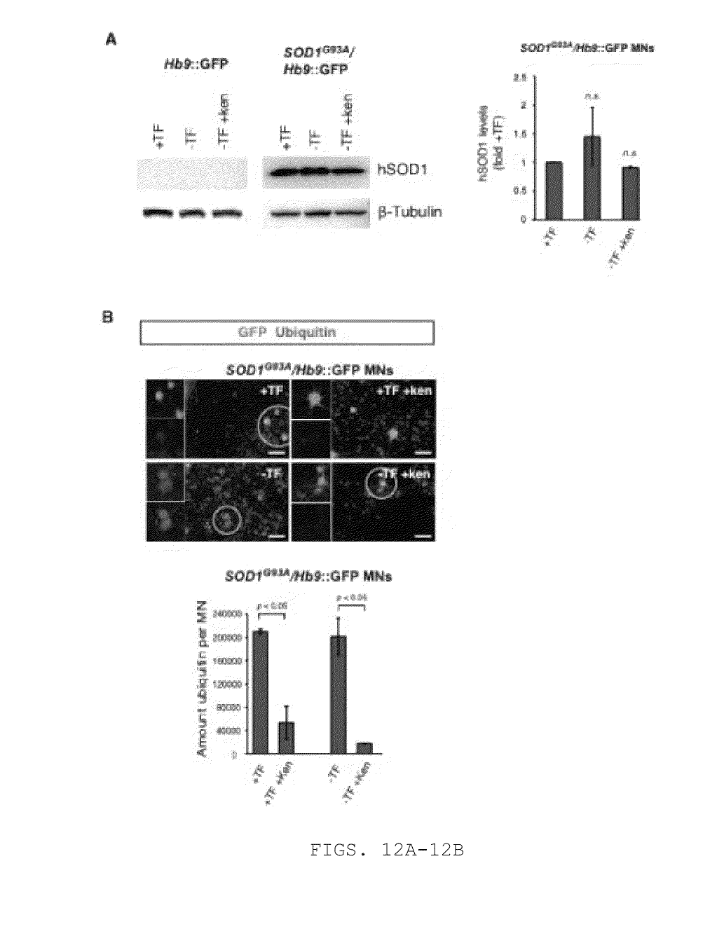

To investigate whether kenpaullone was able to promote survival in a trophic factor deficient environment for extended periods of time, the inventors removed trophic factors and added kenpaullone on day 4, then analyzed survival on days 7, 14, 21 and 28 (FIG. 11A). At all time points examined, kenpaullone improved survival of both HBP::GFP and SOD1^{G93A}/HB9:GFP MNs, but the effect was proportionally greater in SOD1^{G93A}/HB9::GFP cultures at the longer time points (comparing cells grown without trophic support but with kenpaullone to cells grown with trophic support). This demonstrates that, while kenpaullone promotes the survival of normal MNs, it can also have additional beneficial activities that are specific to the mutant SOD1^{G93A} cells.

Under normal culture conditions, even when cells were maintained in neurotrophin-containing medium, there was a basal level of cell death that could be prevented by kenpaullone treatment (FIG. 11B). As previously reported (Di Giorgio et al. 2007), after about 2 weeks, the amount of MN death in SOD1^{G93A}/HB9::GFP cultures exceeded that in HB9::GFP cultures. The inventors found that, during the first two weeks, kenpaullone reduced the death of both MN populations in similar proportions (FIG. 11C). However, after that, the relative death of SOD1^{G93A}/HB9::GFP MNs increased, and the effect of kenpaullone again became relatively greater. This became increasingly striking after 3-4 weeks of culture so that at day 28, there were 8 times the number of SOD1^{G93A}/HB9::GFP MNs in medium with kenpaullone (FIG. 11C).

Kenpaullone Decreases Mutant SOD1 Levels:

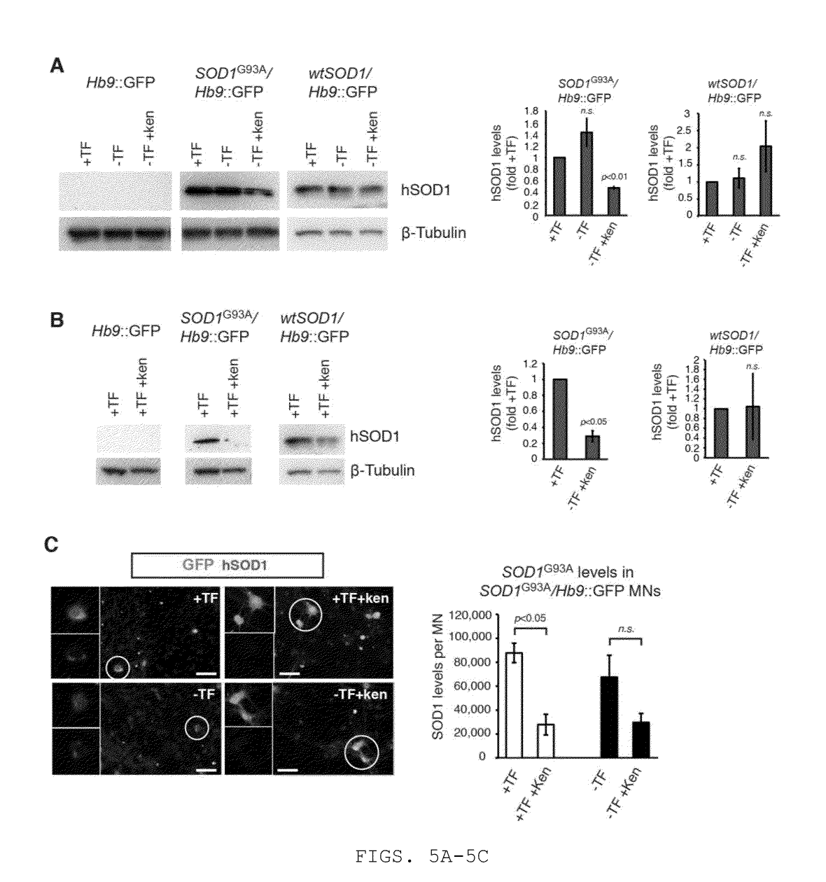

Since the additional death undergone by the SOD1^{G93A}/HB9::GFP MNs seems likely to relate to their expression of high levels of SOD1^{G93A} protein, one possibility is that kenpaullone could act in part by reducing those levels. Kenpaullone was added to cultures on day 4, and the levels of mutant SOD1^{G93A} protein were analyzed by Western blotting on day 21 (FIGS. 5A and 5B). Such treatment, in both the absence (FIG. 5A) and the presence (FIG. 5B) of trophic factors, produced a sharp decrease in the levels of SOD1^{G93A} protein. The endogenous mouse SOD1 protein was not affected in either HB9::GFP or SOD1^{G93A}/HB9::GFP cultures (data not shown). In addition, the inventors discovered that kenpaullone had no effect on the levels of wild-type human SOD1 in cultures produced from an ESC line that overexpresses a wild-type human SOD1 transgene (wtSOD1/Hb9::GFP) (FIGS. SA mad SB). Since the cultures used in these experiments contained a mixture of cell types, kenpaullone's reduction of SOD1^{G93A} protein as seen by Western blot did not necessarily reflect SOD1 protein levels in MNs themselves. However, when the inventors used single cell confocal imaging to examine SOD1^{G93A} levels specifically in GFP⁺ cells, it was clear that the SOD1^{G93A} levels in MNs were reduced, both in the presence and absence of trophic factors (FIG. 5C).

It has been shown that SOD1^{G93A} accumulates in ubiquitinated aggregates after about 2 weeks of culture (Di Giorgio et al., 2007). Ubiquitinated proteins are found to be elevated in MNs of ALS patients and SOD1^{G93A} mice during neural degeneration (Watanabe et al., 2001; Wang et al., 2003). The inventors also observed the appearance of aggregates in their cultures at these later times (FIG. 5C). This led us to examine when differences in SOD1^{G93A} protein levels began to appear. Importantly, the inventors found no differences in mutant protein levels between untreated and kenpaullone treated cultures until sometime after 15 days of treatment (FIG. 12A S5A). The inventors also measured MN levels of ubiquitin by single cell imaging and found it also reduced at both days 21 and 28, but not at day 15 regardless of the presence or absence of trophic factors (FIG. 12B). Thus, the ability of kenpaullone to reduce the later periods of SOD1^{G93A}/HB9::GFP death coincides in time with the decreased levels of SOD1^{G93A} and ubiquitin.

Kenpaullone Promotes Survival of MNs Derived from Human ALS iPSCs:

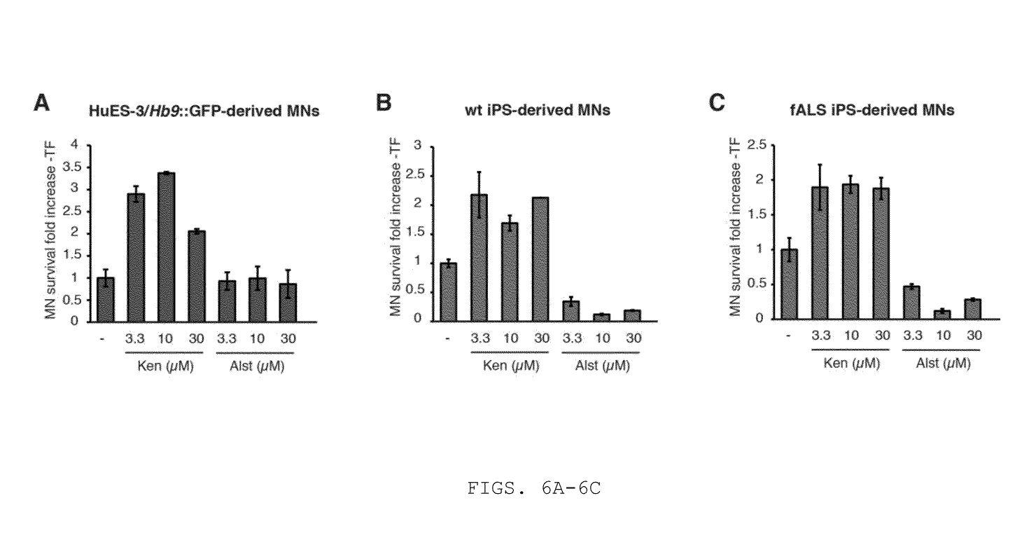

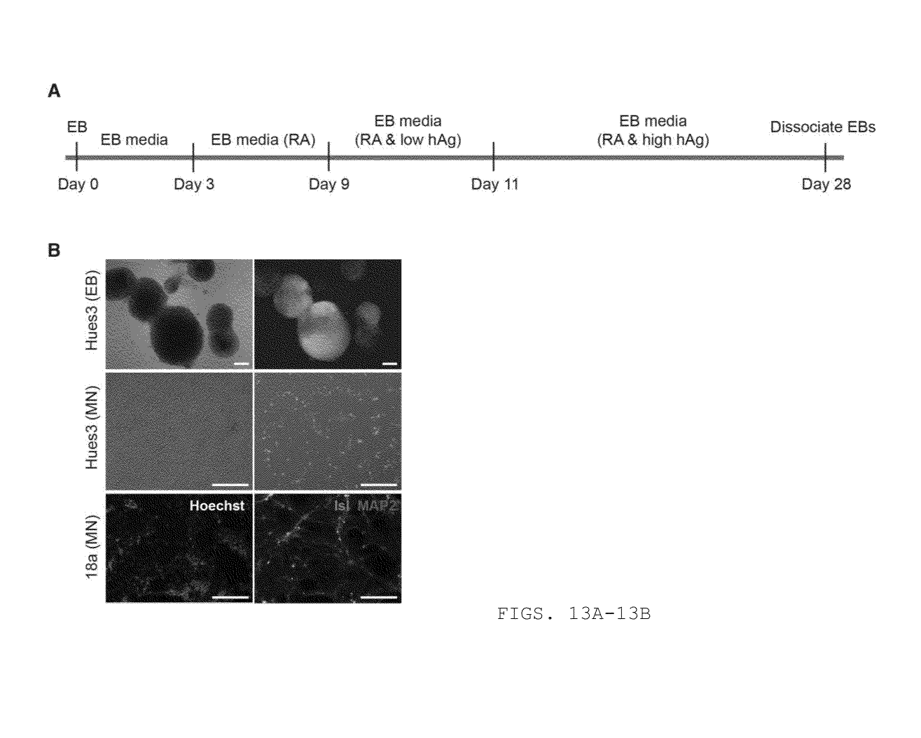

For the mouse screening results to provide information relevant to treating human ALS, it is important to show that compounds that are effective on mouse cells are also effective on human cells. The inventors generated MNs from a human ESC line expressing an HB9::GFP transgene [HuES-3/Hb9::GFP (Di Giorgio et al., 2008)] and FACS purified them to reduce the number of residual progenitor cells and other types of neurons (FIG. 13). The inventors then induced these cells to die by withdrawing trophic factors (FIG. 6A). We found that kenpoullone treatment was able to protect the human neurons from cell death.

The inventors then tested kenpaullone on MNs produced from iPSCs derived from a control individual and from an ALS patient with a confirmed mutation in SOD1 [SOD1L^{L144F}(Boulting et al., 2011)]. The inventors treated cultures with antimitotics to minimize the number of proliferating neuronal progenitors and the number of new neurons that can be generated during the period of trophic factor withdrawal. In both types of cultures, kenpaullone produced a substantial increase in MN survival while the related GSK-3 inhibitor alsterpaullone was either toxic or had no effect (FIGS. 6B and 6C). This confirms the ability of kenpaullone to act on human diseased cells and also suggests that this activity does not result from GSK-3 inhibition alone.

Mechanistic Studies of Kenpaullone's Survival Promoting Activities:

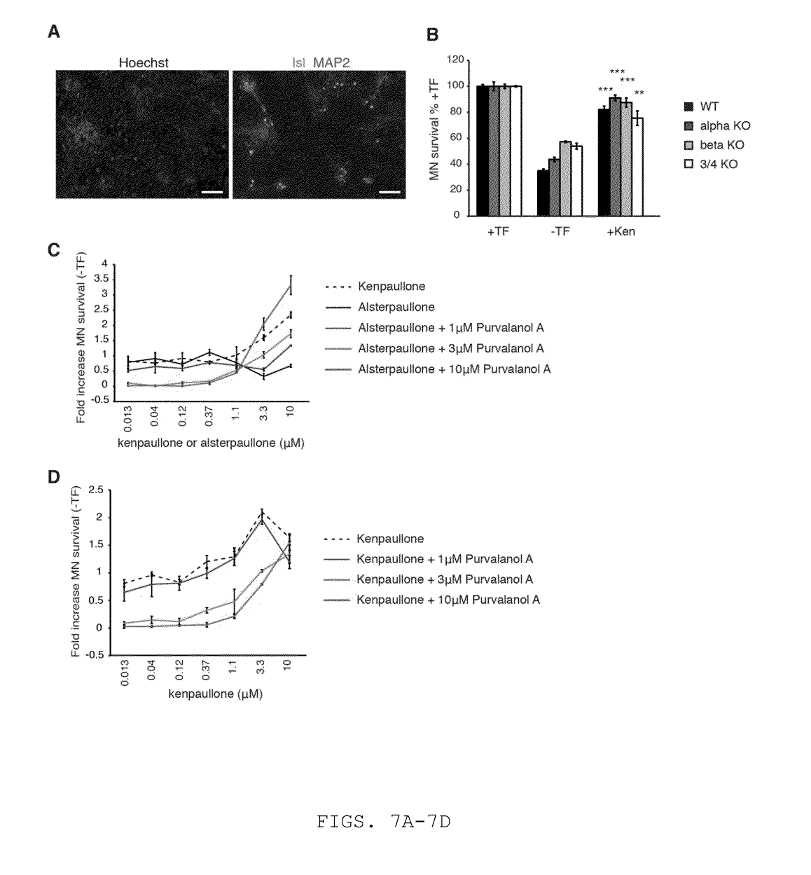

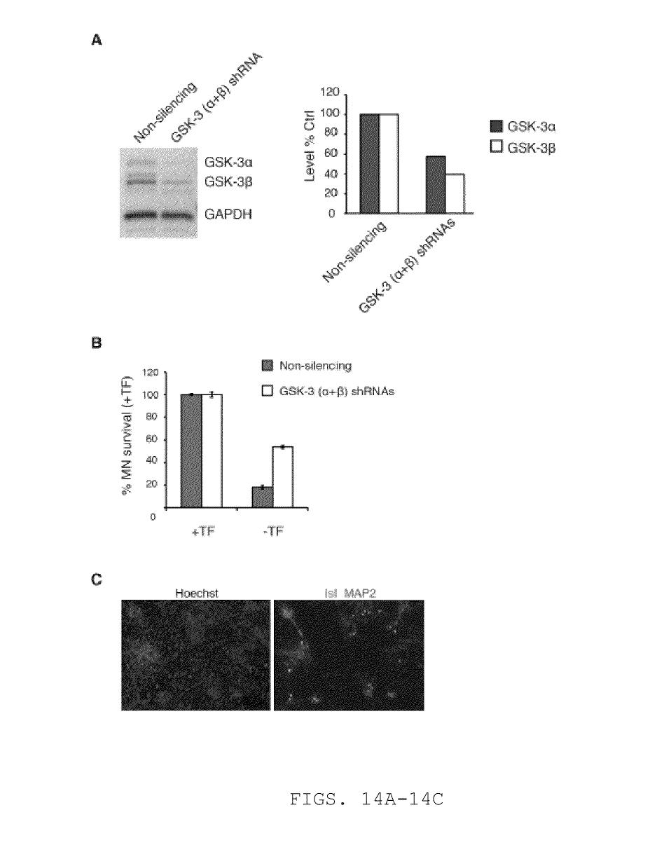

Since kenpaullone is known to inhibit GSK-3, the inventors first sought to determine the role of GSK-3 in MN survival in our culture conditions. It has been shown that most small molecule GSK-3 inhibitors have no isoform selectivity and show similar potencies towards GSK-3α and GSK-3β (Medina and Castro, 2008). In addition, GSK-3α and GSK-3β have been shown to share at least some cellular activities (Doble et al., 2007). Therefore, to eliminate the possibility of functional redundancy, the inventors employed a lentiviral-mediated shRNA knockdown strategy to reduce the expression of both isoforms (FIGS. 7A and S7B). In cultures infected with lentivirus carrying a non-silencing shRNA, fewer than 20% of MNs survived trophic factor withdrawal (FIG. 14B). On the other hand, in cultures receiving GSK-3 shRNAs, more than 50% of MNs survived. This increase in survival was significant, but was still substantially lower than that in control conditions with trophic factors—in other words, much lower than what the inventors achieved with kenpaullone addition. This again demonstrates that kenpaullone can have additional intracellular targets. Alternatively, it remains possible that we were unable to achieve higher levels of survival using shRNAs because of incomplete knockdowns (FIG. 14A).

The inventors used another approach to address the issue of GSK-3 and survival. The inventors produced MNs from the following mouse ESC lines: GSK-3α^{-/-} (GSK-3α-KO), GSK-3^{-/-} (GSK-3β-KO), and GSK-3α^{+/-}/GSK-3β^{-/-} (3/4-KO). Note that ESCs that are GSK-3α^{-/-}/GSK-3β^{-/-} were not able to differentiate into neurons (Doble et al., 2007). Cell death was again initiated by withdrawal of trophic factors and the ability of kenpaullone to promote survival was evaluated. Since these MNs do not carry an Hb9:GFP transgene, the inventors counted MNs as Islet⁺/MAP2⁺ cells (FIGS. 7A and 7B). The inventors validated this staining method using MN cultures prepared from Hb9::GFP ESCs. In these cultures, approximately 70% of Isl⁺/MAP2⁺ cells were GFP⁺ (i.e., MNs). However, after trophic factor withdrawal, because MNs die selectively under these conditions, only about 50% of Isl⁺/MAP2⁺ cells are GFP⁺ MNs. This means that counting Isl⁺/MAP2⁺ cells could overestimate the amount of survival after trophic factor withdrawal by approximately 40%. When deprived of neurotrophins, 35% of wild-type Isl⁺/MAP2⁺ cells survived, while 44%, 57% and 54% of Isl⁺/MAP2⁺ cells in GSK-3α-KO, GSK-3β-KO and 3/4-KO cultures survived. Using the correction factor, this would mean that wild-type MN survival would be approximately 25%, while the KO MNs would have 31%, 41% and 39% survival, respectively. The improved, but not completely restored, survival of cells that lack GSK-3α or/and GSK-3β again demonstrates that kenpaullone enhances survival by interacting with targets in addition to GSK-3.

The inventors also added kenpaullone to GSK-3-deficient cultures to determine whether the compound would still be able to increase survival. Interestingly, kenpaullone was still active, maintaining the number of MNs close to that in cultures with trophic support. These data again suggested that, while GSK-3 inhibition may account for part of kenpaullone's effect, other activities are necessary to explain its potent ability to promote survival.

Additional Targets of Kenpaullone

p21-Activated-Kinases (PAK)

Most commercially available GSK-3 chemical inhibitors have additional intracellular targets (Bain et al., 2007; Obligado et al., 2008; Anastassiadis at al., 2011). The inventors were interested in identifying which might account for kenpaullone's rather unique effects. It was previously shown that kenpaullone inhibits p21-activated-kinases (PAK), specifically PAK 4, 5 and 6, whereas alsterpaullone, CHIR 99021 and other GSK-3 inhibitors, do not (Bain et al., 2007). The inventors decided to test whether the effect of kenpaullone could be reproduced by using alsterpaullone with purvalanol A, a compound that inhibits PAK4/5/6. The inventors chose alsterpaullone because it is a GSK-3 inhibitor, is chemically extremely similar to kenpaullone and has a relatively similar inhibitory profile, but does not possess as potent an effect on MN survival. Purvalanol A was extremely toxic on its own, especially at higher concentrations (3 μM and 10 μM; FIG. 14C). However, when generally ineffective concentrations of alsterpaullone were combined with 3 μM or 10 μM of purvalanol A, they strongly promoted survival, the combination exceeding the activity of kenpaullone (FIG. 7C). Thus, the combination of alsterpaullone plus purvalanol A essentially equaled or exceeded the effects of kenpaullone alone. On the contrary, purvalanol A did not increase the effects of kenpaullone (FIG. 7D). Presumably, since kenpaullone likely already possesses PAK inhibiting activity, adding purvalanol A to it did not further improve its effectiveness.

ALS is a complicated and heterogeneous disease involving cell autonomous (MN) and non-cell autonomous (glial) elements. Because of this and because of the historical lack of availability of large numbers of mouse and human neural cells, therapeutics discovery and development have been hindered. For the data presented herein, the inventors have taken advantage of their ability to produce large numbers of mouse MNs from ESCs and carried out survival screens using both wild-type and SOD1 mutant cells. For these screens, the inventors produced hundreds of millions of MNs from the two different types of ESCs. This is the first time that neuronal screens like this have been undertaken.

Two aspects of the screening strategy that the inventors employed are worthy of discussion. First is the use of a mouse trophic factor withdrawal assay. The inventors chose mouse, rather than human, because it was much simpler for them to carry out a survival assay with cells that can be rapidly produced in large numbers and which carry a reporter gene that makes them simple to identify and quantify. Mouse cultures also have a much smaller number of progenitors than do human cultures, making survival studies more straightforward. Trophic factor withdrawal has been used by others to explore pathways that underlie neuronal cell death (Barthélémy et al., 2004; Kieran et al., 2008), including that of MNs, which die relatively selectively under the conditions of our assay. However, one drawback to this assay is that it is not ALS-specific.

The inventors considered carrying out a screen using SOD1^{G93A}/HB9::GFP neurons cultured on SOD1^{G93A} astrocytes. However, MN death in this system tends to be slow, occurring over a period of 2-3 weeks, and the amount of cell death can be quite variable. This would have a large impact on the quality of a screen that employed approximately 30,000 wells (per MN type). In addition, the inventors were concerned that this assay would provide hits that would be effective only in this particular form of ALS. The inventors also considered using glutamate excitotoxicity as the basis of a survival assay. However, it takes almost 2 weeks for MNs to become susceptible to glutamate-induced cell death (unpublished results), and the inventors' preliminary experiments showed the amount of cell death to be relatively modest even at that point.

Accordingly, the inventors decided to conduct the trophic factor withdrawal assay since cell death is rapid and extensive, making it amenable to large-scale screening. However, the inventors chose to conduct it using two different types of MNs, wild-type and SOD1^{G93A}. The inventors felt that this would give them the best opportunity to identify hits that can act in broadly protective way, thereby giving the inventors a better chance of identifying molecules that might act therapeutically across different patient populations in ALS.

Another component of the inventors' screen was the use of annotated compound libraries, each compound tested at three different concentrations in the primary screen. The inventors have employed this strategy successfully in several recent publications (Makhortova et al., 2011; Annes et al., 2012; Chen et al., 2012; Wang et al., 2012). As the inventors demonstrate here as well, using annotated compounds enabled them to gain some mechanistic insight as to how various classes of hit molecules might act. The inventors' choice of testing multiple concentrations, compared to the more conventional screening strategy of testing only a single high concentration of each compound, was based on the rationale that different compounds in the library can exert their effects at very different concentrations (Makhortova et al., 2011). This concern proved to be valid since significant numbers of hit compounds from the screens only scored at one or two, but not all three, concentrations, and they could have failed to show up as hits if the inventors had screened at a single concentration. Furthermore, SOD1^{G93A}/HB9::GFP MNs were seemingly more sensitive to compound toxicity, and occasionally specific concentrations of compounds that were effective on HB9:GFP cells killed those in SOD1^{G93A}/HB9::GFP cultures. Thus, had the inventors executed their screen at the relatively standard fixed concentration of 10 μM, a significant number of hit compounds would have been missed.

The inventors identified 3 classes of hit molecules: those that were effective on HB9::GFP MNs, those that were effective on SOD1^{G93A}/HB9::GFP MNs, and those that were effective on both. For this study, the inventors chose to follow up on compounds that were active on both types since we hypothesized that they may be most likely to be active in various forms of ALS. Kenpaullone was one of the most active of these and was able to support the long-term survival in the absence of neurotrophic factors, maintaining the integrity of the cells as assessed using electrophysiological and morphological techniques. Furthermore, kenpaullone was also able to decrease the amount of death caused by addition of a PI3-kinase inhibitor. Since PI3-kinase lies downstream of many neurotrophic factors. kenpaullone appears to exert a broadly protective influence on these cells. This became even more apparent in experiments showing that this compound can also reduce death of MNs induced by co-culture with SOD1^{G93A} astrocytes. This is widely thought of as a specific model at least for forms of ALS that are associated with SOD1 mutations (Di Giorgio at al., 2007; Nagai et al., 2007; Marchetto at al., 2008; Ilieva et al., 2009; Haidet-Phillips et al., 2011). Finally, the inventors discovered that kenpaullone can support the survival of human control MNs and of those carrying a familial SOD1 mutation.

Kenpaullone is classified as a GSK-3 inhibitor, and these inhibitors have been shown to affect both neurogenesis and neuronal survival (Hur and Zhou, 2010). However, the inventors provide data that strongly support the notion that kenpaullone, at least, acts primarily on survival under their assay conditions. In many cell types, including neurons, GSK-3 is activated during serum/trophic factor deprivation, and inhibiting GSK-3 has been shown to reduce neuronal apoptosis (Hetman and Xia, 2000; Linseman et al., 2004). The connection between GSK-3 and ALS has been demonstrated in a number of studies, and the therapeutic potential of GSK-3 inhibitors has been discussed. Specifically, expression of GSK-3 has been found to be altered in ALS patients (Chung et al., 2008; Yang et al., 2008; Kihira et al, 2009), and inhibiting GSK-3 has been shown to delay the onset and progression of disease, thereby prolonging survival in mouse models of ALS (Koh at al., 2007; 2011).

However, the inventors' data suggest that kenpaullone acts only in part as a GSK-3 inhibitor. The inventors tested many other GSK-3 inhibitors including those thought to be the most potent and specific, and found that most were weakly active, at best. Furthermore, the inventors carried out several experiments in which they reduced the expression of GSK-3 and found only relatively mild effects on survival. Kenpaullone itself was more active than any of the knockdowns and, in fact, was able to almost fully restore survival even in the knockdown cells. Although these results are not conclusive since there is some residual GSK-3 activity in cells in all of our studies, it is consistent with the possibility that kenpaullone has an additional target. Interestingly, Lyssiotis at al. found that kenpaullone could ad as a Klf-4 replacer in fibroblast reprogramming experiments, probably independent of its GSK-3 activity, although its mechanism of action in that study remained unidentified (Lyssiotis at al., 2009).

To gain insight into the identity of additional kenpaullone cellular targets, the inventors used published data to compare its kinase inhibitory profile to that of alsterpaullone, a highly related compound that had only a weak and variable activity in supporting survival of mouse and human MNs. These data suggest that kenpaullone inhibits several kinases that are not inhibited by alaterpaullone, including the p21-activated kinases (PAK) 4, 5 and 6. The inventors then tested commercially available inhibitors of these additional kinases. One, purvalanol A, annotated as a CDK and PAK inhibitor, induced cell death on its own, as previously described in cerebellar granule neurons (Monaco et al., 2004). However, the combination of purvalanol A plus alsterpaullone had an extremely strong effect, reaching or exceeding the protection levels provided by kenpaullone. This can mean that kenpaullone inhibits the same sets of kinases inhibited by the combination of alsterpaullone and purvalanol A. This synergistic effect provides another venue for treating neurodegenerative disorders.

Previous work has shown that the enhanced death of SOD1^{G93A}/HB9::GFP, relative to HB9::GFP, MNs occurs after 2-3 weeks in culture (Di Giorgio at al., 2007). Kenpaullone protects against this later form of death as well. At approximately the same time, MNs treated with kenpaullone had lower levels of mutant SOD1 protein and of ubiquitin, as assessed by single cell imaging. These decreases were also observed on Western blots, performed on lysates from mixed cell cultures, indicating that they are unlikely to occur in MNs alone. It was noted that SOD1^{G93A} protein inclusions increased in size and density between days 14 and day 28 and that the amount of ubiquitin increased (DI Giorgio et al., 2007). Also, a study of aggregation in three mutant SOD1 mouse models, SOD1^{G93A}, SOD1^{G37R}, and SOD1^{H46R/H48Q} showed that SOD1 aggregates appear in the later stages of disease (P120 mouse spinal cord) in parallel with the progression of motor defects (Johnston et al., 2000). It can be that kenpaullone's ability to increase survival is “upstream” of SOD1 aggregation or it can be that a primary effect of kenpaullone is to prevent protein aggregation, conferring an additional boost to its effects on survival. Generally, GSK-3 inhibition is associated with the opposite effect—namely, increasing protein stability (Xu et al., 2009)—so the effect on SOD1 is probably not related to overall effects on protein processing in neurons. Lowering the levels of mutant SOD1 protein has been introduced as an important strategy for developing therapeutics targeted at the subpopulation of patients who have SOD1 mutations (Saito et al., 2005; Smith et al., 2006; Wang et at, 2010). The data presented herein demonstrates that it is possible to find small drug-like molecules that also have this activity.

HPK1/GCK4-Like Kinase (HGK)

We wished to identify additional molecular targets that might be acted on by kenpaullone. Since kenpaullone is reported to inhibit multiple kinases (Anastassiadis t al., 2011), we carried out preliminary studies employing a Phospho-Kinase Antibody Array to examine the site-specific phosphorylation of a variety of kinases and their protein substrates in the presence or absence of kenpaullone in MN cultures (data not shown, see materials and methods). First, results indicated a significant reduction in relative phosphorylation of GSK-3awl, confirming that kenpaullone inhibits activation of this kinase in our assay. Further, results indicated reduction in relative phosphorylation of both cJun N-terminal kinase (JNK) and c-Jun, respectively. Since phospho-cJun is well known to be associated with stress-induced neuronal apoptosis and can also be activated subsequent to trophic factor withdrawal (Ham et al., 1995; Watson et al., 1998), we investigated this finding more thoroughly.

For this set of experiments, we decided to compare the effects of kenpaullone to those of CHIR99021, reported to be among the most selective of GSK-3 inhibitors, yet still unable to support MN survival under our assay conditions [F 2A; (Bain et al., 2007)]. First, using immunostaining with phospho-specific antibodies, we observed that kenpaullone, but not CHIR99021, decreased the percent of MNs positive for phospho-cJun or phospho-JNK, even at very early time points, confirming results of the Phosho-Kinase Array (FIGS. 6B and 6C). We next examined MKK4, an upstream regulator of this pathway (FIG. 6E) and found that its phosphorylation was also suppressed (FIG. 15B).

We next sought to determine which upstream kinase in this particular pathway could be inhibited directly by kenpaullone. According to prior reports on kinase activity, potential candidates include mixed lineage kinase-3 (MLK3) and HPK1/GCK-like kinase [HGK; also known as MAP4K4; (Anastassiadis at al., 2011)]. We therefore performed in vitro phosphorylation reactions using the two recombinant kinases, with a myelin basic protein peptide as the substrate. Surprisingly, we found that kenpaullone, but not CHIR99021, differentially inhibited HGK, but not MLK3, activity (FIG. 15D). Importantly, HGK is highly expressed in adult mouse motor neurons (Allen Spinal Cord Atlas; imageId-100479716). To confirm and extend these results, we performed the immunostaining studies with a site-specific antibody against phospho-Tak1, an intermediate in the cell death pathway between HGK and MKK4 (Yao at al., 1999), but uninvolved in the pathway activated by MLK3. The proportion of MNs immunopositive for phospho-Tak1 (FIG. 15B) was significantly reduced by kenpaullone but not CHIR99021, incubation. Together, these results indicate that the highly beneficial effects of kenpaullone, when compared to other more specific GSK-3 inhibitors. can be attributed in part to reducing activation of the HGK-Tak1-MKK4-JNK-c-Jun cell death signaling cascade (FIG. 15E).

To gain insight into the identity of additional kenpaullone targets, the inventors carried out an extensive series of biochemical and immunocytochemical experiments. The results described herein demonstrate that kenpaullone inhibits HGK, a kinase expressed in MNs but never studied in the context of MN disease. Inhibition of this kinase prevents the activation of an apoptosis pathway that is associated with increased nuclear levels of phospho-cJun (Yao et al. 1999). Thus, HGK emerges as a new therapeutic target for treating ALS.

In summary, the inventors conducted relatively large-scale survival screens on MNs derived from mouse ESCs. The inventors carried out a large series of studies on one potent hit, kenpaullone, which can support the long-term survival and functional integrity of those cells in the total absence of trophic factors. Kenpaullone seems to act as a multi-kinase inhibitor raising the possibility that this kind of compound can be useful in treating rapidly progressing neurodegenerative disorders, such as ALS, much as they have become more commonly utilized in treating particularly aggressive forms of cancer (Goh et al., 2012; Su et al., 2012).

The data presented herein demonstrates that method of screening using stem cell derived neurons is a new and effective method for finding and testing potential therapeutics for serious neurodegenerative disorders (Rubin, 2008).

In Vitro Clinical Trial

The inventors conducted a small human ALS “in vitro clinical trial” in which kenpaullone activity was compared to that of two compounds that failed in the clinic. Interestingly, olesoxime had been discovered in a wildtype rat primary MN survival screen (Bordet at al., 2007), similar to one of the assays we used here. Dexpramipexole reportedly has not been tested on rodent MN cultures.

Both compounds had been deemed somewhat active in the standard mouse ALS in vivo model (Bordet et al., 2007; Gribkoff and Bozik, 2008; Sunyach et al., 2012), but importantly, neither had been tested on human MNs of any variety. Even this small trial shows that it is possible to detect Important functional differences among these compounds when they are tested on human MNs. For example, dexpramimpexole was totally ineffective on any of the human MN lines, and these results might have predicted its lack of efficacy in the clinic, whereas olesoxime had less consistent activity and somewhat smaller effects. Kenpaullone, therefore, emerges as a preclinical compound of interest.

### REFERENCES

1. Anastassiadis, T., Deacon, S. W., Devarajan, K., Ma, H., and Peterson, J. R. (2011). Comprehensive assay of kinase catalytic activity reveals features of kinase inhibitor selectivity. Nat Biotechnol 29, 1039-1045.
2. Annes, J. P., Ryu, J. H., Lam, K., Carolan, P. J., Utz, K., Hollister-Lock, J., Arvanites, A. C., Rubin, L. L., Weir, G., and Melton, D. A. (2012). Adenosine kinase inhibition selectively promotes rodent and porcine islet β-cell replication. Proc Natl Acad Sci USA 109, 3915-3920.
3. Arai, T., Hasegawa, M., Akiyama, H., Ikeda, K., Nonaka, T., Mori, H., Mann, D., Tsuchiya, K., Yoshida, M., Hashizume, Y., et al. (2006). TDP-43 is a component of ubiquitin-positive tau-negative inclusions in frontotemporal lobar degeneration and amyotrophic lateral sclerosis. Biochem Biophys Res Commun 351, 602-611.
4. Bain, J., Plater, L, Elliott, M., Shpiro, N., Hastie, C J., McLauchlan, H., Klevernic, I., Arthur, J. S. C., Alessi, D. R., and Cohen, P. (2007). The selectivity of protein kinase inhibitors: a further update. Biochem J 408, 297-315.
5. Barthélémy, C., Henderson, C. E., and Pettmann, B. (2004). Foxo3a induces motoneuron death through the Fas pathway in cooperation with JNK. BMC Neurosci 5, 48.
6. Bekkers, J. M. (2009). Synaptic transmission: excitatory autapses find a function?Curr Biol 19, R296-8.
7. Bertel, O., Malessa, S., Slugs, E., and Hornykiewicz, O. (1991). Amyotrophic lateral sclerosis: changes of noradrenergic and serotonergic transmitter systems in the spinal cord. Brain Res 566, 54-60.
8. Boillée, S., Vande Velde, C., and Cleveland, D. W. (2006). ALS: a disease of motor neurons and their nonneuronal neighbors. Neuron 52, 39-59.
9. Bordet, T., Buisson, B., Michaud, M., Drouot, C., Galéa, P., Delaage, P., Akentieva, N. P., Evers, A. S., Covey, D. F., Ostuni, M. A., et al. (2007). Identification and characterization of cholest-4-en-3-one, oxime (TRO19622), a novel drug candidate for amyotrophic lateral sclerosis. J. Pharmacol. Exp. Ther. 322, 709-720.
10. Boulting, O. L., Kiskinis, E., Croft, G. F., Amoroso, M. W., Oakley, D. H., Wainger, B. J., Williams, D J., Kahler, D. J., Yamaki, M., Davidow, L, et al. (2011). A functionally characterized test set of human induced pluripotent stem cells. Nat Biotechnol 29, 279-286.
11. Brandt et al. (2010). Role of the JNK/c-Jun/AP-1 signaling pathway in galectin-1-induced T-cell death. Cell Death and Disease 1, e23.
12. Brown, R. H. (1997). Amyotrophic lateral sclerosis. Insights from genetics. Archives of Neurology 54, 1246-1250.
13. Brunet, A., Datta, S. R., and Greenberg, M. E. (2001). Transcription-dependent and -independent control of neuronal survival by the PI3K-Akt signaling pathway. Current Opinion in Neurobiology 11, 297-305.
14. Chen, P. C., Gaisina, L. N., El-Khodor. B. F., Ramboz, S., Makhortova, N. R., Rubin, L. L., and Kozikowski, A. P. (2012). Identification of a Maleimide-Based Glycogen Synthase Kinase-3 (GSK-3) Inhibitor, BIP-135, that Prolongs the Median Survival Time of Δ7 SMA KO Mouse Model of Spinal Muscular Atrophy. ACS Chemical Neuroscience 3, 5-11.
15. Chung, Y. H., Joo, K. M., Kim, D. J., Kim, S. S., Kim, K. Y., Lee, W. B., and Cha, C. I. (2008). Immunohistochemical study on the distribution of glycogen synthase kinase 3alpha in the central nervous system of SOD1(G93A) transgenic mice. Neurol Res 30, 926-931.
16. Cudkowicz, M., Bozik, M. E., Ingersoll, E. W., Miller, R., Mitsumoto. H., Shefner, J., Moore, D. H., Schoenfeld, D., Mather, J. L., Archibald, D., et al. (2011). The effects of dexpramipexole (KNS-760704) in individuals with amyotrophic lateral sclerosis. Nat Med 17, 1652-1656.
17. Dewil, M., Lambrechts, D., Sciot, R., Shaw, P. J., Ince, P. G., Robberecht, W., and Van den Bosch, L. (2007). Vascular endothelial growth factor counteracts the loss of phospho-Akt preceding motor neurone degeneration in amyotrophic lateral sclerosis. Neuropathology and Applied Neurobiology 33, 499-509.
18. Di Giorgio, F. P., Boulting, G. L., Bobrowicz, S., and Eggan, K. C. (2008). Human embryonic stem cell-derived motor neurons are sensitive to the toxic effect of glial cells carrying an ALS-causing mutation. Cell Stem Cell 3, 637-648.
19. Di Giorgio, F. P., Carrasco, M. A., Siao, M. C., Maniatis, T., and Eggan, K. (2007). Non-cell autonomous effect of glia on motor neurons in an embryonic stem cell-based ALS model. Nat Neurosci 10, 608-614.
20. Díaz-Amarilla, P., Olivea-Bravo, S., Trims, E., Cragnolini, A., Martínez-Palma, L., Cassina, P., Beckman, J., and Barbeto, L. (2011). Phenotypically aberrant astrocytes that promote motoneuron damage in a model of inherited amyotrophic lateral sclerosis. Proc Natl Acad Sci USA 108, 18126-18131.
21. Ding. Q., Lee, Y.-K., Schaefer, E. A. K., Peters, D. T., Veres, A., Kim, K., Kuperwasser, N., Motola, D. L., Meissner, T. B., Hendriks, W. T., et al. (2013). A TALEN Genome-Editing System for Generating Human Stem Cell-Based Disease Models. Cell Stem Cell 12, 238-251.
22. Doble, B. W., Patel, S., Wood, G. A., Kockeritz, L. K., and Woodgett, J. R. (2007). Functional redundancy of GSK-3alpha and GSK-3beta in Wnt/beta-catenin signaling shown by using an allelic series of embryonic stem cell lines. Dev Cell 12, 957-971.
23. Fang. L., Huber-Abel, F., Teuchert. M., Hendrich, C., Dorst, J., Schattauer, D., Zettlmeissel, H., Wlaschek, M., Scharffetter-Kochanek, K., Tumani, H., et al. (2009). Linking neuron and skin: matrix metalloproteinases in amyotrophic lateral sclerosis (ALS). J Neurol Sci 285, 62-66.
24. Fasano, C., Campana, V., Griffiths, B., Kelly, G., Schiavo, G., and Zurzolo, C. (2008). Gene expression profile of quinacrine-cured prion-infected mouse neuronal cells. J Neurochem 105, 239-250.
25. Ferraiuolo, L., Kirby. J., Grierson, A. J., Sendtner, M., and Shaw, P. J. (2011). Molecular pathways of motor neuron injury in amyotrophic lateral sclerosis. Nat Rev Neurol 7, 616-630.
26. Gob, K. C., Novotny-Diermayr, V., Hart, S., Ong, L. C., Loh, Y. K., Cheong. A., Tan, Y. C., Hu, C., Jayaraman, R., William, A. D., et al. (2012). TGO2, a novel oral multi-kinase inhibitor of CDKs, JAK2 and FLT3 with potent anti-leukemic properties. Leukemia 26, 236-243.
27. Gribkoff, V. K., and Bozik, M. E. (2008). KNS-760704 [(6R)-4,5,6,7-tetrahydro-N6-propyl-2,6-benzothiazole-diamine dihydrochloride monohydrate] for the treatment of amyotrophic lateral sclerosis. CNS Neurosci Ther 14, 215-226.
28. Grosskreutz, J., Van Den Bosch, L., and Keller, B. U. (2010). Calcium dysregulation in amyotrophic lateral sclerosis. Cell Calcium 47, 165-174.
29. Haidet-Phillips, A. M., Hester, M. E., Miranda, C. J., Meyer, K., Braun, L., Frakes, A., Song. S., Likhite, S., Murtha, M. J., Foust, K. D., et al. (2011). Astrocytes from familial and sporadic ALS patients are toxic to motor neurons. Nat Biotechnol 29, 824-828. Ham, J., Babij, C., Whitfield, J., Pfarr, C. M., Lallemand, D., Yaniv, M., and Rubin, L L. (1995). A c-Jun dominant negative mutant protects sympathetic neurons against programmed cell death. Neuron 14, 927-939.
30. Hester, M. E., Murtha, M J., Song, S., Rao, M., Miranda, C. J., Meyer, K., Tian, J., Boulting, G., Schaffer, D. V., Zhu, M. X., et al. (2011). Rapid and Efficient Generation of Functional Motor Neurons From Human Pluripotent Stem Cells Using Gene Delivered Transcription Factor Codes. Mol Ther 19, 1905-1912.
31. Hetman, M., and Xia, Z. (2000). Signaling pathways mediating anti-apoptotic action of neurotrophins. Acts Neurobiol Exp (Wars) 60, 531-545.
32. Höing. S., Rudhard, Y., Reinhardt, P., Olatza, M., Stehling, M., Wu, G., Peiker, C., Bcker, A., Parga, J. A., Bunk, E., et al. (2012). Discovery of inhibitors of microglial neurotoxicity acting through multiple mechanisms using a stem-cell-based phenotypic assay. Cell Stem Cell 11, 620-632.
33. Hur, E.-M., and Zhou, F.-Q. (2010). GSK-3 signalling in neural development. Nat Rev Neurosci 11, 539-551.
34. Ilieva, H., Polymenidou, M., and Cleveland, D. W. (2009). Non-cell autonomous toxicity in neurodegenerative disorders: ALS and beyond. J Cell Biol 187, 761-772.
35. Johnston, J. A., Dalton, M J., Gurney, M. E., and Kopito, R. R. (2000). Formation of high molecular weight complexes of mutant Cu, Zn-superoxide dismutase in a mouse model for familial amyotrophic lateral sclerosis. Proc Natl Acad Sci USA 97, 12571-12576.
36. Joyce, P. I., Fratta, P., Fisher, E. M. C., and Acevedo-Arozena, A. (2011). SOD1 and TDP-43 animal models of amyotrophic lateral sclerosis: recent advances in understanding disease toward the development of clinical treatments. Mamm Genome 22, 420-448.
37. Kieran, D., Sebastia, J., Greenway, M. J., King, M. A., Connaughton, D., Concannon, C. G., Fenner, B., Hardiman, O., and Prehn, J. H. M. (2008). Control of motoneuron survival by angiogenin. J Neurosci 28, 14056-14061.
38. Kihira, T., Suzuki, A., Kondo, T., Wakayama, I., Yoshida, S., Hasegawa, K., and Garruto, R. M. (2009). Immunohistochemical expression of IGF-I and GSK in the spinal cord of Kii and Guamanian ALS patients. Neuropathology 29, 548-558.
39. Kim, K., Moore, D. H., Makriyannis, A., and Abood, M. E. (2006). AM1241, a cannabinoid CB2 receptor selective compound, delays disease progression in a mouse model of amyotrophic lateral sclerosis. Eur J Pharmacol 542, 100-105.
40. Kioschis-Schneider, P., and Hafner, M. (2010). Compounds for the Modulation of Huntingtin Aggregation, Methods and Means for Identifying Such Compounds. US Patent App. Pub.
41. Koh, S.-H., Back, W., and Kim, S. H. (2011). Brief review of the role of glycogen synthase kinase-3p in amyotrophic lateral sclerosis. Neurol Res Int 2011, 205761.
42. Koh, S.-H., Kim, Y., Kim, H. Y., Hwang, S., Lee, C. H., and Kim, S. H. (2007). Inhibition of glycogen synthase kinase-3 suppresses the onset of symptoms and disease progression of G93A-SOD1 mouse model of ALS. Exp Neurol 205, 336-346.
43. Koh, S.-H., Kwon, H., Kim, K. S., Kim, J., Kim, M.-H., Yu, H.-J., Kim, M., Lee, K.-W., Do, B. R., Jung H. K., et al. (2004). Epigallocatechin gallate prevents oxidative-stress-induced death of mutant Cu/Zn-superoxide dismutase (G93A) motoneuron cells by alteration of cell survival and death signals. Toxicology 202, 213-225.
44. Krieger, C., Lanius, R. A., Pelech, S. L., and Shaw, C. A. (1996). Amyotrophic lateral sclerosis: the involvement of intracellular Ca2+ and protein kinase C. Trends Pharmacol Sci 17, 114-120.
45. Lange, C., Mix, E., Frahm, J., Glass, A., Müller, J., Schmitt, O., Schmole, A.-C., Klemm, K., Ortinau, S., Hübner, R., et al. (2010). Small molecule GSK-3 inhibitors increase neurogenesis of human neural progenitor cells. Neurosci Let 488, 36-40.
46. Linseman, D. A., Butts, B. D., Precht, T. A., Phelps, R. A., Le, S. S., Laessig, T. A., Bouchard, R. J., Florez-McClure, M. L., and Heidemnreich, K. A. (2004). Glycogen synthase kinase-3beta phosphorylates Bax and promotes its mitochondrial localization during neuronal apoptosis. J Neurosci 24, 9993-10002.
47. Lorenzi, S., Nan, S., Angele, B., Krell, H. W., Gregorio, J., Kiaei, M., Pfister, H.-W., and Beal, M. F. (2006). The matrix metalloprotelnases inhibitor Ro 28-2653 [correction of Ro 26-2853] extends survival in transgenic ALS mice. Exp Neurol 200, 166-171.
48. Lyssiotis, C. A., Foreman, R. K., Staerk, J., Garcia, M., Mathur, D., Markoulaki, S., Hanna, J., Lairson, L L, Charette, B. D., Bouchez, L. C., et al. (2009). Reprogramming of murine fibroblasts to induced pluripotent stem cells with chemical complementation of Klf4. Proc Natl Acad Sci USA 106, 8912-8917.
49. Majounie, E., Renton, A. E., Mok, K., Dopper, E. G., Waie, A., Rollinson, S., Chib, A., Restagno, G., Nicolaou, N., Simon-Sanchez, J., et al. (2012). Frequency of the C9orf72 hexanucleotide repeat expansion in patients with amyotrophic lateral sclerosis and frontotemporal dementia: a cross-sectional study. Lancet Neurol 11, 323-330.
50. Makhortova, N. R., Hayhurst, M., Cerqueira, A., Sinor-Anderson, A. D., Zhao, W.-N., Heiser, P. W., Arvanites, A. C., Davidow, L. S., Waldon, Z. O., Steen, J. A., et al. (2011). A screen for regulators of survival of motor neuron protein levels. Nature Chemical Biology 7, 544-552.
51. Marchetto, M. C. N., Muotri, A. R., Mu, Y., Smith, A. M., Cezar, G. G., and Gage, F. H. (2008). Non-cell-autonomous effect of human SOD1 G37R astrocytes on motor neurons derived from human embryonic stem cells. Cell Stem Cell J, 649-657.
52. Martin, L. J. (2001). Neuronal cell death in nervous system development, disease, and injury (Review). Int J Mol Med 7, 455-478.
53. Mattson, M. P., and Furukawa, K. (1997). Anti-apoptotic actions of cycloheximide: blockade of programmed cell death or induction of programmed cell life? Apoptosis 2, 257-264.
54. Medina, M., and Castro, A. (2008). Glycogen synthase kinase-3 (GSK-3) inhibitors reach the clinic. Curr Opin Drug Discov Devel 11, 533-543.
55. Melisi et al. (2011). Modulation of Pancreatic Cancer Chemoresistance by Inhibition of TAK1. J Natl Cancer Inst. 103(15), 1190-1204.
56. Miles, G. B., Yohn, D. C., Wichterle, H., Jessell, T. M., Rafuse, V. F., and Brownstone, R. M. (2004). Functional properties of motoneurons derived from mouse embryonic stem cells. J Neurosci 24, 7848-7858.
57. Miller, R. G., Mitchell, J. D., Lyon, M., and Moore, D. H. (2007). Riluzole for amyotrophic lateral sclerosis (ALS)/mymotor neuron disease (MND). Cochrane Database Syst Rev CD001447.
58. Monaco, E. A., Beaman-Hall, C. M., Mathur, A., and Vallano, M. L. (2004). Roscovitine, olomoucine, purvalanol: inducers of apoptosis in maturing cerebellar granule neurons. Biochem Pharmacol 67, 1947-1964.
59. Nagai, M., Re, D. B., Nagata, T., Chalazonitis, A., Jessell, T. M., Wichterle, H., and Przedborski, S. (2007). Astrocytes expressing ALS-linked mutated SOD1 release factors selectively toxic to motor neurons. Nat Neurosci 10, 615-622.
60. Neumann, M., Sampathu, D. M., Kwong. L. K., Truax, A. C., Micsenyi, M. C., Chou, T. T., Bruce, J., Schuck, T., Grossman, M., Clark, C. M., et al. (2006). Ubiquitinated TDP-43 in frontotemporal lobar degeneration and amyotrophic lateral sclerosis. Science 314, 130-133.
61. Niebroj-Dobosz, I., Janik, P., Sokolowska, B., and Kwiecinski, H. (2010). Matrix metalloproteinases and their tissue inhibitors in serum and cerebrospinal fluid of patients with amyotrophic lateral sclerosis. Eur J Neurol 17, 226-231.
62. Obligado, S. H., Ibraghimov-Beskrovnaya, O., Zuk, A., Meier, L., and Nelson. P. J. (2008). CDK/GSK-3 inhibitors as therapeutic agents for parenchymal renal diseases. Kidney Int 73, 684-690.
63. Palizvan, M. R., Sohya, K., Kohara, K., Maruyama, A., Yasuda, H., Kimura, F., and Tsumoto, T. (2004). Brain-derived neurotrophic factor increases inhibitory synapses, revealed in solitary neurons cultured from rat visual cortex. Neuroscience 126, 955-966.
64. Polymenidou, M., and Cleveland, D. W. (2011). The Seeds of Neurodegeneration: Prion-like Spreading in ALS. Cell 147, 498-508.
65. Renton, A. E., Mjounie, E., Waite, A., Simón-Sánchez, J., Rollinson, S., Gibbs, J. R., Schymick, J. C., Lasksovirta, H., van Swieten, J. C., Myllykangas, L., et al. (2011). A hexanucleotide repeat expansion in C90RF72 is the cause of chromosome 9p21-linked ALS-FTD. Neuron 72, 257-268.
66. Rubin, L L. (2008). Stem cells and drug discovery: the beginning of a new era? Cell 132, 549-552.
67. Saito, Y., Yokota, T., Mitani, T., Ito, K., Anzai, M., Miyagishi, M., Taira, K., and Mizusawa, H. (2005). Transgenic small interfering RNA halts amyotrophic lateral sclerosis in a mouse model. J Biol Chem 280, 42826-42830.
68. Sandyk, R. (2006). Serotonergic mechanisms in amyotrophic lateral sclerosis. Int J Neurosci 116, 775-126.
69. Shoemaker, J. L., Seely, K. A., Reed, R. L., Crow, J. P., and Prather, P. L. (2007). The CB2 cannabinoid agonist AM-1241 prolongs survival in a transgenic mouse model of amyotrophic lateral sclerosis when initiated at symptom onset. J Neurochem 101, 87-98.
70. Smith, R. A., Miller, T. M., Yamanaka, K., Monia, B. P., Condon, T. P., Hung, G., Lobsiger, C. S., Ward, C. M., McAlonis-Downes, M., Wei, H., et al. (2006). Antisense oligonucleotide therapy for neurodegenerative disorder. J Clin Invest 116, 2290-2296.
71. Son, E. Y., Ichida, J. K., Wainger, B. J., Toma, J. S., Rafuse, V. F., Woolf, C J., and Eggan, K. (2011). Conversion of mouse and human fibroblasts into functional spinal motor neurons. Cell Stem Cell 9, 205-218.
72. Soundararajan, P., Miles, G. B., Rubin, L. L., Brownstone, R. M., and Rafuse, V. F. (2006). Motoneurons derived from embryonic stem cells express transcription factors and develop phenotypes characteristic of medial motor column neurons. J Neurosci 26, 3256-3268.
73. Spilman, P., Lessard, P., Sattavat, M., Bush, C., Tousseyn, T., Huang, E J., Giles, K., Golde, T., Des, P., Fauq, A., et al. (2008). A gamma-secretase inhibitor and quinacrine reduce prions and prevent dendritic degeneration in murine brains. Proc Natl Acad Sci USA 105, 10595-10600.
74. Strippoli R, Benedicto I, Perez Lozano M L, Pellinen T. Sandoval P, et al. (2012) Inhibition of Transforming Growth Factor-Activated Kinase 1 (TAK1) Blocks and Reverses Epithelial to Mesenchymal Transition of Mesothelial Cells. PLoS ONE 7(2): e31492
75. Su, Y., Vilgelm, A. E., Kelley, M. C., Hawkins, O. E., Liu, Y., Boyd, K. L., Kantrow, S., Splittgerber, RIC., Short, S. P., Sobolik, T., et al. (2012). RAF265 Inhibits the Growth of Advanced Human Melanoma Tumors. Clinical Cancer Research 18, 2184-2198.
76. Sunyach, C., Michaud, M., Amrnoux, T., Bernard-Marissal, N., Aebischer, J., Latyszenok, V., Gouarné, C., Raoul, C., Pruss, R. M., Bordet, T., et al. (2012). Olesoxime delays muscle denervation, astrogliosis, microglial activation and motoneuron death in an ALS mouse model. Neuropharmacology 62, 2346-2352.
77. Tradewell, M. L., and Durham, H. D. (2010). Calpastatin reduces toxicity of SOD1G93A in a culture model of amyotrophic lateral sclerosis. Neuroreport 21, 976-979. Ullian. E. M., Harris, B. T., Wu, A., Chan, J. R., and Barres, B. A. (2004). Schwann cells and astrocytes induce synapse formation by spinal motor neurons in culture. Mol Cell Neurosci 25, 241-251.
78. Van Deerlin, V. M., Leverenz, J. B., Bekris, L. M., Bird, T. D., Yuan, W., Elman, L. B., Clay, D., Wood, E. M., Chen-Plotkin, A. S., Martinez-Lage, M., et al. (2008). TARDBP mutations in amyotrophic lateral sclerosis with TDP-43 neuropathology: a genetic and histopathological analysis. Lancet Neurol 7, 409-416.
79. Wang, J., Slunt, H., Gonzales, V., Fromholt, D., Coonfield, M., Copeland, N. G., Jenkins. N. A., and Borchelt, D. R. (2003). Copper-binding-site-null SOD1 causes ALS in transgenic mice: aggregates of non-native SOD1 delineate a common feature. Hum Mol Genet 12, 2753-2764.
80. Wang. L, Grisotti, G., and Roos, R. P. (2010). Mutant SOD1 knockdown in all cell types ameliorates disease in G85R SOD1 mice with a limited additional effect over knockdown restricted to motor neurons. J Neurochem 113, 166-174.
81. Wang. Y., Arvanites, A. C., Davidow, L., Blanchard, J., Lam, K., Yoo, J. W., Coy, S., Rubin, L. L., and McMahon, A. P. (2012). Selective Identification of Hedgehog Pathway Antagonists By Direct Analysis of Smoothened Ciliary Translocation. ACS Chemical Biology. In press. (doi 10.1021/cb300028a).
82. Watanabe, M., Dykes-Hoberg, M., Culotta, V. C., Price, D. L., Wong. P. C., and Rothstein, J. D. (2001). Histological evidence of protein aggregation in mutant SOD1 transgenic mice and in amyotrophic lateral sclerosis neural tissues. Neurobiol Dis 8, 933-941.
83. Watson, A., Eilers, A., Lallemand, D., Kyriakis, J., Rubin, L. L., and Ham, J. (1998). Phosphorylation of c-Jun is necessary for apoptosis induced by survival signal withdrawal in cerebellar granule neurons. J Neurosci 18, 751-762.
84. Wichterle, H., Lieberam, I., Porter, J. A., and Jessell, T. M. (2002). Directed differentiation of embryonic stem cells into motor neurons. Cell 110, 385-397.
85. Wootz, H., Hansson, I., Korhonen, L., and Lindholm, D. (2006). XIAP decreases caspase-12 cleavage and calpain activity in spinal cord of ALS transgenic mice. Exp Cell Res 312, 1890-1898.
86. Wu et a. (2013). Mechanism and In Vitro Pharmacology of TAK1 inhibition by (5Z)-7-Oxozeaenol. ACS Chem Bol, 8(3), 643-50.
87. Xu, C., Kim, N.-G., and Gumbiner, B. M. (2009). Regulation of protein stability by GSK-3 mediated phosphorylation. Cell Cycle 8, 4032-4039.
88. Yang, W., Leystra-Lantz, C., and Strong, M. J. (2008). Upregulation of GSK-3beta expression in frontal and temporal cortex in ALS with cognitive impairment (ALSci). Brain Res 1196, 131-139.
89. Yao, Z., Zhou, G., Wang, X. S., Brown, A., Diener, K, Gan, H., and Tan, T. H. (1999). A novel human STE20-related protein kinase, HGK, that specifically activates the c-Jun N-terminal kinase signaling pathway. J Biol Chem 274, 2118-2125.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method of promoting motor neuron survival, comprising contacting a motor neuron with an effective amount of an agent that inhibits HPK/GC kinase-like kinase (HGK).

2. A method of treating or preventing a neurodegenerative disorder in a subject in need thereof, comprising administering an effective amount of an agent that inhibits HGK to the subject.

3. A method of treating or preventing a disorder characterized by neuronal cell death in a subject in need thereof, comprising administering an effective amount of an agent that inhibits HGK to the subject.

4. A method of treating or preventing amyotrophic lateral sclerosis (ALS) in a subject in need thereof, comprising administering an effective amount of an agent that inhibits HGK to the subject.

5. A method of treating or preventing spinal muscular atrophy (SMA) in a subject in need thereof, comprising administering an effective amount of an agent that inhibits HGK to the subject.

6. A method of promoting motor neuron survival, comprising contacting a motor neuron with an effective amount of an agent that decreases activation of the HGK-Tak1-MKK4-JNK-c-Jun cell death signaling cascade.

7. A method of treating or preventing a neurodegenerative disorder in a subject in need thereof, comprising administering an effective amount of an agent that decreases activation of the HGK-Tak1-MKK4-JNK-c-Jun cell death signaling cascade.

8. A method of treating or preventing a disorder characterized by neuronal cell death in a subject in need thereof, comprising administering an effective amount of an agent that decreases activation of the HGK-Tak1-MKK4-JNK-c-Jun cell death signaling cascade to the subject.

9. A method of treating or preventing amyotrophic lateral sclerosis (ALS) in a subject in need thereof, comprising administering an effective amount of an agent that decreases activation of the HGK-Tak1-MKK4-JNK-c-Jun cell death signaling cascade.

10. A method of treating or preventing spinal muscular atrophy (SMA) in a subject in need thereof, comprising administering an effective amount of an agent that decreases activation of the HGK-Tak1-MKK4-JNK-c-Jun cell death signaling cascade.

11. A method according to any one of claims 1-5, wherein the agent decreases activation of the HGK-Tak1-MKK4-JNK-c-Jun cell death signaling cascade.

12. A method according to any one of claims 1-10, wherein the agent decreases phosphorylation of a protein kinase in the HGK-Tak1-MKK4-JNK-c-Jun cell death signaling cascade.

13. A method according to claim 12, wherein the protein kinase is selected from the group consisting of HGK, Tak1, MKK4, and JNK.

14. A method according to any one of claims 1-13, wherein the agent inhibits HGK.

15. A method according to any one of claims 1-14, wherein the agent is an inhibitor of glycogen synthase kinase 3 (GSK3).

16. A method according to any one of claims 1-15, wherein the agent inhibits GSK3.

17. A method according to any one of claims 1-16, wherein the agent is kenpaullone or an analog or derivative thereof.

18. A method according to any one of claims 1-17, wherein the agent is selected from the group consisting of small organic or inorganic molecules; saccharines; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

19. A method according to any one of claims 1 or 6, further comprising contacting the motor neuron with an additional agent that inhibits GSK3.

20. A method according to any one of claims 2-5 or 6-9, further comprising administering to the subject an effective amount of an additional agent that inhibits GSK3.

21. A method according to claim 3 or 8, wherein the neuronal cell is selected from the group consisting of a motor neuron and a sensory neuron.

22. A method according to any one of claims 1-21, wherein the motor neuron comprises a mutation in a gene encoding survival of motor neuron 1 (SMN1).

23. A method according to any one of claims 1-21, wherein the motor neuron comprises a mutation in a gene encoding superoxide dismutase 1 (SOD1).

24. A method according to claim 23, wherein the mutation is selected from the group consisting of a A4V mutation, a G85R mutation, and a G93A mutation.

25. A method according to any one of claims 1-21, wherein the motor neuron comprises mutations in a gene encoding SMN1 and a gene encoding SOD1.

26. A method according to claims 1 or 6, wherein the contact is in vitro.

27. A method according to claims 1 or 6, wherein the contact is in vivo.

28. A method according to claim 26, wherein the in vivo contact is in a subject selected for treatment of a neurodegenerative disorder or disorder characterized by neuronal cell death.

29. A method according to any one of claims 2-5 or 7-10, wherein the subject is at risk of developing a neurodegenerative disorder or a disorder characterized by neuronal cell death.

30. A method according to any one of claims 2-5 or 7-10, wherein the subject is suspected of having a neurodegenerative disorder or a disorder characterized by neuronal cell death.

31. A method according to any one of claims 2-5, 7-10 or 28-30, wherein the subject is a mammal.

32. A method according to any one of claims 2-5, 7-10 or 28-30, wherein the subject is a human.

33. A method according to claims 2, 7, or 28-30, wherein the neurodegenerative disorder is characterized by mutation of a SMN gene.

34. A method according to claims 2, 7, or 28-30, wherein the neurodegenerative disorder is characterized by decreased levels of SMN protein.

35. A method according to claims 2, 7, or 28-30, wherein the neurodegenerative disorder is characterized by neuronal cell death.

36. A method according to any one of claims 2, 7, or 25-31, wherein the neurodegenerative disorder is ALS.

37. A method according to any one of claims 2, 7, or 25-31, wherein the neurodegenerative disorder is SMA.

38. A method of promoting motor neuron survival, comprising contacting a motor neuron with an effective amount of a compound selected from the group consisting of ethaverine hydrochloride, crinamine, gedunin, pomiferin, dactinomycin, 3-alpha-hydroxydeoxygedinin, totarol, totaral acetate, ginkgetin potassium salt, hygromycin B, blasticidin S, lynestrenol, hippeastrine hydrobromide, and combinations thereof.

39. A composition comprising an effective amount of an HGK inhibitor and an effective amount of a GSK3 inhibitor.

40. A composition according to claim 35, wherein the composition is useful for promoting survival of motor neurons.

41. A composition according to claim 35, wherein the composition is useful for treating or preventing a neurodegenerative disorder.

42. A composition according to claim 37, wherein the neurodegenerative disorder is selected from the group consisting of ALS and SMA.

43. A composition according to any one of claims 35-38, further comprising a pharmaceutically acceptable excipient, diluent or carrier.

44. A method of identifying a candidate agent that promotes motor neuron survival, comprising (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent that promotes motor neuron survival, wherein the test agent is a candidate agent for promoting motor neuron survival if the test agent (i) decreases the level or activity of HGK or (ii) decreases activation of the HGK-Tak1-MKK4-JNK-c-Jun cell death signaling cascade, in the presence of the test agent.

45. A method of identifying a candidate agent for treating or preventing a neurodegenerative disorder, comprising (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent for treating a neurodegenerative disorder, wherein the test agent is a candidate agent for treating a neurodegenerative disorder if the test agent (i) decreases the level or activity of HGK or (ii) decreases activation of the HGK-Tak1-MKK4-JNK-c-Jun cell death signaling cascade, in the presence of the test agent.

46. A method of identifying a candidate agent for treating or preventing ALS, comprising (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent for treating ALS, wherein the test agent is a candidate agent for treating ALS if the test agent (i) decreases the level or activity of HGK or (ii) decreases activation of the HGK-Tak1-MKK4-JNK-c-Jun cell death signaling cascade, in the presence of the test agent.

47. A method of identifying a candidate agent for treating or preventing SMA, comprising (a) contacting a motor neuron with a test agent, and (b) measuring (i) the level or activity of HGK or (ii) activation of the HGK-Tak1-MKK4-JNK-c-Jun cell death signaling cascade, in the presence of the test agent, and (c) identifying the candidate agent for treating SMA, wherein the test agent is a candidate agent for treating SMA if the test agent (i) decreases the level or activity of HGK or (ii) decreases activation of the HGK-Tak1-MKK4-JNK-c-Jun cell death signaling cascade, in the presence of the test agent.

48. A method according to any one of claims 40-43, wherein the contacting is performed in the absence of trophic factors.

49. A method according to any one of claims 40-43, wherein the motor neuron comprises an in vitro-differentiated motor neuron.

50. A method according to claim 45, wherein the motor neurons are derived from pluripotent cells selected from the group consisting of embryonic stem cells (ESCs) and induced pluripotent stem cells (iPSCs).

51. A method according to any one of claims 40-43, wherein the test agent is selected from the group consisting of small organic or inorganic molecules; saccharines; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

52. A method according to any one of claims 40-43, further comprising quantifying the number of motor neurons surviving in the presence of the test agent.

53. A method according to claim 48, wherein the surviving motor neurons express a detectable reporter.

54. A method according to claim 49, wherein the detectable reporter is a fluorescent protein selected from the group consisting of green fluorescent protein (GFP) and red fluorescent protein (RFP).

55. A method of diagnosing a neurodegenerative disorder in a subject, comprising: (a) obtaining a biological sample from the subject comprising neuronal cells; (b) conducting at least one binding assay on the neuronal cells to detect the level or activity of HGK in the neuronal cells; and (c) diagnosing the subject as having the neurodegenerative disorder if the level or activity of HGK in the neuronal cells is increased relative to a control level or activity of HGK.

56. The method of claim 55, wherein the at least one binding assay comprises a protein kinase assay to detect the phosphorylation activity of HGK.

57. The method of claim 55, wherein the at least one binding assay comprises a protein kinase assay to detect the level of phosphorylation of a protein kinase downstream to HGK in the HGK-Tak1-MKK4-JNK-c-Jun cell death signaling cascade.

58. The method of claim 55, further comprising selecting a subject suspected of having a neurodegenerative disorder.

59. The method of claim 55, wherein the neuronal cells comprise motor neurons.

60. The method of claim 55, wherein the neuronal cells comprise sensory neurons.

61. The method of claim 55, wherein the neurodegenerative disorder is ALS.

62. A method of diagnosing a neurodegenerative disorder in a subject, comprising: (a) obtaining a biological sample from the subject comprising neuronal cells; (b) conducting at least one kinase assay on the neuronal cells to detect activation of the HGK-Tak1-MKK4-JNK-c-Jun cell death signaling cascade in the neuronal cells; and (c) diagnosing the subject as having the neurodegenerative disorder if the HGK-Tak1-MKK4-JNK-c-Jun cell death is activated in the neuronal cells.

63. The method of claim 62, wherein the HGK-Tak1-MKK4-JNK-c-Jun cell death signaling cascade in the neuronal cells is activated if the phosphorylation level or activity of a protein kinase in the HGK-Tak1-MKK4-JNK-c-Jun cell death signaling cascade is increased relative to the phosphorylation level or activity of a protein kinase in the HGK-Tak1-MKK4-JNK-c-Jun cell death signaling cascade in a control.

64. The method of claim 62, further comprising selecting a subject suspected of having a neurodegenerative disorder.

65. The method of claim 62, wherein the neuronal cells comprise motor neurons.

66. The method of claim 62, wherein the neuronal cells comprise sensory neurons.

67. The method of claim 62, wherein the neurodegenerative disorder is ALS.

68. A method of diagnosing amyotrophic lateral sclerosis (ALS) in a subject, comprising: (a) obtaining a biological sample from the subject comprising neuronal cells; (b) conducting at least one kinase assay on the neuronal cells to detect activation of the HGK-Tak1-MKK4-JNK-c-Jun cell death signaling cascade in the neuronal cells; and (c) diagnosing the subject as having ALS if the HGK-Tak1-MKK4-JNK-c-Jun cell death is activated in the neuronal cells.

69. The method of claim 62, wherein the HGK-Tak1-MKK4-JNK-c-Jun cell death signaling cascade in the neuronal cells is activated if the phosphorylation level or activity of a protein kinase in the HGK-Tak1-MKK4-JNK-c-Jun cell death signaling cascade is increased relative to the phosphorylation level or activity of a protein kinase in the HGK-Tak1-MKK4-JNK-c-Jun cell death signaling cascade in a control.

70. The method of claim 62, further comprising selecting a subject suspected of having ALS.

71. The method of claim 62, wherein the neuronal cells comprise motor neurons.

72. A method of promoting motor neuron survival, the method comprising contacting a motor neuron with a compound of formula (I): 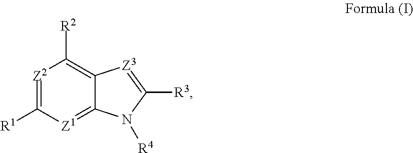
wherein each of Z¹, Z² and Z³ is independently N or CH₂;
R¹ is hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂H, SO₂, CN, CF₃, or SO₂;
R² hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂H, SO₂, CN, CF₃, or SO₂;
R³ is hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂H, SO₂, CN, CF₃, and SO₂;
R⁴ is hydrogen, optionally substituted linear or branched alkyl, optionally substituted linear or branched alkenyl, or optionally substituted linear or branched alkynyl; and enantiomers, derivatives and pharmaceutically acceptable salts thereof,
and an additional agent.

73. The method of claim 72, wherein the compound of formula (I) is an inhibitor of a p21 activated kinase (PAK) or a cyclin dependent kinase (CDK).

74. The method of claim 73, wherein the p21 activated kinase is selected from the group consisting of PAK4, PAK5, PAK6, and any combinations thereof, or the cyclin dependent kinase is selected from the group consisting of Cdk 1, Cdk 2, Cdk 3, Cdk 4, Cdk 5, Cdk 6, Cdk 7, Cdk 8, Cdk 9, Cdk 11, and any combinations thereof.

75. The method of any of claims 72-74, the additional agent is selected from the group consisting of small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

76. The method of any of claims 72-75, wherein the additional agent modulates a biological pathway or a target, wherein the biological pathway is selected from the group consisting of PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and combinations thereof, and wherein the target is selected from the group consisting of Na⁺/K⁺ channel, MAPK, cannobinoid receptor, GPCR, Ca²⁺ channel, K⁺ channel, PDE5, GSK/CDK, PKR, CDK2, IKK-2, proteasome, BMP/TGFbeta receptor, dopamine receptor, and any combinations thereof.

77. The method of any of claims 72-76, wherein the additional agent inhibits GSK-3.

78. The method of any of claims 72-77, wherein the additional agent is alsterpaullone.

79. The method of any of claims 72-78, wherein the compound of formula (I) and the additional agent are in ratio of 20:1 to 1:20 by weight or by moles.

80. The method of any of claims 72-79, wherein the motor neuron comprises a mutation in gene encoding SMN1.

81. The method of any of claims 72-80, wherein the motor neuron comprises a mutation in gene encoding superoxide mutase 1 (SOD1).

82. The method of any of claims 72-80, wherein the motor neuron comprises a G->A mutation at position 93 of gene encoding SOD1.

83. The method of any of claims 72-82, wherein the contact is in vitro.

84. The method of any of claims 72-82, wherein the contact is in vivo.

85. The method of claim 72-84, wherein in vivo contact is in a mammal.

86. The method of claim 72-84, wherein in vivo contact is in a subject, where the subject is selected for treatment of a neurodegenerative disorder characterized by degeneration of motor neurons.

87. The method of claim 86, wherein the subject is human.

88. The method of claim 86 or 86, wherein the neurodegenerative disorder is characterized by a mutation in the SMN gene.

89. The method of any of claims 86-88, wherein the neurodegenerative disorder is characterized by diminished levels of SMN protein.

90. The method of any of claims 86-89, wherein the neurodegenerative disorder is amyotrophic lateral sclerosis (ALS).

91. The method of any of claims 86-89, wherein the neurodegenerative disorder is spinal muscular atrophy (SMA).

92. The method of any of claims 72-91, wherein the inhibitor of PAK inhibits motor neuron survival when used alone.

93. The method of any of claims 72-92, wherein the additional agent does not increase motor neuron survival when used alone.

94. The method of any of claims 72-93, wherein the compound of formula (I) and the additional agent act synergistically in increasing motor neuron survival.

95. The method of any of claims 72-94, wherein the combination of the compound of formula (I) and the additional agent increases motor neuron survival by at least 5% as compared to increase in motor neuron survival by the compound of formula (I) and additional agent when the compound of formula (I) and the additional agents are used alone.

96. The method of any of claims 72-95, wherein the motor neuron is contacted with the additional agent within 6 hours of contacting with the compound of formula (I).

97. The method of any of claims 72-96, wherein the motor neuron is contacted first with the compound of formula (I).

98. The method of any of claims 72-96, wherein the motor neuron is contacted first with the additional agent.

99. The method of any of claims 72-98, wherein the motor neuron is contacted substantially simultaneously with the compound of formula (I) and the additional agent.

100. The method of any of claims 72-99, wherein the compound of formula (I) is purvalanol A.

101. The method of any of claims 72-100, wherein the compound of formula (I) is purvalanol A and the additional agent is allstepaullone.

102. A synergistic composition for increasing motor neuron survival comprising an a compound of formula (I): 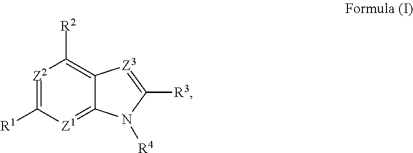
wherein each of Z¹, Z² and Z³ is independently N or CH₂;
R¹ is hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂H, SO₂, CN, CF₃, or SO₂;
R² hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂H, SO₂, CN, CF₃, or SO₂;
R³ is hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂H, SO₂, CN, CF₃, and SO₂;
R⁴ is hydrogen, optionally substituted linear or branched alkyl, optionally substituted linear or branched alkenyl, or optionally substituted linear or branched alkynyl; and enantiomers, derivatives and pharmaceutically acceptable salts thereof, and an additional agent, wherein the additional agent modulates a biological pathway or a target, wherein the biological pathway is selected from the group consisting of PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and combinations thereof, and wherein the target is selected from the group consisting of Na⁺/K⁺ channel, MAPK, cannobinoid receptor, GPCR, Ca²⁺ channel, K⁺ channel, PDE5, GSK/CDK, PKR, CDK2, IKK-2, proteasome, BMP/TGFbeta receptor, dopamine receptor, and any combinations thereof.

103. The synergistic composition of claim 102, wherein the compound of formula (I) is an inhibitor of a p21 activated kinase (PAK) or a cyclin dependent kinase (CDK).

104. The synergistic composition of claim 103, wherein the p21 activated kinase is selected from the group consisting of PAK4, PAK5, PAK6, and any combinations thereof, or the cyclin dependent kinase is selected from the group consisting of Cdk1, Cdk 2, Cdk 3, Cdk 4, Cdk 5, Cdk 6, Cdk 7, Cdk 8, Cdk 9, Cdk 11, and any combinations thereof.

105. The synergistic composition of any of claims 102-104, wherein the additional agent is selected from the group consisting of small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

106. The synergistic composition of any of claims 102-105, wherein the additional agent inhibits GSK-3.

107. The synergistic composition of any of claims 102-106, wherein the additional agent is alsterpaullone.

108. The synergistic composition of any of claims 102-107, wherein the compound of formula (I) and the additional agent are in ratio of 20:1 to 1:20 by weight or by moles.

109. The synergistic composition of any of claims 102-108, wherein the compound of formula (I) is purvalanol A.

110. The synergistic composition of any of claims 102-109, wherein the compound of formula (I) is purvalanol A and the additional agent is allsterpaullone.

111. A pharmaceutical composition comprising a synergistic composition of any of claims 102-109 and a pharmaceutically acceptable carrier.

112. A method for treating a neurodegenerative disorder or disorder associated with neuronal cell death in subject, the method comprising co-administering a compound of formula (I): 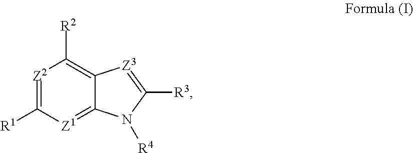
wherein each of Z¹, Z² and Z³ is independently N or CH₂;
R¹ is hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂H, SO₂, CN, CF₃, or SO₂;
R² hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂H, SO₂, CN, CF₃, or SO₂;
R³ is hydrogen, halogen, optionally substituted linear or branched alkyl, optionally substituted linear or branched alkenyl, optionally substituted linear or branched alkynyl, optionally substituted alkoxy, optionally substituted aryl, optionally substituted heteroaryl, optionally substituted cyclyl, optionally substituted heterocyclyl, amino, alkyl amino, CO₂H, SO₂, CN, CF₃, and SO₂;
R⁴ is hydrogen, optionally substituted linear or branched alkyl, optionally substituted linear or branched alkenyl, or optionally substituted linear or branched alkynyl; and enantiomers, derivatives and pharmaceutically acceptable salts thereof, and an additional agent to subject in need thereof, wherein the additional agent modulates a biological pathway or a target, wherein the biological pathway is selected from the group consisting of PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and combinations thereof, and wherein the target is selected from the group consisting of Na⁺/K+ channel, MAPK, cannobinoid receptor, GPCR, Ca²⁺ channel, K+ channel, PDE5, GSK/CDK, PKR, CDK2, IKK-2, proteasome, BMP/TGFbeta receptor, dopamine receptor, and any combinations thereof.

113. The method of claim 112, wherein the compound of wherein the compound of formula (I) is an inhibitor of a p21 activated kinase (PAK) or a cyclin dependent kinase (CDK).

114. The method of claim 113, wherein the p21 activated kinase is selected from the group consisting of PAK4, PAK5, PAK6, and any combinations thereof, or the cyclin dependent kinase is selected from the group consisting of Cdk 1, Cdk 2, Cdk 3, Cdk 4, Cdk 5, Cdk 6, Cdk 7, Cdk 8, Cdk 9, Cdk 11, and any combinations thereof.

115. The method of any of claims 113-114, wherein the additional agent is selected from the group consisting of small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

116. The method of any of claims 112-115, wherein the additional agent modulates a biological pathway or a target, wherein the biological pathway is selected from the group consisting of PI-3K signaling pathway, Akt signaling pathway, MAPK signaling pathway, PDGF pathway, RAS pathway, eIF2 pathway, GSK signaling pathway, PKR pathway, Insulin Receptor Pathway, mTOR pathway, EGF pathway, NGF pathway, FGF pathway, TGF pathway, BMP pathway, receptor tyrosine kinase (RTK) pathway, and combinations thereof, and wherein the target is selected from the group consisting of Na⁺/K⁺ channel, MAPK, cannobinoid receptor, GPCR, Ca²⁺ channel, K⁺ channel, PDE5, GSK/CDK, PKR, CDK2, IKK-2, proteasome, BMP/TGFbeta receptor, dopamine receptor, and any combinations thereof.

117. The method of any of claims 112-116, wherein the additional agent inhibits GSK-3.

118. The method of any of claims 112-117, wherein the additional agent is kenpaullone, or alsterpaullone.

119. The method of any of claims 112-118, wherein the compound of formula (I) and the additional agent are in ratio of 20:1 to 1:20 by weight or by moles.

120. The method of any of claims 112-119, wherein the subject comprises a mutation in gene encoding SMN 1.

121. The method of any of claims 112-120, wherein the subject comprises a mutation in gene encoding superoxide mutase 1 (SOD1).

122. The method of any of claims 112-121, wherein the subject comprises a G->A mutation at position 93 of gene encoding SOD1.

123. The method of any of claims 112-122, wherein the neurodegenerative disorder is characterized by degeneration of motor neurons.

124. The method of any of claims 112-123, wherein the neurodegenerative disorder is characterized by diminished levels of SMN protein.

125. The method of any of claims 112-124, wherein the neurodegenerative disorder is amyotrophic lateral sclerosis (ALS).

126. The method of any of claims 112-124, wherein the neurodegenerative disorder is spinal muscular atrophy (SMA).

127. The method of any of claims 112-126, wherein the compound of formula (I) inhibits motor neuron survival when used alone.

128. The method of any of claims 112-127, wherein the additional agent does not increase motor neuron survival when used alone.

129. The method of any of claims 112-128, wherein the compound of formula (I) and the additional agent act synergistically in increasing motor neuron survival.

130. The method of any of claims 112-129, wherein the combination of compound of formula (I) and the additional agent increases motor neuron survival by at least 5% as compared to increase in motor neuron survival by compound of formula (I) or additional agent when the compound of formula (I) or the additional agents are used alone.

131. The method of any of claims 112-130, wherein the compound of formula (I) and the additional agent are co-administered within 6 hours of each other.

132. The method of any of claims 112-131, wherein the compound of formula (I) and the additional agent are co-administered in the same composition.

133. The method of any of claims 112-132, wherein the compound of formula (I) is administered first.

134. The method of any of claims 112-133, wherein the additional agent is administered first.

135. The method of any of claims 112-134, wherein the compound of formula (I) is purvalanol.

136. The method of any of claims 112-135, wherein the additional agent is allsterpaullone.

137. The method of any of claims 112-136, wherein the subject is administered a composition of any of claims 39-43, a synergistic composition of any of claims 102-110 or a pharmaceutical composition of claim 111.

138. An assay for identifying a compound that increases motor neuron survival, the method comprising:
(i) withdrawing at least one trophic factor from the plated motor neurons, wherein the motor neurons comprise a transgenic reporter gene, and wherein the transgenic reporter gene comprises a fluorescent protein;
(ii) incubating the plated motor neurons with a test agent; and
(iii) counting cells expressing the fluorescent protein cells,
wherein a higher number of fluorescent protein expressing cells relative to a control indicates that the compound increases motor neuron survival.

139. The assay of claim 138, further comprising selecting the compound that increases motor neuron survival.

140. The assay of claim 138 or 139, wherein the motor neuron is an embryonic stem cell-derived motor neuron.

141. The assay of any of claims 138-140, wherein the expression of the fluorescent protein is driven by promoter elements from Hb9 gene.

142. The assay of any of claims 138-141, wherein the fluorescent protein is a green fluorescent protein (GFP).

143. The assay of any of claims 138-142, wherein the transgenic reporter gene expresses a green fluorescent protein, wherein expression of the green fluorescent protein (GFP) is driven by promoter elements from Hb9 gene (Hb9::GFP).

144. The assay of any of claims 138-143, wherein the motor neurons are plated at a density of 1,000 to 20,000 cells/well.

145. The assay of any of claims 138-144, wherein the test agent is incubated for at least three days counting the fluorescent protein expressing cells.

146. The assay of any of claims 138-145, wherein the test agent is incubated at a final concentration of from 0.01 nm to about 10 mM.

147. The assay of any of claims 138-146, wherein the test agent is tested at 2 or more different concentrations.

148. The assay of claim 147, wherein the test agent is tested at 2 more or different concentration in a 100-1000 fold range.

149. The assay of claim 147 or 148, wherein the test agent is tested at 3 concentrations.

150. The assay of any of claims 147-149, wherein the test agent is tested at a final concentration of 0.1 μM, 1 μM, and 10 μM.

151. The assay of any of claims 138-150, wherein the test agent is selected from the group consisting of small organic or inorganic molecules; saccharines; oligosaccharides; polysaccharides; biological macromolecules, e.g., peptides, proteins, and peptide analogs and derivatives; peptidomimetics; nucleic acids and nucleic acid analogs and derivatives (including but not limited to siRNAs, shRNAs, antisense RNAs, a ribozymes, and apatamers); an extract made from biological materials such as bacteria, plants, fungi, or animal cells; animal tissues; naturally occurring or synthetic compositions; and any combinations thereof.

152. The assay of any of claims 138-151, wherein the motor neuron comprises a mutation in gene encoding SMN1.

153. The assay of any of claims 138-152, wherein the motor neuron comprises a mutation in gene encoding superoxide mutase 1 (SOD1).

154. The assay of any of claims 138-153, wherein the motor neuron comprises a G->A mutation at position 93 of gene encoding SOD1.

155. The assay of any of claims 138-154, wherein the number of cells expressing the fluorescent protein is at least 50% higher relative to the control.

156. The assay of any of claims 138-155, wherein the assay comprising plating the motor neurons at a density of about 8,000 cells/well in a 384-well plate; growing the motor neurons for four days; withdrawing the trophic factors; incubating the motor neurons with test agent for three days; and counting the fluorescent protein expressing motor neurons.

157. The assay of any of claims 138-156, wherein the assay is a high throughput screening (HTS) assay.
